(19) United States
(12) Patent Application Publication
(10) Pub. No.: US 2026/0250284 A1
(43) Pub. Date: Aug. 27, 2026

(54) PHENANTHROLINE COMPOUND AND ORGANIC LIGHT-EMITTING DEVICE COMPRISING SAME

(71) Applicant: SFC CO., LTD., Cheongju-si (KR)

(72) Inventors: Sung Hoon JOO, Cheongju-si (KR); Bong-Ki Shin, Cheongju-si (KR); Byung-Sun Yang, Cheongju-si (KR); Ji-Hwan Kim, Cheongju-si (KR); Hyeon Jun Jo, Cheongju-si (KR); Sungeun Choi, Cheongju-si (KR)

(21) Appl. No.: 19/650,005

(22) Filed: Apr. 16, 2026

Related U.S. Application Data
(62) Division of application No. 17/789,697, filed on Mar. 11, 2021.

(30) Foreign Application Priority Data
Mar. 12, 2020 (KR) 10-2020-0030819

(51) Int. Cl. C07D 471/04; H10K 85/60

(52) U.S. Cl. CPC C07D 471/04; H10K 85/6572; H10K 85/6574; H10K 85/6576

(57) ABSTRACT

The present invention relates to a novel phenanthroline-based compound and an organic light-emitting device having high efficiency characteristics comprising same. More particularly, the present invention relates to a phenanthroline-based compound exhibiting high efficiency by including a phenanthroline-based compound having a specific structure as a material for a charge generation layer in an organic light-emitting device, and to an organic light-emitting device comprising same, wherein [Chemical Formula A] is the same as described in the detailed description of the invention.

### CROSS-REFERENCE TO RELATED APPLICATIONS

This application is a Division of U.S. application Ser. No. 17/789,697, filed on Jun. 28, 2022, which in turn claims the benefit of PCT/KR2021/003048, filed on Mar. 11, 2021, and Korean Patent Application No. 10-2020-0030819, filed on Mar. 12, 2020. The entire disclosures of all these applications are hereby incorporated by reference.

### TECHNICAL FIELD

The present disclosure relates to a novel phenanthroline-based compound and an organic light-emitting diode including same and, more specifically, to a phenanthroline-based compound in a specific structure which provides improved properties for an organic light-emitting diode when used as a material for a charge generation layer therein, and an organic light-emitting diode including same.

### BACKGROUND ART

Organic light-emitting diodes (OLEDS), based on self-luminescence, are used to create digital displays with the advantage of having a wide viewing angle and being able to be made thinner and lighter than liquid crystal displays. In addition, an OLED display exhibits a very fast response time. Accordingly, OLEDs find applications in the full color display field or the illumination field.

In general, the term “organic light-emitting phenomenon” refers to a phenomenon in which electrical energy is converted to light energy by means of an organic material. An organic light-emitting diode using the organic light-emitting phenomenon has a structure usually including an anode, a cathode, and an organic material layer interposed therebetween.

In this regard, the organic material layer may have, for the most part, a multilayer structure consisting of different materials, for example, a hole injection layer, a hole transport layer, a light-emitting layer, an electron transport layer, and an electron injection layer in order to enhance the efficiency and stability of the organic light-emitting diode. In the organic light-emitting diode having such a structure, application of a voltage between the two electrodes injects a hole from the anode and an electron from the cathode to the organic layer. In the luminescent zone, the hole and the electron recombine to produce an exciton. When the exciton returns to the ground state from the excited state, the molecule of the organic layer emits light. Such an organic light-emitting diode is known to have characteristics such as self-luminescence, high luminance, high efficiency, low driving voltage, a wide viewing angle, high contrast, and high-speed response.

Materials used as organic layers in OLEDs may be divided according to functions into luminescent materials and charge transport materials, for example, a hole injection material, a hole transport material, an electron transport material, and an electron injection material and, as needed, further into an electron-blocking material or a hole-blocking material.

OLEDs that emit white light find applications in various fields including illuminations, thin light sources, backlight units of liquid crystal displays, full-color displays with color filters, etc.

In such white OLEDs, properties of high efficiency, long lifespan, color purity, color stability against changes in current and voltage, and ease of device fabrication are important considerations. A structural criterion divides white OLEDs into a single-layered emission structure and a multi-layered emission structure. For a white OLED with a long lifespan, a tandem structure in which a fluorescent blue emitting layer and a phosphorescent yellow emitting layer are stacked is mostly adopted.

For example, given a tandem structure in which a first emission part including a fluorescent blue emitting layer and a second emission part including a phosphorescent yellow-green emitting layer are vertically stacked, an OLED can provide white light as the light from the fluorescent blue emitting layer and the light from the phosphorescent green-yellow emitting layer are mixed.

OLEDs in such a tandem structure are typically provided with a charge generation layer between the first emission part and the second emission part to increase current efficiency and improve charge distribution wherein the current and charge are generated from the light-emitting layer. The charge generation layer may have a P-N junction structure of an N-type charge generation layer and a P-type charge generation layer.

With regard to related arts of charge generation layers in OLEDs, reference may be made to Korean Patent No. 10-2018-0003220 A (issued Jan. 9, 2018), which discloses the use of an organic compound in a charge generation layer wherein the organic compound includes a phenanthroline core combinable with an alkali metal or alkaline earth metal and has an excellent electron transport property and to Korean Patent No. 10-2018-0009856 (issued Jan. 30, 2018), which discloses an OLED in which a charge generation layer includes a metal compound and generates and supplies charges to light-emitting units, thereby improving the driving efficiency.

In spite of extensive attempts of various types of method for fabricating OLEDs in related art including the above literatures, there is a need for development of a charge generating layer material to provide an OLED exhibiting improved light-emitting efficiency.

### DISCLOSURE

### Technical Problem

Therefore, an aspect of the present disclosure is to provide a novel structure compound available as a material for a charge generation layer in an organic light-emitting diode, and a novel organic light-emitting diode (OLED) that includes same as an N-type charge generation layer material and as such, exhibits high efficiency and long lifespan properties.

### Technical Solution

The present disclosure provides a phenanthroline-based compound represented by the following Chemical Formula A:

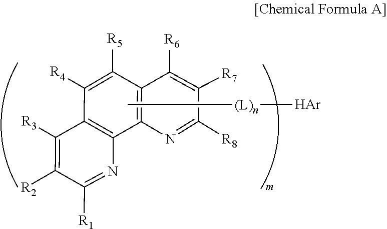

wherein,
L functions as a linker and is at least one selected from a single bond, a substituted or unsubstituted arylene of 6 to 50 carbon atoms, and a substituted or unsubstituted heteroarylene of 2 to 50 carbon atoms,
R₁ to R₈, which are same or different from each other, are each independently at least one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted alkenyl of 2 to 30 carbon atoms, a substituted or unsubstituted alkynyl of 2 to 20 carbon atoms, a substituted or unsubstituted cycloalkyl of 3 to 30 carbon atoms, a substituted or unsubstituted cycloalkenyl of 5 to 30 carbon atoms, a substituted or unsubstituted heteroaryl of 2 to 50 carbon atoms, a substituted or unsubstituted heterocycloalkyl of 2 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 6 to 30 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, a substituted or unsubstituted arylsilyl of 6 to 30 carbon atoms, a cyano, and a halogen,
wherein any one of R₅ to R₈ is a single bond connected to the linker L,
n is an integer of 1 to 3 wherein when n is 2 or greater, the L's are same or different,
m is an integer of 1 or 2,
wherein when m is 2, the corresponding

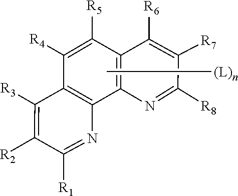

moieties are same or different,
HAr is any one substituent represented by the following Structural Formulas 1 to 5:

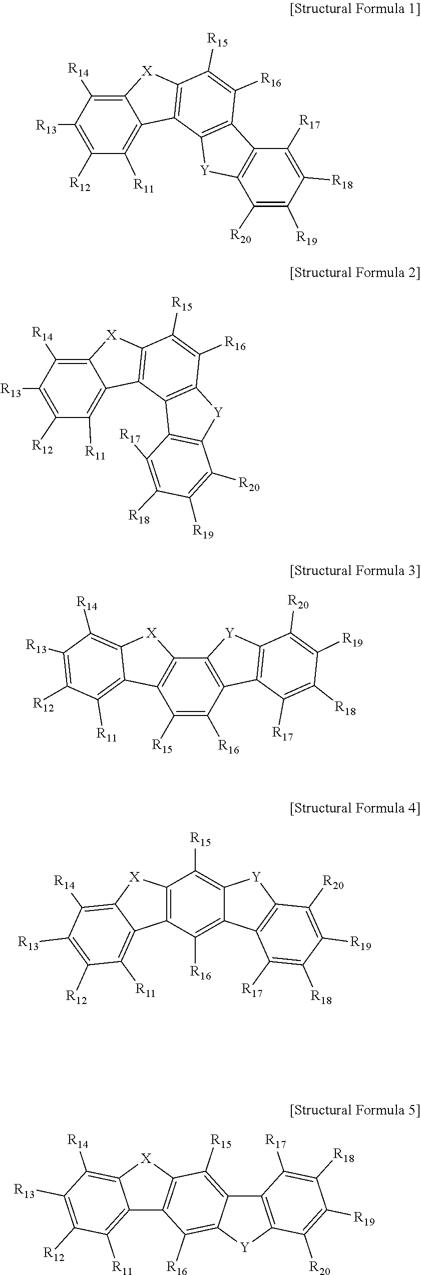

wherein,
R₁₁ to R₂₀, which ae same or different, are each independently any one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted alkenyl of 2 to 30 carbon atoms, a substituted or unsubstituted alkynyl of 2 to 20 carbon atoms, a substituted or unsubstituted cycloalkyl of 3 to 30 carbon atoms, a substituted or unsubstituted cycloalkenyl of 5 to 30 carbon atoms, a substituted or unsubstituted heteroaryl of 2 to 50 carbon atoms, a substituted or unsubstituted heterocycloalkyl of 2 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 6 to 30 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, a substituted or unsubstituted arylsilyl of 6 to 30 carbon atoms, a cyano, and a halogen,
wherein when m is 1, one of R₁₁ to R₂₀ is a single bond connected to the linker L,
wherein when m is 2, two of R₁₁ to R₂₀ are each a single bond connected to the linker L,
X and Y, which are same or different, are each independently at least one selected from CR₂₁R₂₂, NR₂₃, O, S, Se, and Te,
wherein R₂₁ to R₂₃, which are same or different, are each independently at least one selected from a hydrogen atom, a deuterium atom, a substituted or unsubstituted alkyl of 1 to 30 carbon atoms, a substituted or unsubstituted cycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 5 to 30 carbon atoms, a cyano, and a halogen,
R₂₁ and R₂₂ may be linked to each other to form a mono- or polycyclic aliphatic or aromatic ring,
wherein the term “substituted” in the expression “substituted or unsubstituted” used for the compound of Chemical Formula A means having at least one substituent selected from the group consisting of a deuterium atom, a cyano, a halogen, a hydroxy, a nitro, an alkyl of 1 to 24 carbon atoms, a halogenated alkyl of 1 to 24 carbon atoms, cycloalkyl of 3 to 30 carbon atoms, an alkenyl of 2 to 24 carbon atoms, an alkynyl of 2 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, a diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms.

### Advantageous Effects

When used as a material for an N-type charge generation layer in an organic light-emitting diode with a tandem structure including a charge generation layer, the phenanthroline-based compound according to the present disclosure provides improved longevity and high emission efficiency for the organic light-emitting diode (OLED).

In particular, high emission efficiency and longevity can be brought about in the organic light-emitting diode including a P-type charge generation layer and a N-type charge generation layer wherein the phenanthroline-based compound according to the present disclosure is used in the N-type charge generation layer.

### BRIEF DESCRIPTION OF THE DRAWINGS

FIG. 1 is a schematic diagram of an exemplary organic light-emitting diode including one charge generation layer according to some embodiments of the present disclosure.

FIG. 2 is a schematic diagram of an exemplary organic light-emitting diode including two charge generation layers according to some embodiments of the present disclosure.

### MODE FOR CARRYING OUT THE INVENTION

Below, a detailed description will be given of the present disclosure. In each drawing of the present disclosure, sizes or scales of components may be enlarged or reduced from their actual sizes or scales for better illustration, and known components may not be depicted therein to clearly show features of the present disclosure. Therefore, the present disclosure is not limited to the drawings. When describing the principle of the embodiments of the present disclosure in detail, details of well-known functions and features may be omitted to avoid unnecessarily obscuring the presented embodiments.

In the drawing, for convenience of description, sizes of components may be exaggerated for clarity. For example, since sizes and thicknesses of components in drawings are arbitrarily shown for convenience of description, the sizes and thicknesses are not limited thereto. Furthermore, throughout the description, the terms “on” and “over” are used to refer to the relative positioning, and mean not only that one component or layer is directly disposed on another component or layer but also that one component or layer is indirectly disposed on another component or layer with a further component or layer being interposed therebetween. Also, spatially relative terms, such as “below”, “beneath”, “lower”, and “between” may be used herein for ease of description to refer to the relative positioning.

The present disclosure provides a phenanthroline-based compound represented by the following Chemical Formula A:

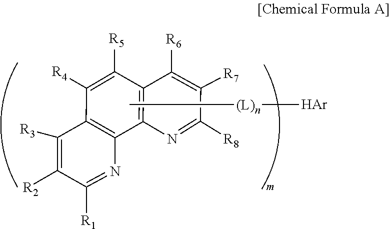

wherein,
L functions as a linker and is at least one selected from a single bond, a substituted or unsubstituted arylene of 6 to 50 carbon atoms, and a substituted or unsubstituted heteroarylene of 2 to 50 carbon atoms,
R₁ to R₈, which are same or different from each other, are each independently at least one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted alkenyl of 2 to 30 carbon atoms, a substituted or unsubstituted alkynyl of 2 to 20 carbon atoms, a substituted or unsubstituted cycloalkyl of 3 to 30 carbon atoms, a substituted or unsubstituted cycloalkenyl of 5 to 30 carbon atoms, a substituted or unsubstituted heteroaryl of 2 to 50 carbon atoms, a substituted or unsubstituted heterocycloalkyl of 2 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 6 to 30 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, a substituted or unsubstituted arylsilyl of 6 to 30 carbon atoms, a cyano, and a halogen,
wherein any one of R₅ to R₈ is a single bond connected to the linker L,
n is an integer of 1 to 3 wherein when n is 2 or greater, the L's are same or different,
m is an integer of 1 or 2,
wherein when m is 2, the corresponding

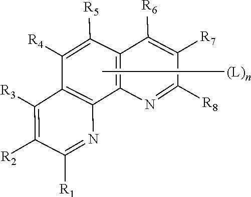

moieties are same or different,
HAr is any one substituent represented by the following Structural Formulas 1 to 5:

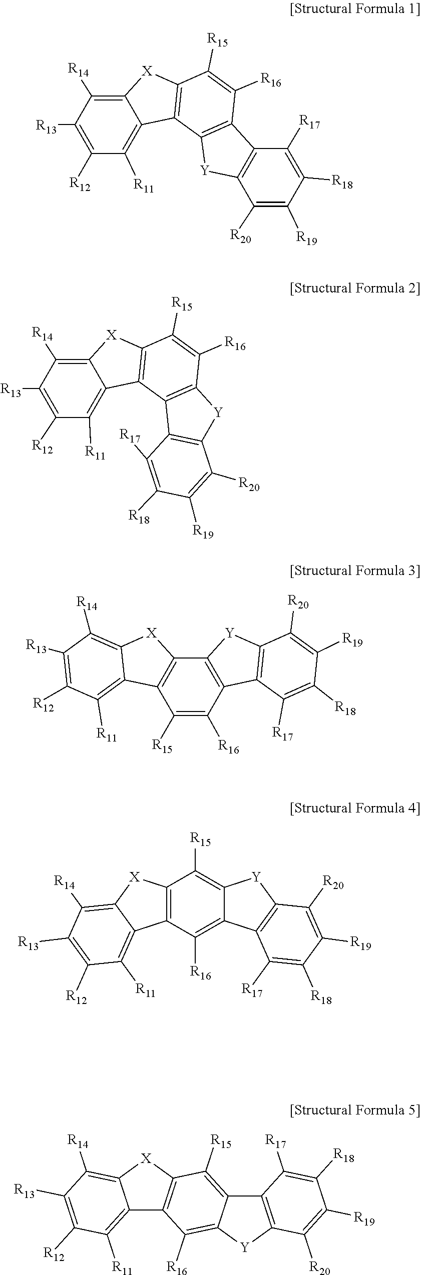

wherein,
R₁₁ to R₂₀, which ae same or different, are each independently any one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted alkenyl of 2 to 30 carbon atoms, a substituted or unsubstituted alkynyl of 2 to 20 carbon atoms, a substituted or unsubstituted cycloalkyl of 3 to 30 carbon atoms, a substituted or unsubstituted cycloalkenyl of 5 to 30 carbon atoms, a substituted or unsubstituted heteroaryl of 2 to 50 carbon atoms, a substituted or unsubstituted heterocycloalkyl of 2 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 6 to 30 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, a substituted or unsubstituted arylsilyl of 6 to 30 carbon atoms, a cyano, and a halogen,
wherein when m is 1, one of R₁₁ to R₂₀ is a single bond connected to the linker L,
wherein when m is 2, two of R₁₁ to R₂₀ are each a single bond connected to the linker L,
X and Y, which are same or different, are each independently at least one selected from CR₂₁R₂₂, NR₂₃, O, S, Se, and Te,
wherein R₂₁ to R₂₃, which are same or different, are each independently at least one selected from a hydrogen atom, a deuterium atom, a substituted or unsubstituted alkyl of 1 to 30 carbon atoms, a substituted or unsubstituted cycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 5 to 30 carbon atoms, a cyano, and a halogen,
R₂₁ and R₂₂ may be linked to each other to form a mono- or polycyclic aliphatic or aromatic ring,
wherein the term “substituted” in the expression “substituted or unsubstituted” used for the compound of Chemical Formula A means having at least one substituent selected from the group consisting of a deuterium atom, a cyano, a halogen, a hydroxy, a nitro, an alkyl of 1 to 24 carbon atoms, a halogenated alkyl of 1 to 24 carbon atoms, cycloalkyl of 3 to 30 carbon atoms, an alkenyl of 2 to 24 carbon atoms, an alkynyl of 2 to 24 carbon atoms, a heteroalkyl of 1 to 24 carbon atoms, an aryl of 6 to 24 carbon atoms, an arylalkyl of 7 to 24 carbon atoms, an alkylaryl of 7 to 24 carbon atoms, a heteroaryl of 2 to 24 carbon atoms, a heteroarylalkyl of 2 to 24 carbon atoms, an alkoxy of 1 to 24 carbon atoms, an alkylamino of 1 to 24 carbon atoms, a diarylamino of 12 to 24 carbon atoms, a diheteroarylamino of 2 to 24 carbon atoms, an aryl(heteroaryl)amino of 7 to 24 carbon atoms, an alkylsilyl of 1 to 24 carbon atoms, an arylsilyl of 6 to 24 carbon atoms, an aryloxy of 6 to 24 carbon atoms, and an arylthionyl of 6 to 24 carbon atoms.

The expression indicating the number of carbon atoms, such as “a substituted or unsubstituted alkyl of 1 to 24 carbon atoms”, “a substituted or unsubstituted aryl of 6 to 50 carbon atoms”, etc. means the total number of carbon atoms of, for example, the alkyl or aryl radical or moiety alone, exclusive of the number of carbon atoms of substituents attached thereto. For instance, a phenyl group with a butyl at the para position falls within the scope of an aryl of 6 carbon atoms, even though it is substituted with a butyl radical of 4 carbon atoms.

As used herein, the term “aryl” means an organic radical derived from an aromatic hydrocarbon by removing one hydrogen that is bonded to the aromatic hydrocarbon. It may be a single or a fused aromatic system, and when it comes to the latter, the aromatic system may include a fused ring that is formed by adjacent substituents on the aryl radical.

Examples of the aryl include phenyl, o-biphenyl, m-biphenyl, p-biphenyl, o-terphenyl, m-terphenyl, p-terphenyl, naphthyl, anthryl, phenanthryl, pyrenyl, indenyl, fluorenyl, tetrahydronaphthyl, perylenyl, chrysenyl, naphthacenyl, and fluoranthenyl, but are not limited thereto. At least one hydrogen atom of the aryl may be substituted by a deuterium atom, a halogen atom, a hydroxy, a nitro, a cyano, a silyl, an amino (—NH₂, —NH(R), —N(R′)(R″) wherein R′ and R″ are each independently an alkyl of 1 to 10 carbon atoms, in this case, called “alkylamino”), an amidino, a hydrazine, a hydrazone, a carboxyl, a sulfonic acid, a phosphoric acid, an alkyl of 1 to 24 carbon atoms, a halogenated alkyl of 1 to 24 carbon atoms, an alkenyl of 2 to 24 carbon atoms, an alkynyl of 2 to 24 carbon atoms, a heteroalkyl of 1 to 24 carbon atoms, an aryl of 6 to 24 carbon atoms, an arylalkyl of 6 to 24 carbon atoms, a heteroaryl of 2 to 24 carbon atoms, or a heteroarylalkyl of 2 to 24 carbon atoms.

The term “heteroaryl” substituent used in the compounds of the present disclosure refers to a hetero aromatic radical of 2 to 50 carbon atoms and particularly 2 to 24 carbon atoms bearing 1, 2, or 3 heteroatoms selected from among N, O, P, Si, S, Ge, Se, and Te. In the aromatic radical, two or more rings may be fused. One or more hydrogen atoms on the heteroaryl may be substituted by the same substituents as on the aryl.

In addition, the term “heteroaromatic ring”, as used herein, refers to an aromatic hydrocarbon ring bearing as a ring member at least one heteroatom and particularly 1 to 3 heteroatoms selected from among N, O, P, Si, S, Ge, Se, and Te.

As used herein, the term “alkyl” refers to an alkane missing one hydrogen atom and includes linear or branched structures. Examples of the alkyl substituent useful in the present disclosure include methyl, ethyl, propyl, isopropyl, isobutyl, sec-butyl, tert-butyl, pentyl, iso-amyl, hexyl, and the like. At least one hydrogen atom of the alkyl may be substituted by the same substituent as in the aryl.

The term “cyclo” as used in substituents of the compounds of the present disclosure, such as cycloalkyl, etc., refers to a structure responsible for a mono- or polycyclic ring of saturated hydrocarbons. Concrete examples of cycloalkyl radicals include cyclopropyl, cyclobutyl, cyclopentyl, cyclohexyl, methylcyclopentyl, methylcyclohexyl, ethylcyclopentyl, ethylcyclohexyl, adamantyl, dicyclopentadienyl, decahydronaphthyl, norbornyl, bornyl, isobornyl, and so on. One or more hydrogen atoms on the cycloalkyl may be substituted by the same substituents as on the aryl.

The term “alkoxy” as used in the compounds of the present disclosure refers to an alkyl or cycloalkyl singularly bonded to oxygen. Concrete examples of the alkoxy include methoxy, ethoxy, propoxy, isobutoxy, sec-butoxy, pentoxy, iso-amyloxy, hexyloxy, cyclobutyloxy, cyclopentyloxy, adamantyloxy, dicyclopentyloxy, bornyloxy, isobornyloxy, and the like. One or more hydrogen atoms on the alkoxy may be substituted by the same substituents as on the aryl.

Concrete examples of the arylalkyl used in the compounds of the present disclosure include phenylmethyl (benzyl), phenylethyl, phenylpropyl, naphthylmethyl, naphthylethyl, and the like. One or more hydrogen atoms on the arylalkyl may be substituted by the same substituents as on the aryl.

Concrete examples of the silyl radicals used in the compounds of the present disclosure include trimethylsilyl, triethylsilyl, triphenylsilyl, trimethoxysilyl, dimethoxyphenylsilyl, diphenylmethylsilyl, diphenylvinlysilyl, methylcyclobutylsilyl, and dimethyl furylsilyl. One or more hydrogen atoms on the silyl may be substituted by the same substituents as on the aryl.

As used herein, the term “alkenyl” refers to an unsaturated hydrocarbon group that contains a carbon-carbon double bond between two carbon atoms and the term “alkynyl” refers to an unsaturated hydrocarbon group that contains a carbon-carbon triple bond between two carbon atoms.

As used herein, the term “alkylene” refers to an organic aliphatic radical regarded as derived from a linear or branched saturated hydrocarbon alkane by removal of two hydrogen atoms from different carbon atoms. Concrete examples of the alkylene include methylene, ethylene, propylene, isopropylene, isobutylene, sec-butylene, tert-butylene, pentylene, iso-amylene, hexylene, and so on. One or more hydrogen atoms on the alkylene may be substituted by the same substituents as on the aryl.

As used herein, the term “diarylamino” refers to an amine group having aforementioned, two identical or different aryl radicals bonded to the nitrogen atom thereof, the term “diheteroarylamino” to an amine group having two identical or different heteroaryl radicals bonded to the nitrogen atom thereof, and the term “aryl(heteroaryl)amino” to an amine group having the aryl radical and the heteroaryl radical each bonded to the nitrogen atom thereof.

As more particular examples accounting for the term “substituted” in the expression “substituted or unsubstituted” used for compounds of Chemical Formula A the compounds may be substituted by at least one substituent selected from the group consisting of a deuterium atom, a cyano, a halogen, a hydroxy, a nitro, an alkyl of 1 to 12 carbon atoms, a halogenated alkyl of 1 to 12 carbon atoms, an alkenyl of 2 to 12 carbon atoms, an alkynyl of 2 to 12 carbon atoms, a cycloalkyl of 3 to 12 carbon atoms, a heteroalkyl of 1 to 12 carbon atoms, an aryl of 6 to 18 carbon atoms, an arylalkyl of 7 to 20 carbon atoms, an alkylaryl of 7 to 20 carbon atoms, a heteroaryl of 2 to 18 carbon atoms, a heteroarylalkyl of 2 to 18 carbon atoms, an alkoxy of 1 to 12 carbon atoms, an alkylamino of 1 to 12 carbon atoms, a diarylamino of 12 to 18 carbon atoms, a diheteroarylamino of 2 to 18 carbon atoms, an aryl(heteroaryl)amino of 7 to 18 carbon atoms, an alkylsilyl of 1 to 12 carbon atoms, an arylsilyl of 6 to 18 carbon atoms, an aryloxy of 6 to 18 carbon atoms, and an arylthionyl of 6 to 18 carbon atoms.

The phenanthroline-based compound, represented by Chemical Formula A, according to the present disclosure is structurally characterized by the central polycondensed ring moiety of “6-membered aromatic ring-X-bearing pentagonal ring-6-membered aromatic ring-Y-bearing pentagonal ring-6-membered aromatic ring”, selected from the following Structural Formulas 1 to 5:

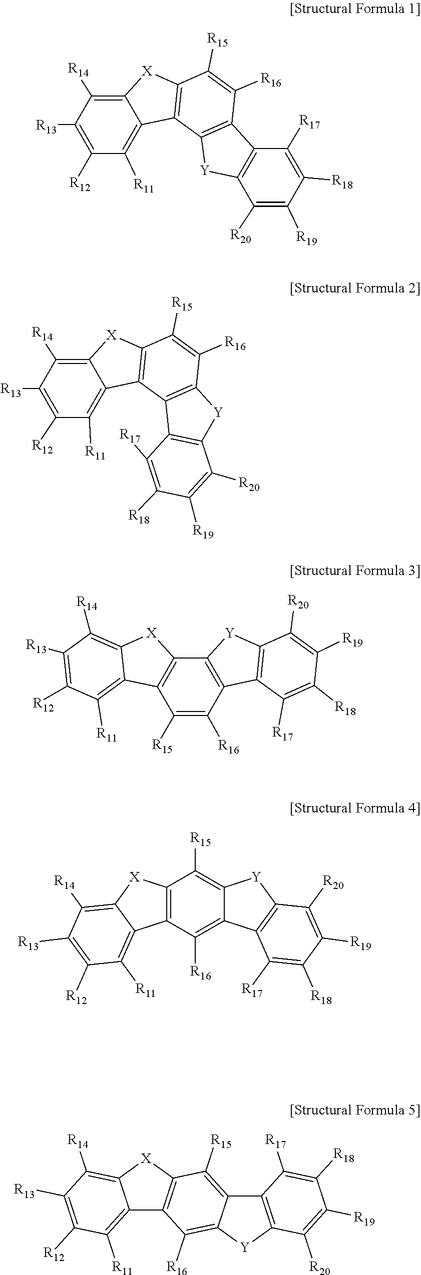

wherein one or two aromatic carbon atoms of the three 6-membered aromatic rings in the polycondensed ring moiety s bonded to corresponding one or two aromatic carbon atoms of the 1,10-phenanthroline structured monovalent radical possessing R₁ to R₈ (hereinafter referred as to “substituted or unsubstituted phenanthroline radical”) or to the linker L connected to the substituted or unsubstituted phenanthroline radical, whereby the organic light-emitting diode can exhibit high emission efficiency and long lifespan properties compared to conventional organic light-emitting diodes.

In an embodiment, R₈ in Chemical Formula A may be a single bond linked to the linker L.

In Chemical Formula A according to an embodiment, n may be 1 or m may be 1, and n and m may each be preferably 1.

When n and m in Chemical Formula A are each 1, the phenanthroline-based compound represented by Chemical Formula A has a polycondensed ring moiety represented by one of Structural Formulas 1 to 5 in which one aromatic carbon atom of the 6-membered aromatic rings is bonded to the linker L which is bonded to the substituted or unsubstituted phenantroline radical.

In an embodiment, the linker L in Chemical Formula A may be selected from a single bond, a substituted or unsubstituted arylene of 6 to 18 carbon atoms, and a substituted or unsubstituted heteroarylene of 2 to 18 carbon atoms.

In an embodiment, the linker L in Chemical Formula A may be a single bone, or a substituted or unsubstituted arylene of 6 to 18 carbon atoms or heteroarylene of 2 to 18 carbon atoms represented by the following Structural Formulas 22 to 34:

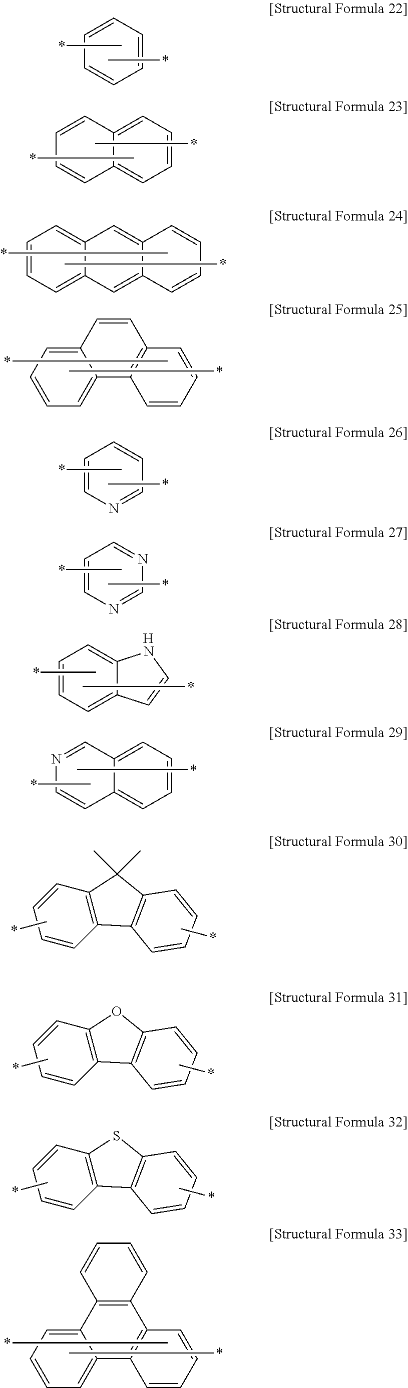

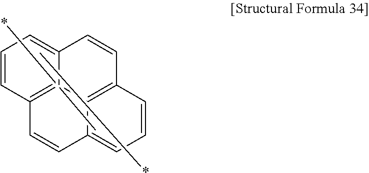

In the linker, hydrogen or deuterium may be positioned on each carbon atom of the aromatic ring.

In an embodiment, R21 to R23 for X and Y in Structural Formulas 1 to 5 are same or different and may each be independently selected from a substituted or unsubstituted alkyl of 1 to 18 carbon atoms, a substituted or unsubstituted cycloalkyl of 3 to 18 carbon atoms, and a substituted or unsubstituted aryl of 6 to 18 carbon atoms.

In an embodiment, at least one of X and Y in Structural Formulas 1 to 5 in Chemical Formula A may be CR21R22.

In an embodiment, R16 in Structural Formulas 1 and 2 in Chemical Formula A may be a single bond connected to the linker L.

Concrete Examples of the phenanthroline-based compound represented by Chemical Formula A according to the present disclosure include <Compound 1> to <Compound 132>, below:

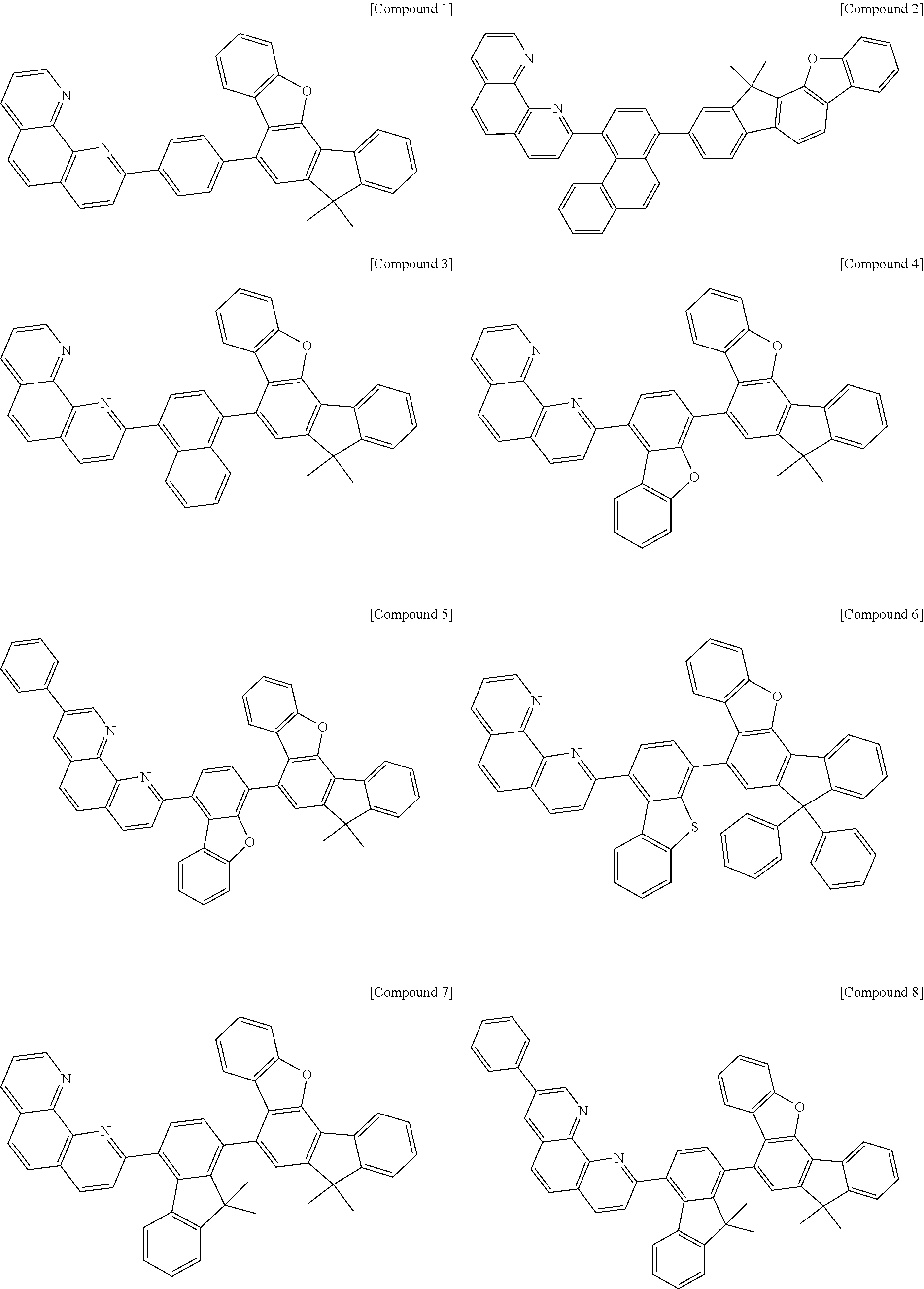

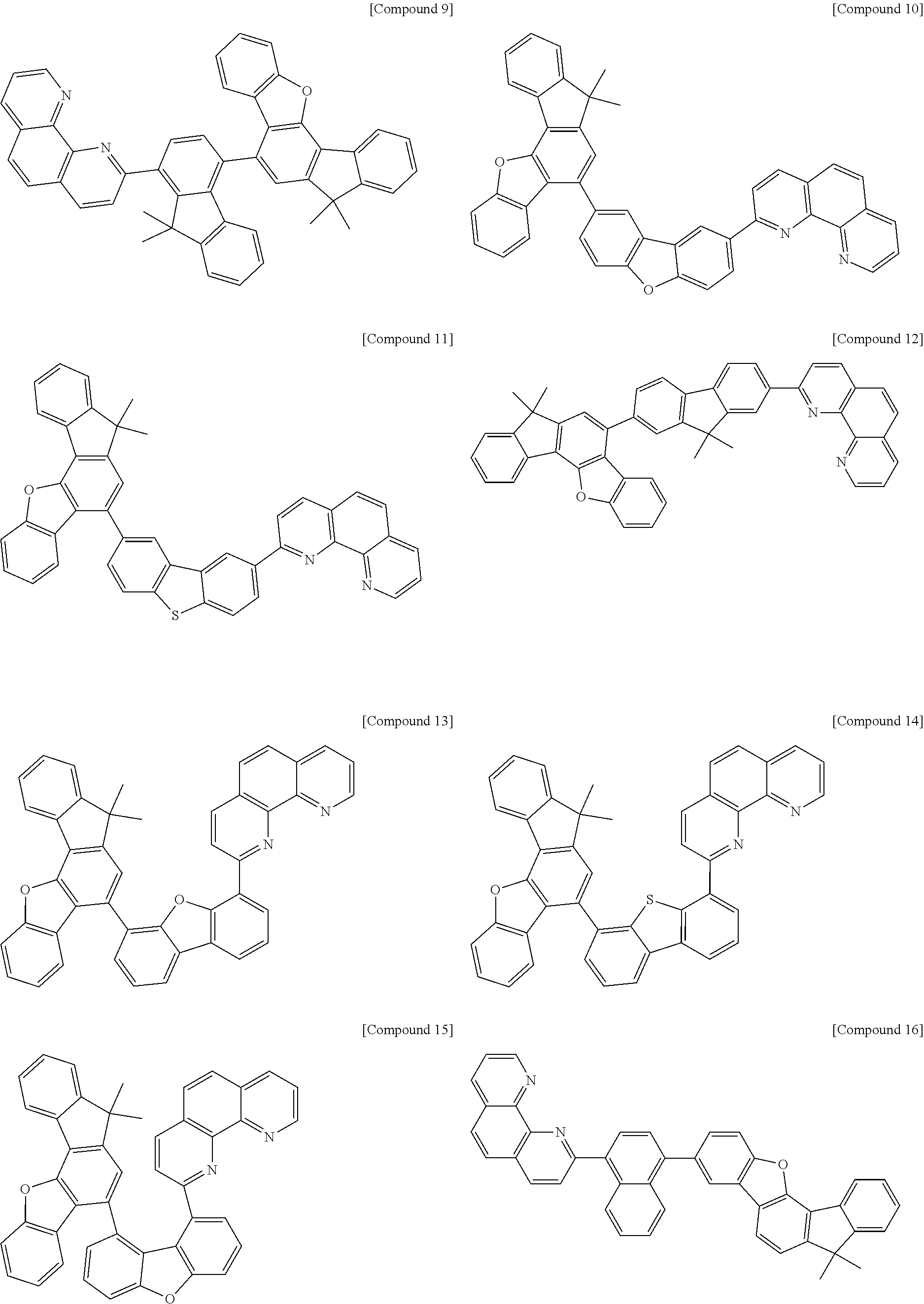

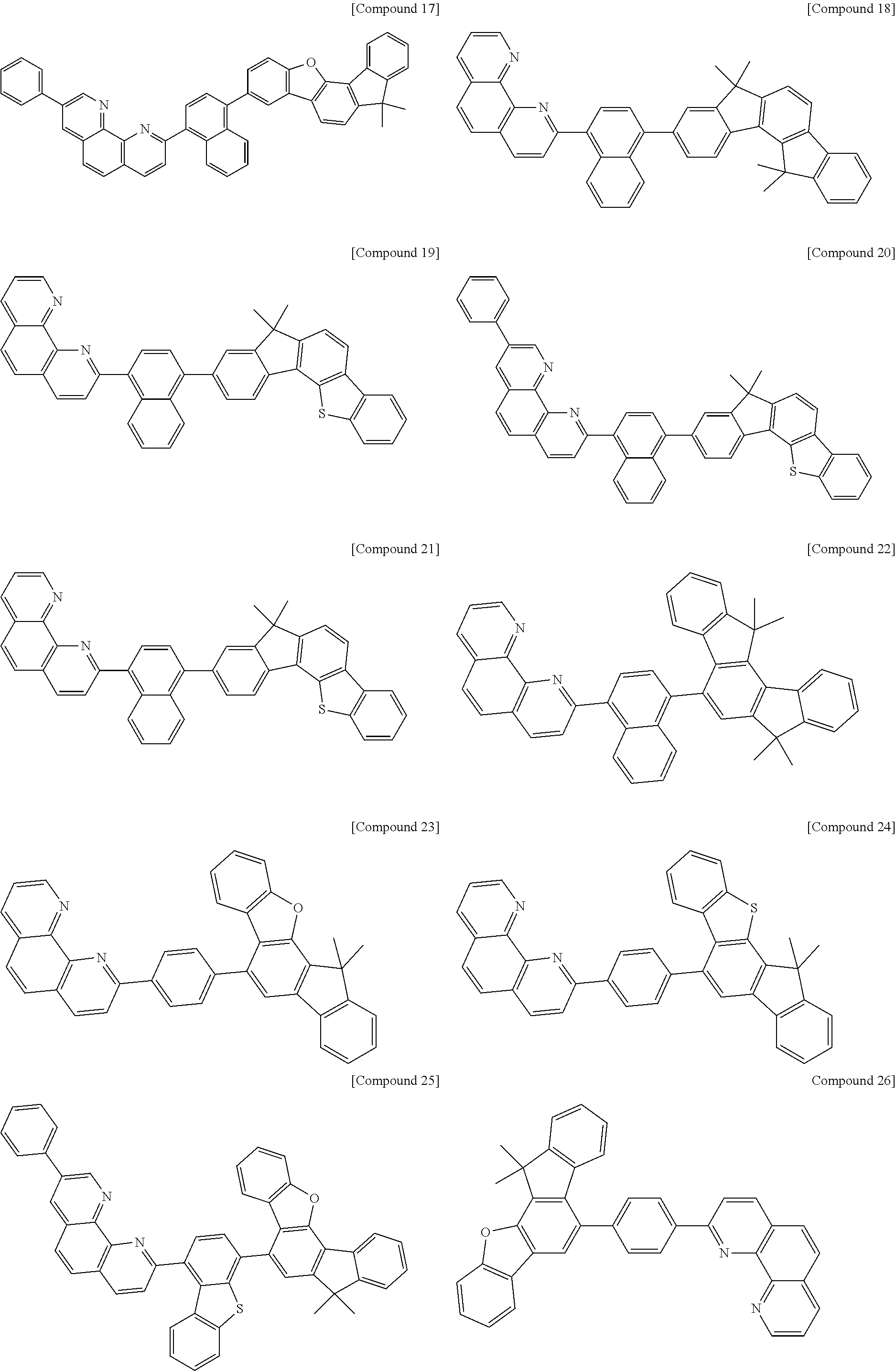

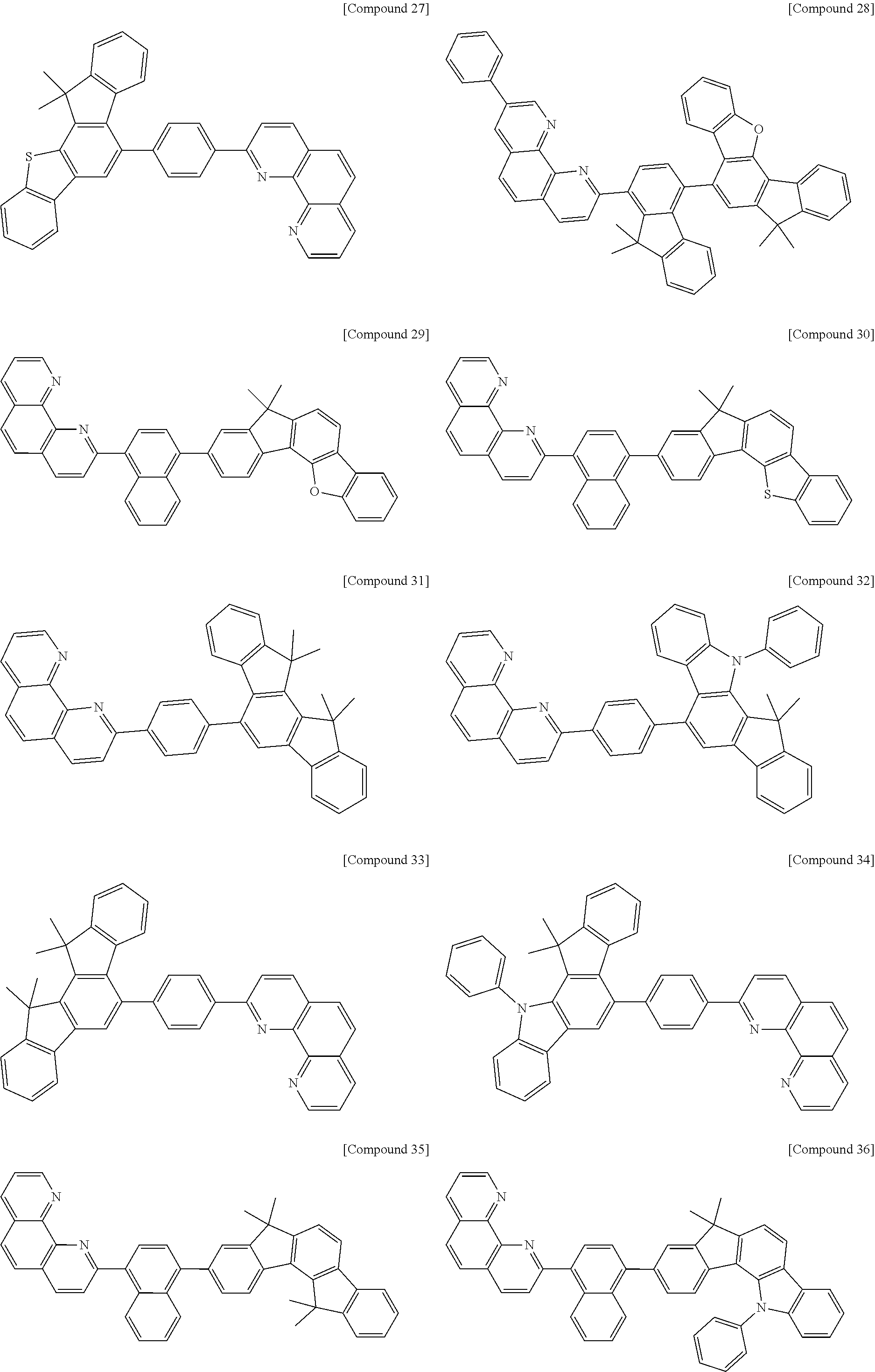

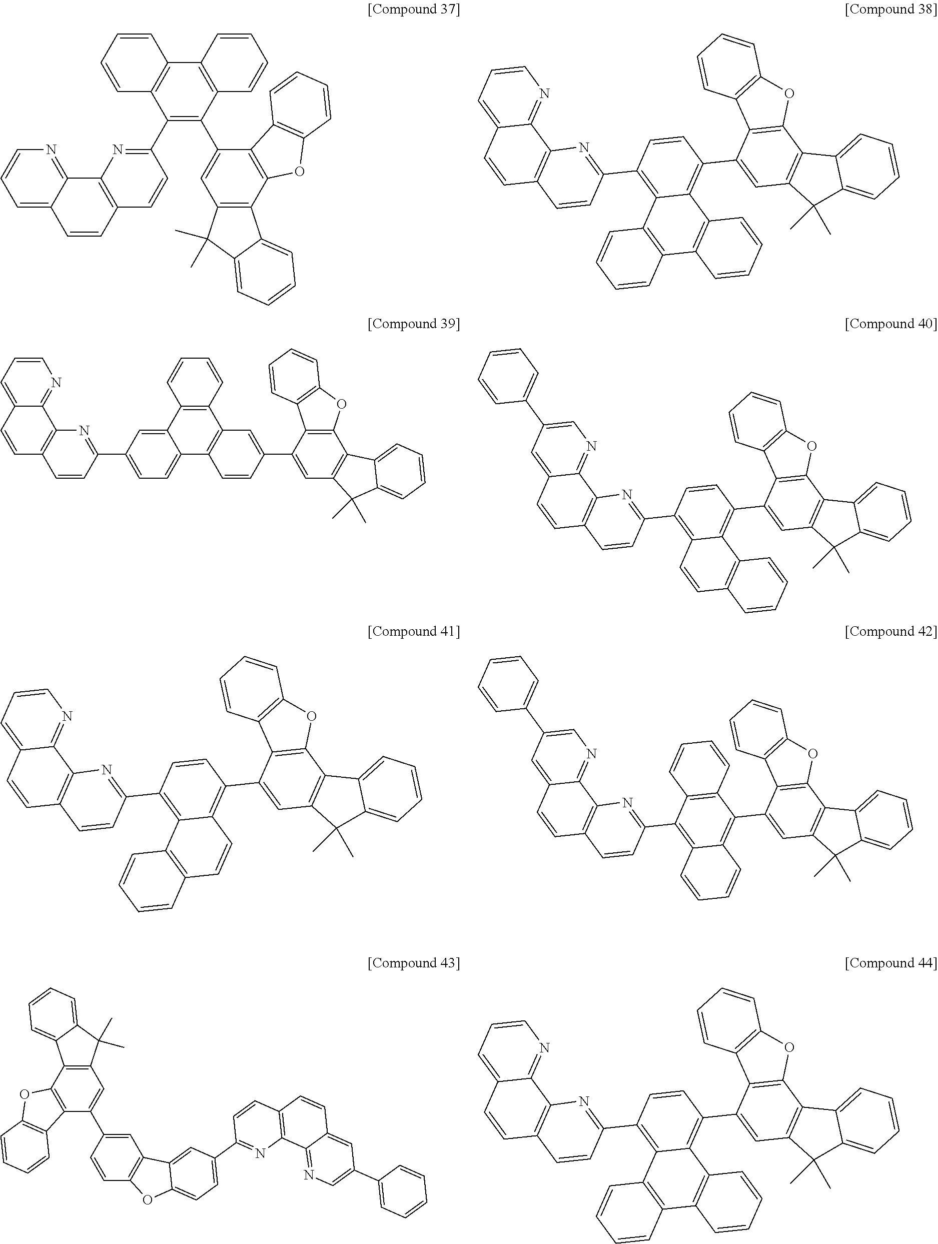

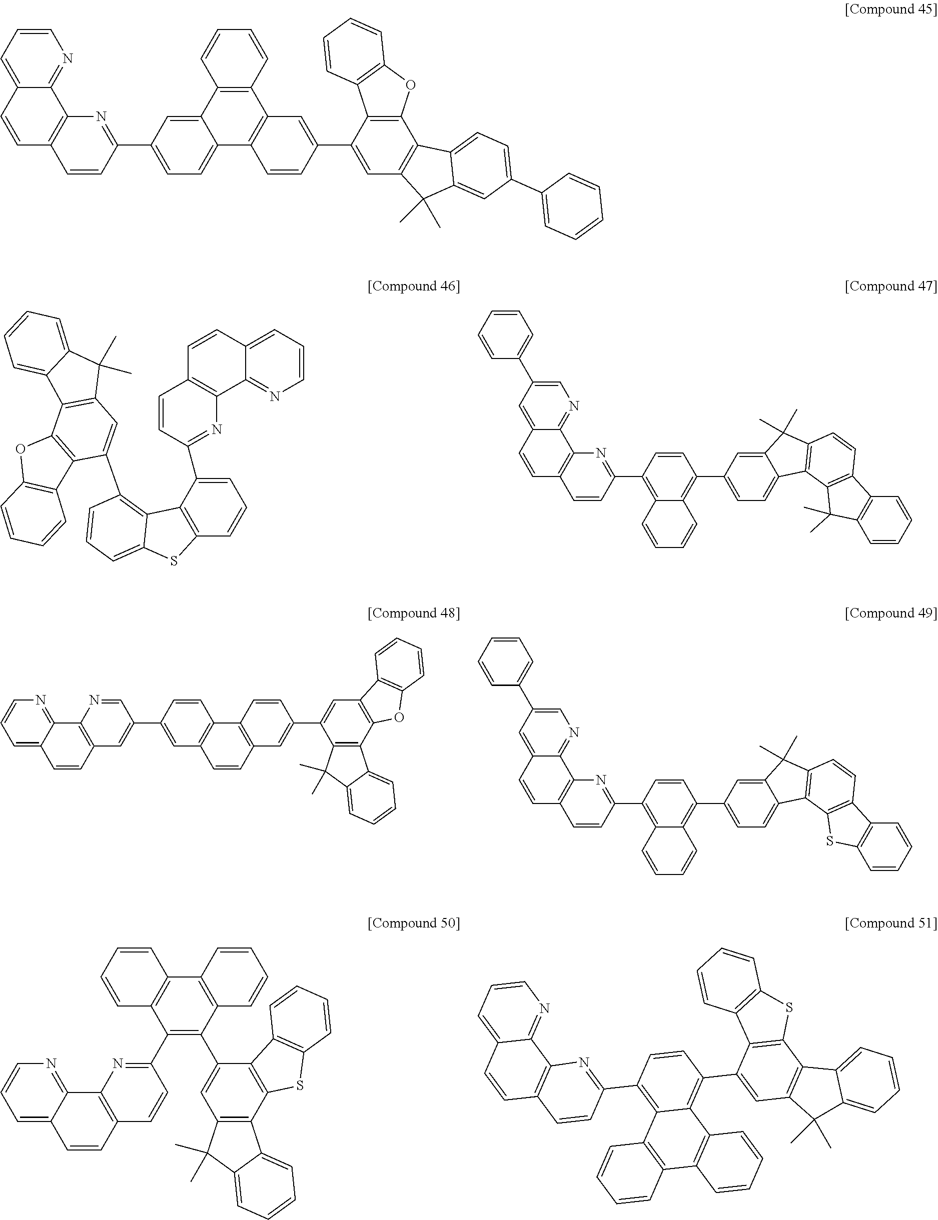

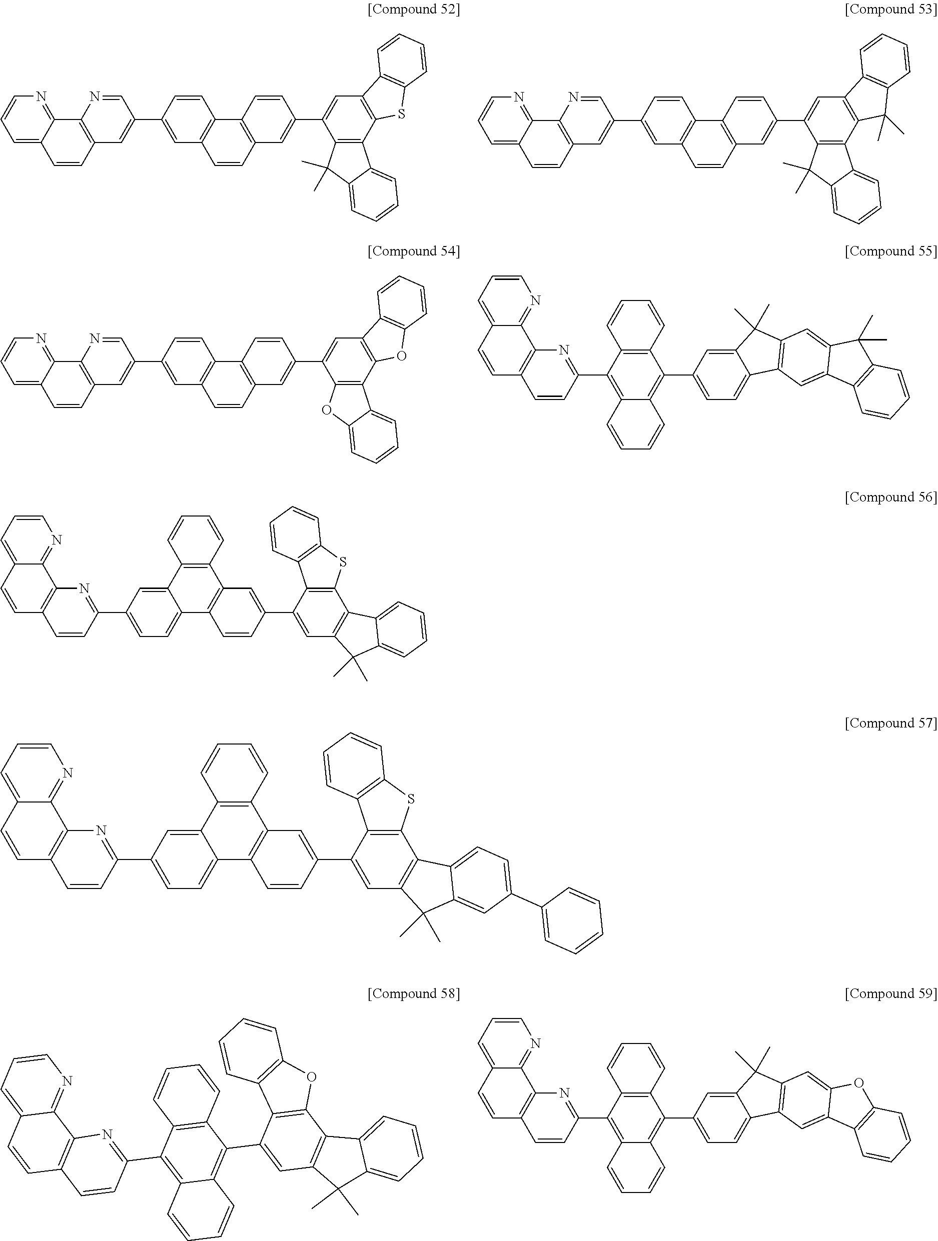

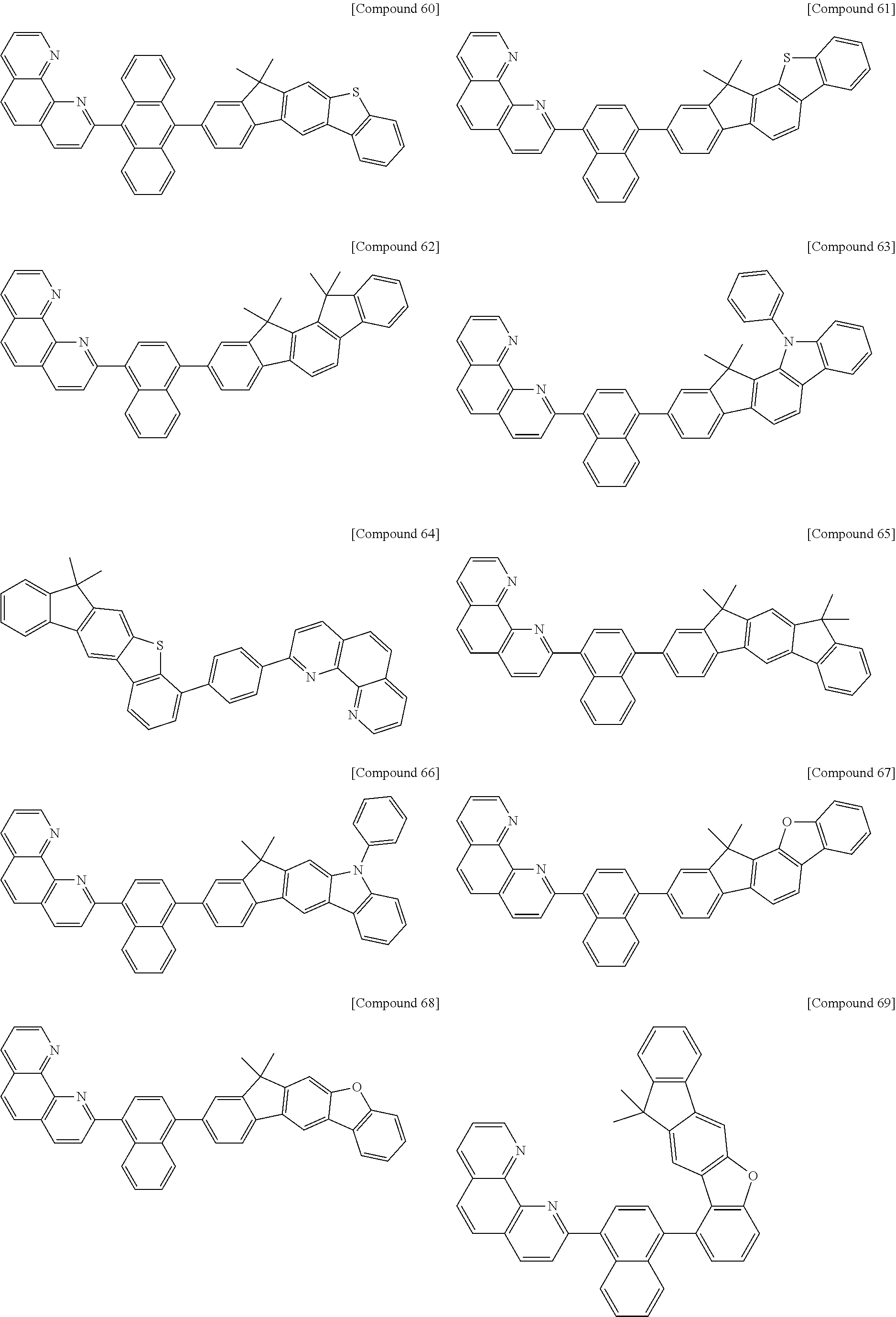

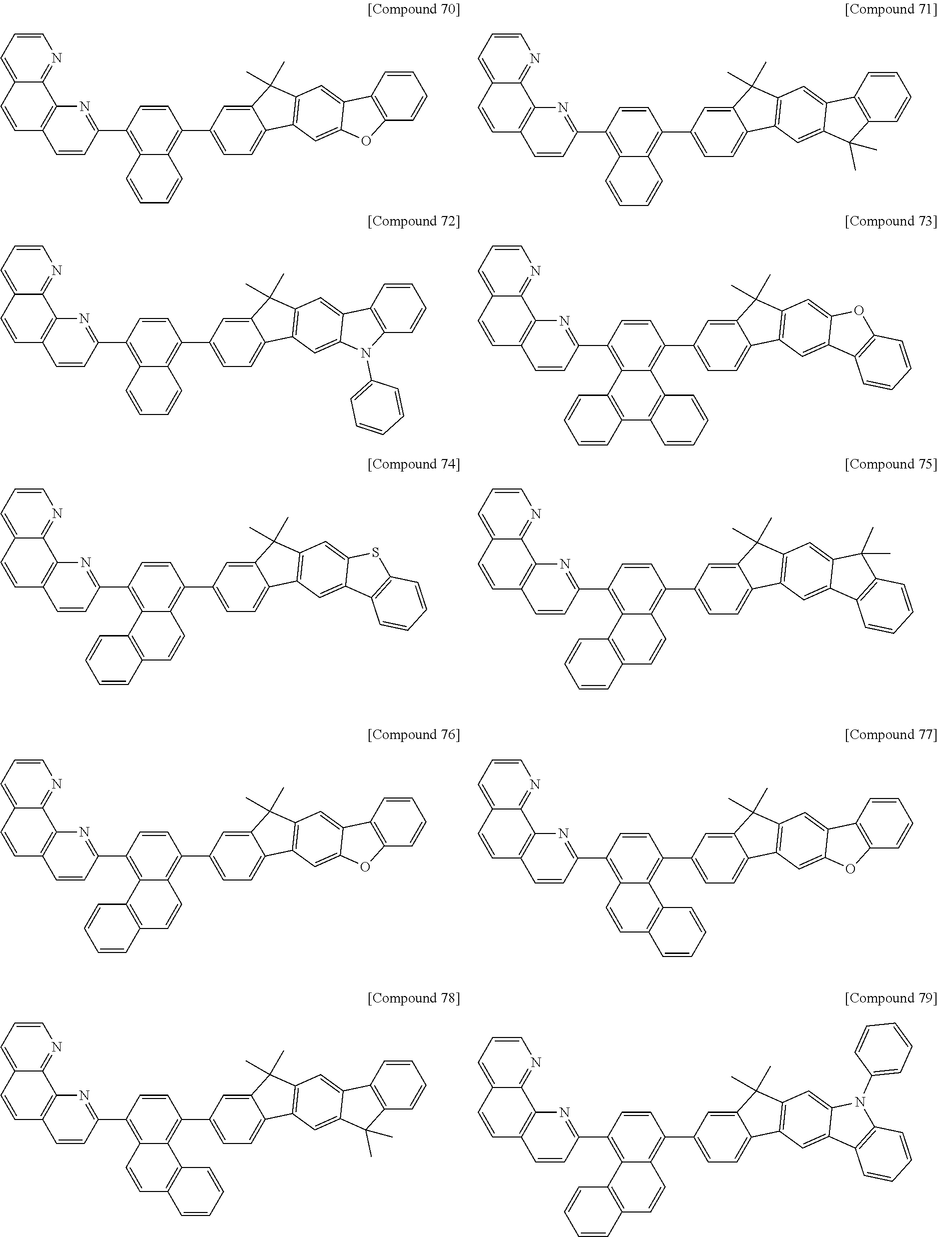

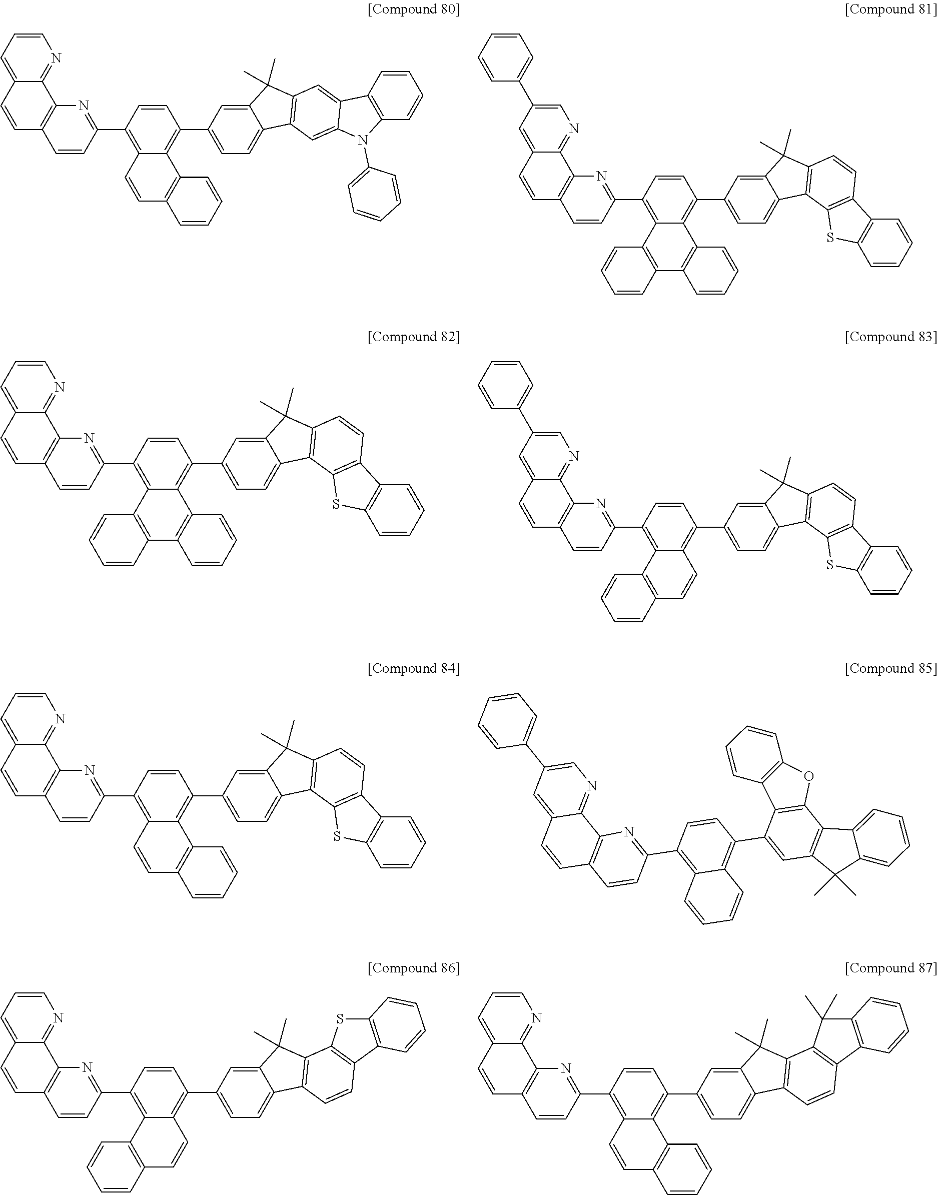

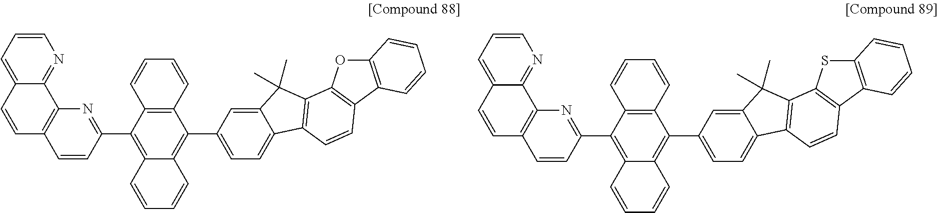 [SMILES: CC1(C)c2cc(-c3c4ccccc4c(-c4ccc5ccc6cccnc6c5n4)c4ccccc34)ccc2-c2ccc3c(oc4ccccc43)c21 ; CC1(C)c2cc(-c3c4ccccc4c(-c4ccc5ccc6cccnc6c5n4)c4ccccc34)ccc2-c2ccc3c(sc4ccccc43)c21]

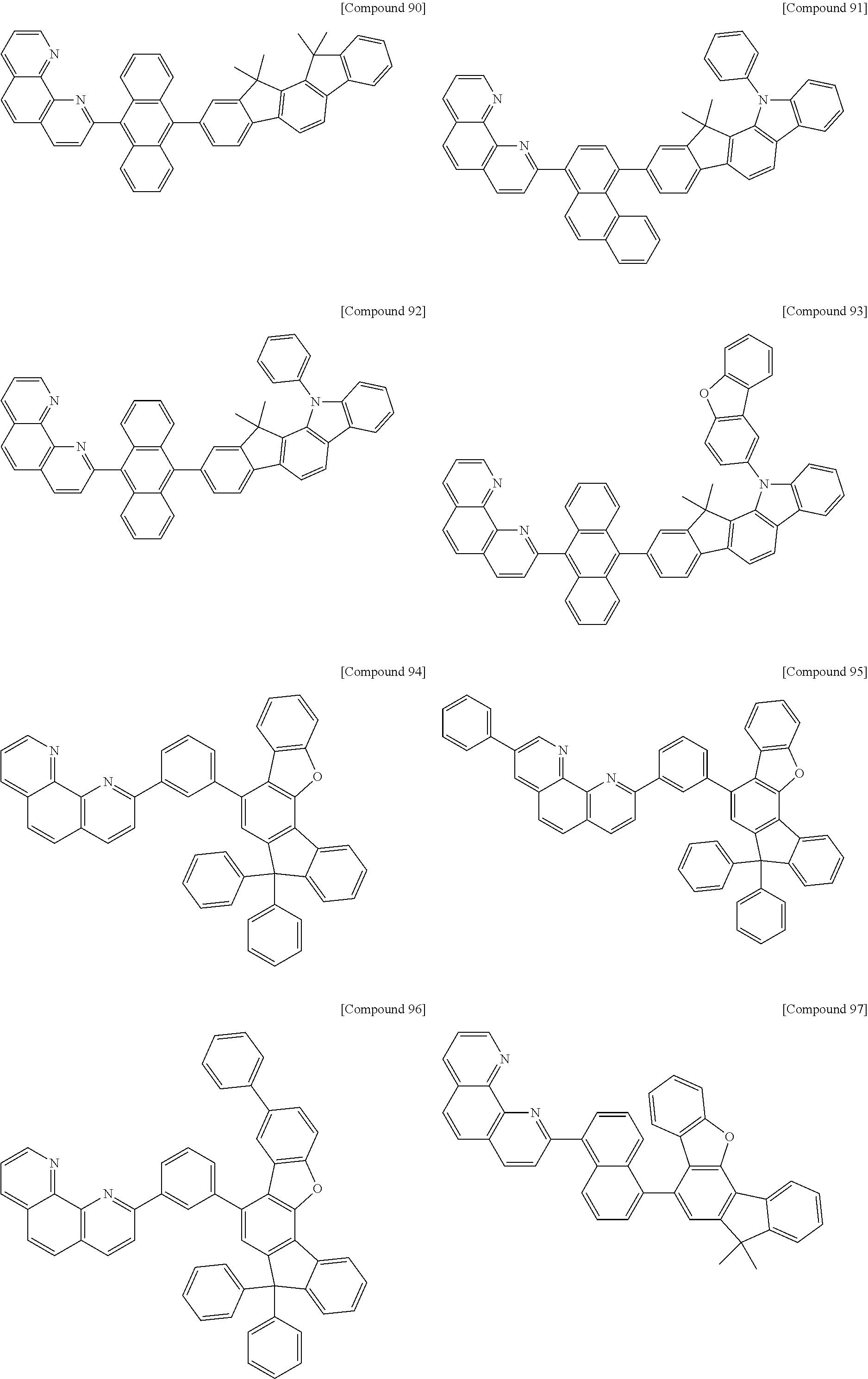

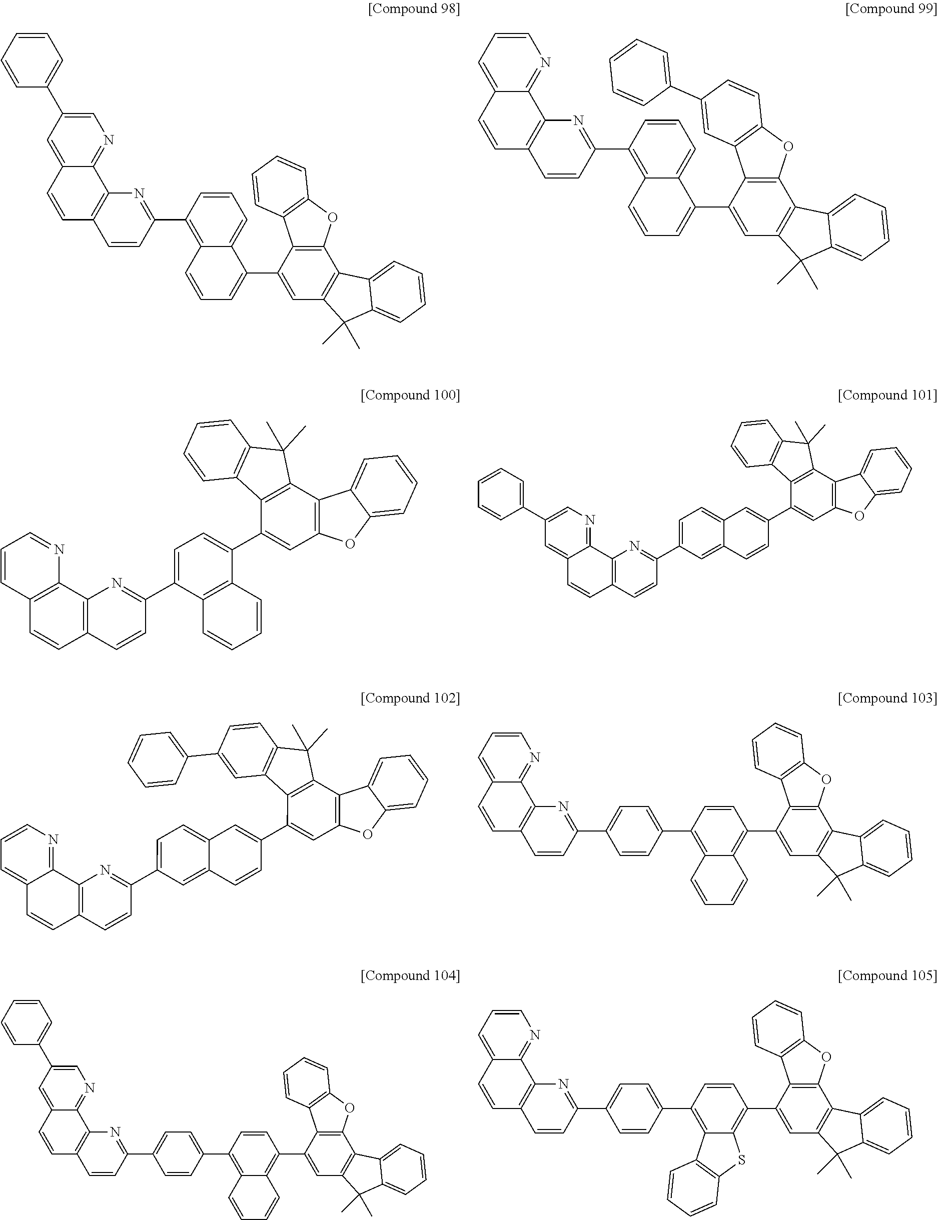

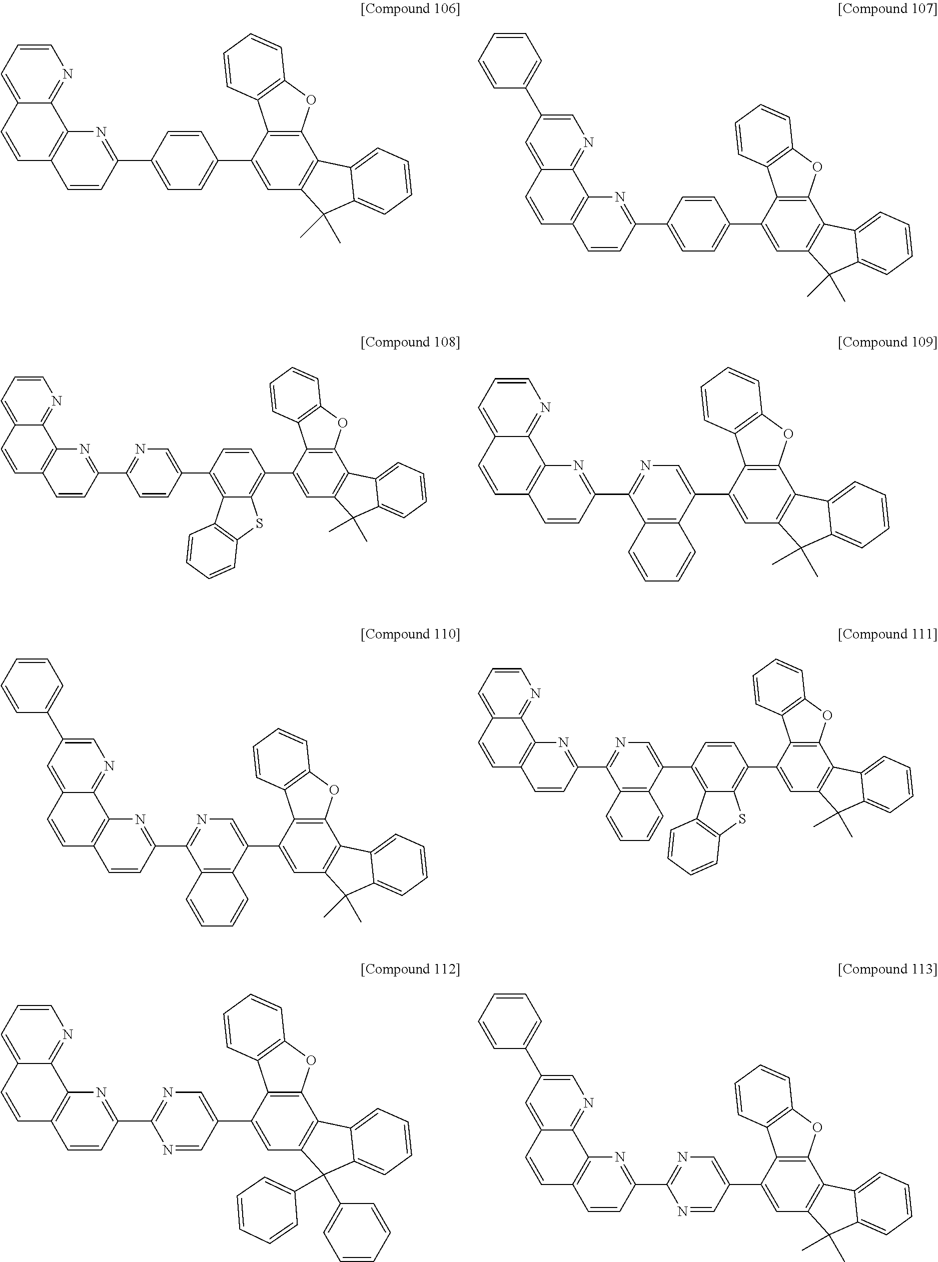

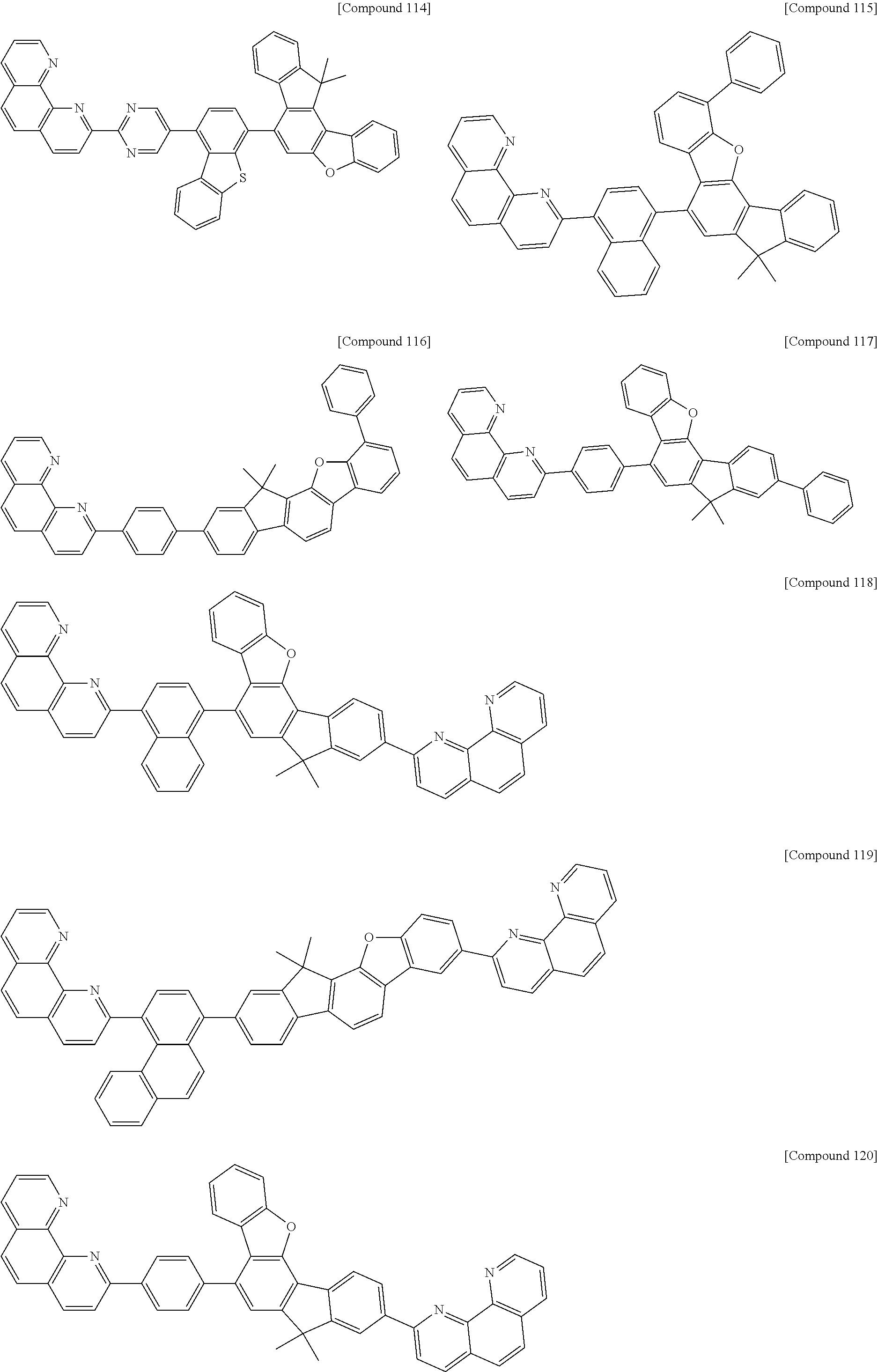

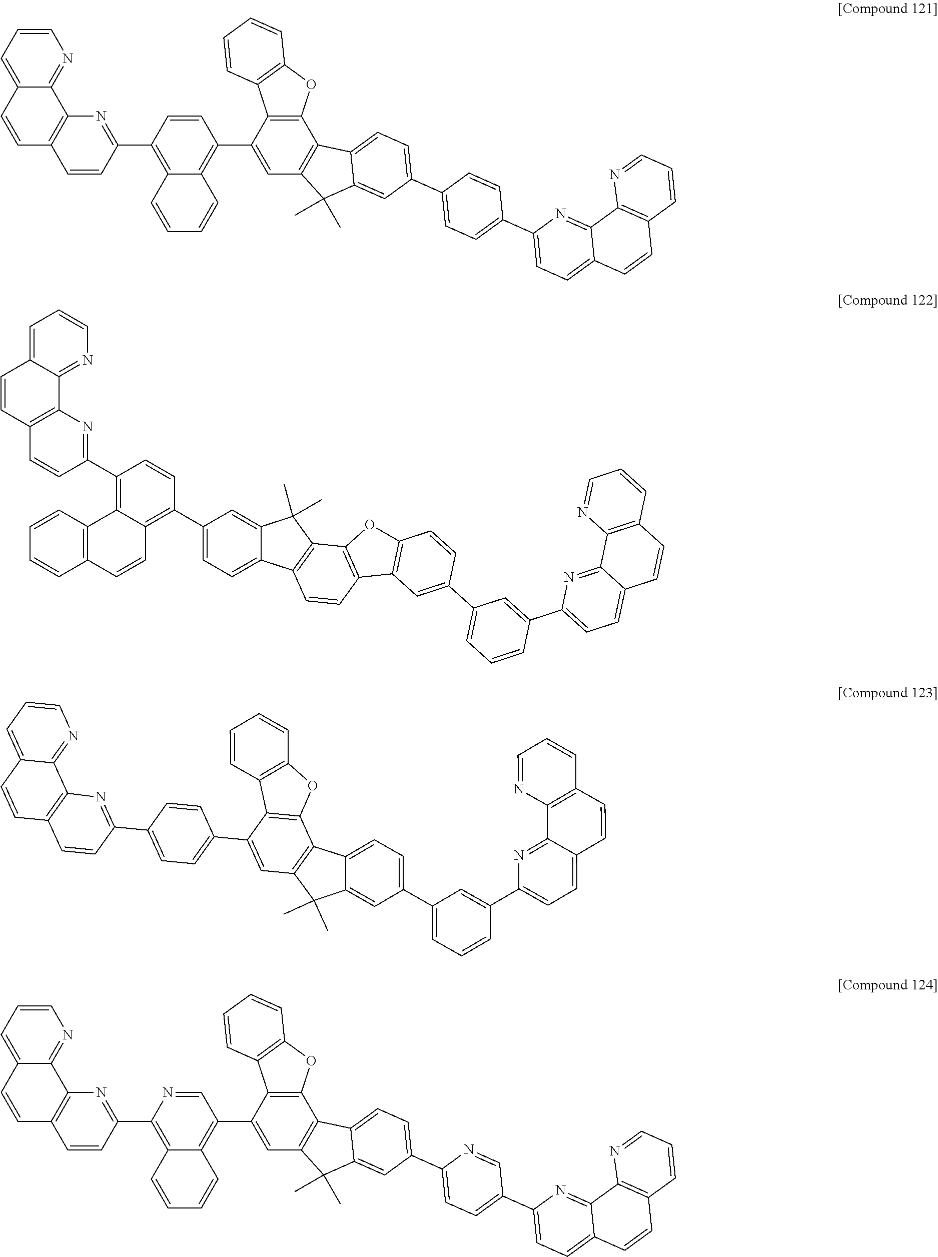 [SMILES: CC1(C)c2cc(-c3ccc(-c4ccc5ccc6cccnc6c5n4)cc3)ccc2-c2c1cc(-c1ccc(-c3ccc4ccc5cccnc5c4n3)c3ccccc13)c1c2oc2ccccc21 ; CC1(C)c2cc(-c3ccc(-c4ccc5ccc6cccnc6c5n4)c4c3ccc3ccccc34)ccc2-c2ccc3c(oc4ccc(-c5cccc(-c6ccc7ccc8cccnc8c7n6)c5)cc43)c21 ; CC1(C)c2cc(-c3cccc(-c4ccc5ccc6cccnc6c5n4)c3)ccc2-c2c1cc(-c1ccc(-c3ccc4ccc5cccnc5c4n3)cc1)c1c2oc2ccccc21 ; CC1(C)c2cc(-c3ccc(-c4ccc5ccc6cccnc6c5n4)cn3)ccc2-c2c1cc(-c1cnc(-c3ccc4ccc5cccnc5c4n3)c3ccccc13)c1c2oc2ccccc21]

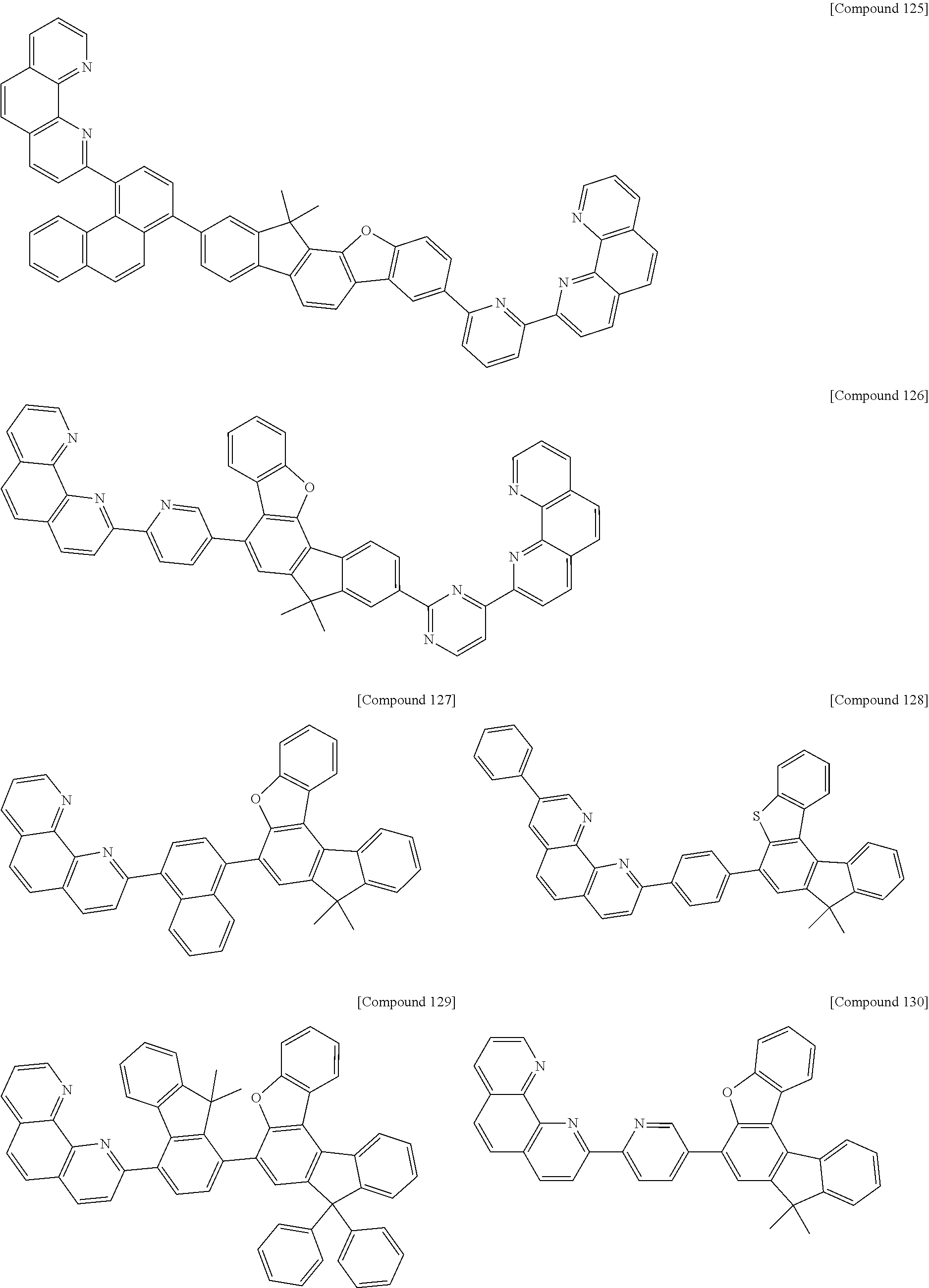

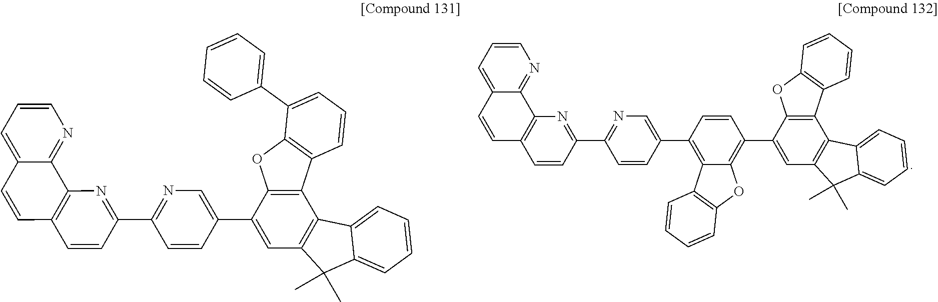 [SMILES: CC1(C)c2ccccc2-c2c1cc(-c1ccc(-c3ccc4ccc5cccnc5c4n3)nc1)c1oc3c(-c4ccccc4)cccc3c21 ; CC1(C)c2ccccc2-c2c1cc(-c1ccc(-c3ccc(-c4ccc5ccc6cccnc6c5n4)nc3)c3c1oc1ccccc13)c1oc3ccccc3c21]

In some particular embodiments thereof, the present disclosure provides an organic light-emitting diode comprising: a first electrode; a second electrode facing the second electrode; and an organic layer interposed between the first electrode and the second electrode, wherein the organic layer contains at least one of the phenanthroline-based compounds represented by Chemical Formula A. In this regard, the organic layer includes a light-emitting layer and a charge generation layer, wherein the charge generation layer includes a P-type and an N-type charge generation layer and the phenanthroline-based compound represented by Chemical Formula A can be used as a material for the N-type charge generation layer. With this structural feature, the organic light-emitting diode according to the present disclosure can exhibit long lifespan and high emission efficiency properties.

Throughout the description of the present disclosure, the phrase “(an organic layer) includes at least one organic compound” may be construed to mean that “(an organic layer) may include a single organic compound species or two or more difference species of organic compounds falling within the scope of the present disclosure”.

In this context, the organic light-emitting diode according to the present disclosure may further include at least one of a light-emitting layer, a hole injection layer, a hole transport layer, a functional layer capable of both hole injection and hole transport, an electron barrier layer, an electron transport layer, a charge generation layer, an electron injection layer, and a capping layer.

The organic light-emitting diode according to the present disclosure may include: a first electrode; a second electrode facing the first electrode; and an organic layer interposed between the first electrode and the second electrode, wherein the organic layer includes: a first light-emitting layer; a charge generation layer; and a second light-emitting layer, the charge generation layer containing at least one of the phenanthroline-based compounds represented by Chemical Formula A.

In greater detail, the organic light-emitting diode may include: an anode as the first electrode; a cathode as the second electrode; and an organic layer interposed between the anode and the cathode, wherein the organic layer includes a light-emitting layer and a charge generation layer inclusive of a P-type charge generation layer and an N-type charge generation layer, and the phenanthroline-based compound represented by Chemical Formula A is used as a material for the N-type charge generation layer.

The organic light-emitting diode according to the present disclosure may include two or more charge generation layers therein.

That is, the organic light-emitting diode according to the present disclosure comprises: a first electrode; a second electrode facing the first electrode; and an organic layer interposed between the first electrode and the second electrode, wherein the organic layer may include: a first light-emitting layer; a first charge generation layer; a second light-emitting layer; a second charge generation layer; and a third light-emitting layer, at least one of the first charge generation layer and the second charge generation layer containing at least one of the phenanthroline-based compounds represented by Chemical Formula A.

In greater detail, the organic light-emitting diode according to the present disclosure comprises: a first electrode; a second electrode facing the first electrode; and an organic layer interposed between the first electrode and the second electrode, wherein the organic layer may include: a first light-emitting layer; a first charge generation layer; and a second light-emitting layer; and may further include a second charge generation layer and a third light-emitting layer between the second light-emitting layer and the second electrode, the first charge generation layer containing at least one of the phenanthroline-based compounds represented by Chemical Formula A, the second charge generation layer including an N-type charge generation layer and a P-type charge generation layer, with the phenanthroline-based compound represented by Chemical formula A being also used as a material for the N-type charge generation layer in the second charge generation layer.

Herein, depending on the structure and environment of the organic light-emitting diode to be used and the emission efficiency and lifespan to be achieved, it is determined whether the phenanthroline-based compound represented by Chemical Formula A according to the present disclosure is employed in either or both of the first charge generation layer and/or the second charge generation layer.

According to an embodiment, the light-emitting layer in the organic light-emitting diode of the present disclosure contains a host and a dopant wherein an anthracene derivative represented by the following Chemical Formula C may be used as the host, but with no limitations thereto:

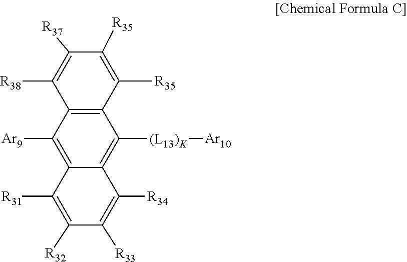

wherein,
R₃₁ to R₃₈, which are same or different, are each independently any one selected from a hydrogen atom, a deuterium atom, a substituted or unsubstituted alkyl of 1 to 30 carbon atoms, an alkenyl of 2 to 24 carbon atoms, an alkynyl of 2 to 24 carbon atoms, a substituted or unsubstituted aryl of 6 to 50 carbon atoms, a substituted or unsubstituted cycloalkyl of 3 to 30 carbon atoms, a substituted or unsubstituted heterocycloalkyl of 1 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 6 to 30 carbon atoms, a substituted or unsubstituted alkylamine of 1 to 30 carbon atoms, a substituted or unsubstituted arylamine of 6 to 30 carbon atoms, a substituted or unsubstituted alkylsilyl of 1 to 30 carbon atoms, a substituted or unsubstituted arylsilyl of 6 to 30 carbon atoms, a nitro, a cyano, and a halogen,
Ar₉ and Ar₁₀, which are same or different, are each independently any one selected from a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, and a substituted or unsubstituted heteroaryl of 2 to 50 carbon atoms;
L₁₃, which functions as a linker, is a single bond or is selected from a substituted or unsubstituted arylene of 6 to 20 carbon atoms and a substituted or unsubstituted heteroarylene of 2 to 20 carbon atoms; and
k is an integer of 1 to 3, wherein when k is 2 or greater, the corresponding L₁₃'s are same or different.

For a more exemplary host, Arg in Chemical Formula C may be a substituent represented by the following Chemical Formula C-1:

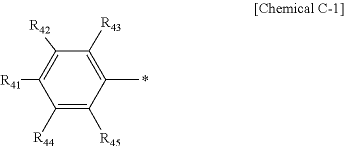

wherein, R₄₁ to R₄₅, which may be same or different, are as defined for R₃₁ to R₃₈ above; and may each be linked to an adjacent one to form a saturated or unsaturated cyclic ring. In this case, L₁₃ may be a single bond or a substituted or unsubstituted arylene of 6 to 20 carbon atoms, and k may be 1 or 2, with the proviso that when k is 2, corresponding L₁₃'s may be same or different.

According to one embodiment, the anthracene derivative may be any one selected from the compounds represented by the following [Chemical Formula 22] to [Chemical Formula 63], but with no limitations thereto:

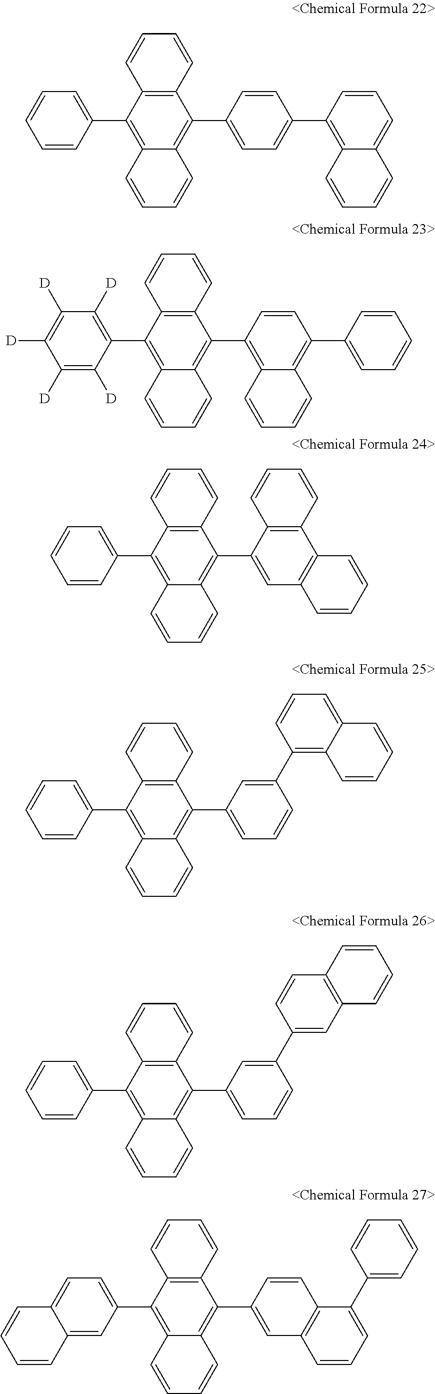

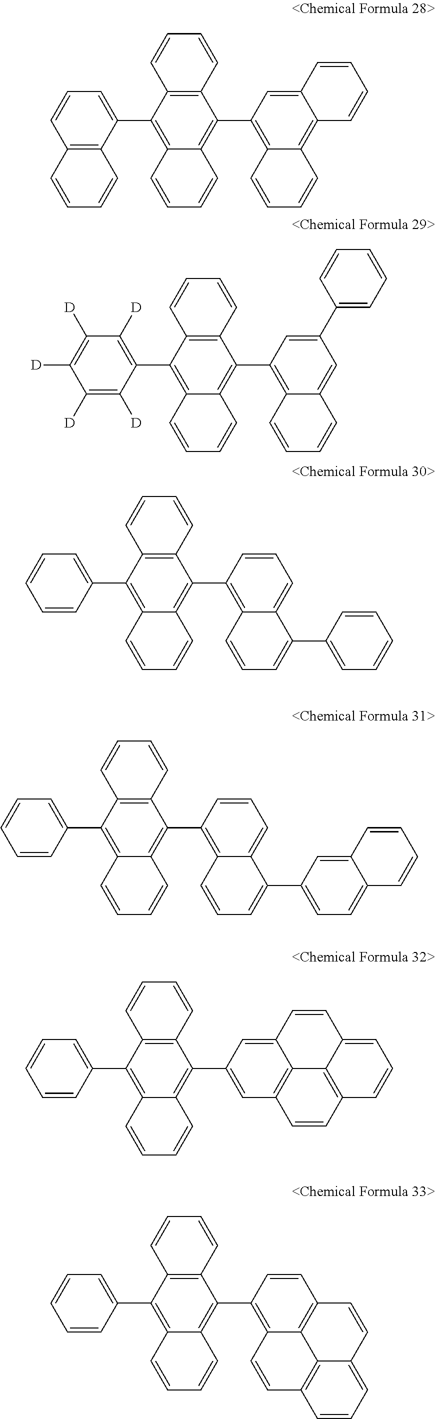

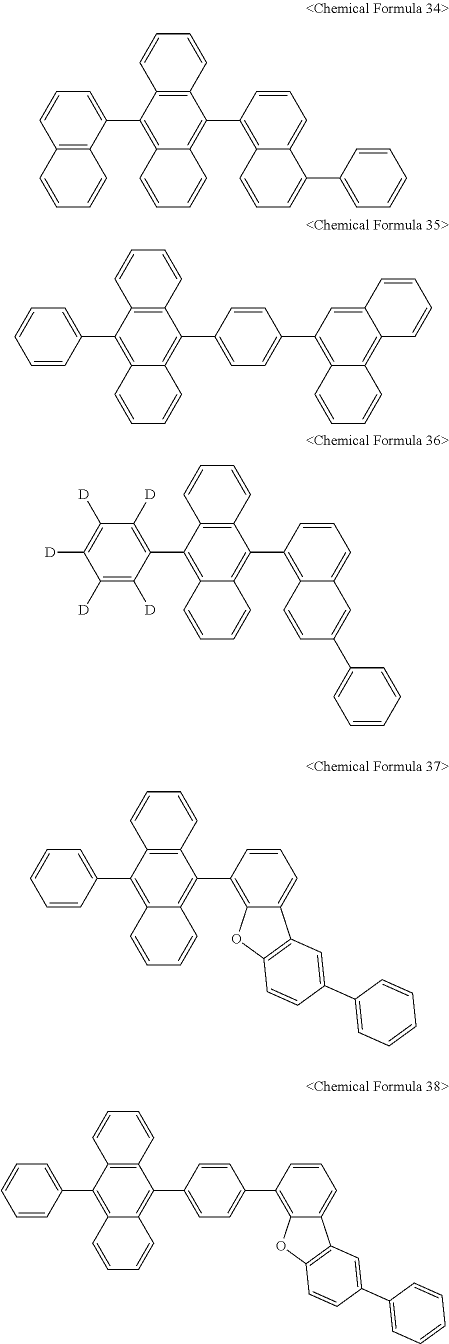

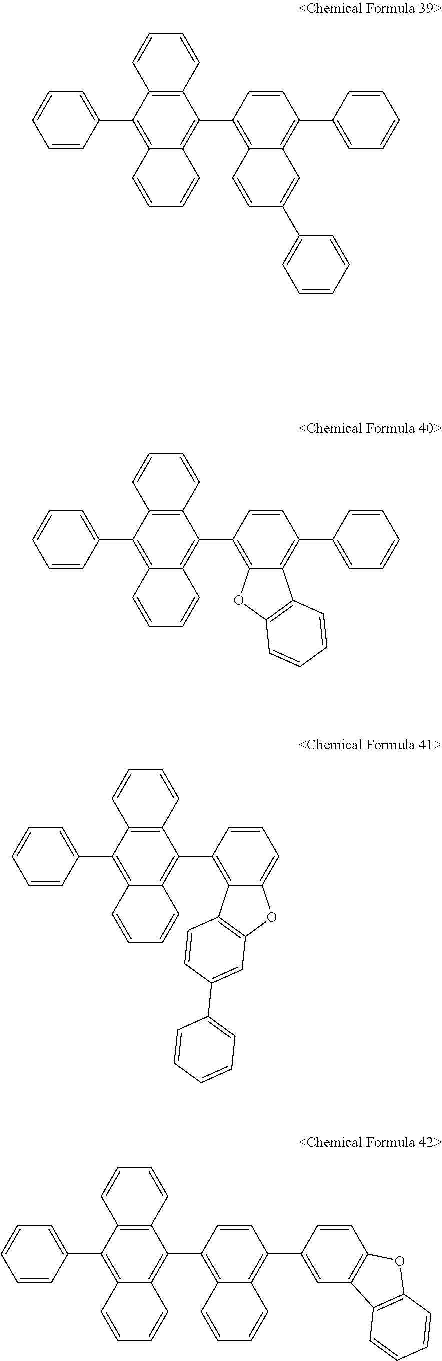

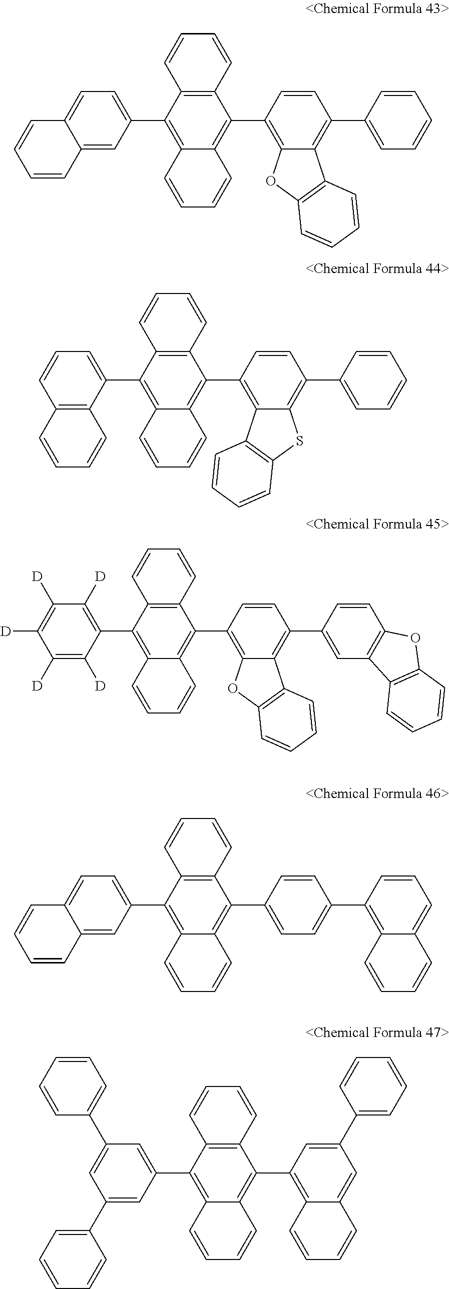

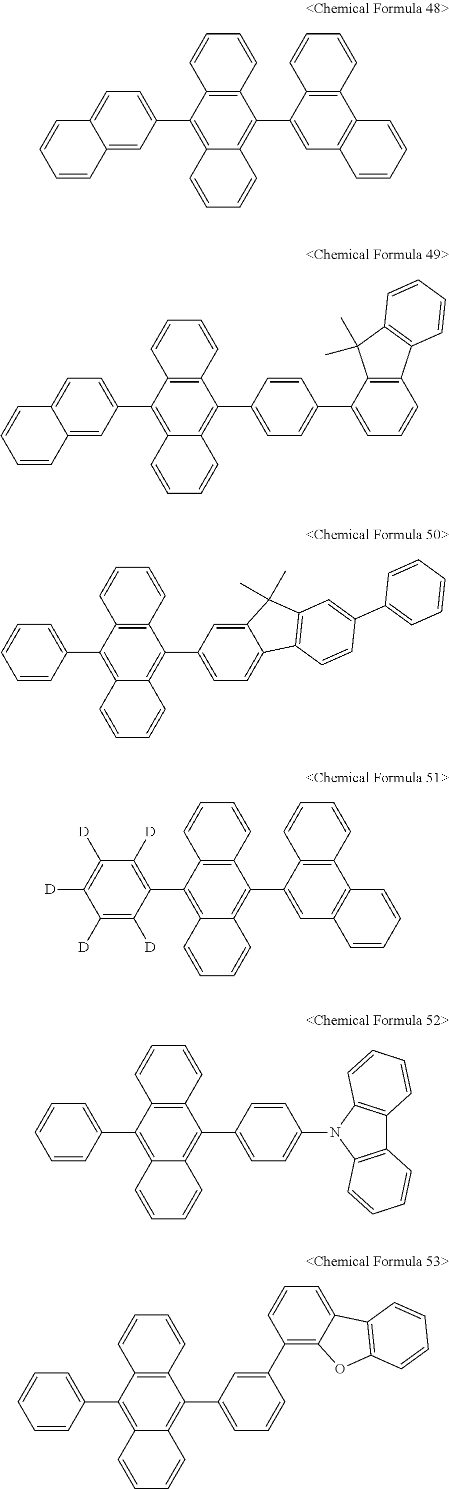

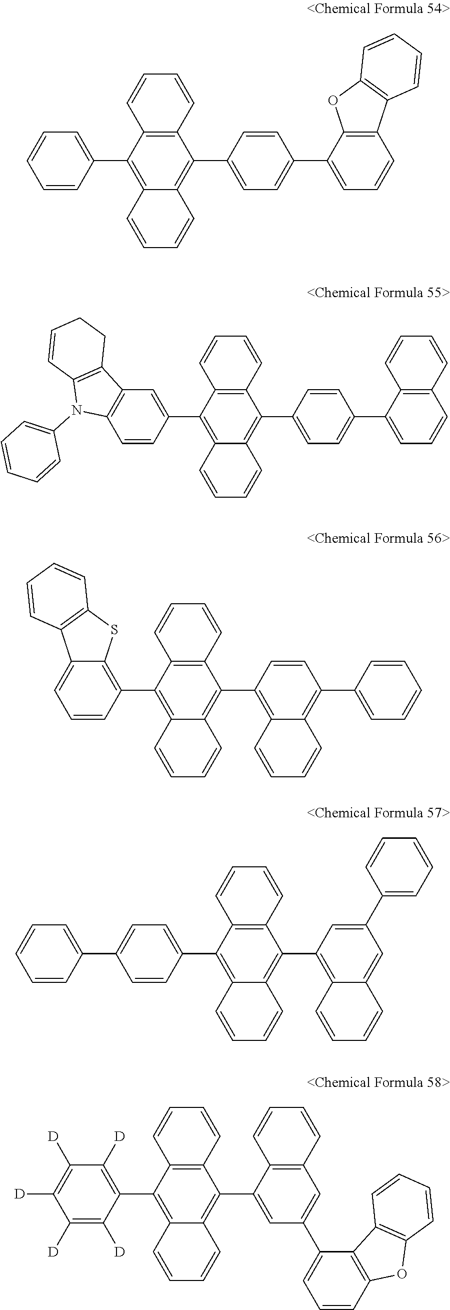

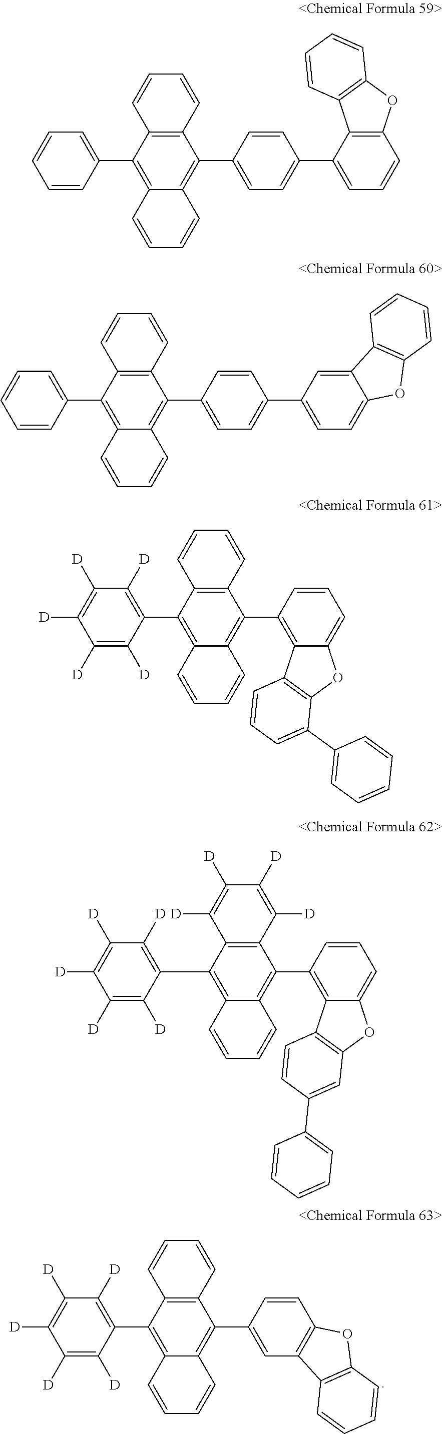 [SMILES: c1ccc(-c2c3ccccc3c(-c3ccc(-c4cccc5oc6ccccc6c45)cc3)c3ccccc23)cc1 ; c1ccc(-c2c3ccccc3c(-c3ccc(-c4ccc5oc6ccccc6c5c4)cc3)c3ccccc23)cc1 ; [2H]c1c([2H])c([2H])c(-c2c3ccccc3c(-c3cccc4oc5c(-c6ccccc6)cccc5c34)c3ccccc23)c([2H])c1[2H] ; [2H]c1c([2H])c([2H])c(-c2c3ccccc3c(-c3cccc4oc5cc(-c6ccccc6)ccc5c34)c3c([2H])c([2H])c([2H])c([2H])c23)c([2H])c1[2H] ; [2H]c1c([2H])c([2H])c(-c2c3ccccc3c(-c3ccc4oc5ccccc5c4c3)c3ccccc23)c([2H])c1[2H]]

In addition, the dopant compound used in the light-emitting layer may include at least one compound represented by the following [Chemical Formula D1] to [Chemical Formula D8]:

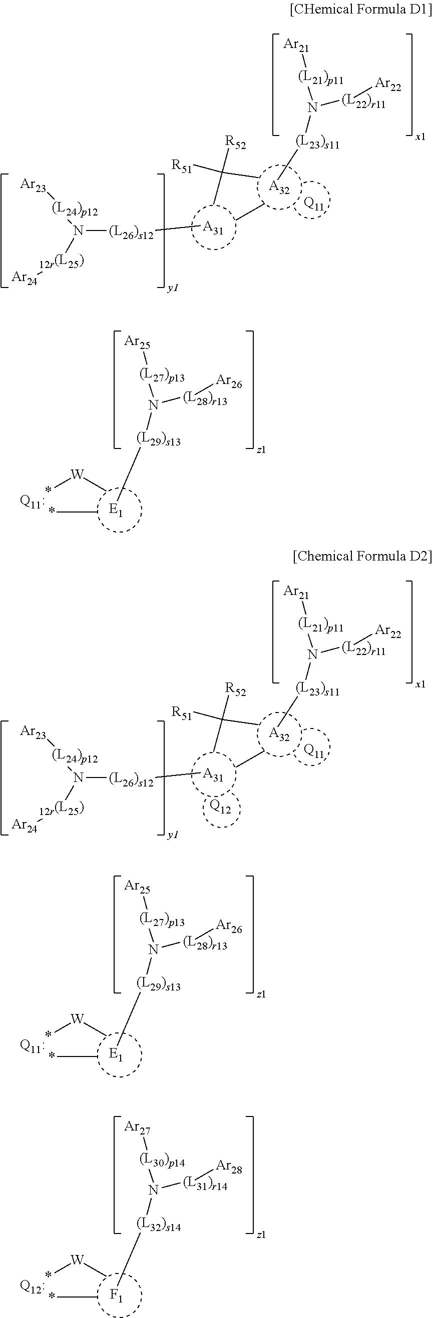

wherein,
A₃₁, A₃₂, E₁, and F₁ may be same or different and are each independently a substituted or unsubstituted aromatic hydrocarbon ring of 6 to 50 carbon atoms, or a substituted or unsubstituted heteroaromatic ring of 2 to 40 carbon atoms wherein two adjacent carbon atoms of the aromatic ring A₃₁ and two adjacent carbon atoms of the aromatic ring A₃₂ form a 5-membered fused ring together with a carbon atom to which substituents R₅₁ and R₅₂ are bonded;
linkers L₂₁ to L₃₂ may be same or different, and are each independently selected from among a direct bond, a substituted or unsubstituted alkylene of 1 to 60 carbon atoms, a substituted or unsubstituted alkenylene of 2 to 60 carbon atoms, a substituted or unsubstituted alkynylene of 2 to 60 carbon atoms, a substituted or unsubstituted cycloalkylene of 3 to 60 carbon atoms, a substituted or unsubstituted heterocycloalkylene of 2 to 60 carbon atoms, a substituted or unsubstituted arylene of 6 to 60 carbon atoms, and a substituted or unsubstituted heteroarylene of 2 to 60 carbon atoms;
W is any one selected from among N—R₅₃, CR₅₄R₅₅, SiR₅₆R₅₇, GeF₅₈R₅₉, O, S, and Se;
R₅₁ to R₅₉, and Ar₂₁ to Ar₂₈ may be same or different, and are each independently any one selected from among a hydrogen atom, a deuterium atom, a substituted or unsubstituted alkyl of 1 to 30 carbon atoms, a substituted or unsubstituted aryl of 6 to 50 carbon atoms, a substituted or unsubstituted alkenyl of 2 to 30 carbon atoms, a substituted or unsubstituted alkynyl of 2 to 20 carbon atoms, a substituted or unsubstituted cycloalkyl of 3 to 30 carbon atoms, a substituted or unsubstituted cycloalkenyl of 5 to 30 carbon atoms, a substituted or unsubstituted heteroaryl of 2 to 50 carbon atoms, a substituted or unsubstituted heterocycloalkyl of 2 to 30 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a substituted or unsubstituted alkylgermanium of 1 to 30 carbon atoms, a substituted or unsubstituted arylgermanium of 1 to 30 carbon atoms, a cyano, a nitro, and a halogen, with the proviso that R₅₁ and R₅₂ together may form a mono- or polycyclic aliphatic or aromatic ring that may be a heterocyclic ring containing a heteroatom selected from among N, O, P, Si, S, Ge, Se, and Te as a ring member;
p11 to p14, r₁₁ to r₁₄, and s₁₁ to s₁₄ are each independently an integer of 1 to 3, with the proviso that when any of them is 2 or greater, the corresponding linkers may be the same or different,
x1 is an integer of 1 or 2, and y1 and z1 may be same or different and are each independently an integer of 0 to 3; and
Ar₂₁ may form a ring with Ar₂₂, Ar₂₃ may form a ring with Ar₂₄, Ar₂₅ may form a ring with Ar₂₆, and Ar₂₇ may form a ring with Ar₂₈;
two adjacent carbon atoms of the A₃₂ ring moiety of Chemical Formula D1 occupy respective positions * of Structural Formula Q₁₁ to form a fused ring, and
two adjacent carbon atoms of the A₃₁ ring moiety of Chemical Formula D2 occupy respective positions * of structural Formula Q₁₂ to form a fused ring, and two adjacent carbon atoms of the A₃₂ ring moiety of Chemical Formula D2 occupy respective positions * of structural Formula Q₁₁ to form a fused ring,

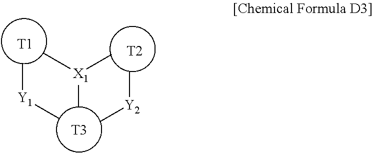

wherein,
X₁ is any one selected from B, P, and P═O,
T1 to T3, which are same or different, are each independently a substituted or unsubstituted aromatic hydrocarbon ring of 6 to 50 carbon atoms, or a substituted or unsubstituted heteroaromatic ring of 2 to 40 carbon atoms;
Y₁ is any one selected from among N—R₆₁, CR₆₂R₆₃, O, S, and SiR₆₄R₆₅;
Y₂ is any one selected from among N—R₆₆, CR₆₇R₆₈, O, S, SiR₆₉R₇₀ wherein R₆₁ to R₇₀, which may be same or different, are each independently any one selected from among a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a cyano, and a halogen and wherein at least one of R₆₁ to R₇₀ may be connected to at least one of T1 to T3 to form an additional mono- or polycyclic aliphatic or aromatic ring;

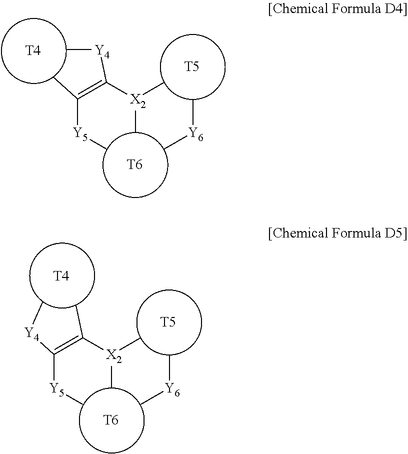

wherein,
X₂ is any one selected from among B, P, and P═O,
T4 to T6 are as defined for T1 to T3 in Chemical Formula D3,
Y₄ to Y₆ are as defined for Y₁ to Y₂ in Chemical Formula D3;

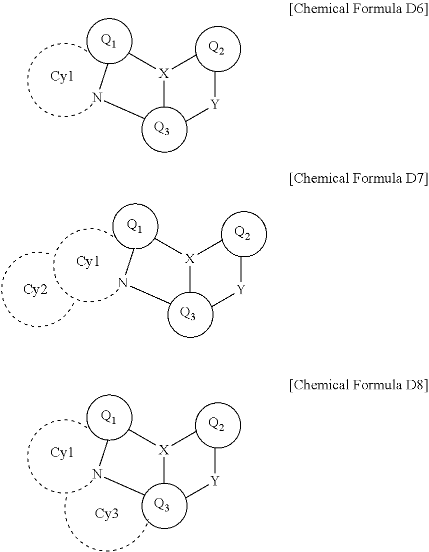

wherein,
Q₁ to Q₃, which may be same or different, are each independently a substituted or unsubstituted aromatic hydrocarbon ring of 6 to 50 carbon atoms, or a substituted or unsubstituted heteroaromatic ring of 2 to 50 carbon atoms,
Y is any one selected from N—R₃, CR₄R₅, O, S, and Se,
X is any one selected from B, P, and P═O,
R₃ to R₅ may each be connected to the Q₂ or Q₃ ring moiety to form an additional mono- or polycyclic aliphatic or aromatic,
R₃ to R₅, which may be same or different, are each independently any one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkoxy of 1 to 30 carbon atoms, a substituted or unsubstituted aryloxy of 6 to 30 carbon atoms, a substituted or unsubstituted alkylthioxy of 1 to 30 carbon atoms, a substituted or unsubstituted arylthioxy of 5 to 30 carbon atoms, a substituted or unsubstituted alkylamine of 1 to 30 carbon atoms, a substituted or unsubstituted arylamine of 5 to 30 carbon atoms, a substituted or unsubstituted alkylsilyl of 1 to 30 carbon atoms, a substituted or unsubstituted arylsilyl of 5 to 30 carbon atoms, a nitro, a cyano, and a halogen, and
R₄ and R₅ may be connected to each other to form an additional mono- or polycyclic aliphatic or aromatic ring,
the ring formed by Cy1 is a substituted or unsubstituted alkylene of 1 to 10 carbon atoms, except for the nitrogen (N) atom, the aromatic carbon atom of Q₁ to which the nitrogen (N) atom is connected, and the aromatic carbon atom of Q₁ to which Cy1 is to bond,
Cy2 in Chemical Formula D7 forms a saturated hydrocarbon ring added to Cy1 wherein the ring formed by Cy2 is a substituted or unsubstituted alkylene of 1 to 10 carbon atoms, except for the carbon atoms included in Cy1, and
the ring formed by Cy3 in Chemical Formula D8 is a substituted or unsubstituted alkylene of 1 to 10 carbon atoms, except for the aromatic carbon atom of Q₃ to which Cy3 is to bond, the aromatic carbon atom of Q₃ to which the nitrogen (N) atom is connected, the nitrogen (N) atom, and the carbon atom of Cy1 to which the nitrogen (N) atom is connected,
wherein the term “substituted” in the expression “substituted or unsubstituted” used for compounds of Chemical Formulas D1 to D8 means having at least one substituent selected from the group consisting of a deuterium atom, a cyano, a halogen, a hydroxy, a nitro, an alkyl of 1 to 24 carbon atoms, a halogenated alkyl of 1 to 24 carbon atoms, an alkenyl of 2 to 24 carbon atoms, an alkynyl of 2 to 24 carbon atoms, a heteroalkyl of 1 to 24 carbon atoms, an aryl of 6 to 24 carbon atoms, an arylalkyl of 7 to 24 carbon atoms, a heteroaryl of 2 to 24 carbon atoms or heteroarylalkyl of 2 to 24 carbon atoms, an alkoxy of 1 to 24 carbon atoms, an alkylamino of 1 to 24 carbon atoms, an arylamino of 6 to 24 carbon atoms, a heteroarylamino of 1 to 24 carbon atoms, an alkylsilyl of 1 to 24 carbon atoms, an arylsilyl of 6 to 24 carbon atoms, and an aryloxy of 6 to 24 carbon atoms.

In [Chemical Formula D6] to [Chemical Formula D8], “Cy1” is linked to the nitrogen (N) atom and an aromatic carbon atom of the Q₁ ring to form a ring with the nitrogen (N) atom, the aromatic carbon atom of the Q₁ ring to which the nitrogen (N) atom is boned, and the aromatic carbon atom of the Q₁ ring to which Cy1 is to bond, and the ring formed by Cy1 may be a substituted or unsubstituted alkylene of 1 to 10 carbon atoms, particularly a substituted or unsubstituted alkylene of 2 to 7 carbon atoms, and more particularly a substituted or unsubstituted alkylene of 2 to 5 carbon atoms, except for the nitrogen (N) atom, the aromatic carbon atom of Q₁ to which the nitrogen (N) atom is bonded, and the aromatic carbon atom of Q₁ to which Cy1 is to bond.

In addition, the ring formed by “Cy2” in Chemical Formula D7 is a substituted or unsubstituted alkylene of 1 to 10 carbon atoms, particularly a substituted or unsubstituted alkylene of 2 to 7 carbon atoms, and more particularly a substituted or unsubstituted alkylene of 2 to 5 carbon atoms, except for the carbon atoms shared by Cy1.

In addition, ‘Cy3’ in Chemical Formula D8 is linked to both the carbon atom boned to the nitrogen atom in Cy1 and the aromatic carbon atom of Q₃ ring to which Cy3 is to bond so as to form a fused ring with the aromatic carbon atom of Q₃ ring to which Cy3 is to bond, the nitrogen (N) atom, and the carbon atom of Cy1 to which the nitrogen (N) atom is bonded, and the ring formed by Cy3 is a substituted or unsubstituted alkylene of 1 to 10 carbon atoms, particularly a substituted or unsubstituted alkylene of 2 to 7 carbon atoms, and more particularly a substituted or unsubstituted alkylene of 2 to 5 carbon atoms, except for the aromatic carbon atom of Q₃ to which Cy3 is to bond, the aromatic carbon atom of Q₃ to which the nitrogen (N) atom is bonded, the nitrogen (N) atom, and the carbon atom of Cy1 to which the nitrogen (N) atom is bonded.

Among the dopant compounds according to the present disclosure, the boron compound represented by [Chemical Formula D3] to [Chemical Formula D8] may have, on the aromatic hydrocarbon rings or heteroaromatic rings of T1 to T6 or on the aromatic hydrocarbon rings or heteroaromatic rings of Q₁ to Q₃, a substituent selected from a deuterium atom, an alkyl of 1 to 24 carbon atoms, an aryl of 6 to 24 carbon atoms, an alkylamino of 1 to 24 carbon atoms, an arylamino of 6 to 24 carbon atoms, wherein the alkyl radicals or the aryl radicals in the alkylamino of 1 to 24 carbon atoms and the arylamino of 6 to 24 carbon atoms on the rings may be linked to each other, and particularly a substituent selected from an alkyl of 1 to 12 carbon atoms, an aryl of 6 to 18 carbon atoms, an alkylamino of 1 to 12 carbon atoms, and an arylamino of 6 to 18 carbon atoms wherein the alkyl radicals or aryl radicals in the alkylamino of 1 to 12 carbon atoms and the arylamino of 6 to 18 carbon atoms on the rings may be linked to each other.

In addition, concrete examples of the dopant compounds of [Chemical Formula D1] and [Chemical Formula D2] used in the light-emitting layer include the compounds of the following Chemical Formulas d1 to d239:

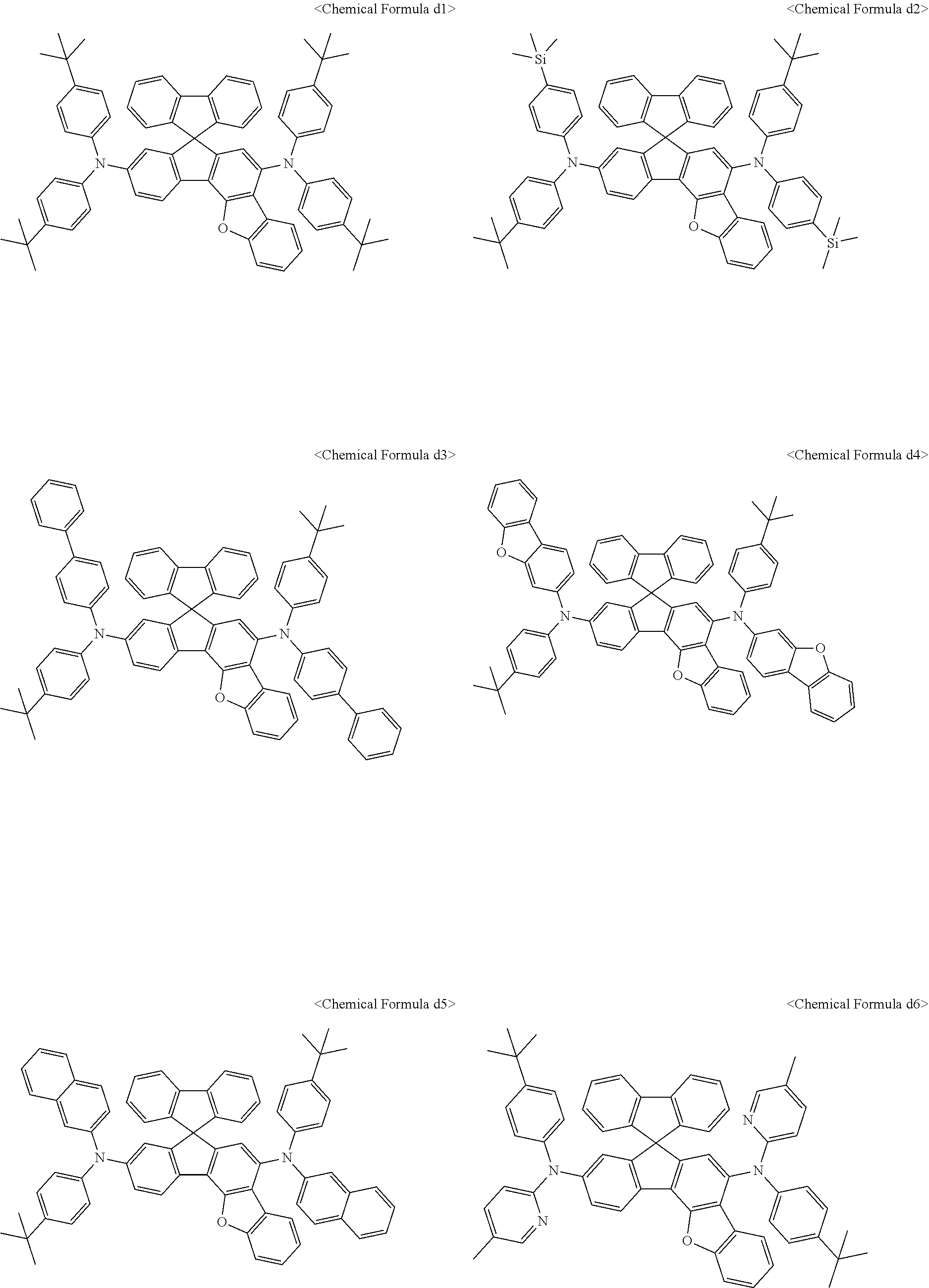

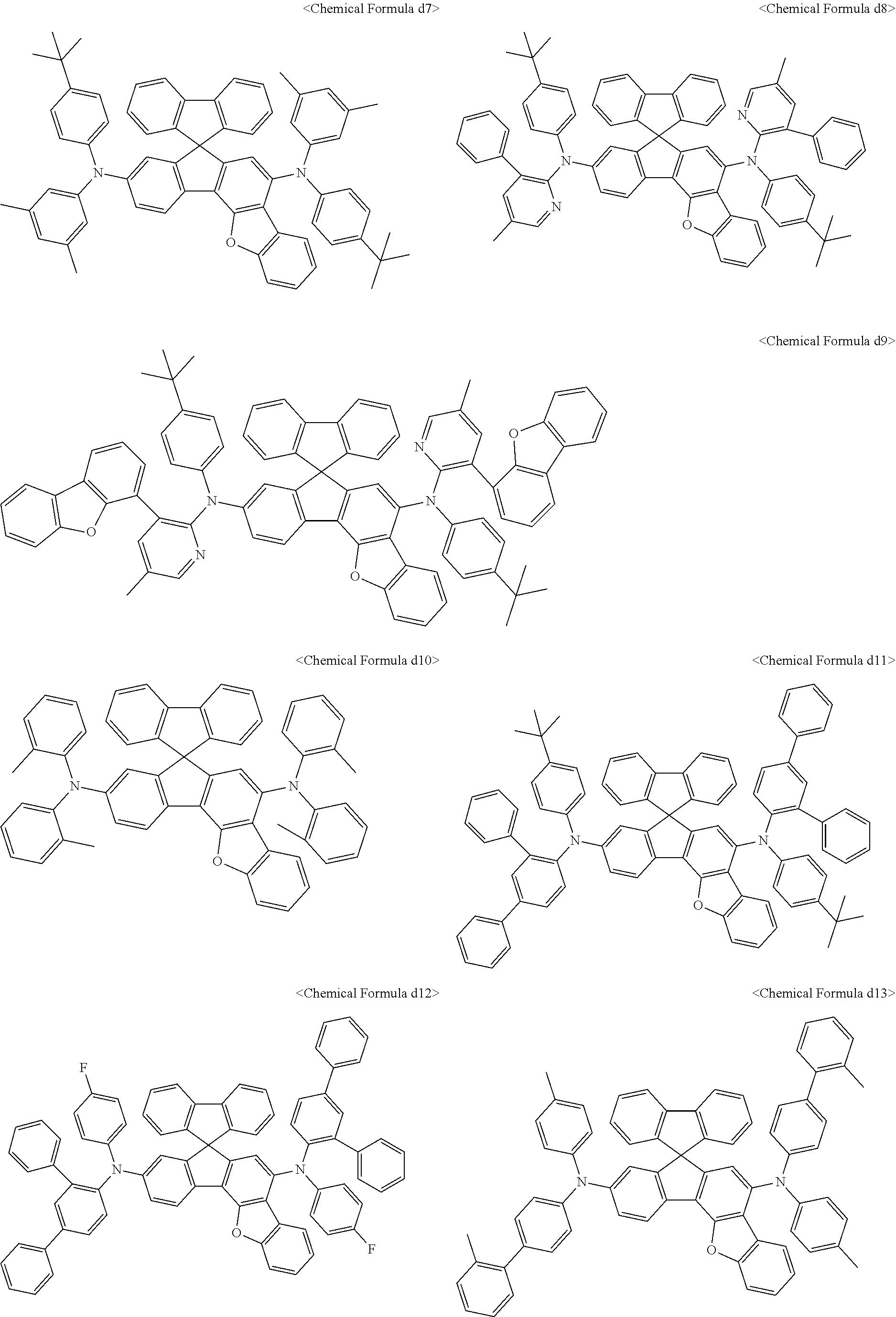

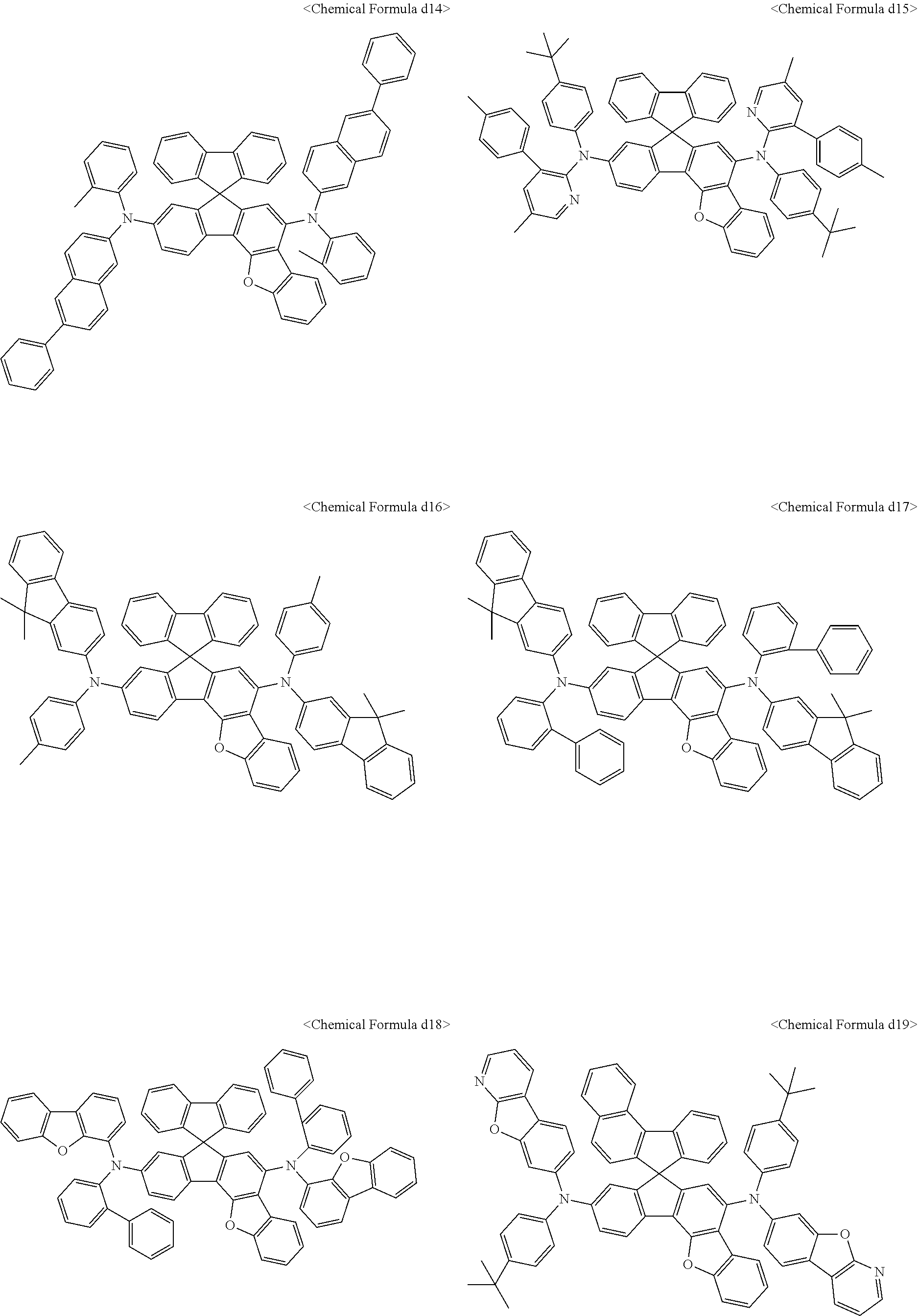

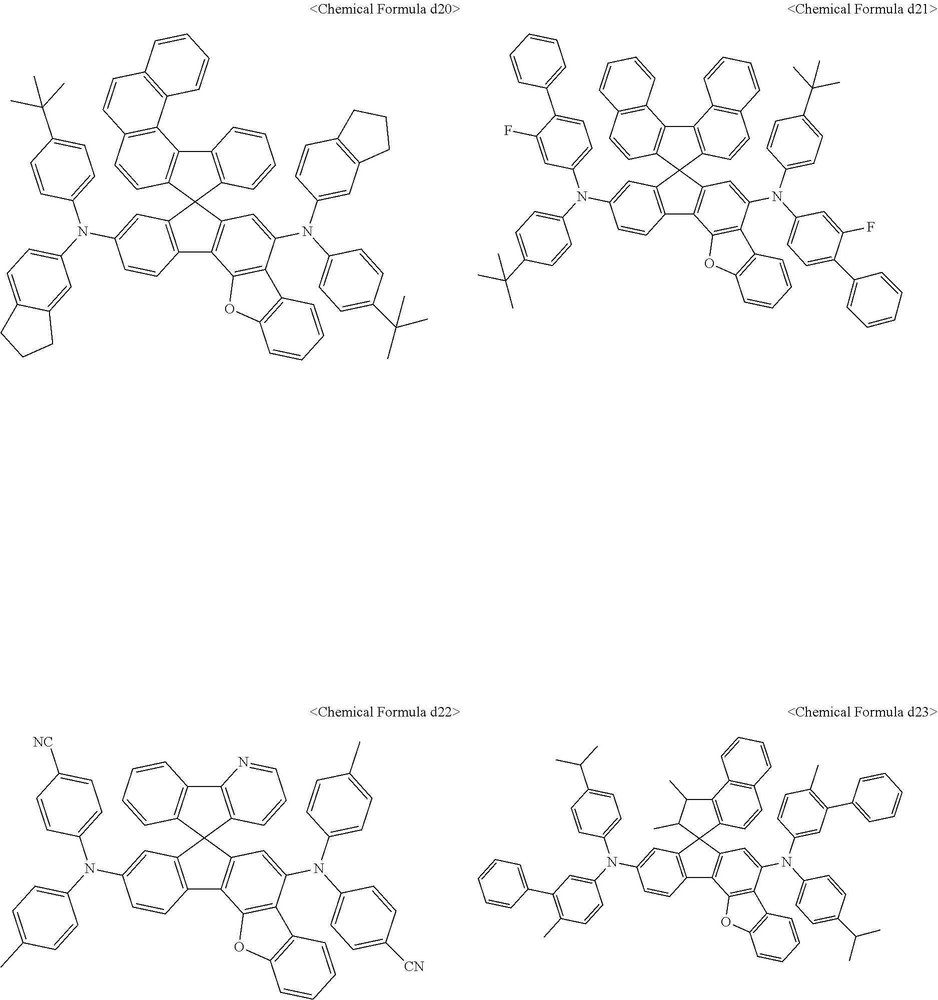

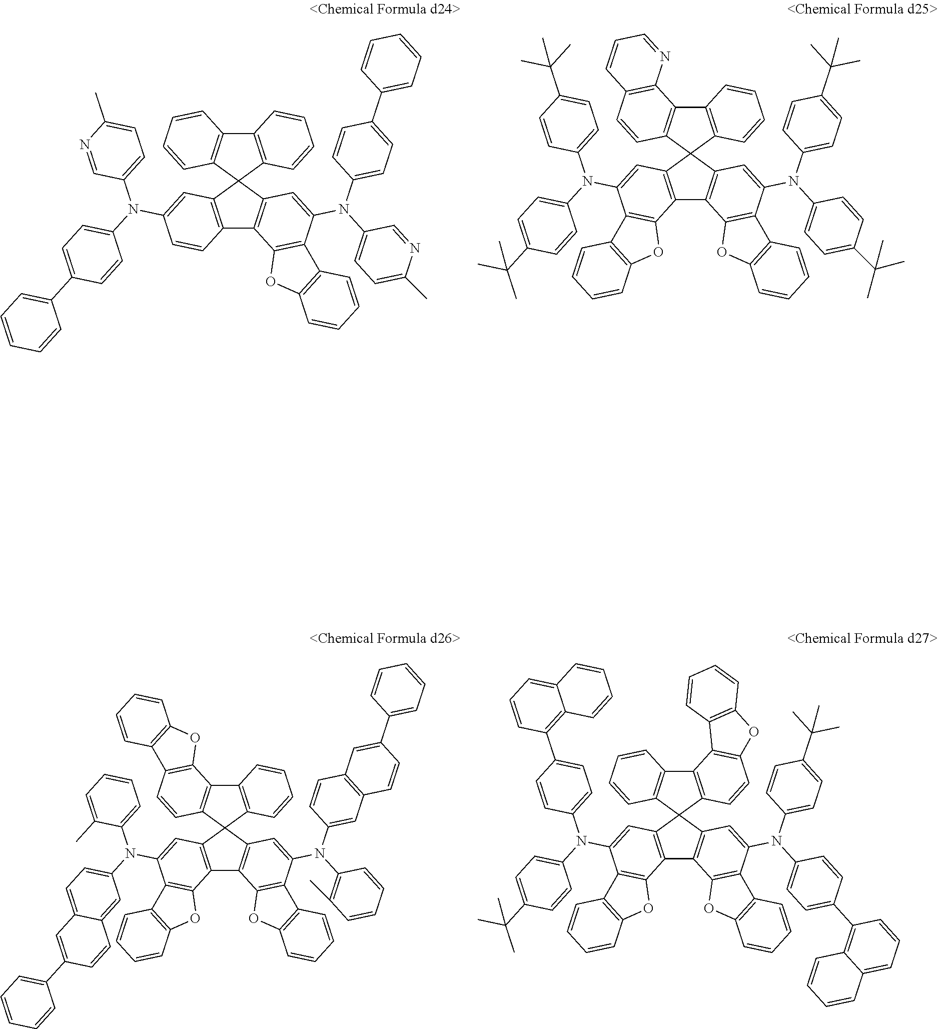

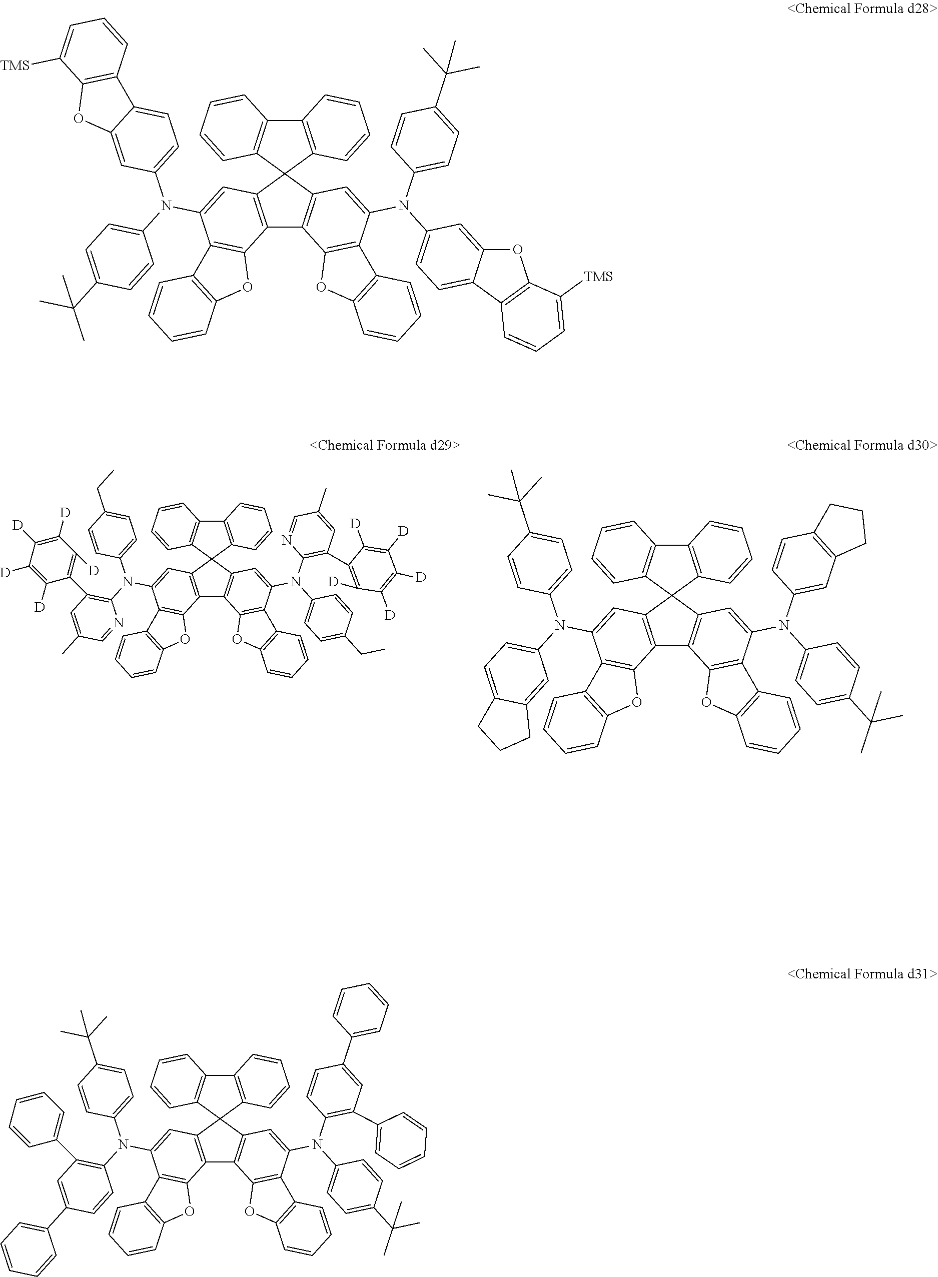

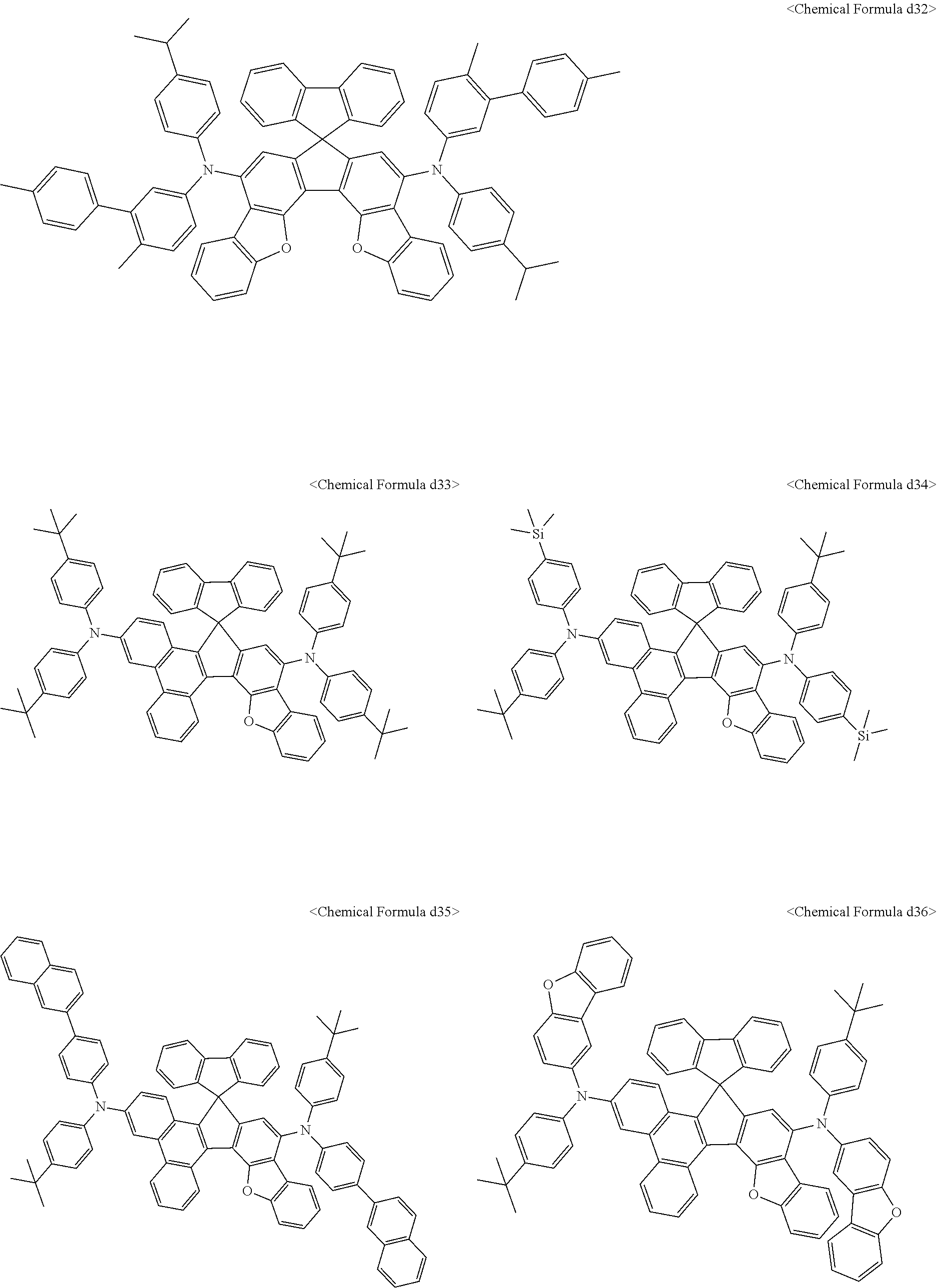

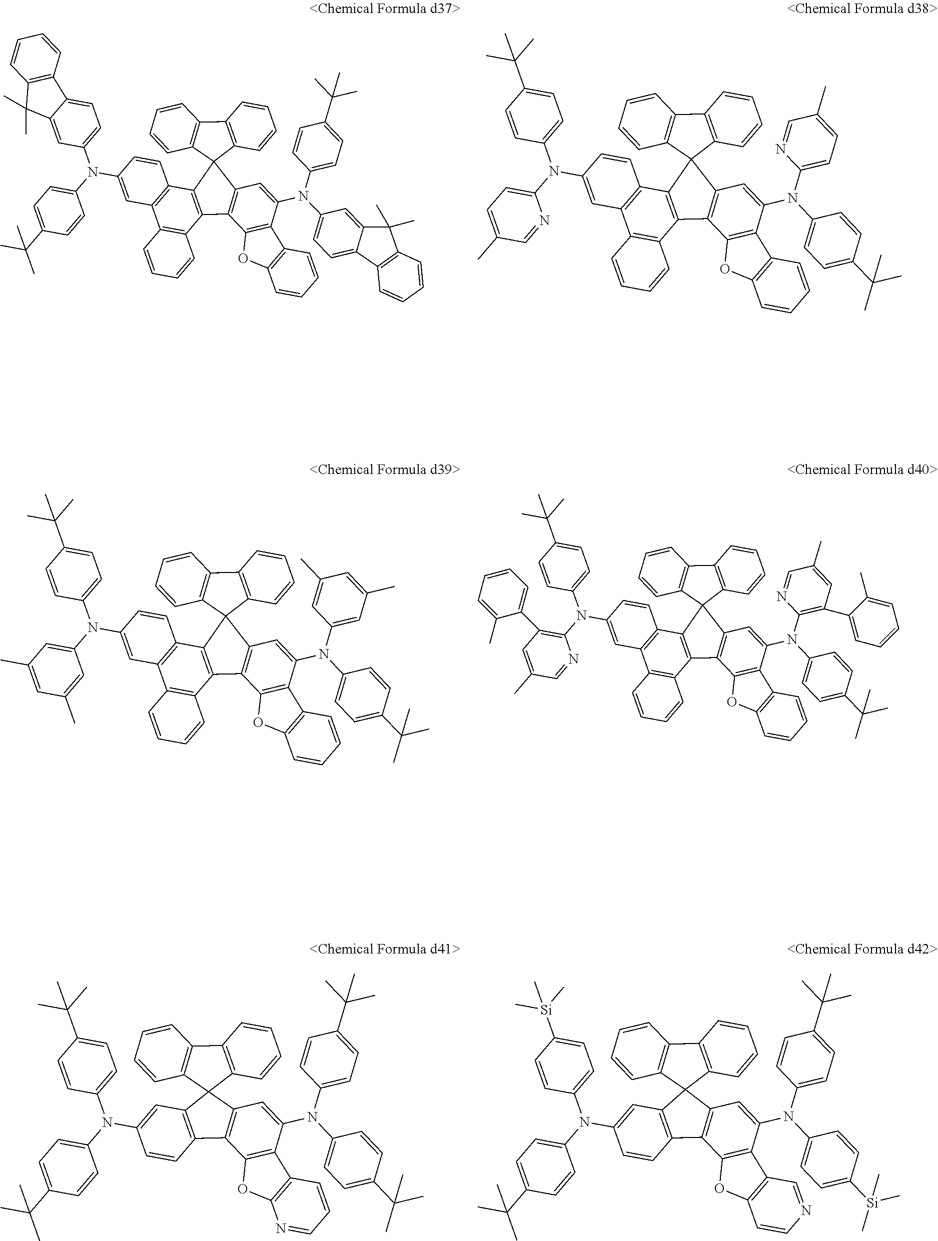

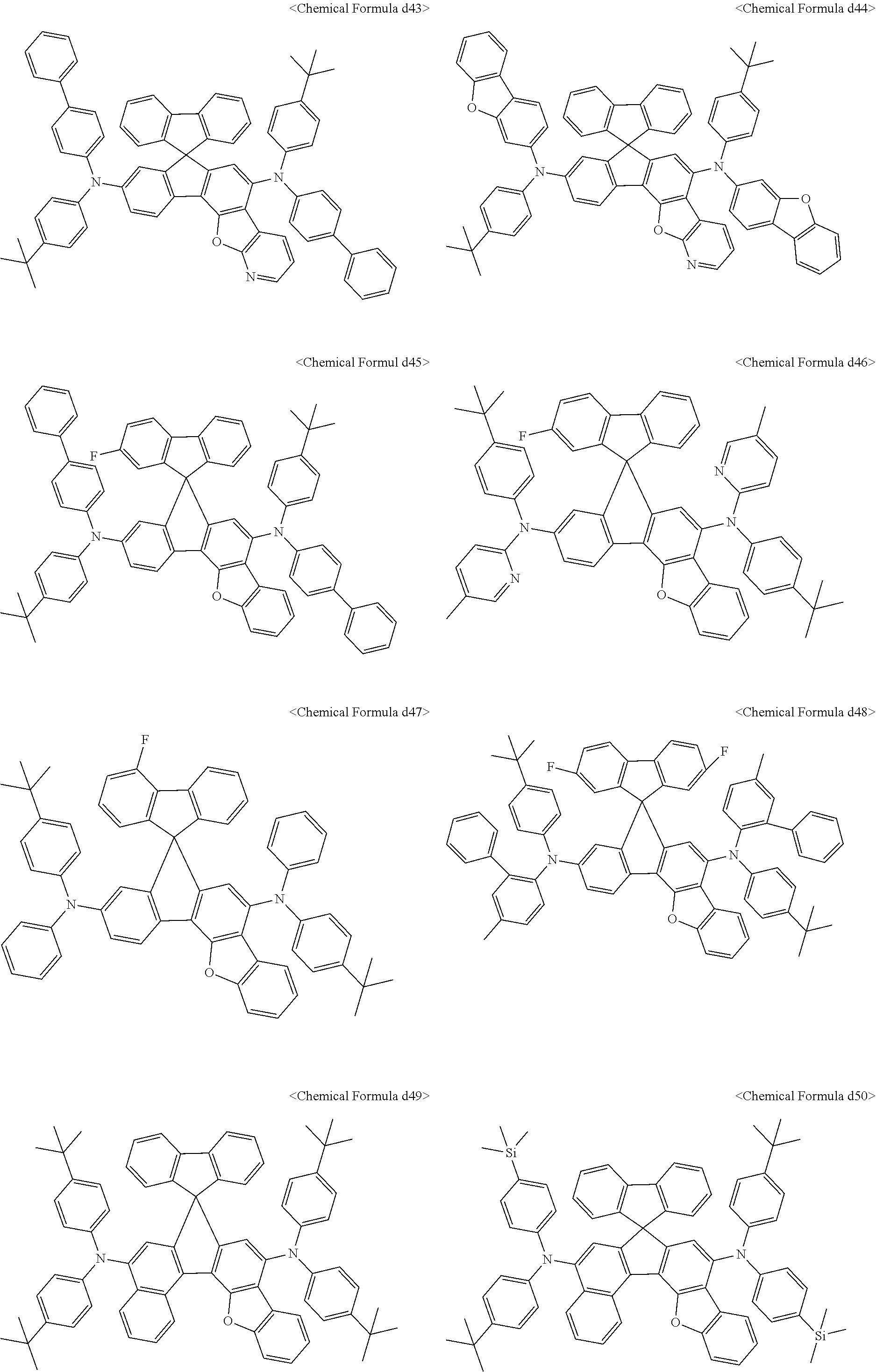

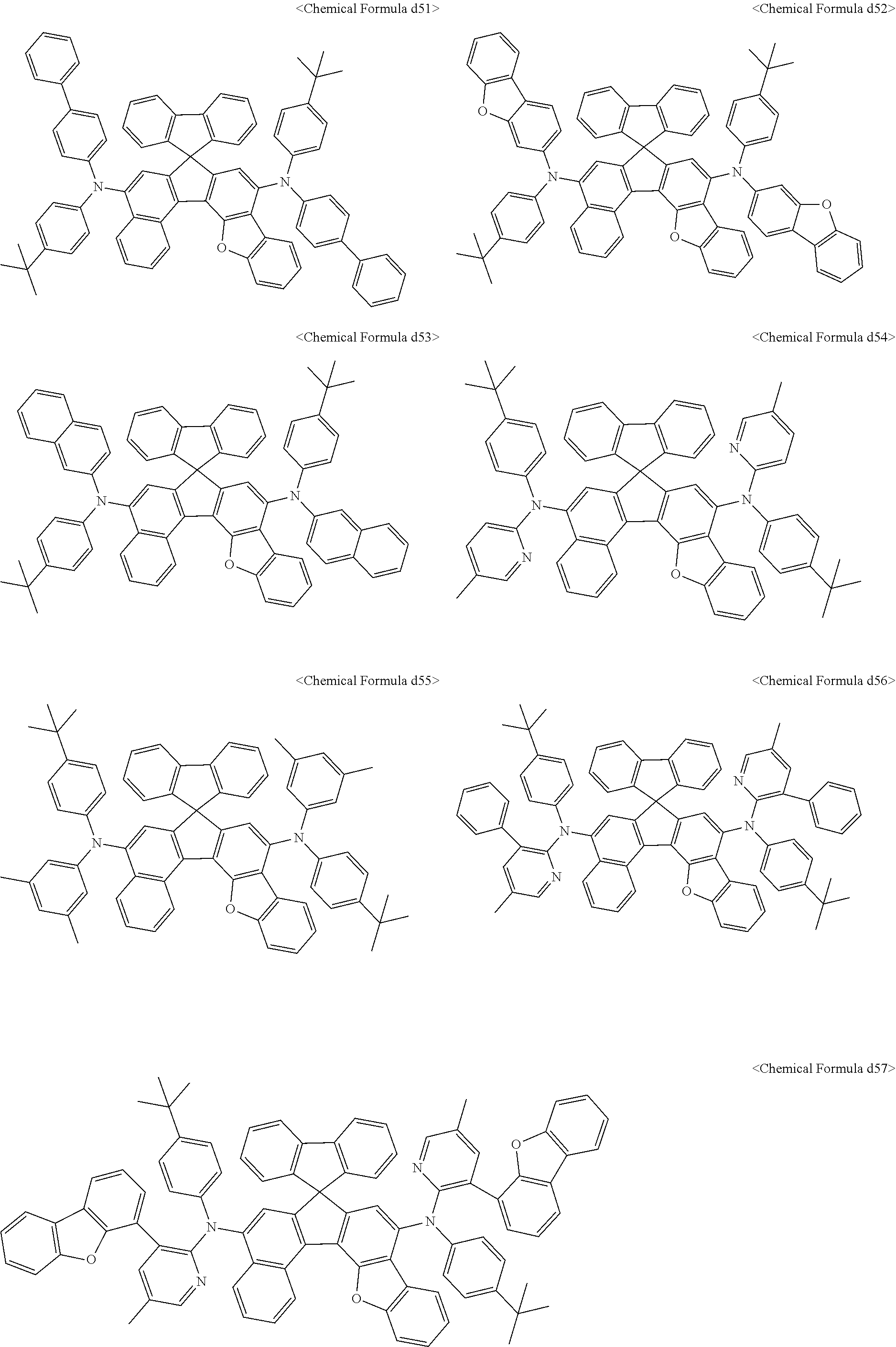

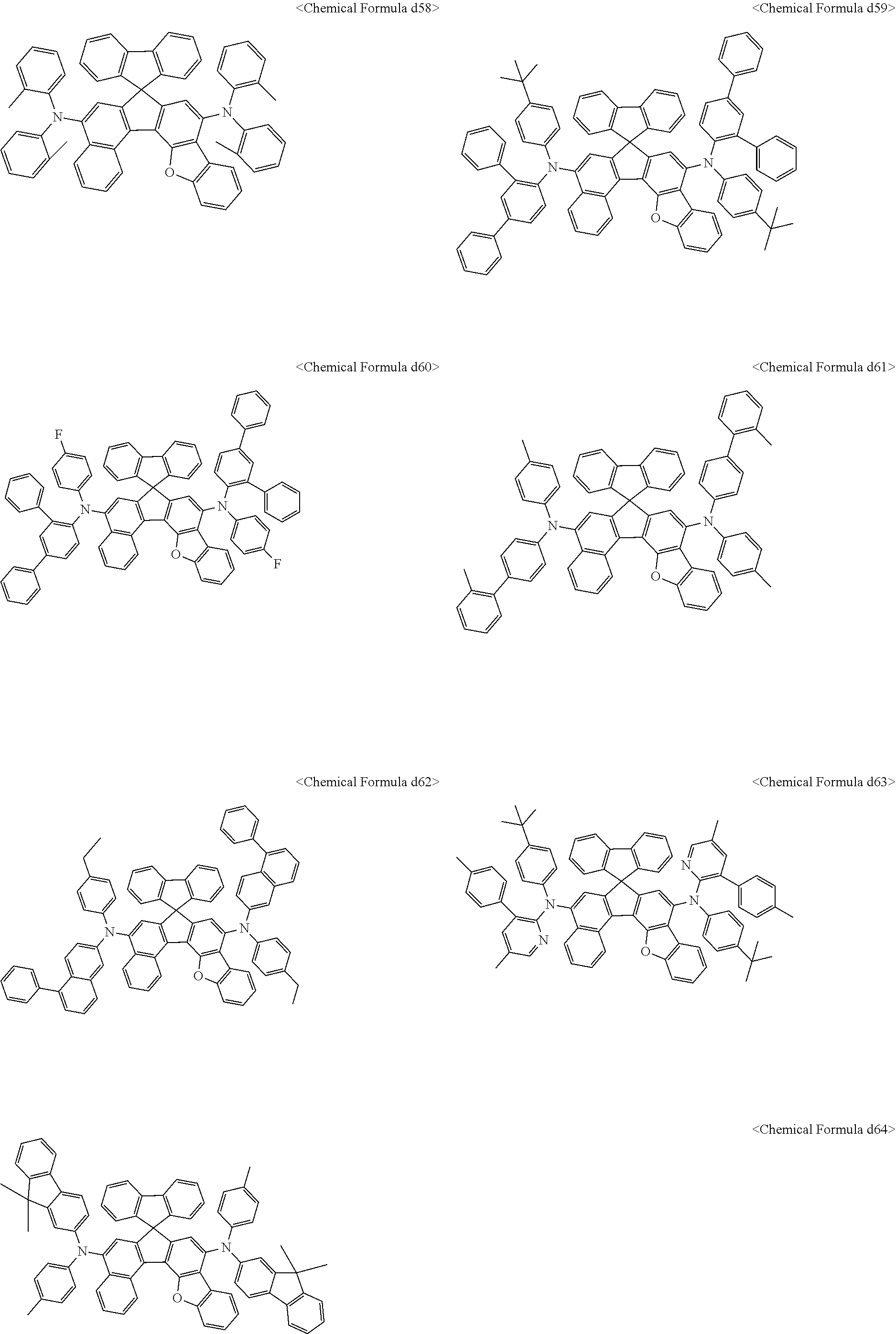

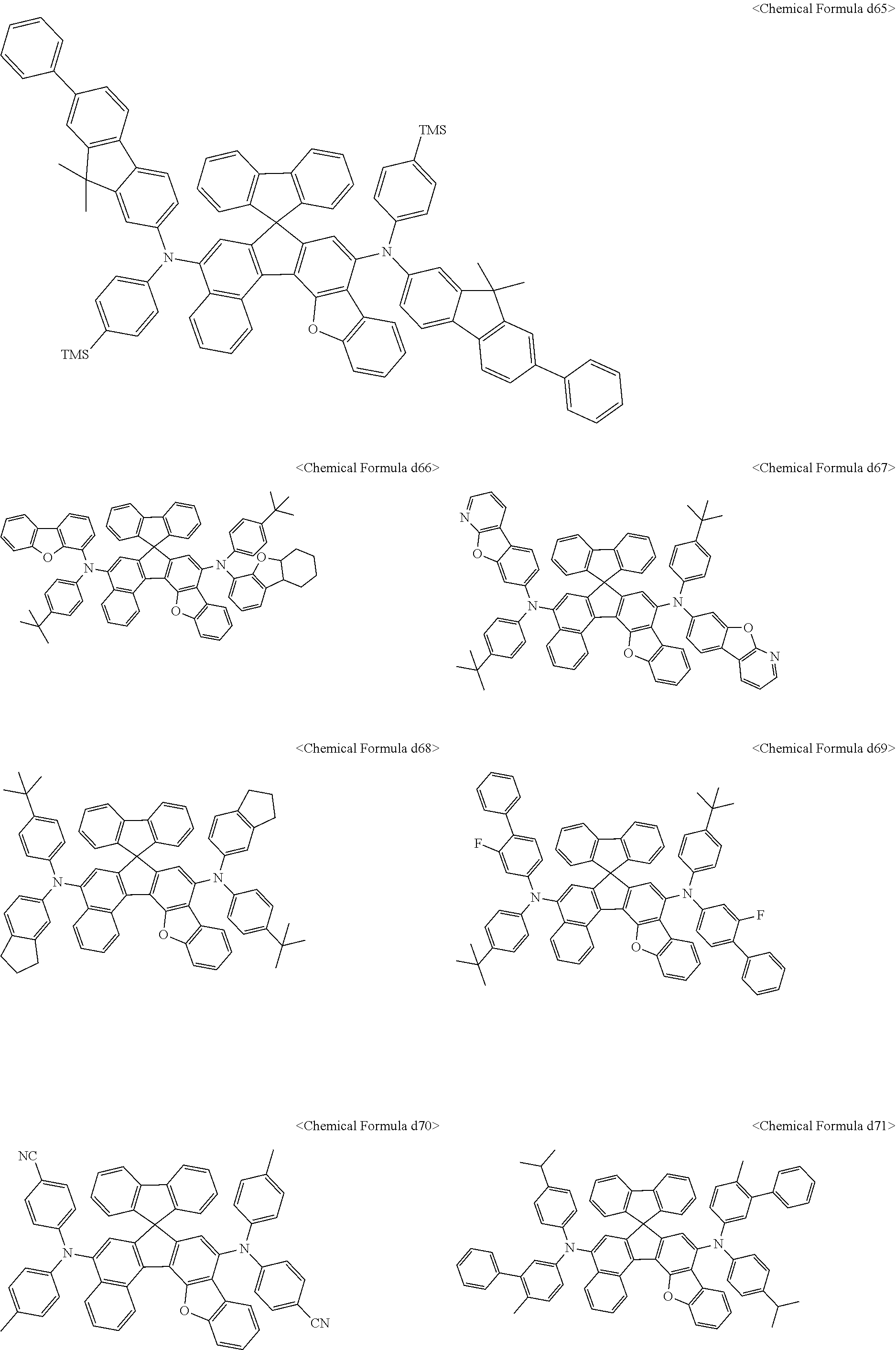

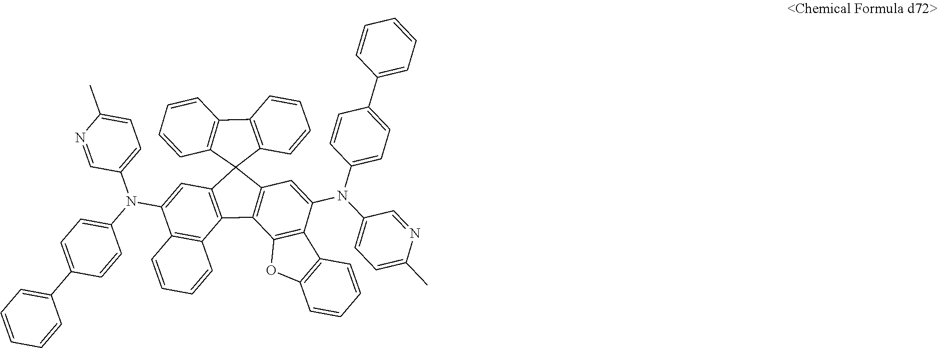 [SMILES: Cc1ccc(N(c2ccc(-c3ccccc3)cc2)c2cc3c(c4ccccc24)-c2c(cc(N(c4ccc(-c5ccccc5)cc4)c4ccc(C)nc4)c4c2oc2ccccc24)C32c3ccccc3-c3ccccc32)cn1]

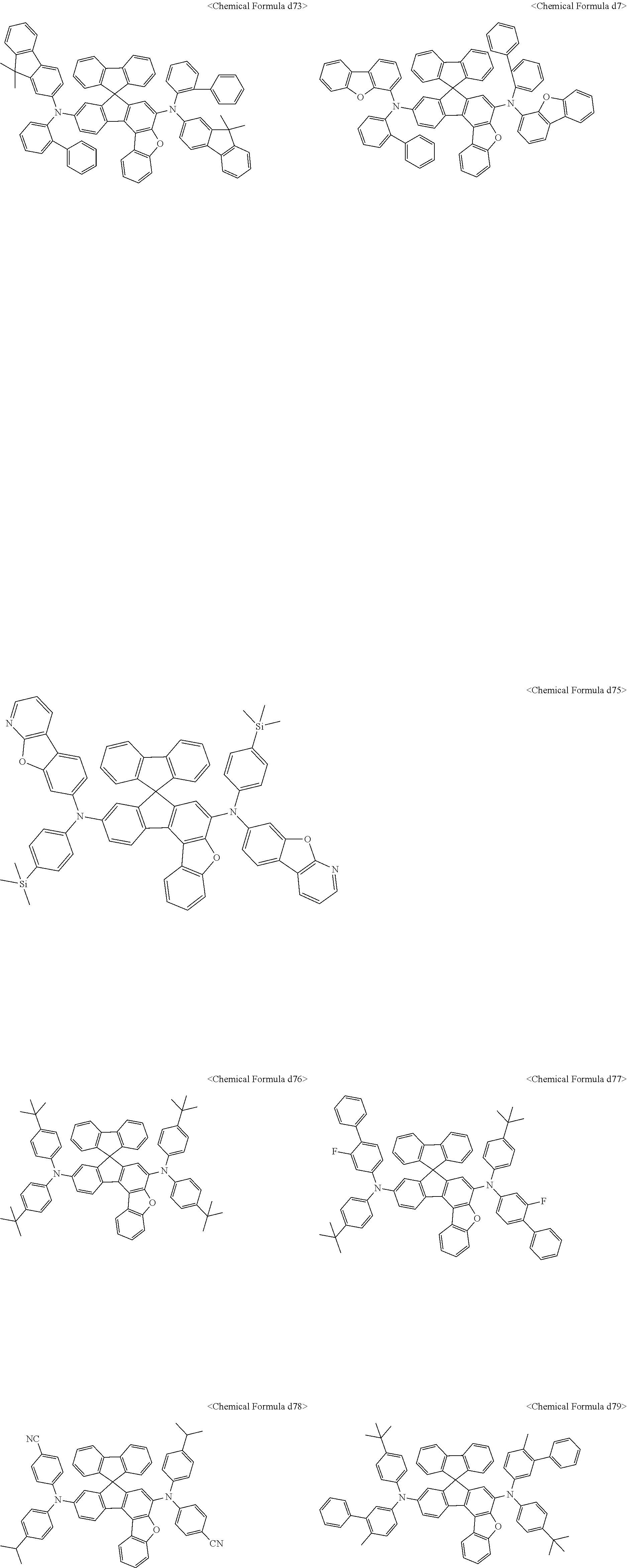

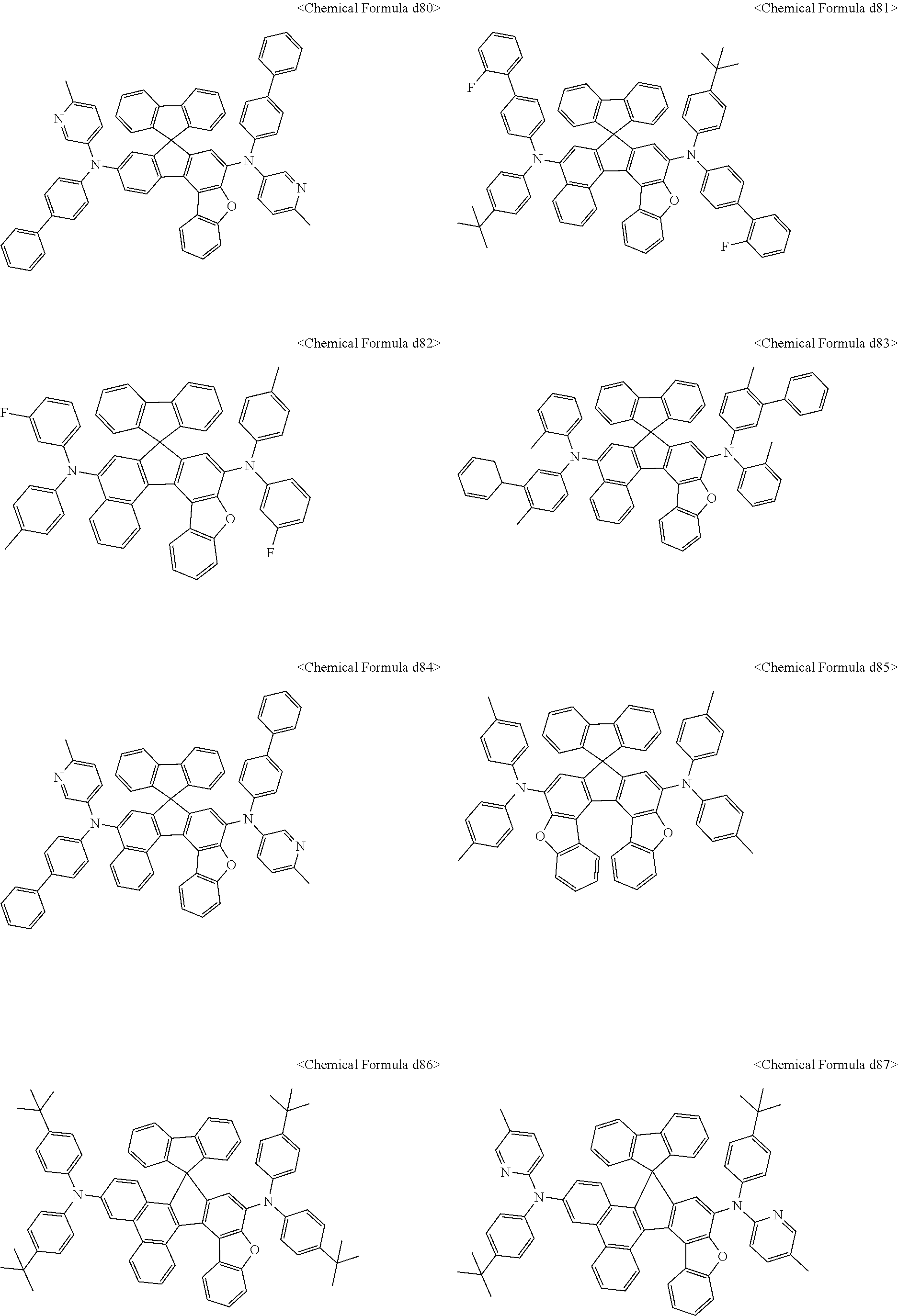

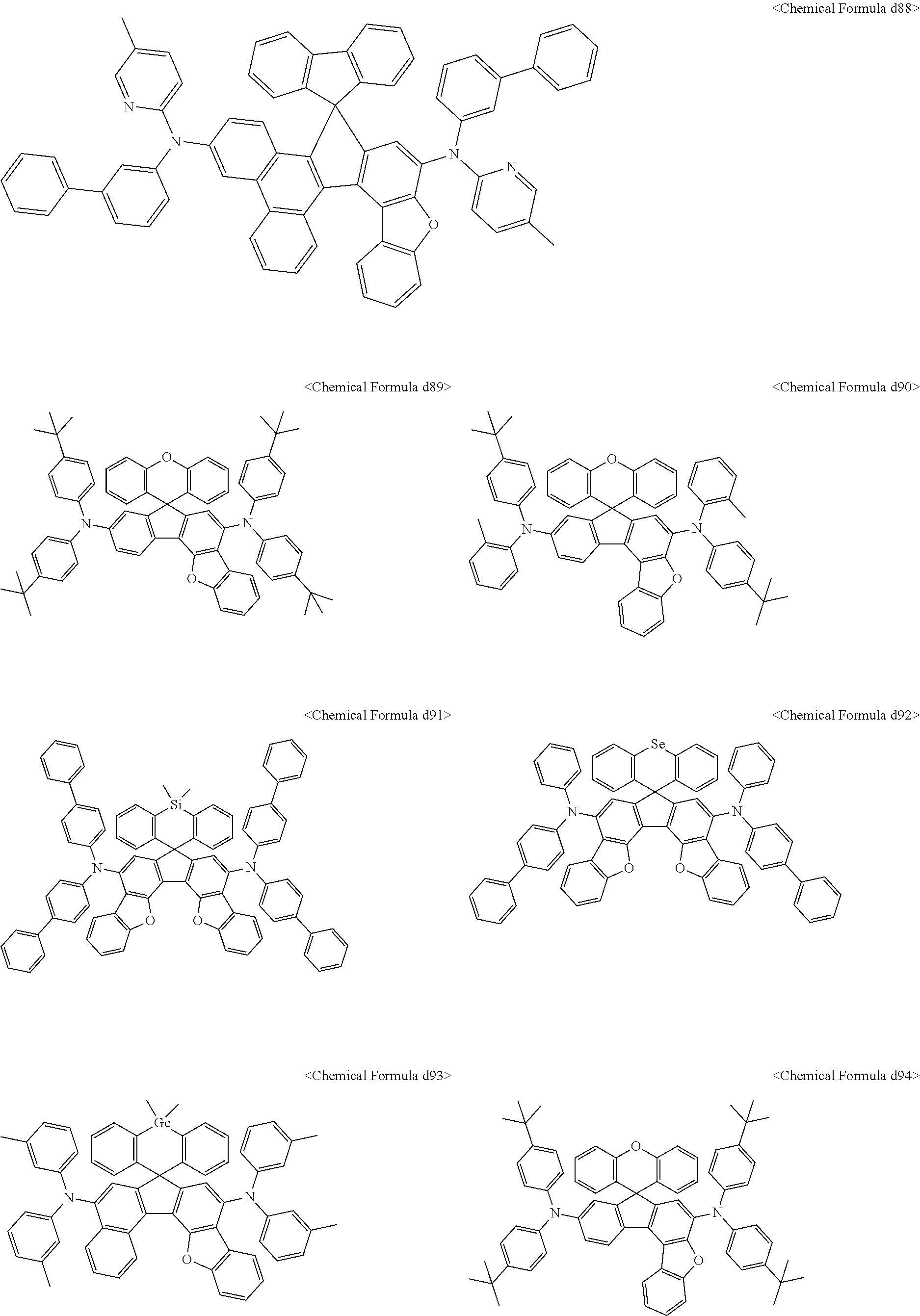

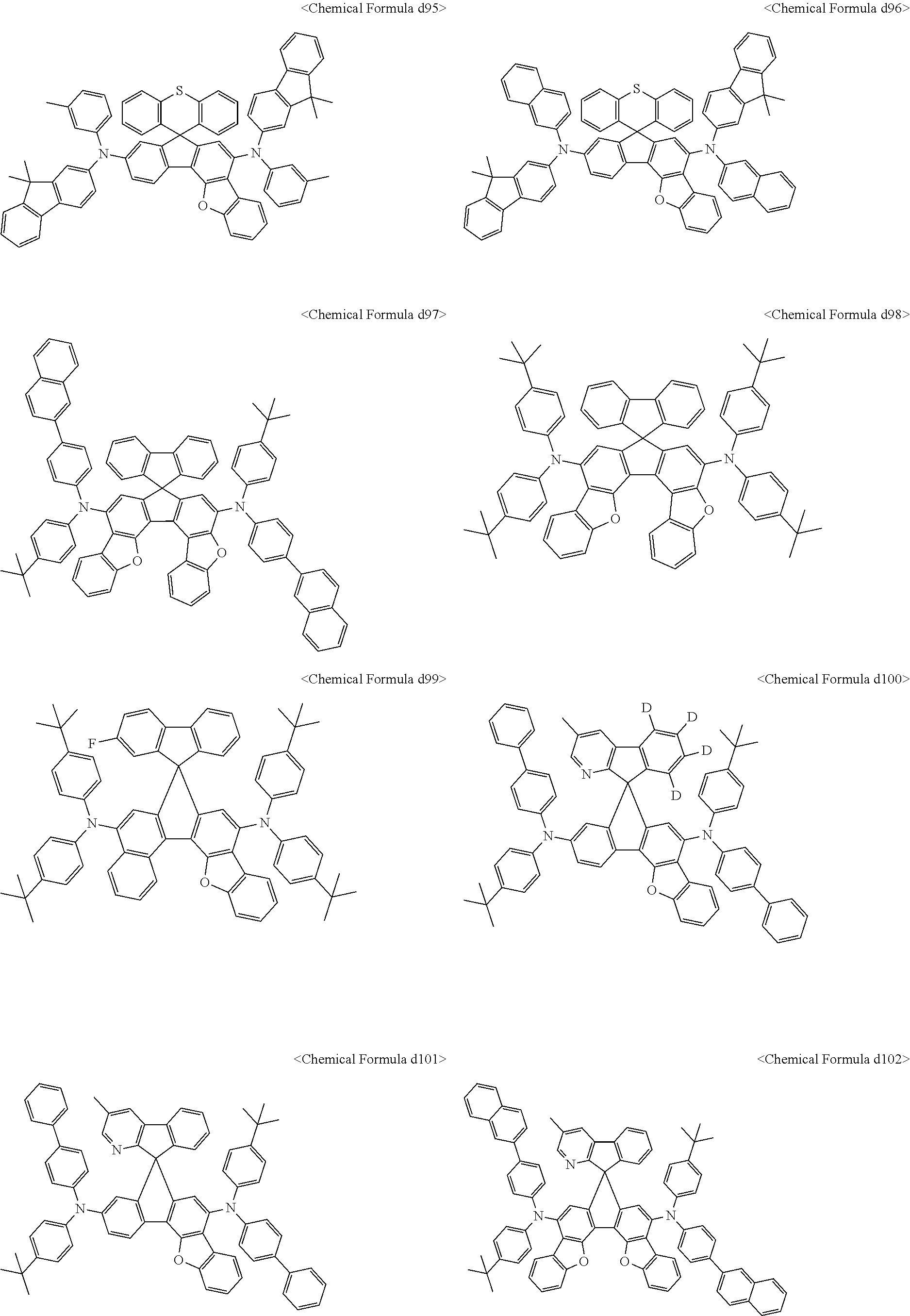

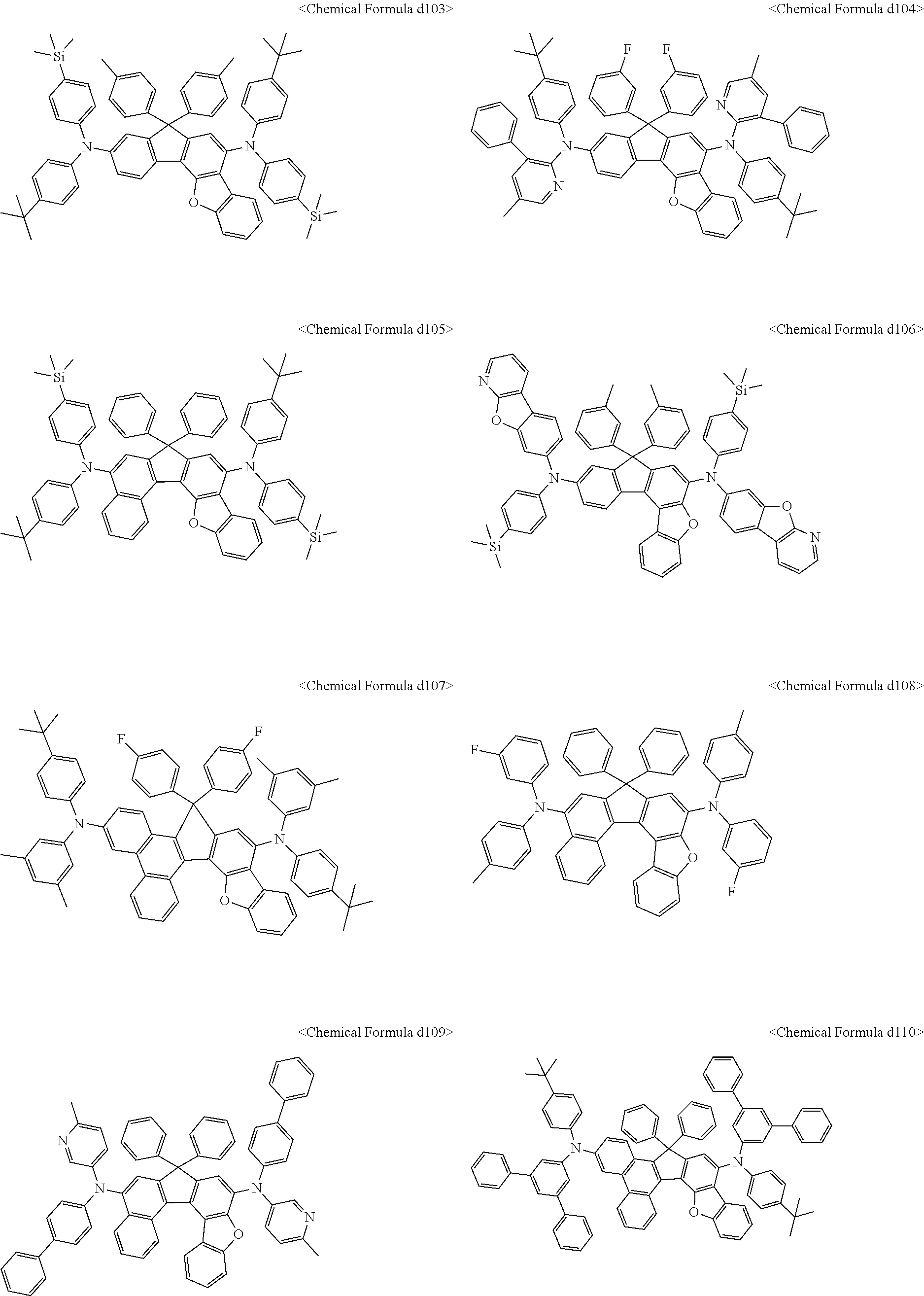

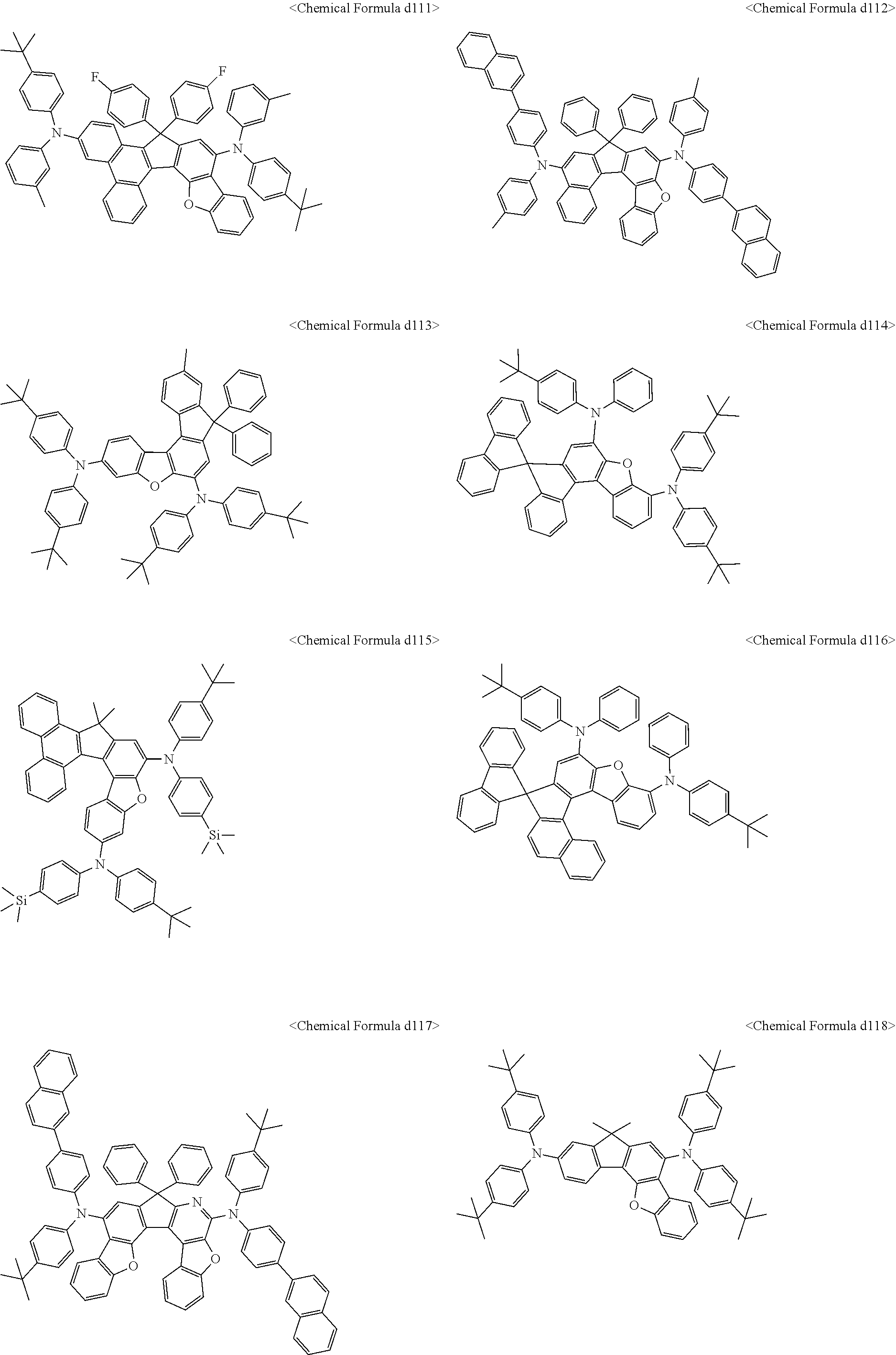

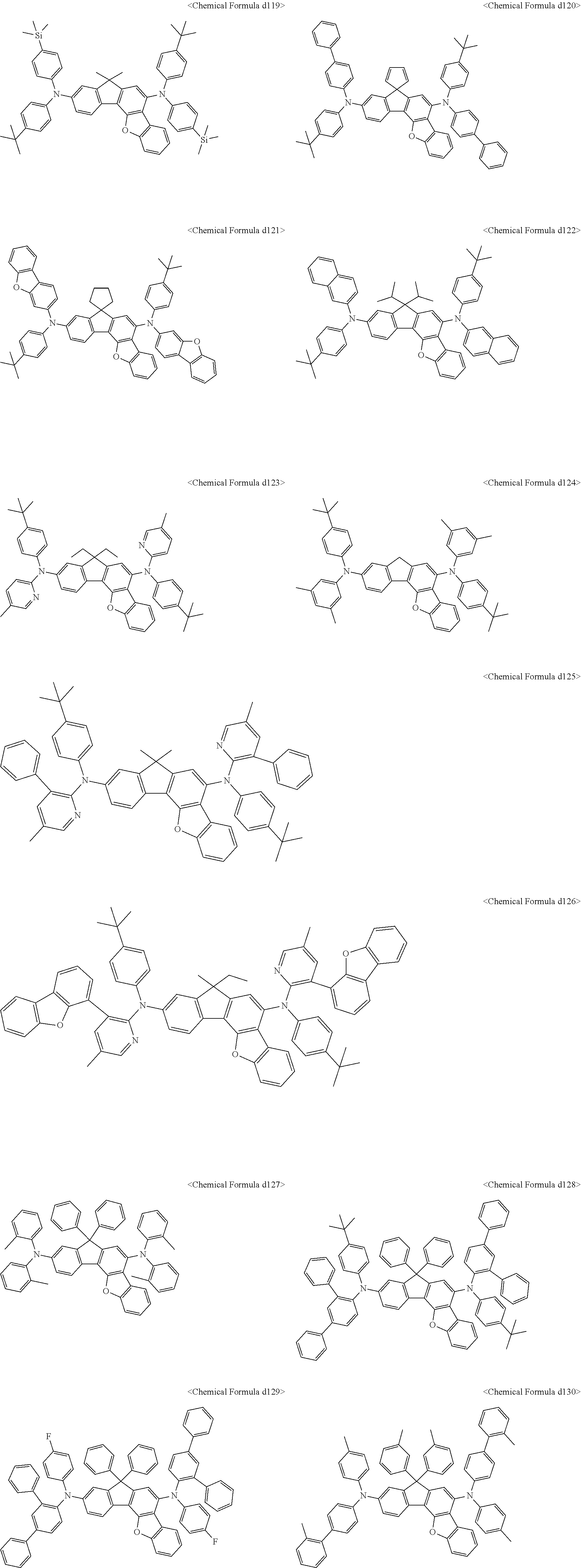

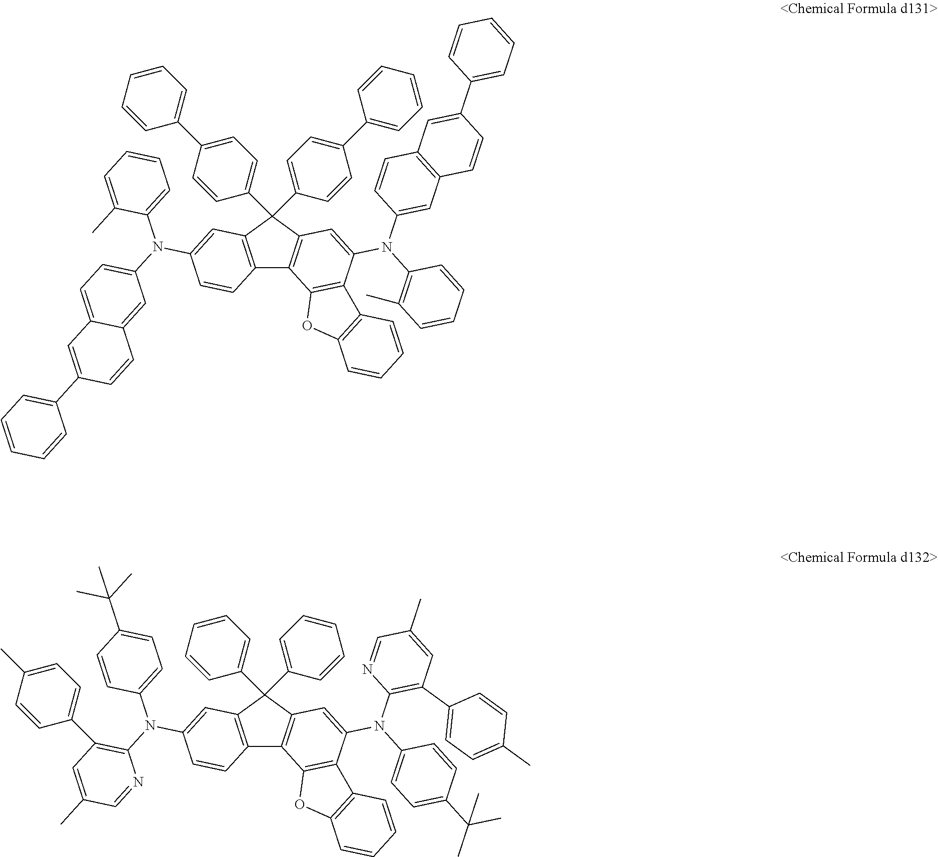

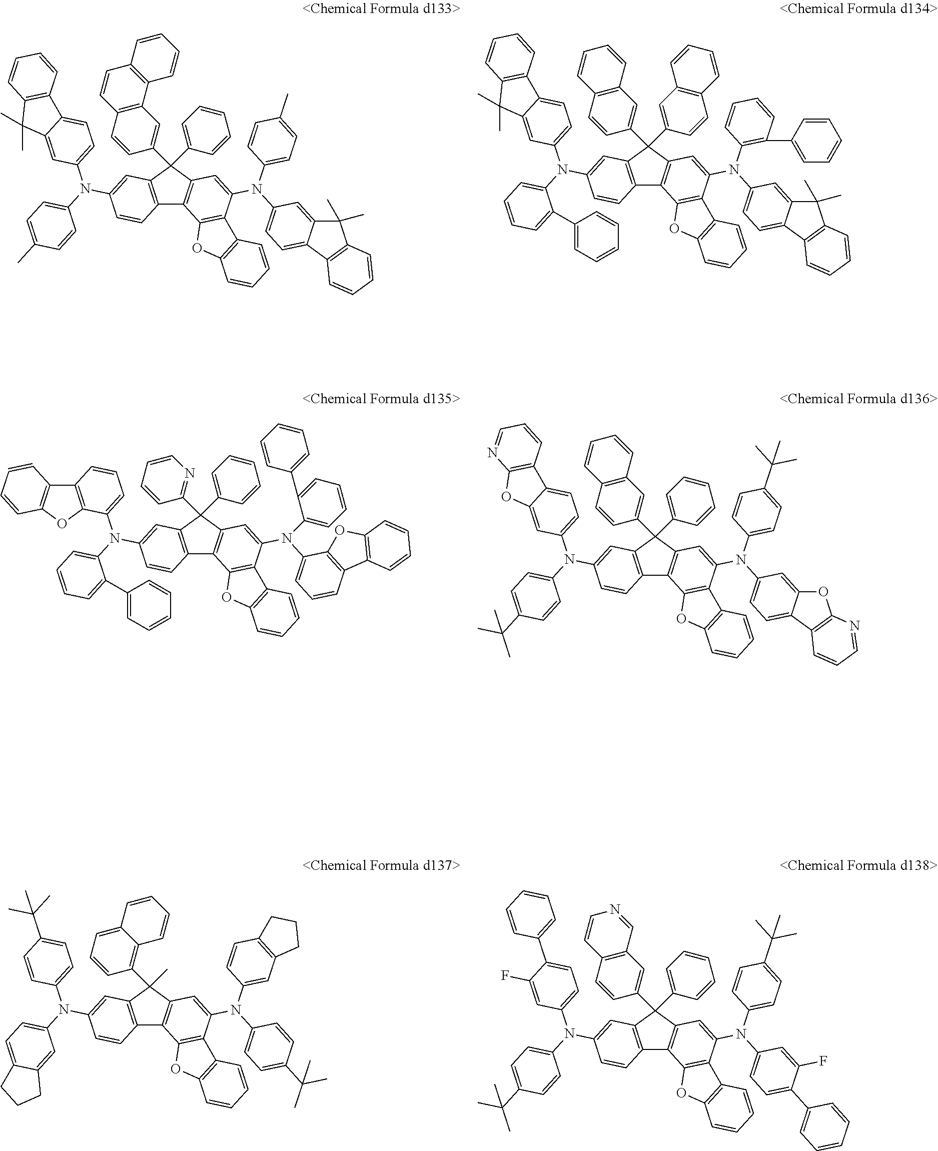

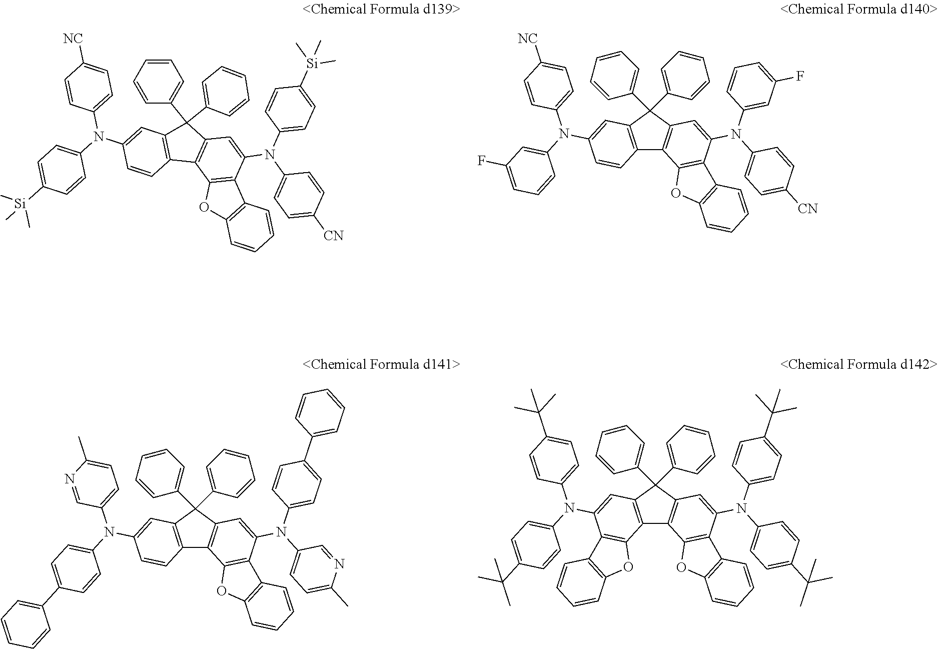

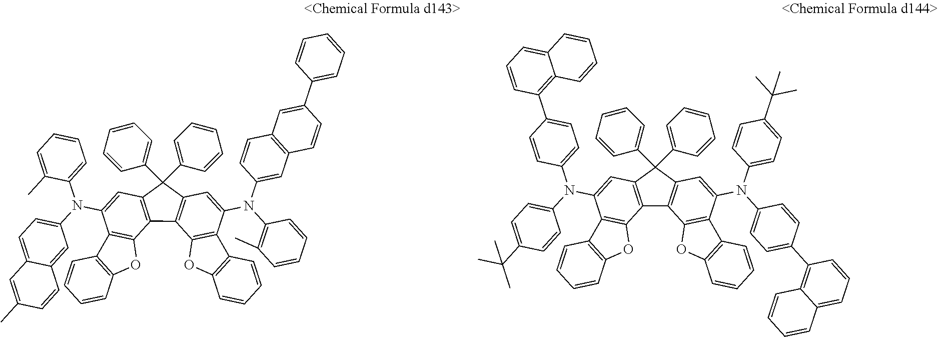 [SMILES: Cc1ccc2cc(N(c3ccccc3C)c3cc4c(c5oc6ccccc6c35)-c3c(cc(N(c5ccc6cc(-c7ccccc7)ccc6c5)c5ccccc5C)c5c3oc3ccccc35)C4(c3ccccc3)c3ccccc3)ccc2c1 ; CC(C)(C)c1ccc(N(c2ccc(-c3cccc4ccccc34)cc2)c2cc3c(c4oc5ccccc5c24)-c2c(cc(N(c4ccc(-c5cccc6ccccc56)cc4)c4ccc(C(C)(C)C)cc4)c4c2oc2ccccc24)C3(c2ccccc2)c2ccccc2)cc1]

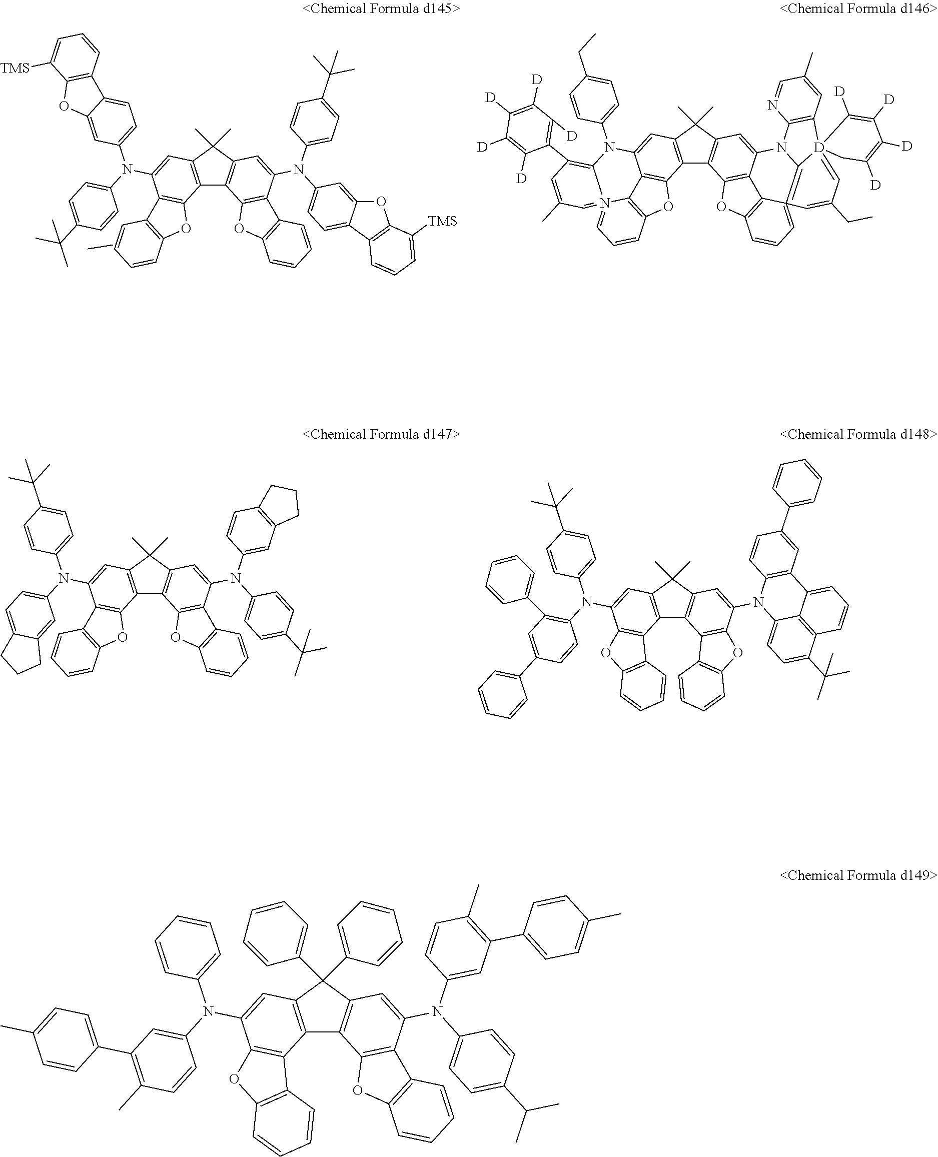

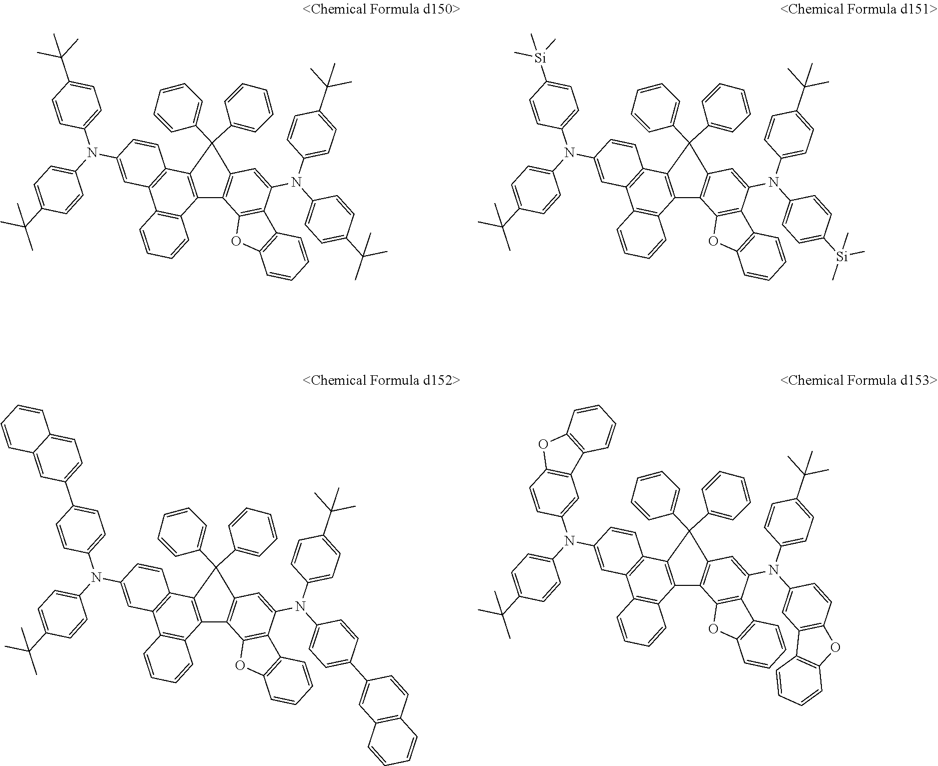

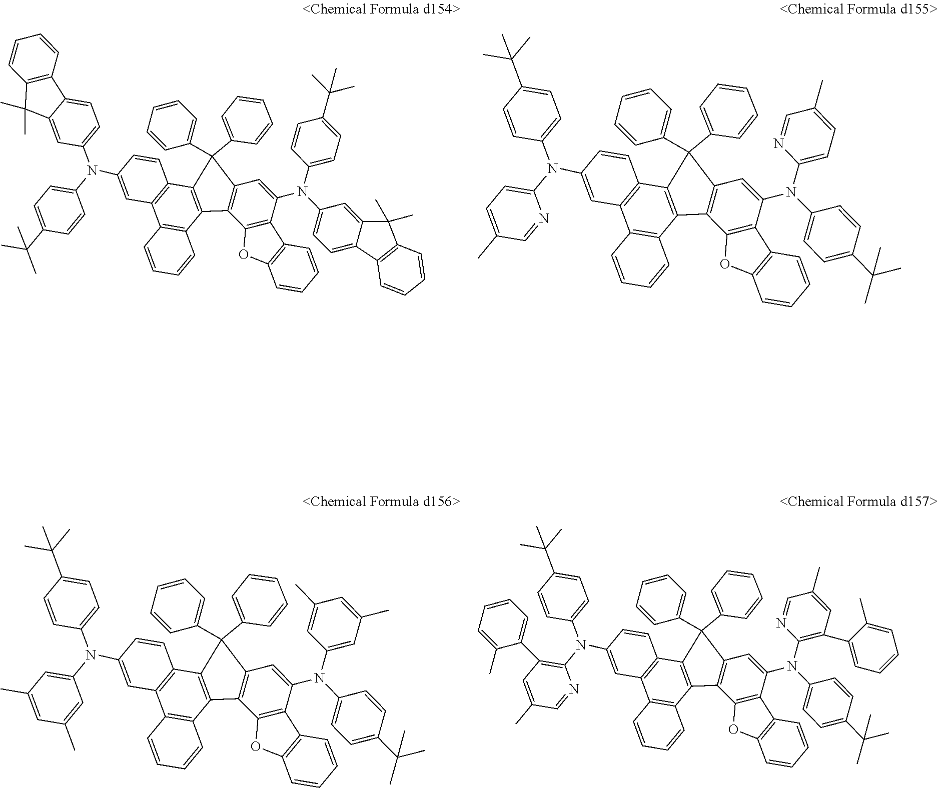

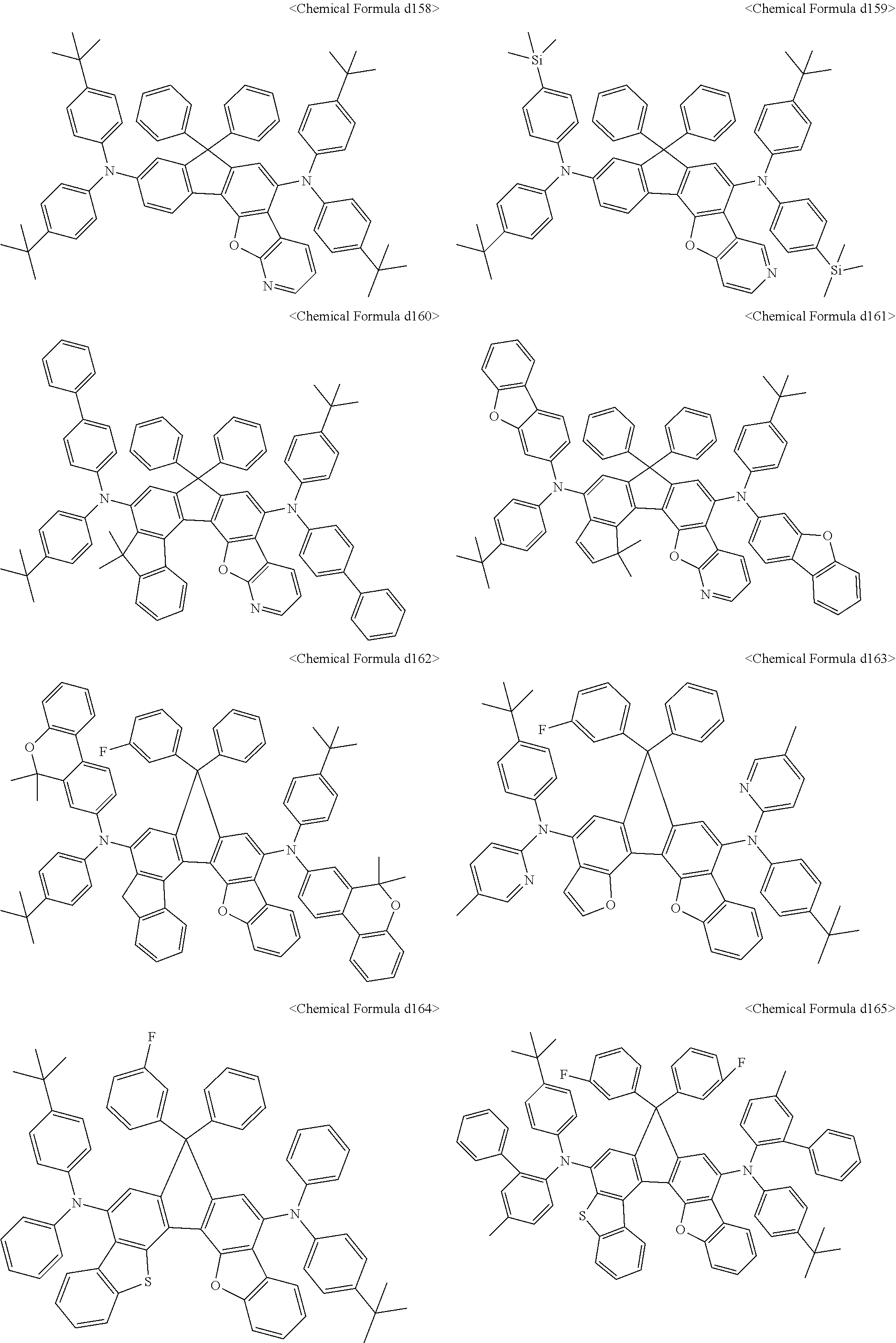

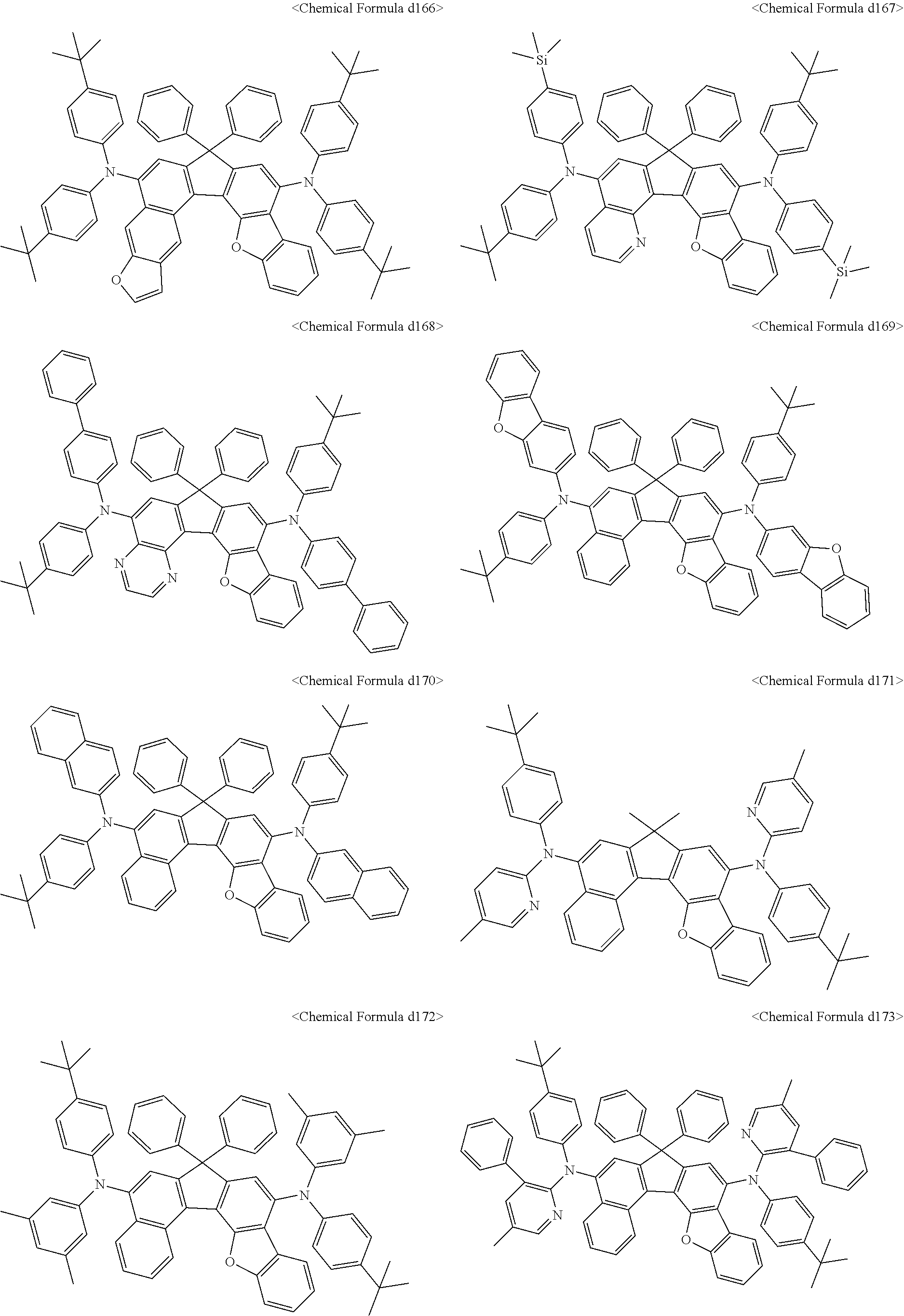

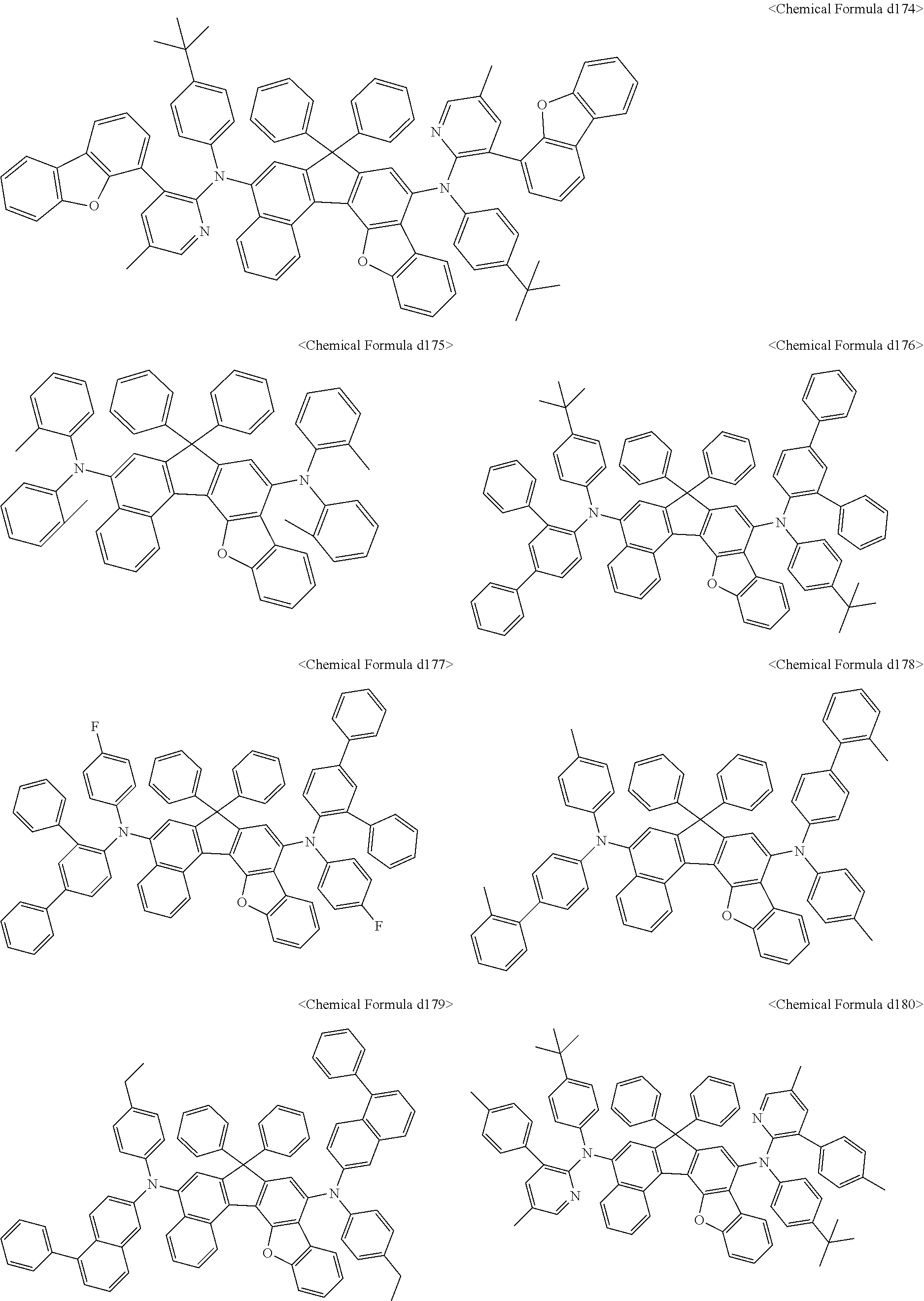

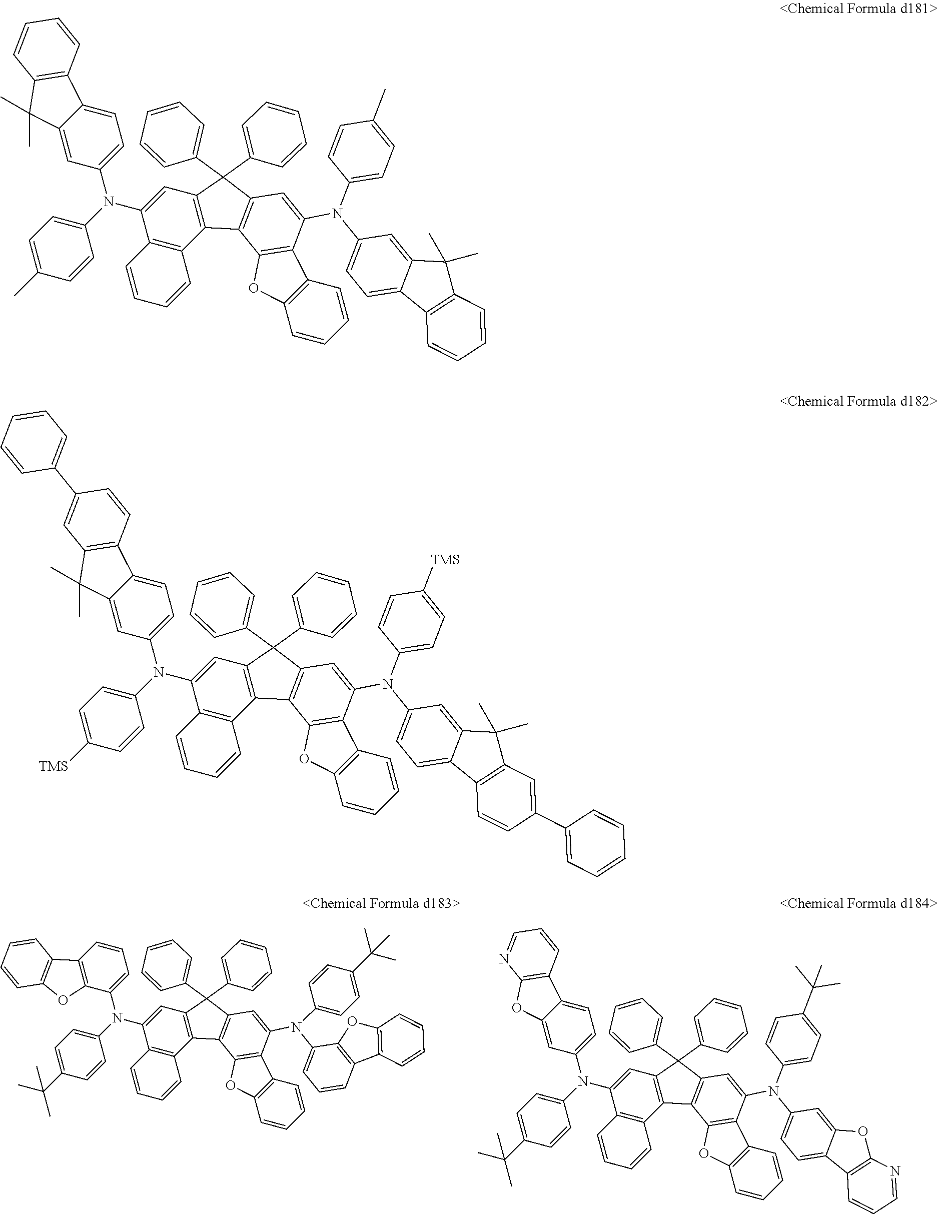

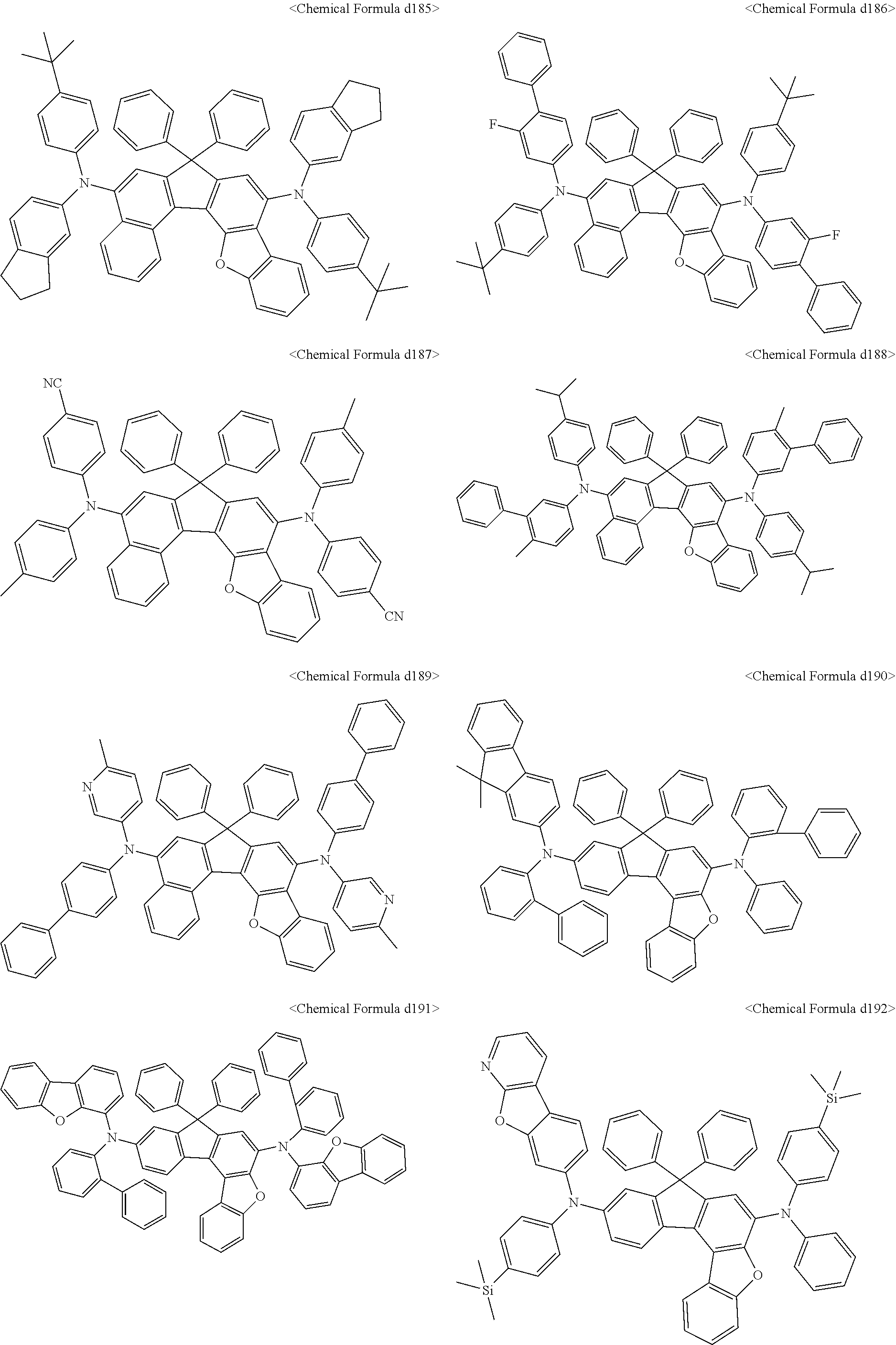

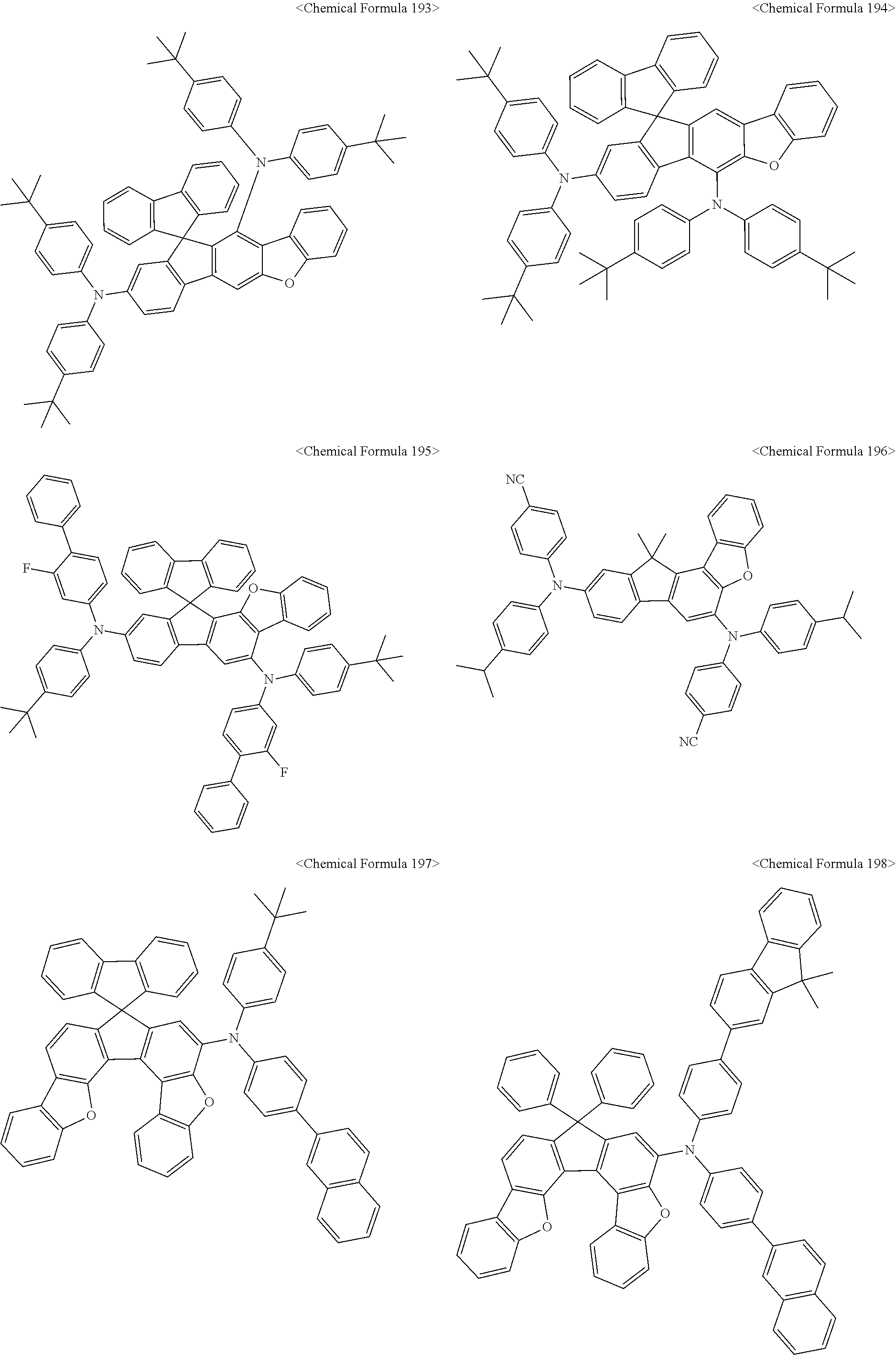

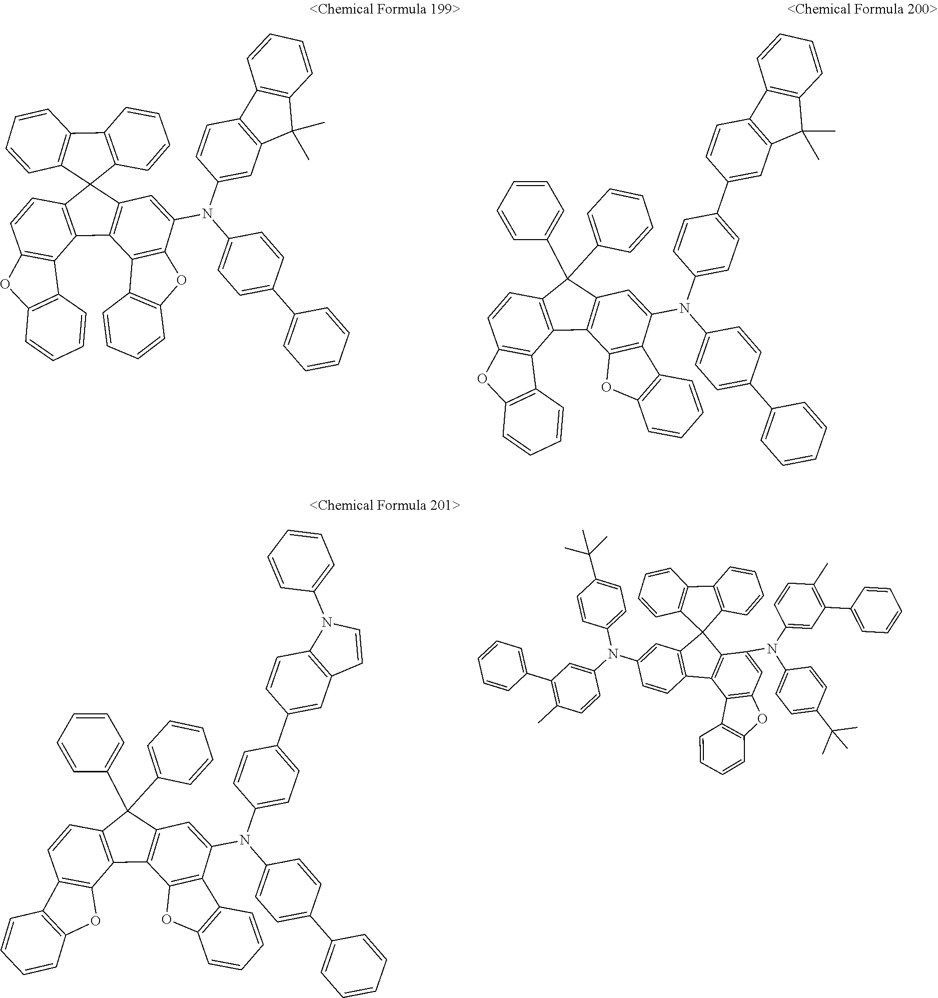

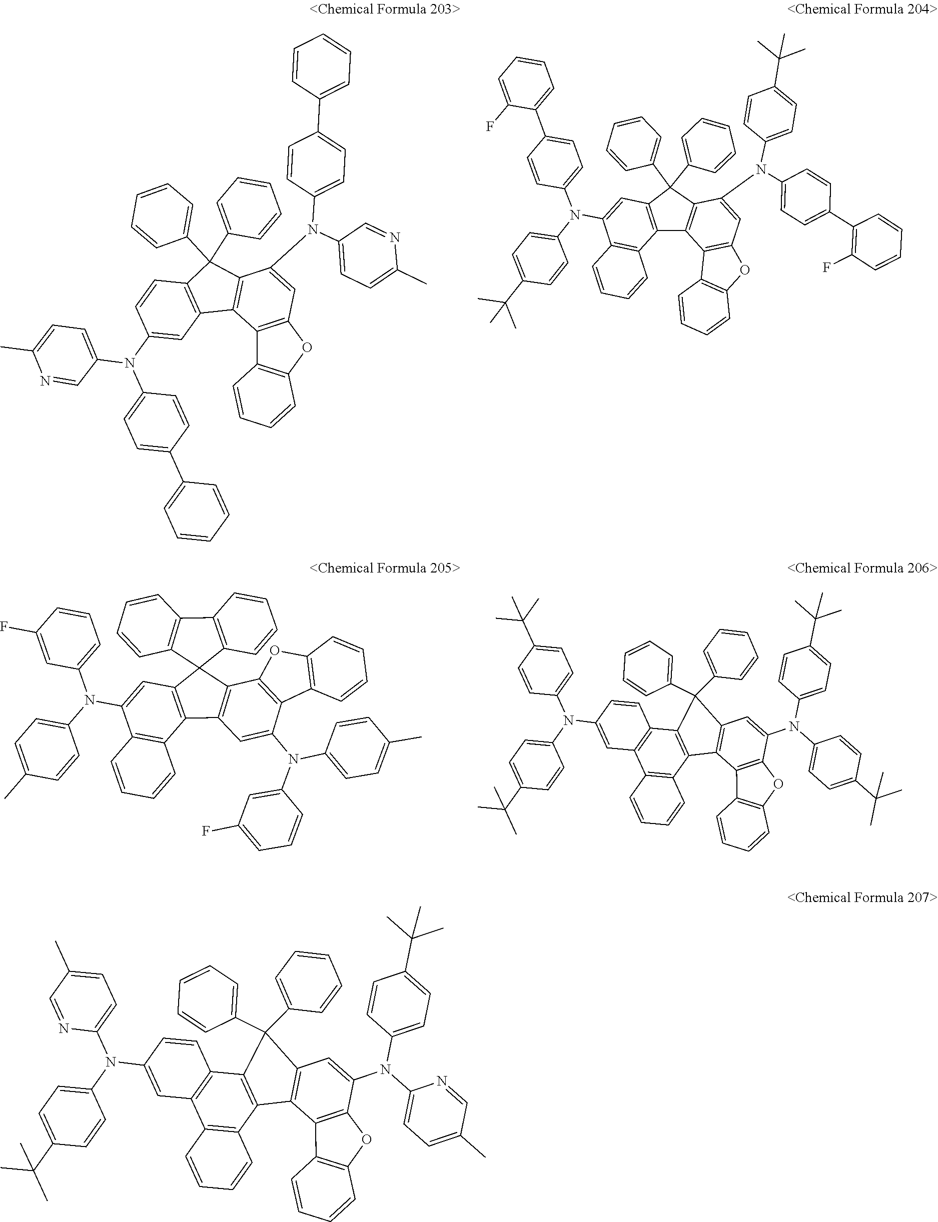

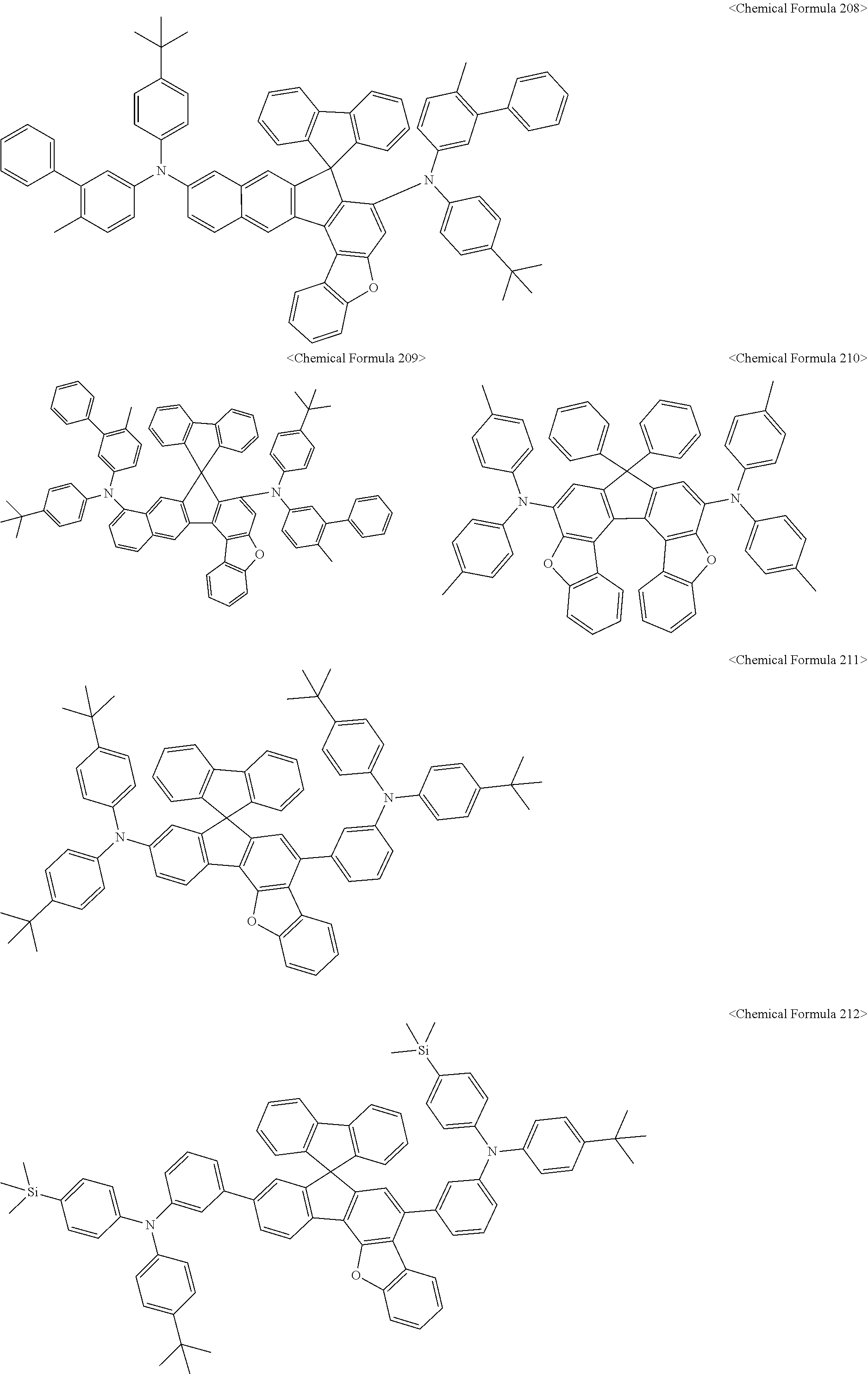

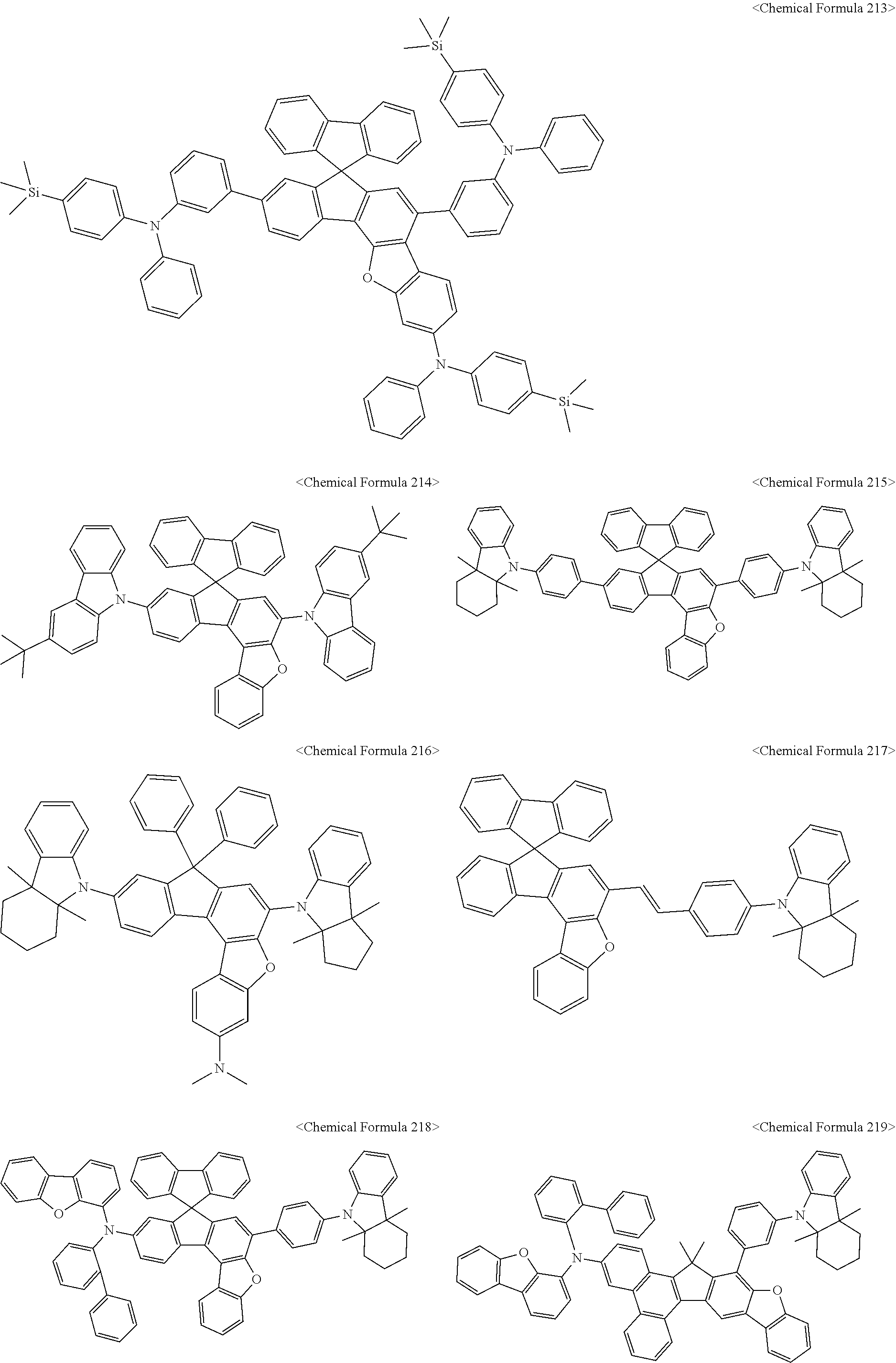

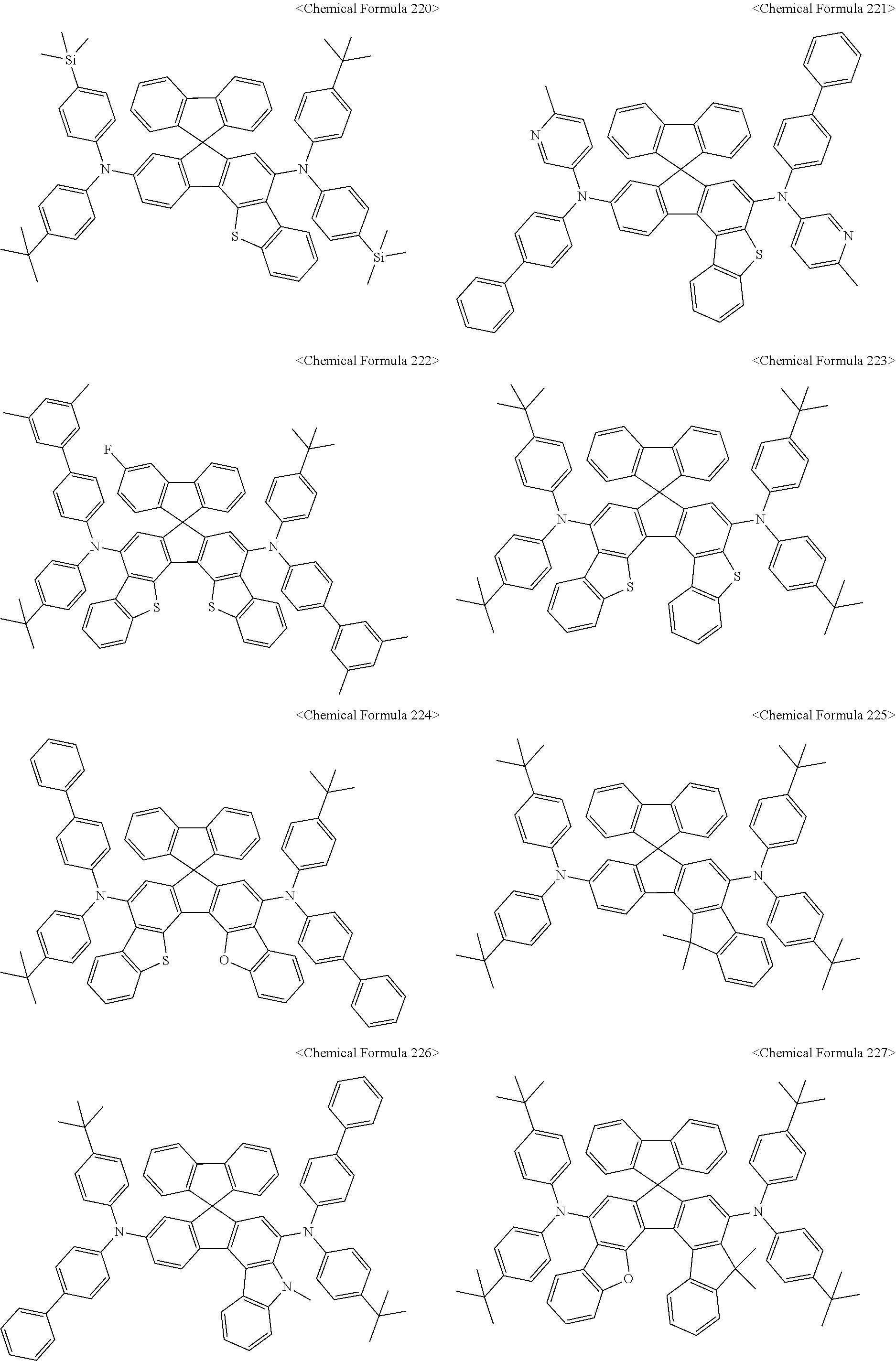

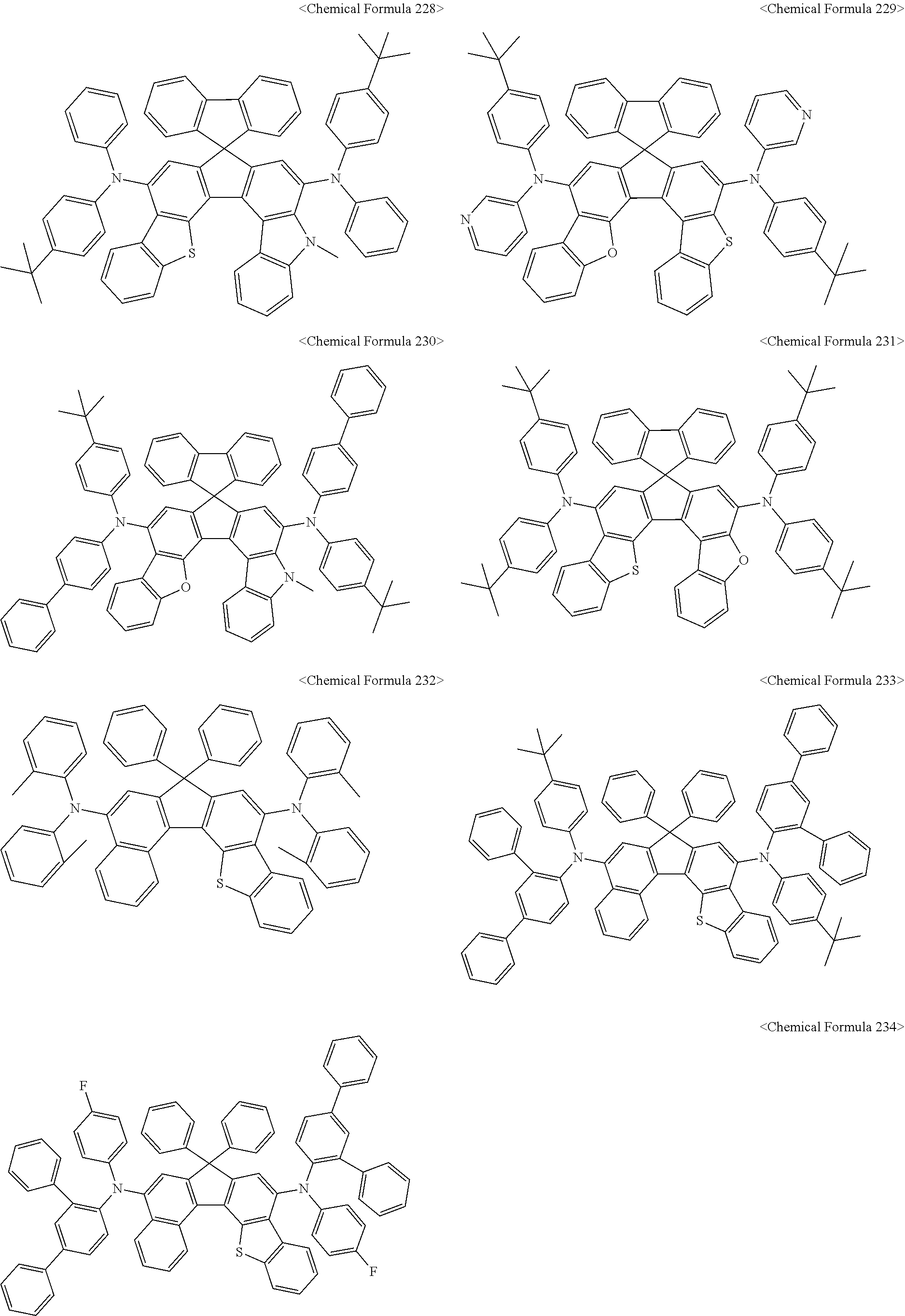

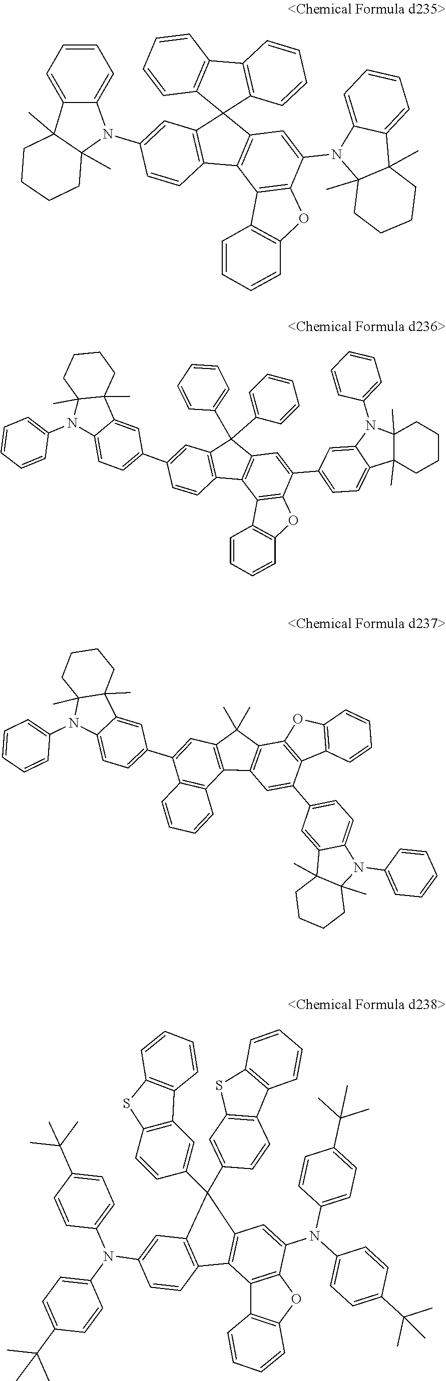

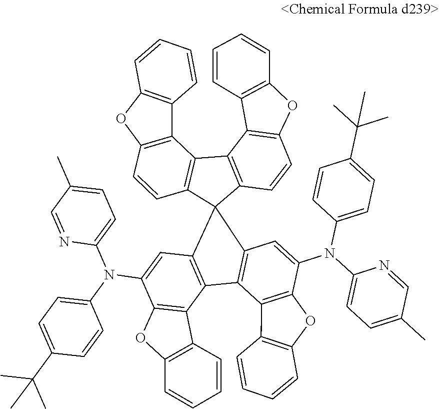 [SMILES: Cc1ccc(N(c2ccc(C(C)(C)C)cc2)c2cc3c(c4c2oc2ccccc24)-c2c(cc(N(c4ccc(C(C)(C)C)cc4)c4ccc(C)cn4)c4oc5ccccc5c24)C32c3ccc4oc5ccccc5c4c3-c3c2ccc2oc4ccccc4c32)nc1]

Examples of the compound represented by [Chemical Formula D3] include the compounds of the following <Chemical Formula D101> to <Chemical Formula D130>:

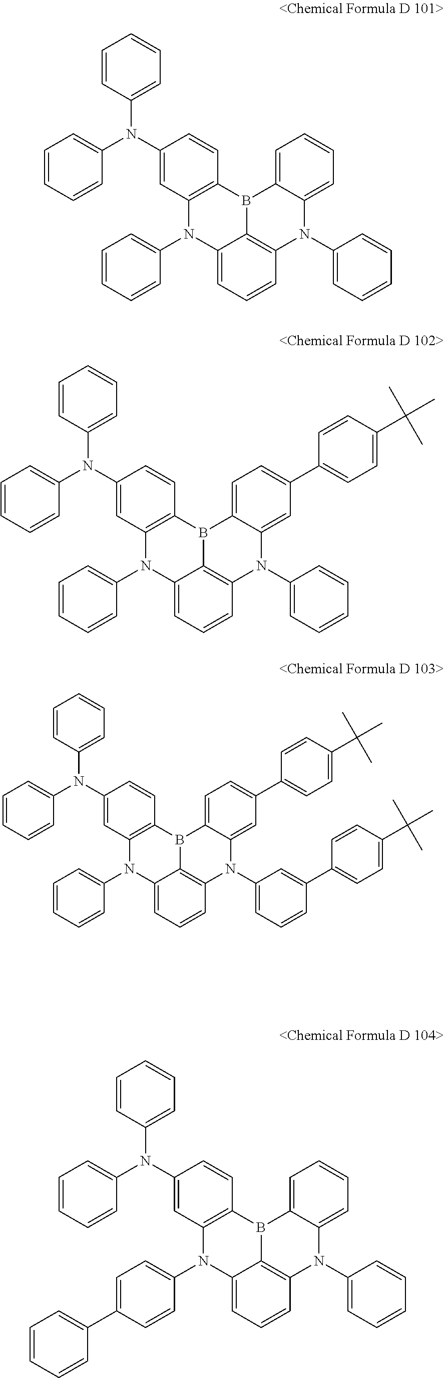 [SMILES: c1ccc(N(c2ccccc2)c2ccc3c(c2)N(c2ccccc2)c2cccc4c2B3c2ccccc2N4c2ccccc2)cc1 ; CC(C)(C)c1ccc(-c2ccc3c(c2)N(c2ccccc2)c2cccc4c2B3c2ccc(N(c3ccccc3)c3ccccc3)cc2N4c2ccccc2)cc1 ; CC(C)(C)c1ccc(-c2cccc(N3c4cc(-c5ccc(C(C)(C)C)cc5)ccc4B4c5ccc(N(c6ccccc6)c6ccccc6)cc5N(c5ccccc5)c5cccc3c54)c2)cc1 ; c1ccc(-c2ccc(N3c4cc(N(c5ccccc5)c5ccccc5)ccc4B4c5ccccc5N(c5ccccc5)c5cccc3c54)cc2)cc1]

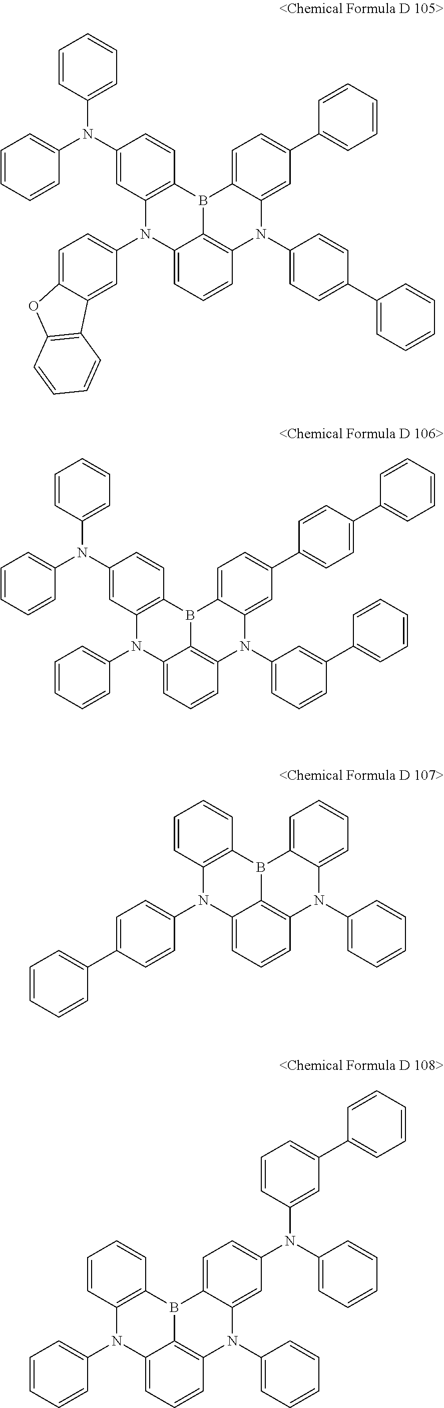 [SMILES: c1ccc(-c2ccc(N3c4cc(-c5ccccc5)ccc4B4c5ccc(N(c6ccccc6)c6ccccc6)cc5N(c5ccc6oc7ccccc7c6c5)c5cccc3c54)cc2)cc1 ; c1ccc(-c2ccc(-c3ccc4c(c3)N(c3cccc(-c5ccccc5)c3)c3cccc5c3B4c3ccc(N(c4ccccc4)c4ccccc4)cc3N5c3ccccc3)cc2)cc1 ; c1ccc(-c2ccc(N3c4ccccc4B4c5ccccc5N(c5ccccc5)c5cccc3c54)cc2)cc1 ; c1ccc(-c2cccc(N(c3ccccc3)c3ccc4c(c3)N(c3ccccc3)c3cccc5c3B4c3ccccc3N5c3ccccc3)c2)cc1]

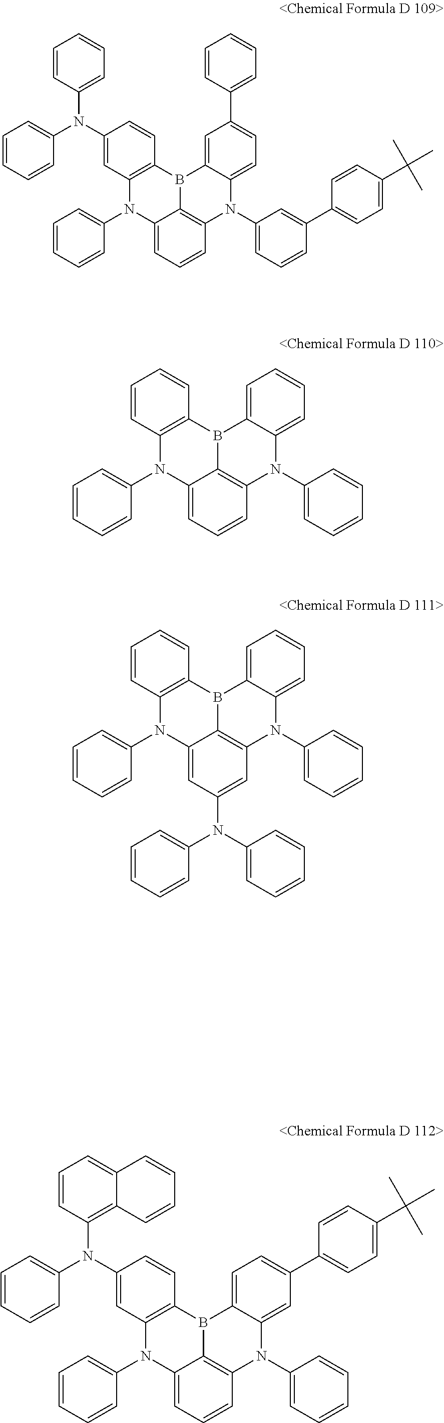 [SMILES: CC(C)(C)c1ccc(-c2cccc(N3c4ccc(-c5ccccc5)cc4B4c5ccc(N(c6ccccc6)c6ccccc6)cc5N(c5ccccc5)c5cccc3c54)c2)cc1 ; c1ccc(N2c3ccccc3B3c4ccccc4N(c4ccccc4)c4cccc2c43)cc1 ; c1ccc(N(c2ccccc2)c2cc3c4c(c2)N(c2ccccc2)c2ccccc2B4c2ccccc2N3c2ccccc2)cc1 ; CC(C)(C)c1ccc(-c2ccc3c(c2)N(c2ccccc2)c2cccc4c2B3c2ccc(N(c3ccccc3)c3cccc5ccccc35)cc2N4c2ccccc2)cc1]

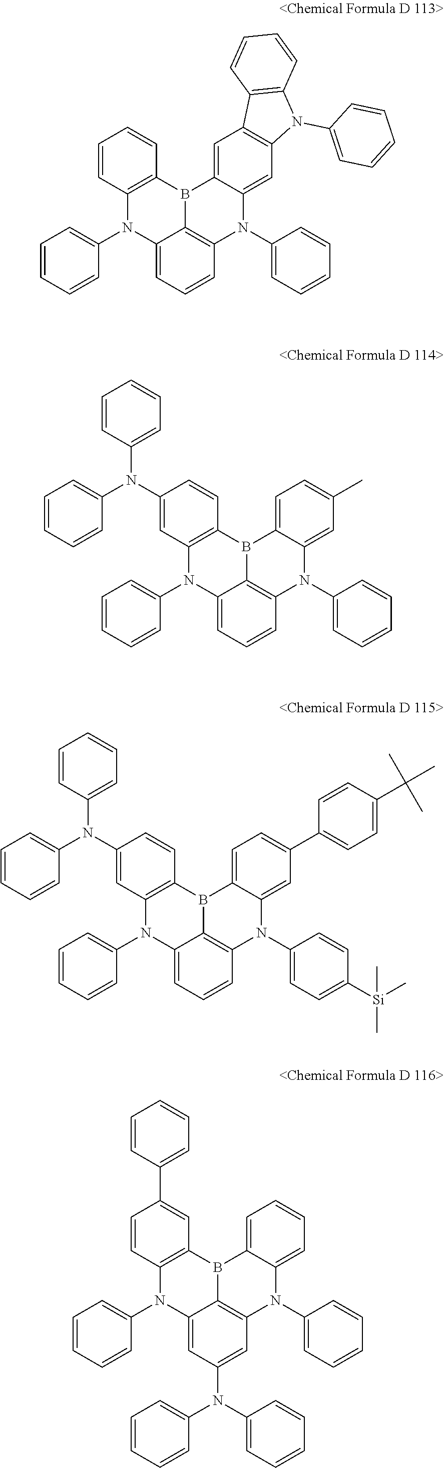 [SMILES: c1ccc(N2c3ccccc3B3c4cc5c6ccccc6n(-c6ccccc6)c5cc4N(c4ccccc4)c4cccc2c43)cc1 ; Cc1ccc2c(c1)N(c1ccccc1)c1cccc3c1B2c1ccc(N(c2ccccc2)c2ccccc2)cc1N3c1ccccc1 ; CC(C)(C)c1ccc(-c2ccc3c(c2)N(c2ccc([Si](C)(C)C)cc2)c2cccc4c2B3c2ccc(N(c3ccccc3)c3ccccc3)cc2N4c2ccccc2)cc1 ; c1ccc(-c2ccc3c(c2)B2c4ccccc4N(c4ccccc4)c4cc(N(c5ccccc5)c5ccccc5)cc(c42)N3c2ccccc2)cc1]

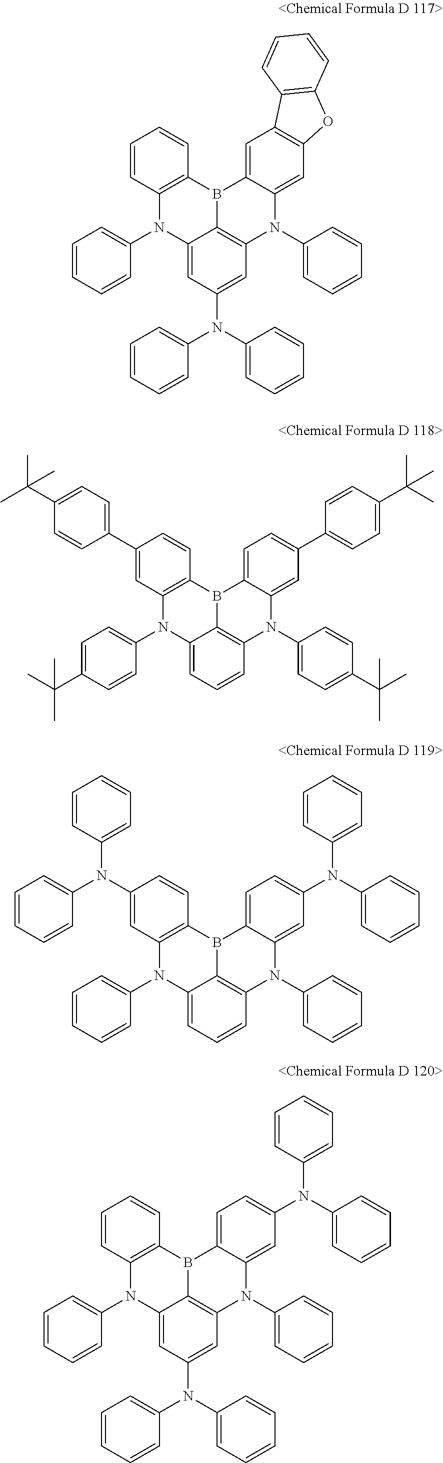 [SMILES: c1ccc(N(c2ccccc2)c2cc3c4c(c2)N(c2ccccc2)c2cc5oc6ccccc6c5cc2B4c2ccccc2N3c2ccccc2)cc1 ; CC(C)(C)c1ccc(-c2ccc3c(c2)N(c2ccc(C(C)(C)C)cc2)c2cccc4c2B3c2ccc(-c3ccc(C(C)(C)C)cc3)cc2N4c2ccc(C(C)(C)C)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cc(N(c4ccccc4)c4ccccc4)cc4c2B3c2ccccc2N4c2ccccc2)cc1]

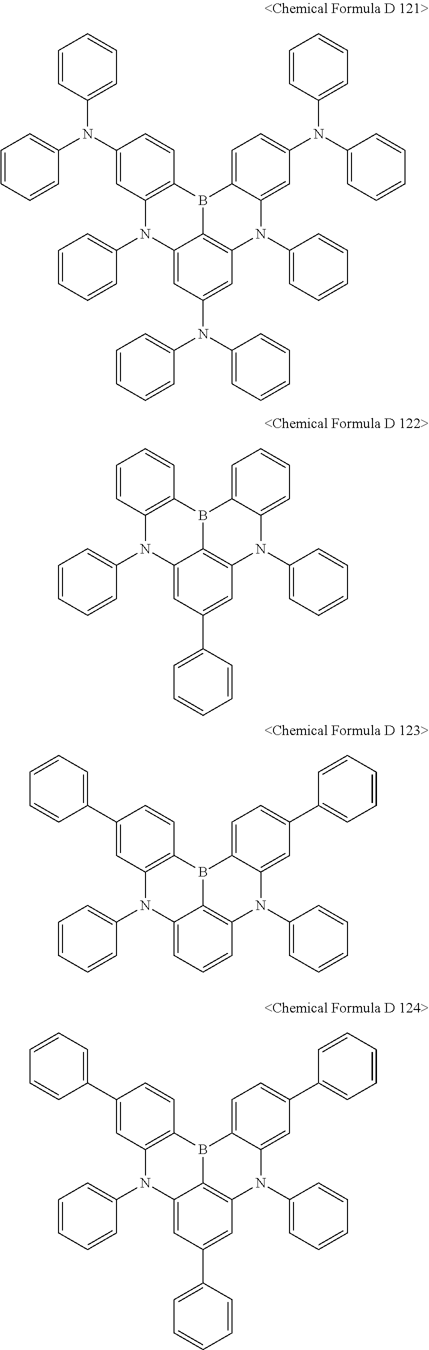 [SMILES: c1ccc(N(c2ccccc2)c2ccc3c(c2)N(c2ccccc2)c2cc(N(c4ccccc4)c4ccccc4)cc4c2B3c2ccc(N(c3ccccc3)c3ccccc3)cc2N4c2ccccc2)cc1 ; c1ccc(-c2cc3c4c(c2)N(c2ccccc2)c2ccccc2B4c2ccccc2N3c2ccccc2)cc1 ; c1ccc(-c2ccc3c(c2)N(c2ccccc2)c2cccc4c2B3c2ccc(-c3ccccc3)cc2N4c2ccccc2)cc1 ; c1ccc(-c2ccc3c(c2)N(c2ccccc2)c2cc(-c4ccccc4)cc4c2B3c2ccc(-c3ccccc3)cc2N4c2ccccc2)cc1]

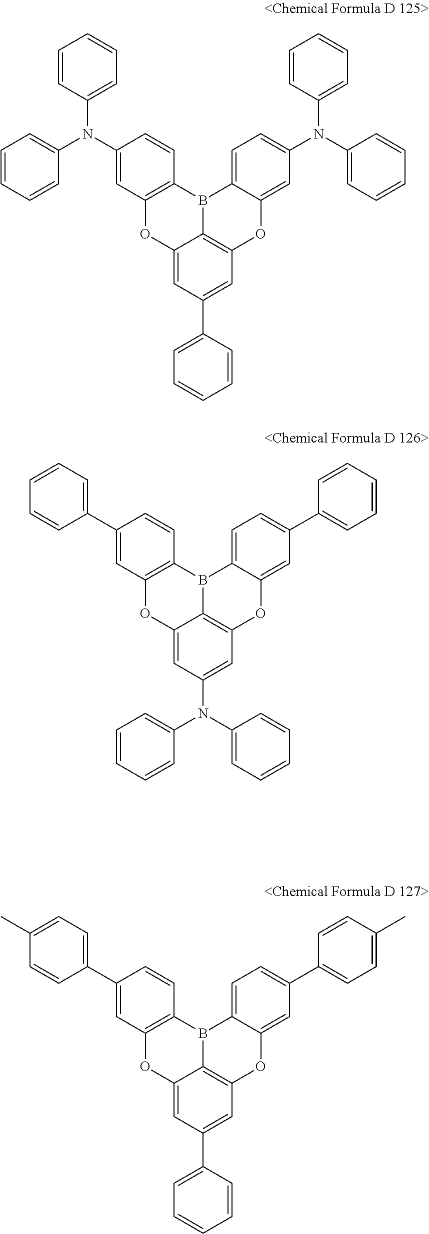

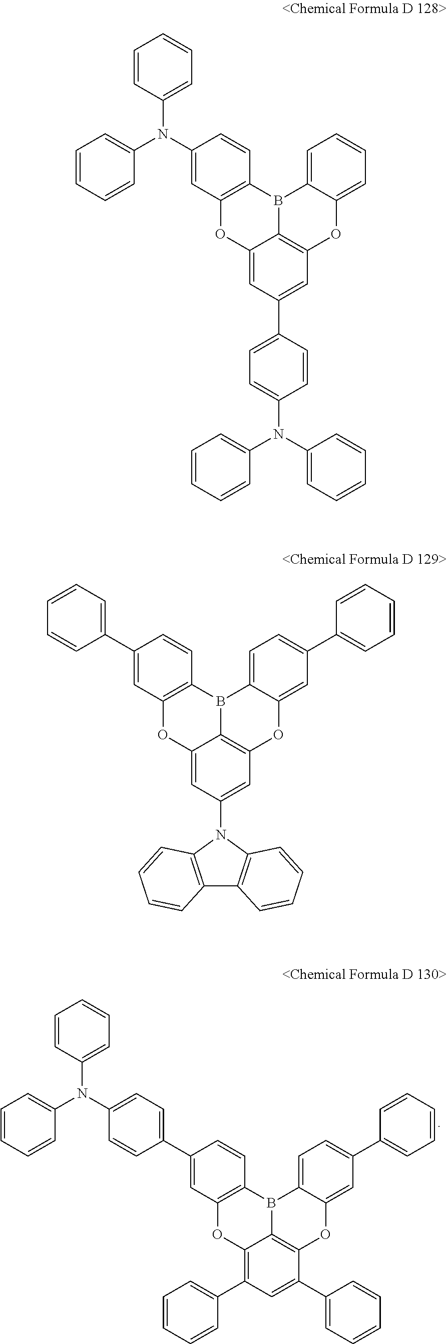 [SMILES: c1ccc(N(c2ccccc2)c2ccc(-c3cc4c5c(c3)Oc3cc(N(c6ccccc6)c6ccccc6)ccc3B5c3ccccc3O4)cc2)cc1 ; c1ccc(-c2ccc3c(c2)Oc2cc(-n4c5ccccc5c5ccccc54)cc4c2B3c2ccc(-c3ccccc3)cc2O4)cc1 ; c1ccc(-c2ccc3c(c2)Oc2c(-c4ccccc4)cc(-c4ccccc4)c4c2B3c2ccc(-c3ccc(N(c5ccccc5)c5ccccc5)cc3)cc2O4)cc1]

Examples of the compounds of [Chemical Formula D4] and [Chemical Formula D5] include the compounds of the following <Chemical Formula D201> to <Chemical Formula D280>:

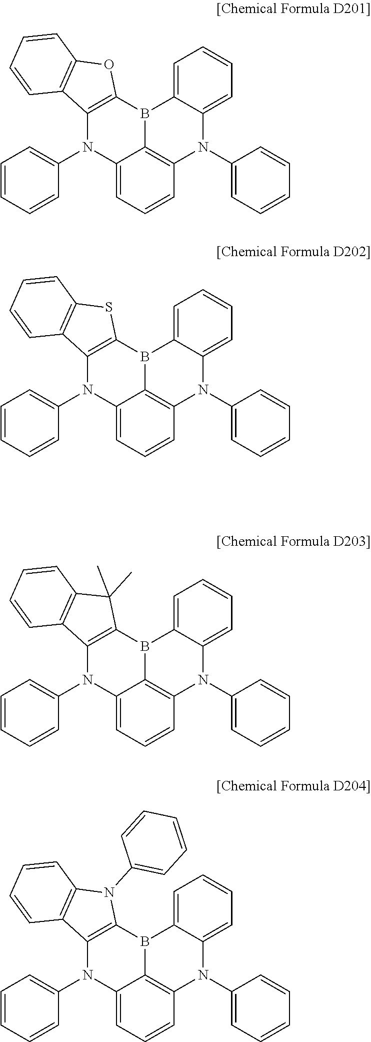 [SMILES: c1ccc(N2c3ccccc3B3c4oc5ccccc5c4N(c4ccccc4)c4cccc2c43)cc1 ; c1ccc(N2c3ccccc3B3c4sc5ccccc5c4N(c4ccccc4)c4cccc2c43)cc1 ; CC1(C)C2=C(c3ccccc31)N(c1ccccc1)c1cccc3c1B2c1ccccc1N3c1ccccc1 ; c1ccc(N2c3ccccc3B3c4c2cccc4N(c2ccccc2)c2c3n(-c3ccccc3)c3ccccc23)cc1]

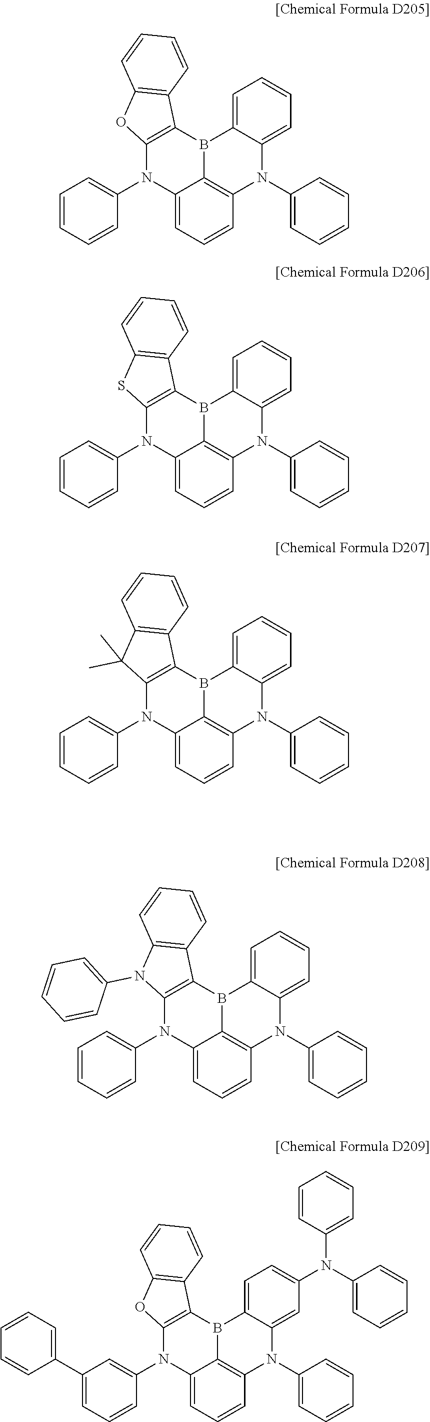 [SMILES: c1ccc(N2c3ccccc3B3c4c2cccc4N(c2ccccc2)c2oc4ccccc4c23)cc1 ; c1ccc(N2c3ccccc3B3c4c2cccc4N(c2ccccc2)c2sc4ccccc4c23)cc1 ; CC1(C)C2=C(B3c4ccccc4N(c4ccccc4)c4cccc(c43)N2c2ccccc2)c2ccccc21 ; c1ccc(N2c3ccccc3B3c4c2cccc4N(c2ccccc2)c2c3c3ccccc3n2-c2ccccc2)cc1 ; c1ccc(-c2cccc(N3c4cccc5c4B(c4ccc(N(c6ccccc6)c6ccccc6)cc4N5c4ccccc4)c4c3oc3ccccc43)c2)cc1]

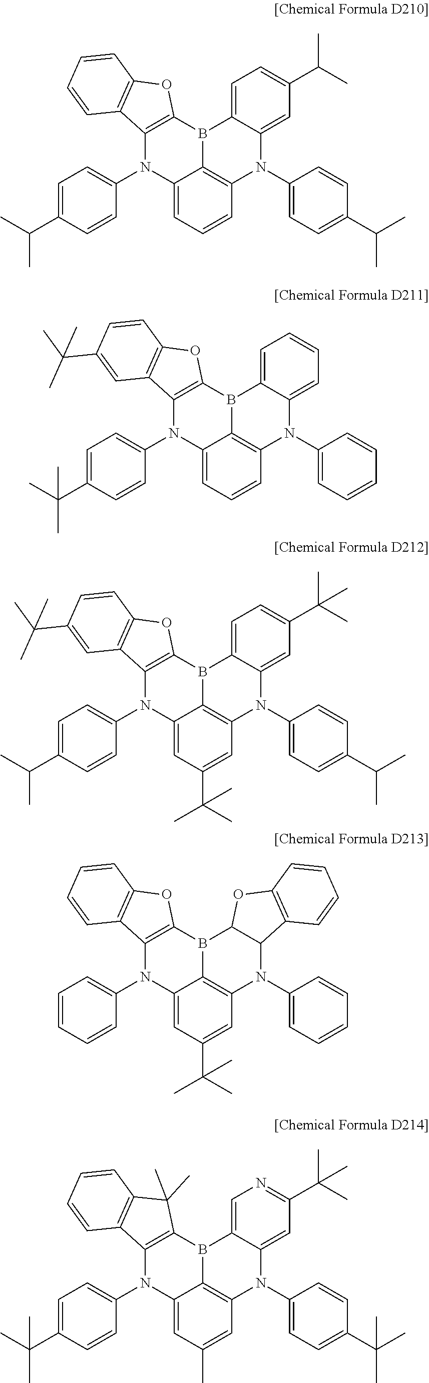

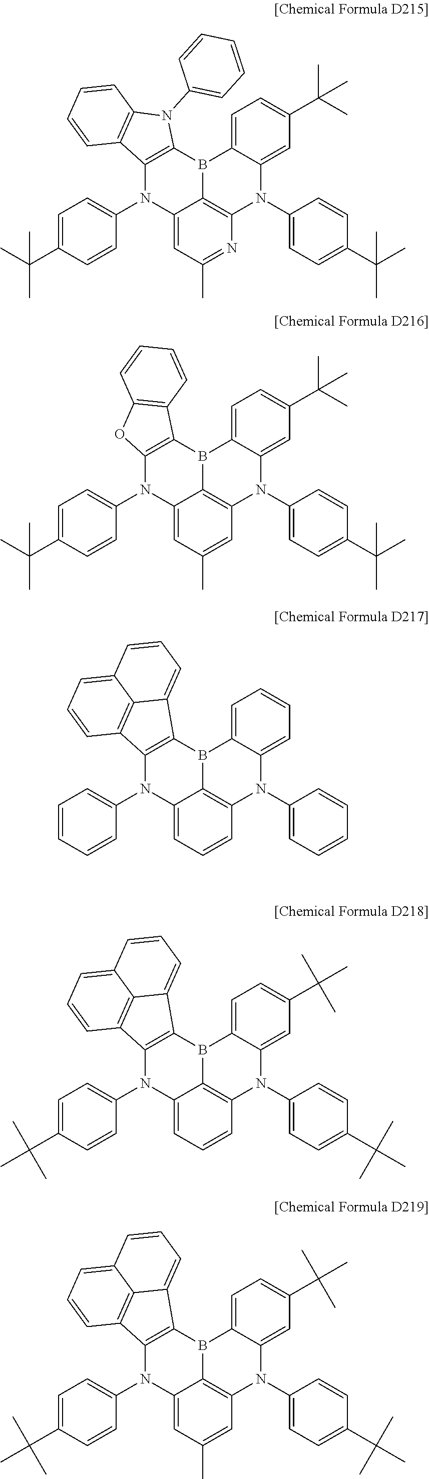

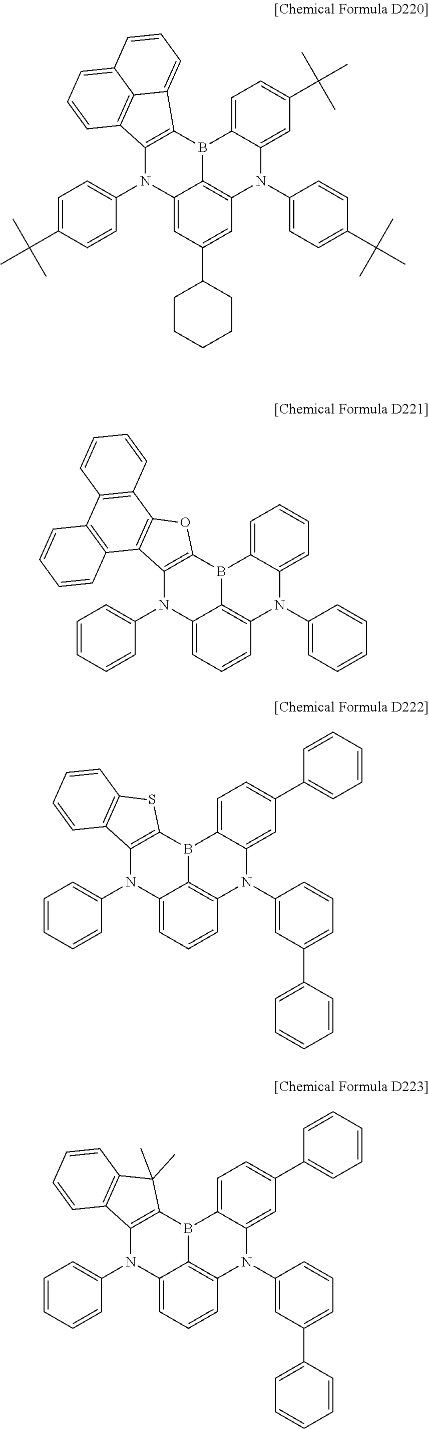 [SMILES: CC(C)(C)c1ccc(N2C3=C(B4c5ccc(C(C)(C)C)cc5N(c5ccc(C(C)(C)C)cc5)c5cc(C6CCCCC6)cc2c54)c2cccc4cccc3c24)cc1 ; c1ccc(N2c3ccccc3B3c4oc5c6ccccc6c6ccccc6c5c4N(c4ccccc4)c4cccc2c43)cc1 ; c1ccc(-c2cccc(N3c4cc(-c5ccccc5)ccc4B4c5sc6ccccc6c5N(c5ccccc5)c5cccc3c54)c2)cc1 ; CC1(C)C2=C(c3ccccc31)N(c1ccccc1)c1cccc3c1B2c1ccc(-c2ccccc2)cc1N3c1cccc(-c2ccccc2)c1]

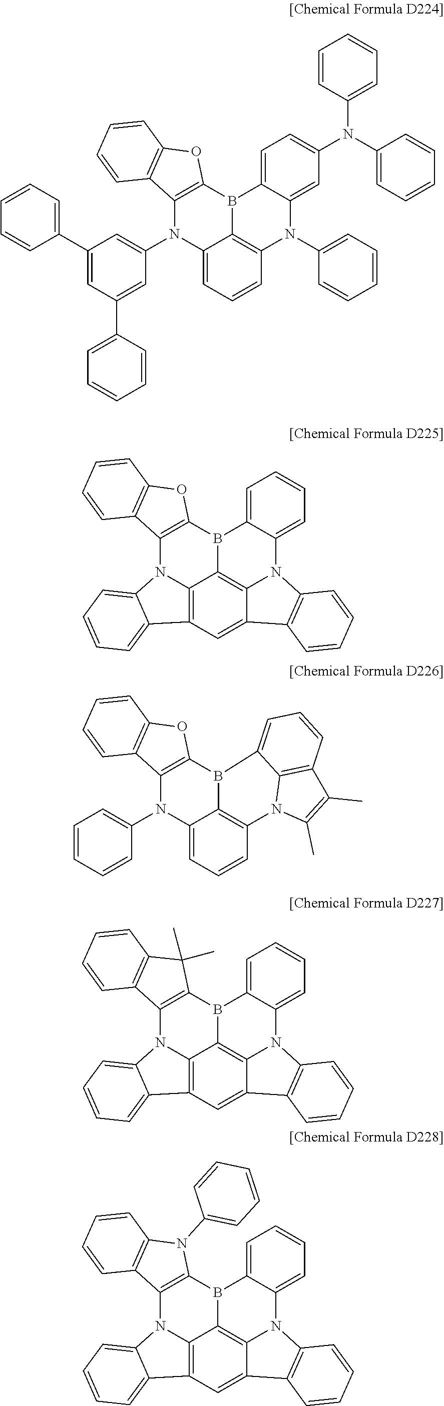

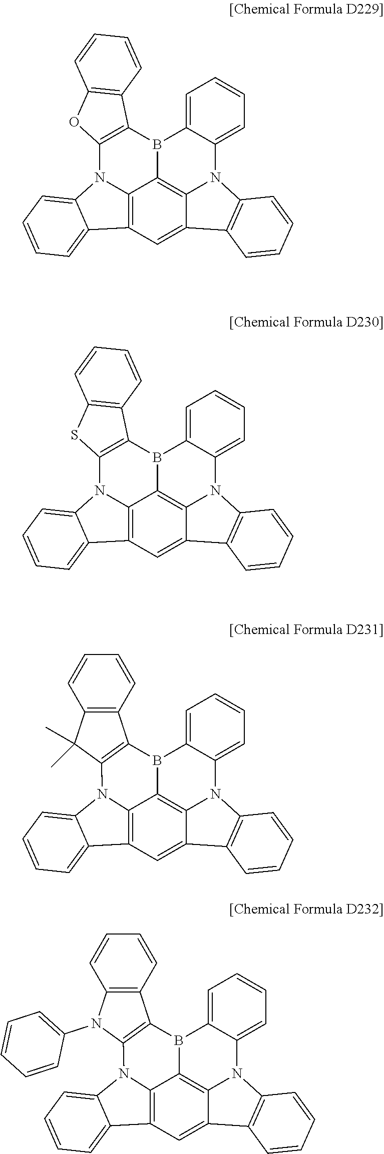 [SMILES: c1ccc2c(c1)B1c3c(oc4ccccc34)-n3c4ccccc4c4cc5c6ccccc6n-2c5c1c43 ; c1ccc2c(c1)B1c3c(sc4ccccc34)-n3c4ccccc4c4cc5c6ccccc6n-2c5c1c43 ; CC1(C)C2=C(B3c4ccccc4-n4c5ccccc5c5cc6c7ccccc7n2c6c3c54)c2ccccc21 ; c1ccc(-n2c3c(c4ccccc42)B2c4ccccc4-n4c5ccccc5c5cc6c7ccccc7n-3c6c2c54)cc1]

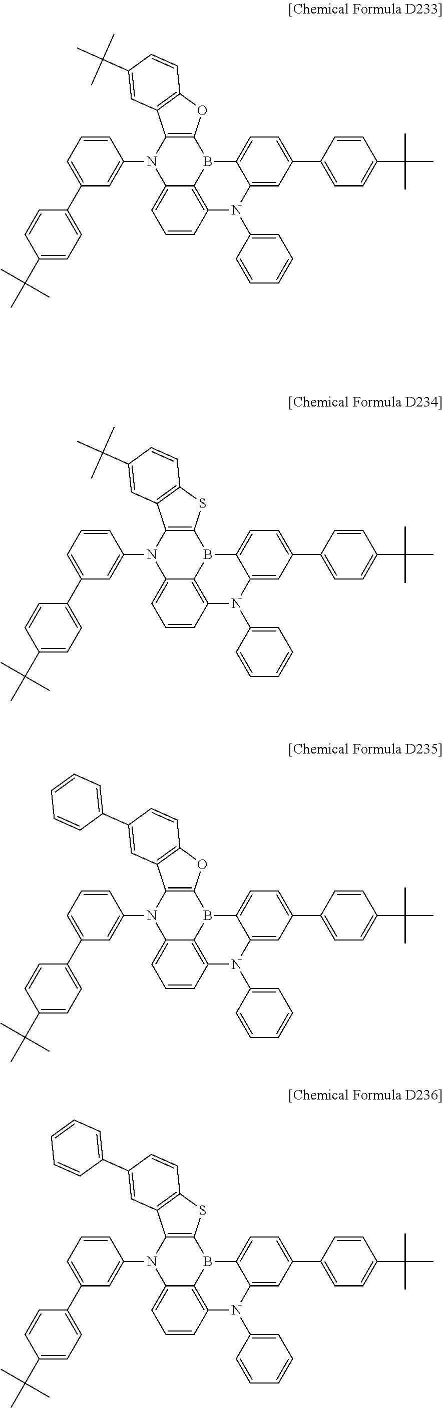

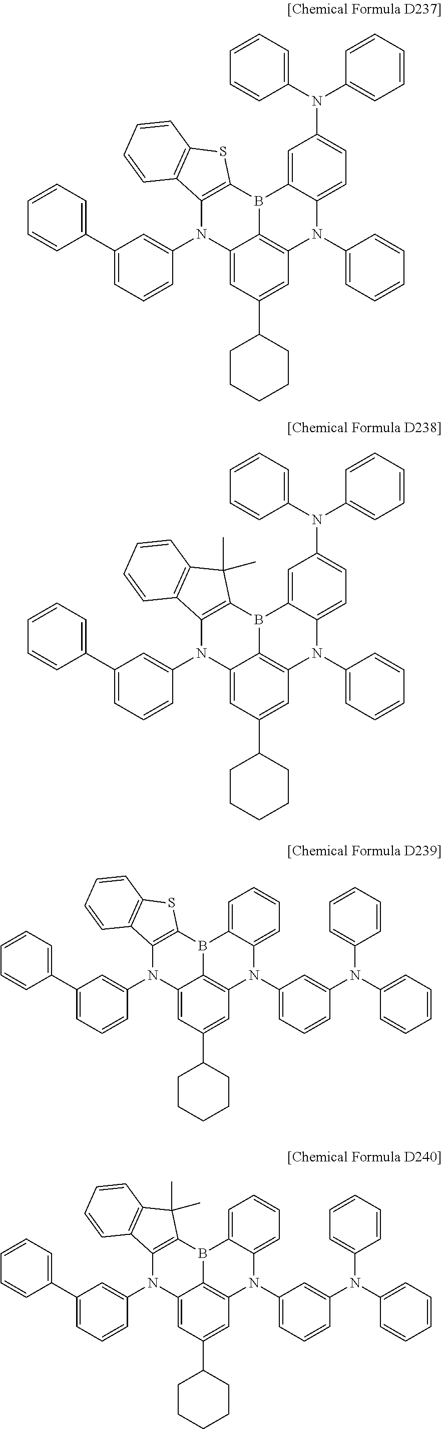 [SMILES: c1ccc(-c2cccc(N3c4cc(C5CCCCC5)cc5c4B(c4cc(N(c6ccccc6)c6ccccc6)ccc4N5c4ccccc4)c4sc5ccccc5c43)c2)cc1 ; CC1(C)C2=C(c3ccccc31)N(c1cccc(-c3ccccc3)c1)c1cc(C3CCCCC3)cc3c1B2c1cc(N(c2ccccc2)c2ccccc2)ccc1N3c1ccccc1 ; c1ccc(-c2cccc(N3c4cc(C5CCCCC5)cc5c4B(c4ccccc4N5c4cccc(N(c5ccccc5)c5ccccc5)c4)c4sc5ccccc5c43)c2)cc1 ; CC1(C)C2=C(c3ccccc31)N(c1cccc(-c3ccccc3)c1)c1cc(C3CCCCC3)cc3c1B2c1ccccc1N3c1cccc(N(c2ccccc2)c2ccccc2)c1]

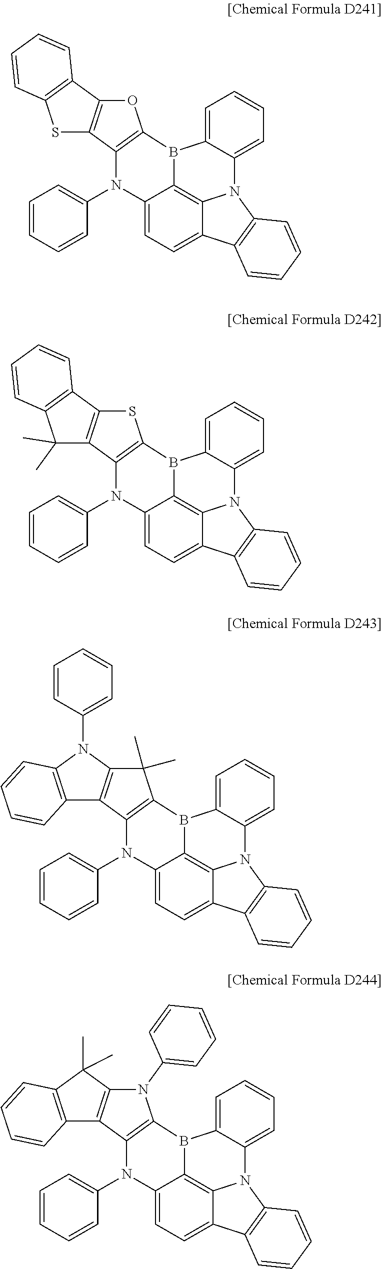 [SMILES: c1ccc(N2c3ccc4c5ccccc5n5c4c3B(c3ccccc3-5)c3oc4c(sc5ccccc54)c32)cc1 ; CC1(C)c2ccccc2-c2sc3c(c21)N(c1ccccc1)c1ccc2c4ccccc4n4c2c1B3c1ccccc1-4 ; CC1(C)C2=C(c3c1n(-c1ccccc1)c1ccccc31)N(c1ccccc1)c1ccc3c4ccccc4n4c3c1B2c1ccccc1-4 ; CC1(C)c2ccccc2-c2c3c(n(-c4ccccc4)c21)B1c2ccccc2-n2c4ccccc4c4ccc(c1c42)N3c1ccccc1]

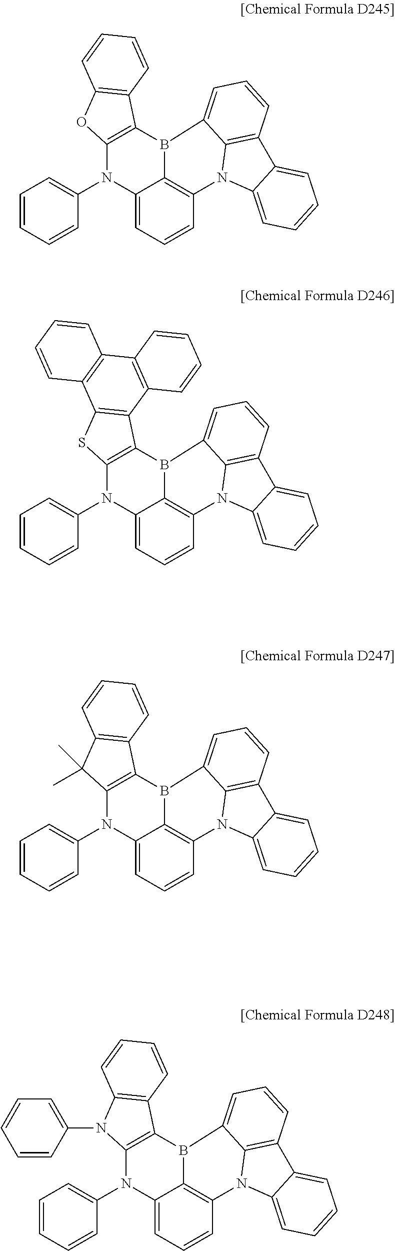 [SMILES: c1ccc(N2c3cccc4c3B(c3c2oc2ccccc32)c2cccc3c5ccccc5n-4c23)cc1 ; c1ccc(N2c3cccc4c3B(c3c2sc2c5ccccc5c5ccccc5c32)c2cccc3c5ccccc5n-4c23)cc1 ; CC1(C)C2=C(B3c4c(cccc4-n4c5ccccc5c5cccc3c54)N2c2ccccc2)c2ccccc21 ; c1ccc(N2c3cccc4c3B(c3c2n(-c2ccccc2)c2ccccc32)c2cccc3c5ccccc5n-4c23)cc1]

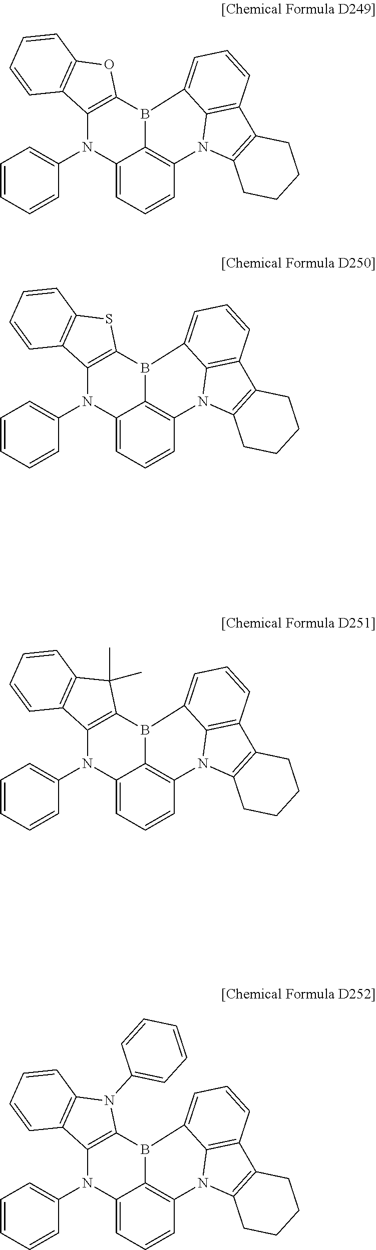 [SMILES: c1ccc(N2c3cccc4c3B(c3oc5ccccc5c32)c2cccc3c5c(n-4c23)CCCC5)cc1 ; c1ccc(N2c3cccc4c3B(c3sc5ccccc5c32)c2cccc3c5c(n-4c23)CCCC5)cc1 ; CC1(C)C2=C(c3ccccc31)N(c1ccccc1)c1cccc3c1B2c1cccc2c4c(n-3c12)CCCC4 ; c1ccc(N2c3cccc4c3B(c3cccc5c6c(n-4c35)CCCC6)c3c2c2ccccc2n3-c2ccccc2)cc1]

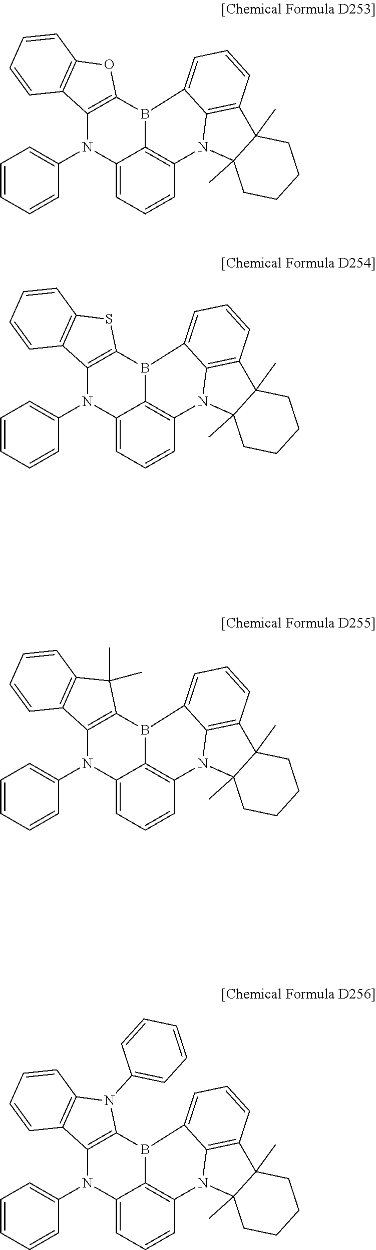 [SMILES: CC12CCCCC1(C)N1c3cccc4c3B(c3cccc2c31)c1oc2ccccc2c1N4c1ccccc1 ; CC12CCCCC1(C)N1c3cccc4c3B(c3cccc2c31)c1sc2ccccc2c1N4c1ccccc1 ; CC1(C)C2=C(c3ccccc31)N(c1ccccc1)c1cccc3c1B2c1cccc2c1N3C1(C)CCCCC21C ; CC12CCCCC1(C)N1c3cccc4c3B(c3cccc2c31)c1c(c2ccccc2n1-c1ccccc1)N4c1ccccc1]

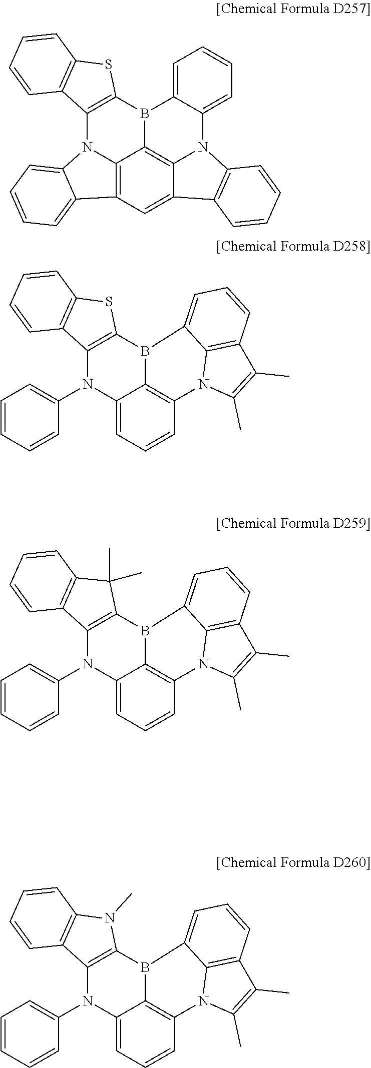

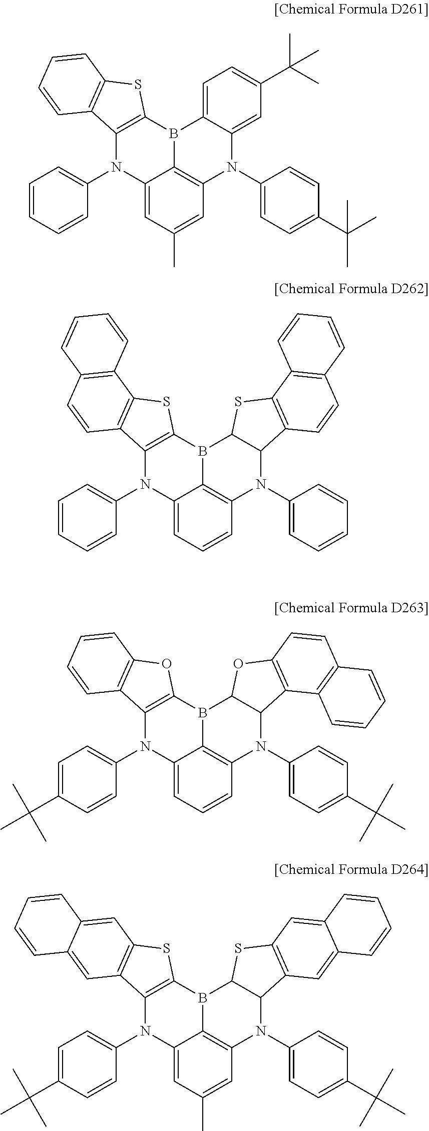

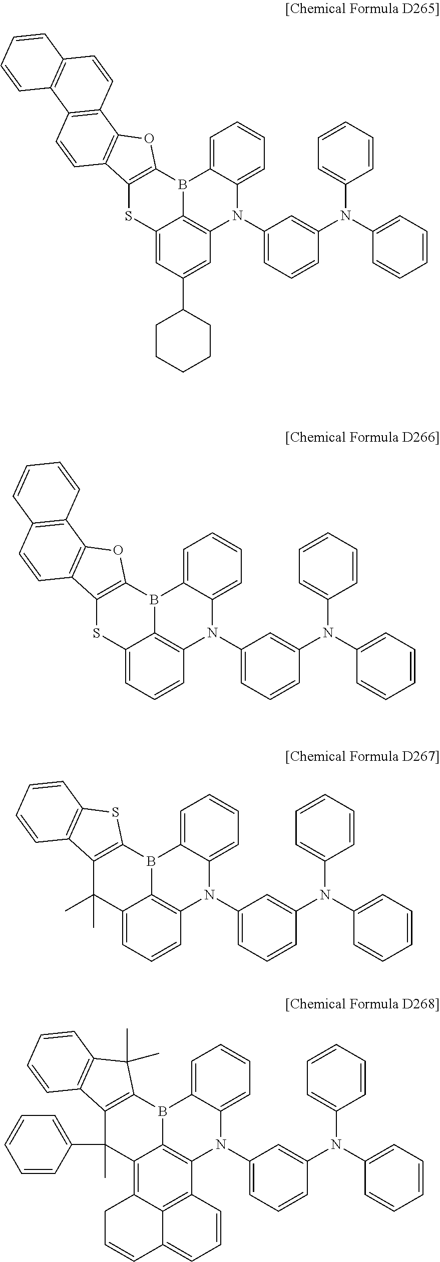 [SMILES: c1ccc(N(c2ccccc2)c2cccc(N3c4ccccc4B4c5oc6c(ccc7c8ccccc8ccc76)c5Sc5cc(C6CCCCC6)cc3c54)c2)cc1 ; c1ccc(N(c2ccccc2)c2cccc(N3c4ccccc4B4c5oc6c(ccc7ccccc76)c5Sc5cccc3c54)c2)cc1 ; CC1(C)c2cccc3c2B(c2ccccc2N3c2cccc(N(c3ccccc3)c3ccccc3)c2)c2sc3ccccc3c21 ; CC1(C)C2=C(c3ccccc31)C(C)(c1ccccc1)c1c3c(c4cccc5c4c1CC=C5)N(c1cccc(N(c4ccccc4)c4ccccc4)c1)c1ccccc1B23]

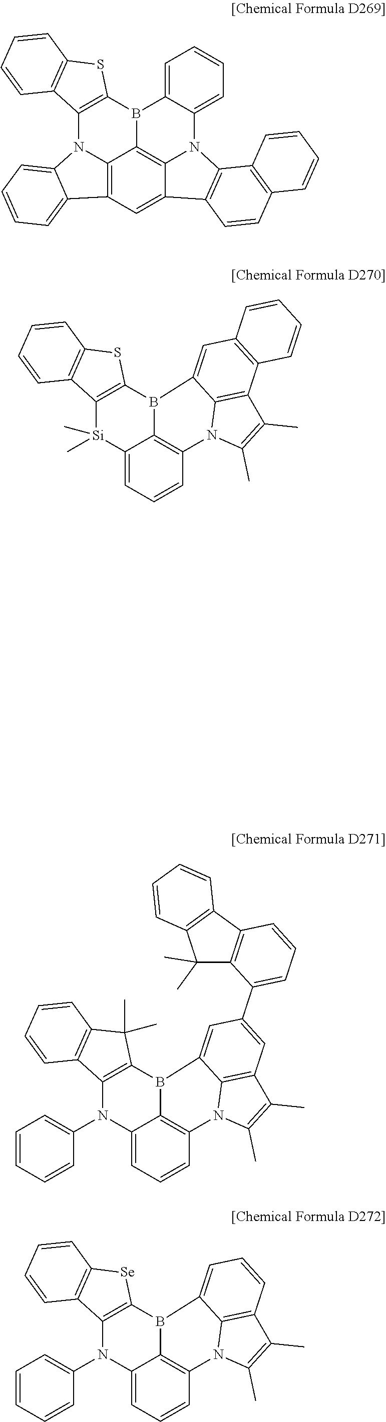 [SMILES: c1ccc2c(c1)B1c3sc4ccccc4c3-n3c4ccccc4c4cc5c6ccc7ccccc7c6n-2c5c1c43 ; Cc1c(C)n2c3c(cc4ccccc4c13)B1c3sc4ccccc4c3[Si](C)(C)c3cccc-2c31 ; Cc1c(C)n2c3c(cc(-c4cccc5c4C(C)(C)c4ccccc4-5)cc13)B1C3=C(c4ccccc4C3(C)C)N(c3ccccc3)c3cccc-2c31 ; Cc1c(C)n2c3c(cccc13)B1c3[se]c4ccccc4c3N(c3ccccc3)c3cccc-2c31]

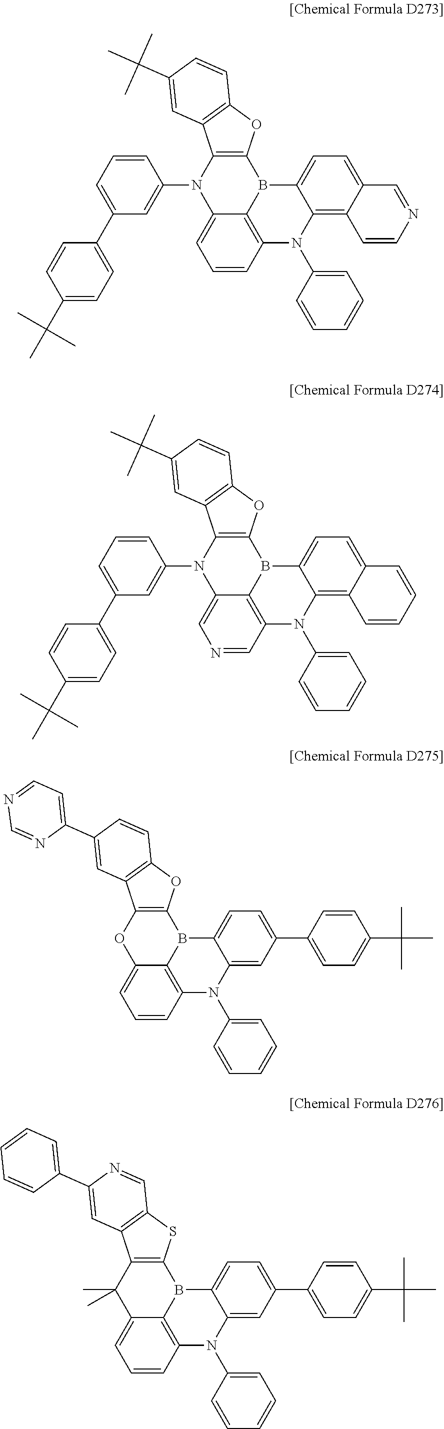

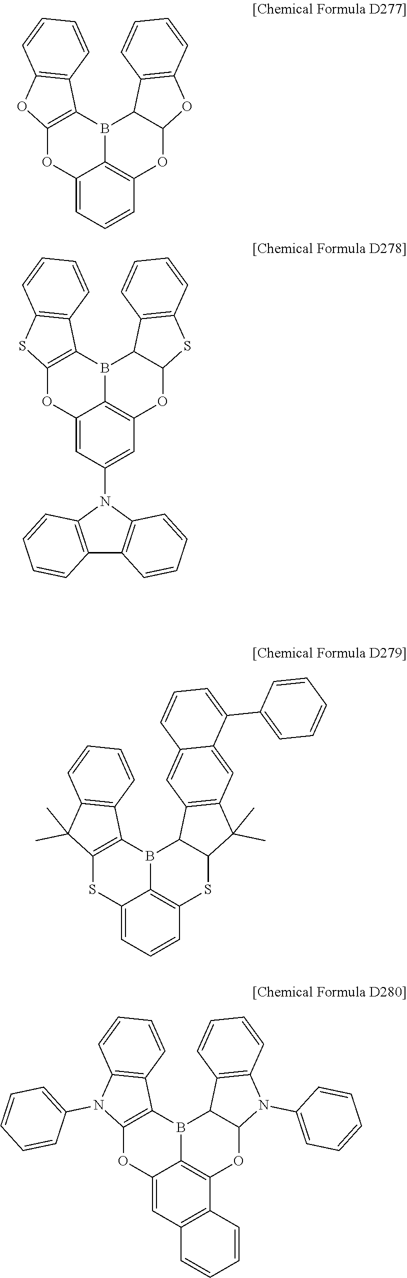

Examples of the compound represented by any one of [Chemical Formula D6] to [Chemical Formula D8] include the compounds of the following <Chemical Formula D301> to <Chemical Formula 432>:

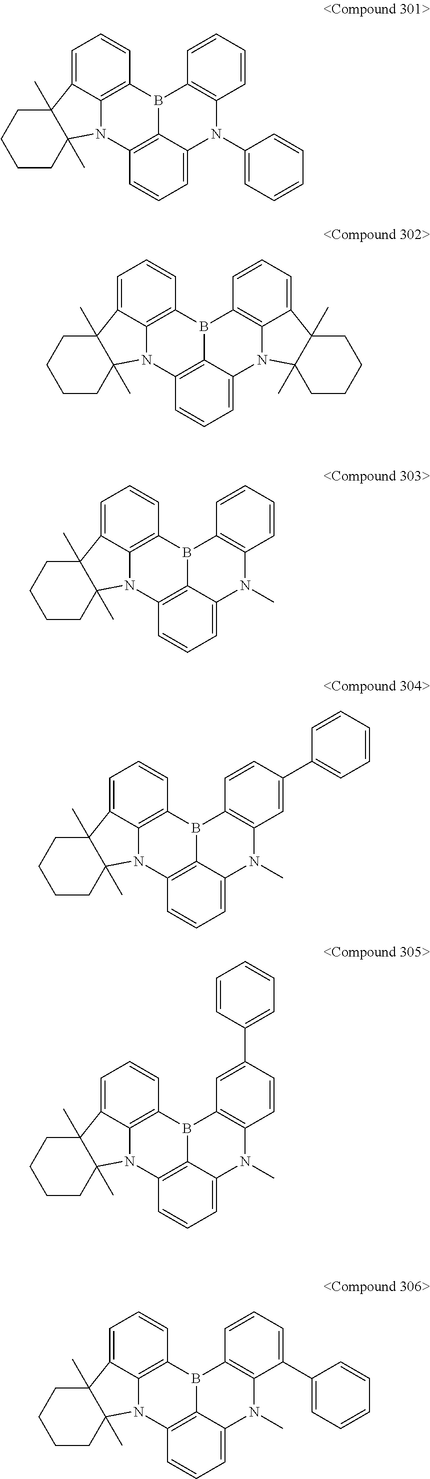 [SMILES: CC12CCCCC1(C)N1c3cccc4c3B(c3ccccc3N4c3ccccc3)c3cccc2c31 ; CC12CCCCC1(C)N1c3cccc4c3B(c3cccc2c31)c1cccc2c1N4C1(C)CCCCC21C ; CN1c2ccccc2B2c3cccc4c3N(c3cccc1c32)C1(C)CCCCC41C ; CN1c2cc(-c3ccccc3)ccc2B2c3cccc4c3N(c3cccc1c32)C1(C)CCCCC41C ; CN1c2ccc(-c3ccccc3)cc2B2c3cccc4c3N(c3cccc1c32)C1(C)CCCCC41C ; CN1c2cccc3c2B(c2cccc(-c4ccccc4)c21)c1cccc2c1N3C1(C)CCCCC21C]

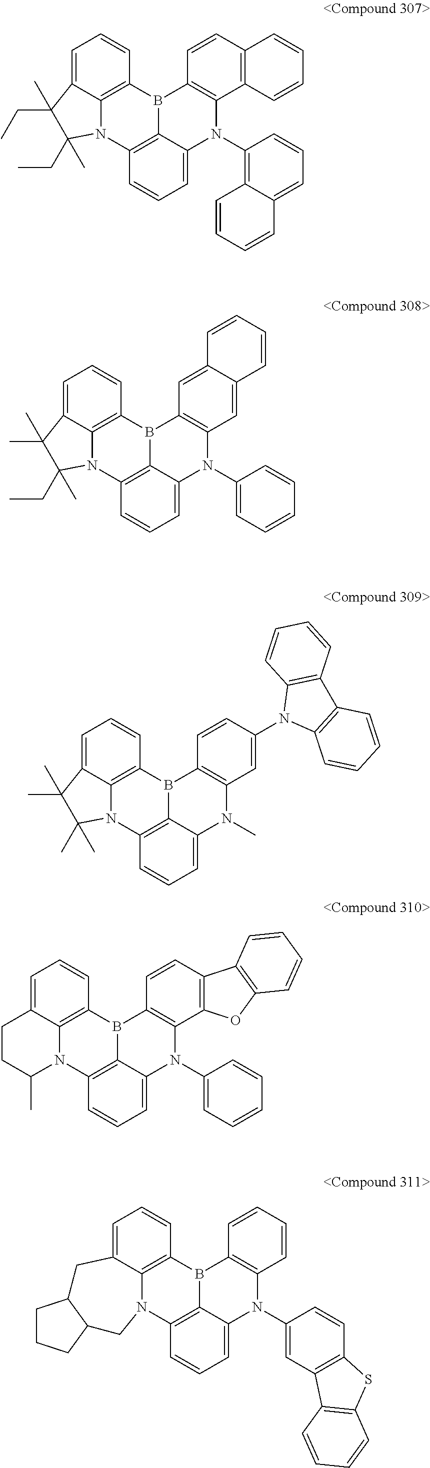 [SMILES: CCC1(C)c2cccc3c2N(c2cccc4c2B3c2ccc3ccccc3c2N4c2cccc3ccccc23)C1(C)CC ; CCC1(C)N2c3cccc4c3B(c3cc5ccccc5cc3N4c3ccccc3)c3cccc(c32)C1(C)C ; CN1c2cc(-n3c4ccccc4c4ccccc43)ccc2B2c3cccc4c3N(c3cccc1c32)C(C)(C)C4(C)C ; CC1CCc2cccc3c2N1c1cccc2c1B3c1ccc3c(oc4ccccc43)c1N2c1ccccc1 ; c1ccc2c(c1)B1c3cccc4c3N(CC3CCCC3C4)c3cccc(c31)N2c1ccc2sc3ccccc3c2c1]

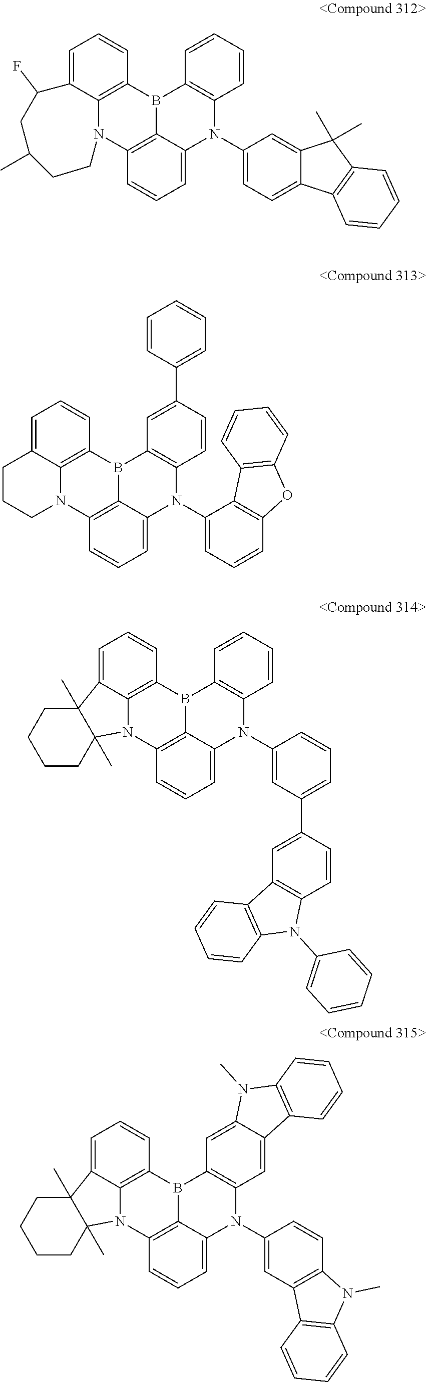 [SMILES: CC1CCN2c3cccc4c3B(c3ccccc3N4c3ccc4c(c3)C(C)(C)c3ccccc3-4)c3cccc(c32)C(F)C1 ; c1ccc(-c2ccc3c(c2)B2c4cccc5c4N(CCC5)c4cccc(c42)N3c2cccc3oc4ccccc4c23)cc1 ; CC12CCCCC1(C)N1c3cccc4c3B(c3ccccc3N4c3cccc(-c4ccc5c(c4)c4ccccc4n5-c4ccccc4)c3)c3cccc2c31 ; Cn1c2ccccc2c2cc(N3c4cc5c6ccccc6n(C)c5cc4B4c5cccc6c5N(c5cccc3c54)C3(C)CCCCC63C)ccc21]

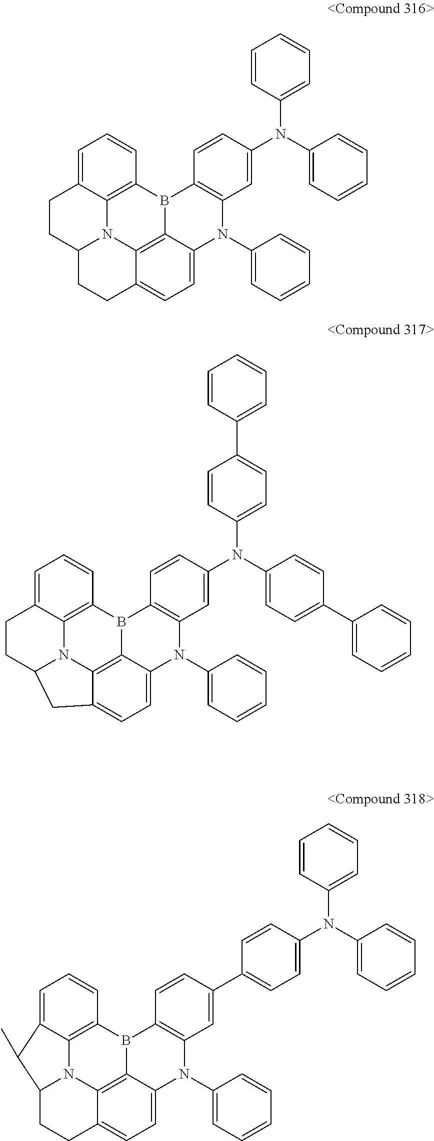 [SMILES: c1ccc(N(c2ccccc2)c2ccc3c(c2)N(c2ccccc2)c2ccc4c5c2B3c2cccc3c2N5C(CC3)CC4)cc1 ; c1ccc(-c2ccc(N(c3ccc(-c4ccccc4)cc3)c3ccc4c(c3)N(c3ccccc3)c3ccc5c6c3B4c3cccc4c3N6C(CC4)C5)cc2)cc1 ; CC1c2cccc3c2N2c4c(ccc5c4B3c3ccc(-c4ccc(N(c6ccccc6)c6ccccc6)cc4)cc3N5c3ccccc3)CCC12]

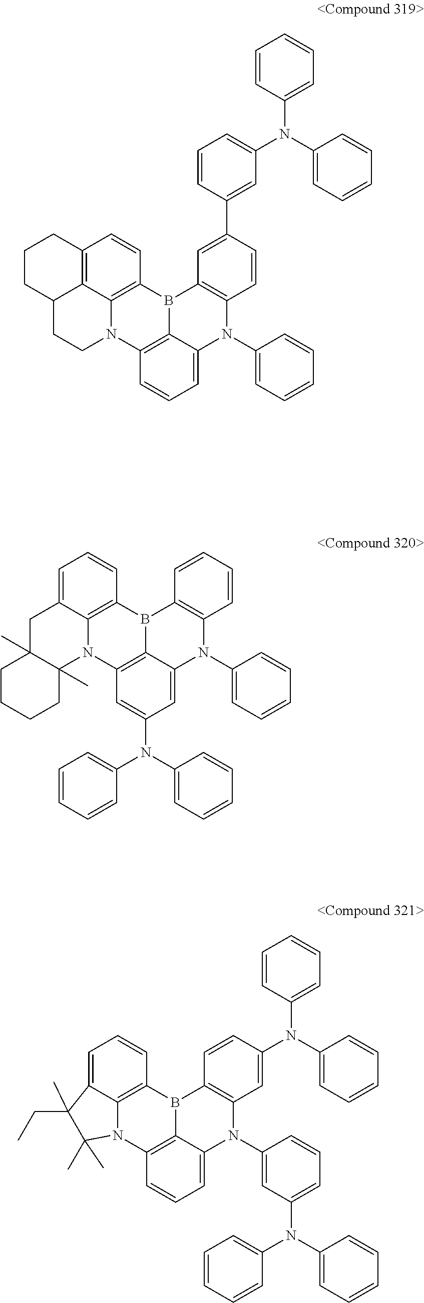 [SMILES: c1ccc(N(c2ccccc2)c2cccc(-c3ccc4c(c3)B3c5ccc6c7c5N(CCC7CCC6)c5cccc(c53)N4c3ccccc3)c2)cc1 ; CC12CCCCC1(C)N1c3cc(N(c4ccccc4)c4ccccc4)cc4c3B(c3ccccc3N4c3ccccc3)c3cccc(c31)C2 ; CCC1(C)c2cccc3c2N(c2cccc4c2B3c2ccc(N(c3ccccc3)c3ccccc3)cc2N4c2cccc(N(c3ccccc3)c3ccccc3)c2)C1(C)C]

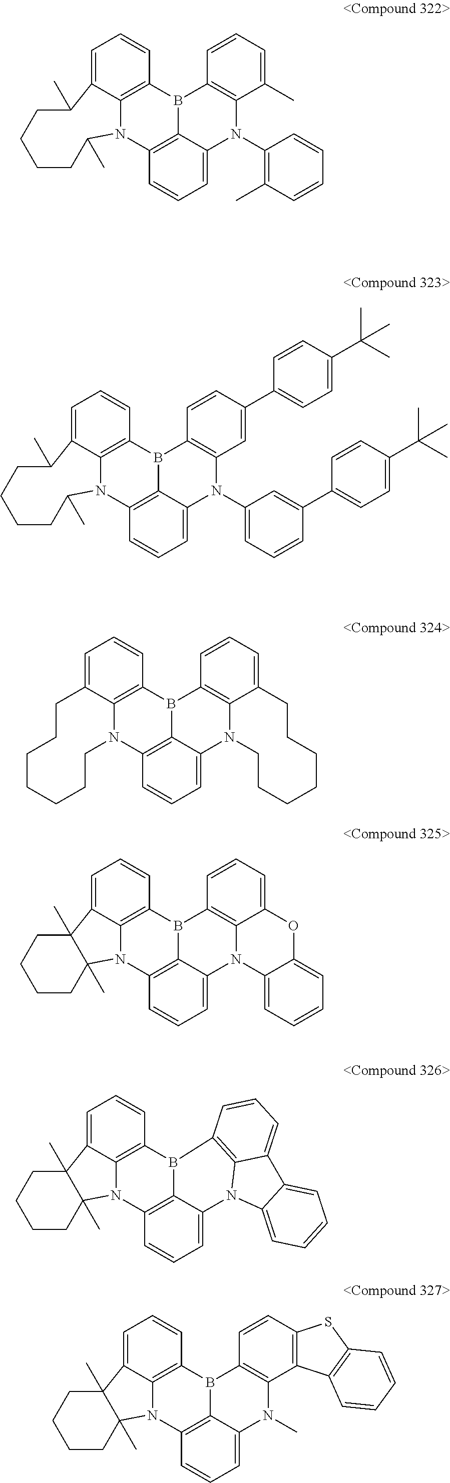 [SMILES: Cc1ccccc1N1c2cccc3c2B(c2cccc(C)c21)c1cccc2c1N3C(C)CCCCC2C ; CC1CCCCC(C)N2c3cccc4c3B(c3ccc(-c5ccc(C(C)(C)C)cc5)cc3N4c3cccc(-c4ccc(C(C)(C)C)cc4)c3)c3cccc1c32 ; c1cc2c3c(c1)B1c4cccc5c4N(CCCCCCC5)c4cccc(c41)N3CCCCCCC2 ; CC12CCCCC1(C)N1c3cccc4c3B(c3cccc5c3N4c3ccccc3O5)c3cccc2c31 ; CC12CCCCC1(C)N1c3cccc4c3B(c3cccc2c31)c1cccc2c3ccccc3n-4c12 ; CN1c2cccc3c2B(c2cccc4c2N3C2(C)CCCCC42C)c2ccc3sc4ccccc4c3c21]

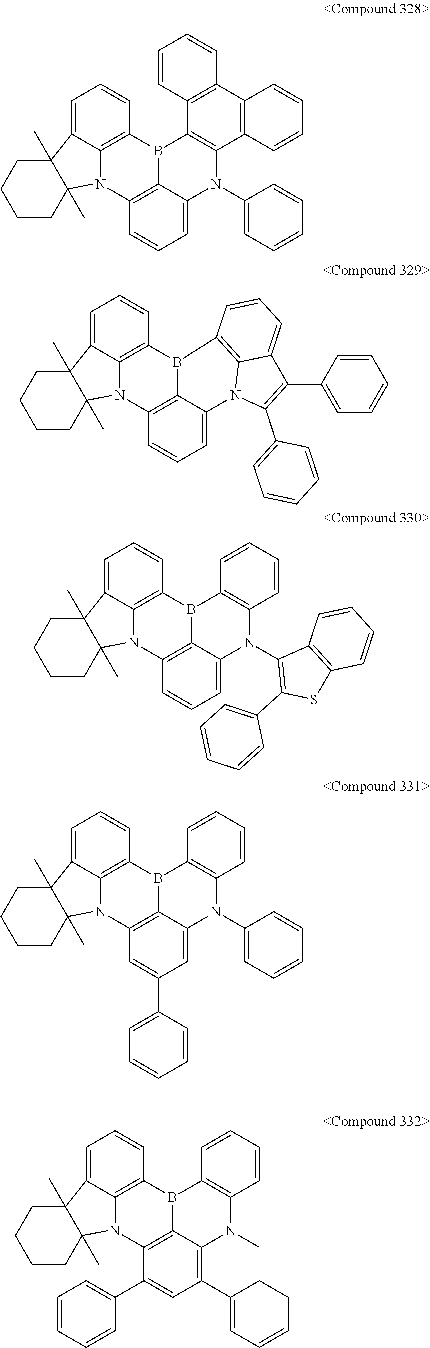 [SMILES: CC12CCCCC1(C)N1c3cccc4c3B(c3cccc2c31)c1c(c2ccccc2c2ccccc12)N4c1ccccc1 ; CC12CCCCC1(C)N1c3cccc4c3B(c3cccc2c31)c1cccc2c(-c3ccccc3)c(-c3ccccc3)n-4c12 ; CC12CCCCC1(C)N1c3cccc4c3B(c3ccccc3N4c3c(-c4ccccc4)sc4ccccc34)c3cccc2c31 ; CC12CCCCC1(C)N1c3cc(-c4ccccc4)cc4c3B(c3ccccc3N4c3ccccc3)c3cccc2c31 ; CN1c2ccccc2B2c3cccc4c3N(c3c(-c5ccccc5)cc(C5=CC=CCC5)c1c32)C1(C)CCCCC41C]

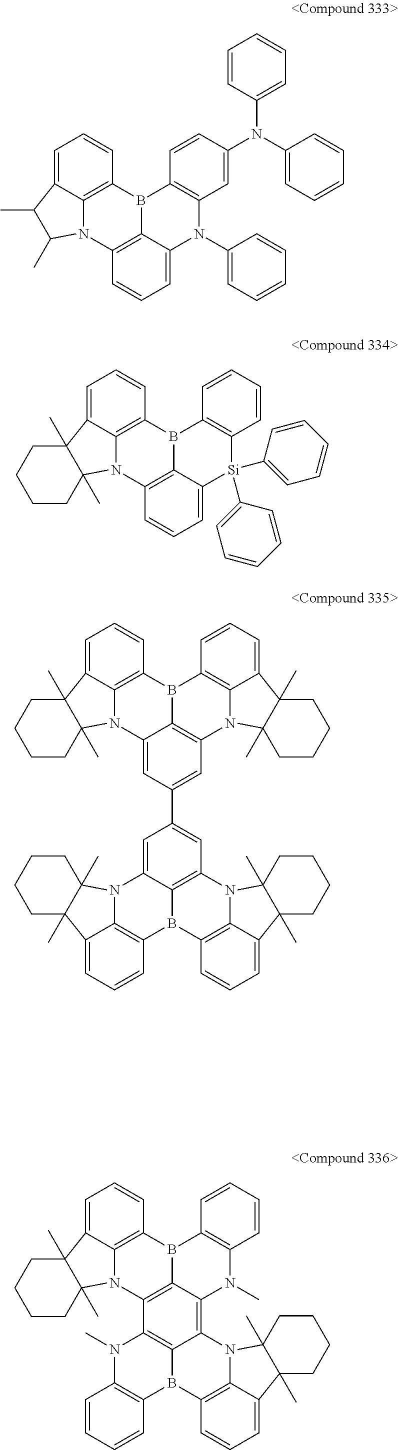 [SMILES: CC1c2cccc3c2N(c2cccc4c2B3c2ccc(N(c3ccccc3)c3ccccc3)cc2N4c2ccccc2)C1C ; CC12CCCCC1(C)N1c3cccc4c3B(c3ccccc3[Si]4(c3ccccc3)c3ccccc3)c3cccc2c31 ; CC12CCCCC1(C)N1c3cc(-c4cc5c6c(c4)N4c7c(cccc7C7(C)CCCCC47C)B6c4cccc6c4N5C4(C)CCCCC64C)cc4c3B(c3cccc2c31)c1cccc2c1N4C1(C)CCCCC21C ; CN1c2ccccc2B2c3cccc4c3N(c3c2c1c1c2c3N(C)c3ccccc3B2c2cccc3c2N1C1(C)CCCCC31C)C1(C)CCCCC41C]

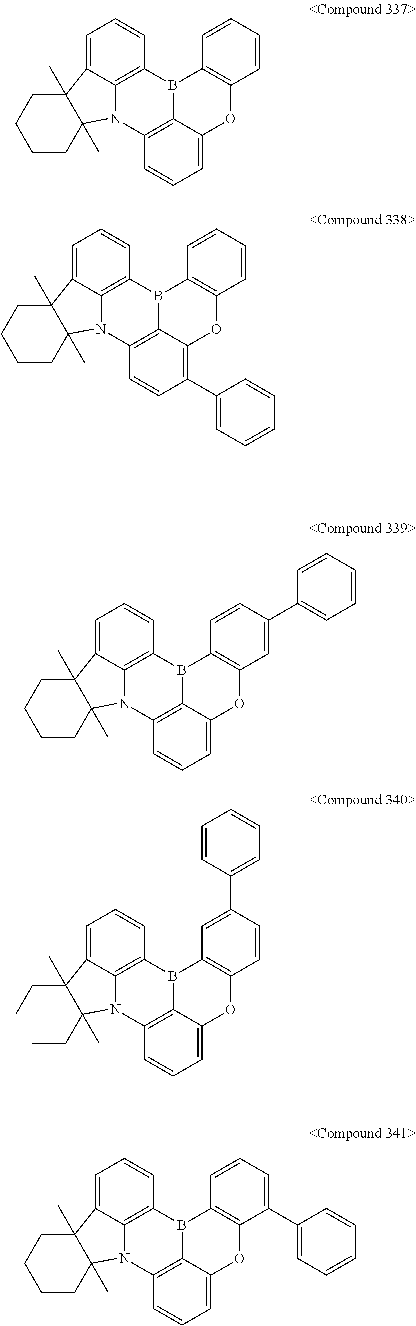 [SMILES: CC12CCCCC1(C)N1c3cccc4c3B(c3ccccc3O4)c3cccc2c31 ; CC12CCCCC1(C)N1c3ccc(-c4ccccc4)c4c3B(c3ccccc3O4)c3cccc2c31 ; CC12CCCCC1(C)N1c3cccc4c3B(c3ccc(-c5ccccc5)cc3O4)c3cccc2c31 ; CCC1(C)c2cccc3c2N(c2cccc4c2B3c2cc(-c3ccccc3)ccc2O4)C1(C)CC ; CC12CCCCC1(C)N1c3cccc4c3B(c3cccc(-c5ccccc5)c3O4)c3cccc2c31]

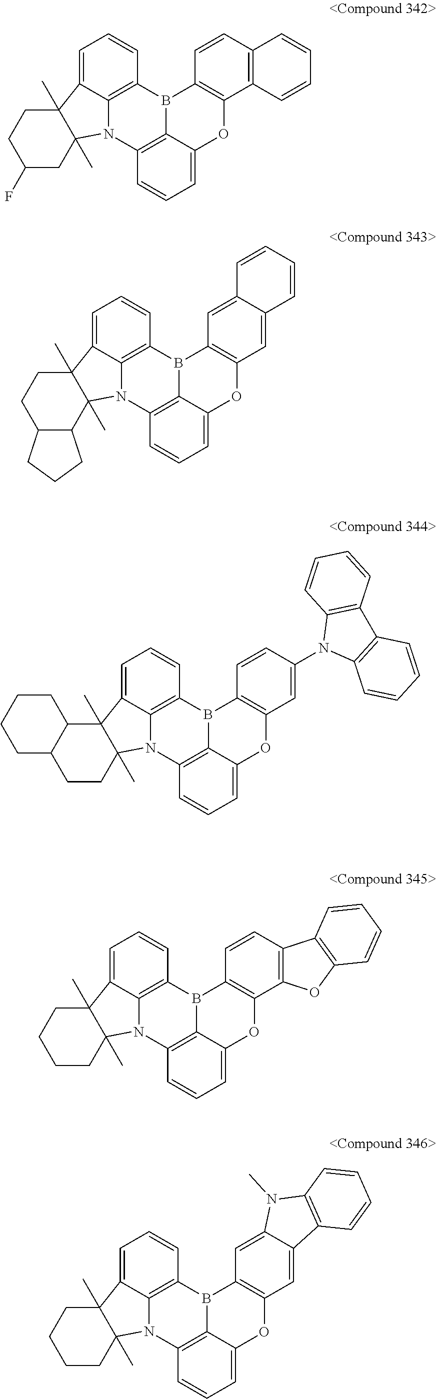 [SMILES: CC12CCC(F)CC1(C)N1c3cccc4c3B(c3cccc2c31)c1ccc2ccccc2c1O4 ; CC12CCC3CCCC3C1(C)N1c3cccc4c3B(c3cc5ccccc5cc3O4)c3cccc2c31 ; CC12c3cccc4c3N(c3cccc5c3B4c3ccc(-n4c6ccccc6c6ccccc64)cc3O5)C1(C)CCC1CCCCC12 ; CC12CCCCC1(C)N1c3cccc4c3B(c3cccc2c31)c1ccc2c(oc3ccccc32)c1O4 ; Cn1c2ccccc2c2cc3c(cc21)B1c2cccc4c2N(c2cccc(c21)O3)C1(C)CCCCC41C]

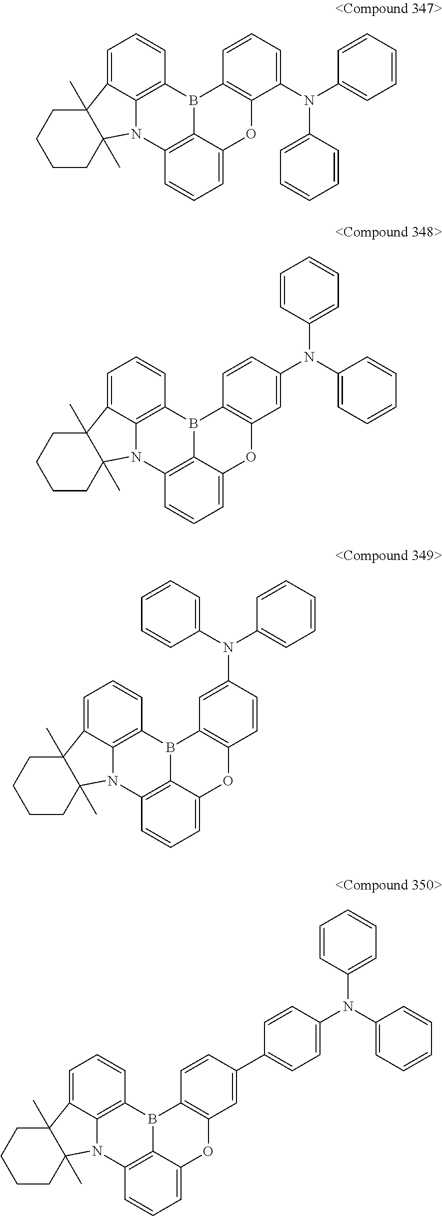 [SMILES: CC12CCCCC1(C)N1c3cccc4c3B(c3cccc(N(c5ccccc5)c5ccccc5)c3O4)c3cccc2c31 ; CC12CCCCC1(C)N1c3cccc4c3B(c3ccc(N(c5ccccc5)c5ccccc5)cc3O4)c3cccc2c31 ; CC12CCCCC1(C)N1c3cccc4c3B(c3cc(N(c5ccccc5)c5ccccc5)ccc3O4)c3cccc2c31 ; CC12CCCCC1(C)N1c3cccc4c3B(c3ccc(-c5ccc(N(c6ccccc6)c6ccccc6)cc5)cc3O4)c3cccc2c31]

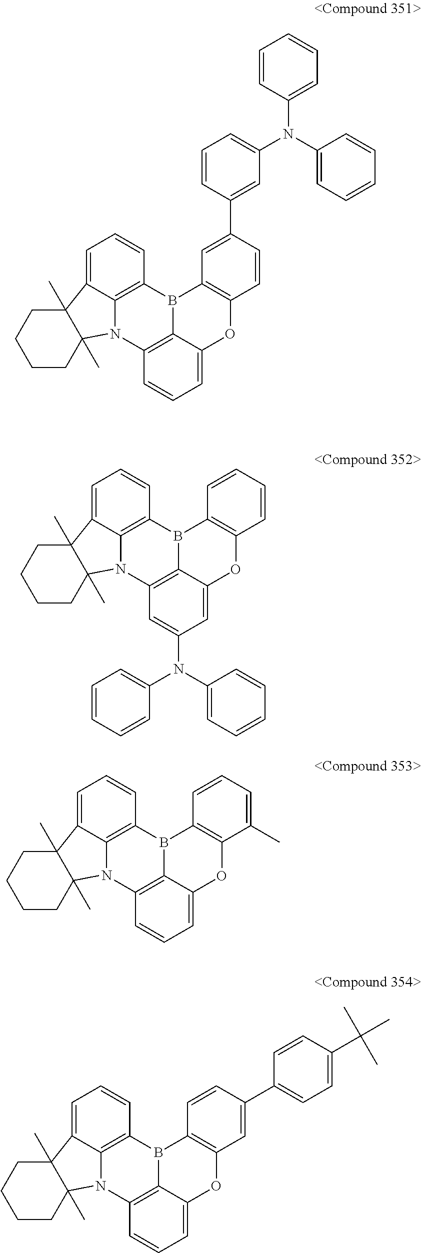

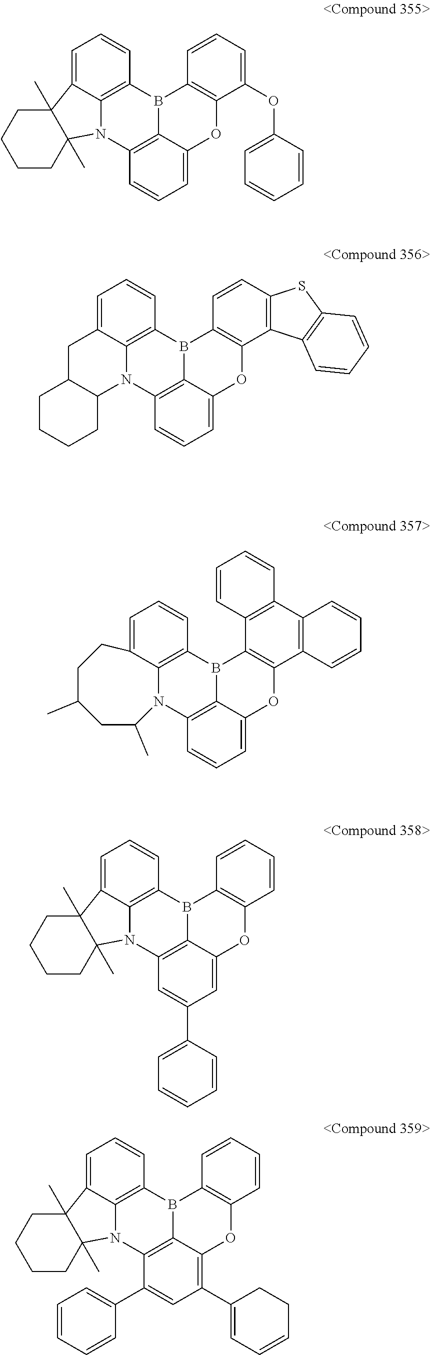 [SMILES: CC12CCCCC1(C)N1c3cccc4c3B(c3cccc(Oc5ccccc5)c3O4)c3cccc2c31 ; c1cc2c3c(c1)B1c4ccc5sc6ccccc6c5c4Oc4cccc(c41)N3C1CCCCC1C2 ; CC1CCc2cccc3c2N(c2cccc4c2B3c2c(c3ccccc3c3ccccc23)O4)C(C)C1 ; CC12CCCCC1(C)N1c3cc(-c4ccccc4)cc4c3B(c3ccccc3O4)c3cccc2c31 ; CC12CCCCC1(C)N1c3c(cccc32)B2c3ccccc3Oc3c(C4=CC=CCC4)cc(-c4ccccc4)c1c32]

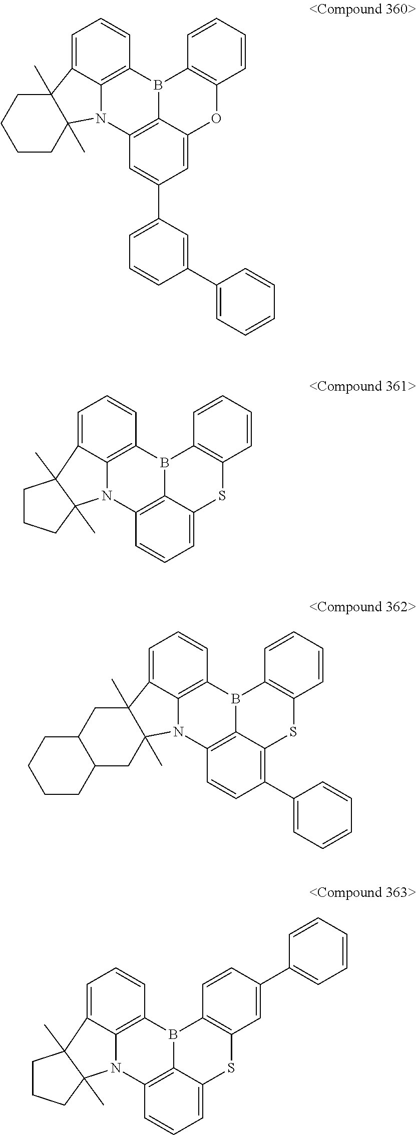 [SMILES: CC12CCCCC1(C)N1c3cc(-c4cccc(-c5ccccc5)c4)cc4c3B(c3ccccc3O4)c3cccc2c31 ; CC12CCCC1(C)N1c3cccc4c3B(c3ccccc3S4)c3cccc2c31 ; CC12CC3CCCCC3CC1(C)N1c3ccc(-c4ccccc4)c4c3B(c3ccccc3S4)c3cccc2c31 ; CC12CCCC1(C)N1c3cccc4c3B(c3ccc(-c5ccccc5)cc3S4)c3cccc2c31]

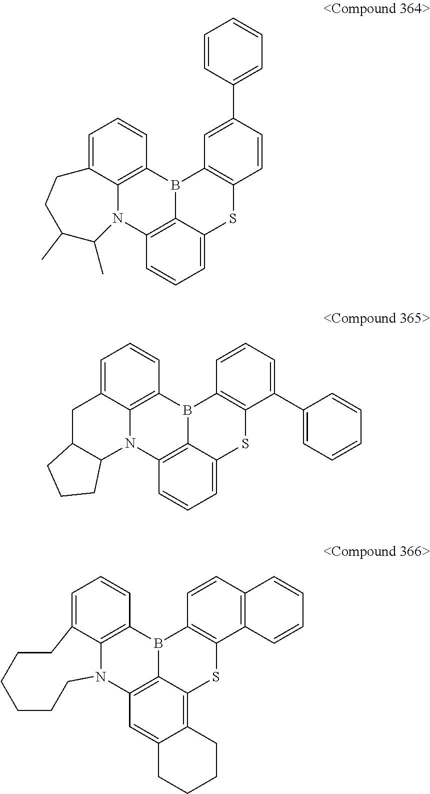 [SMILES: CC1CCc2cccc3c2N(c2cccc4c2B3c2cc(-c3ccccc3)ccc2S4)C1C ; c1ccc(-c2cccc3c2Sc2cccc4c2B3c2cccc3c2N4C2CCCC2C3)cc1 ; c1cc2c3c(c1)B1c4ccc5ccccc5c4Sc4c5c(cc(c41)N3CCCCCC2)CCCC5]

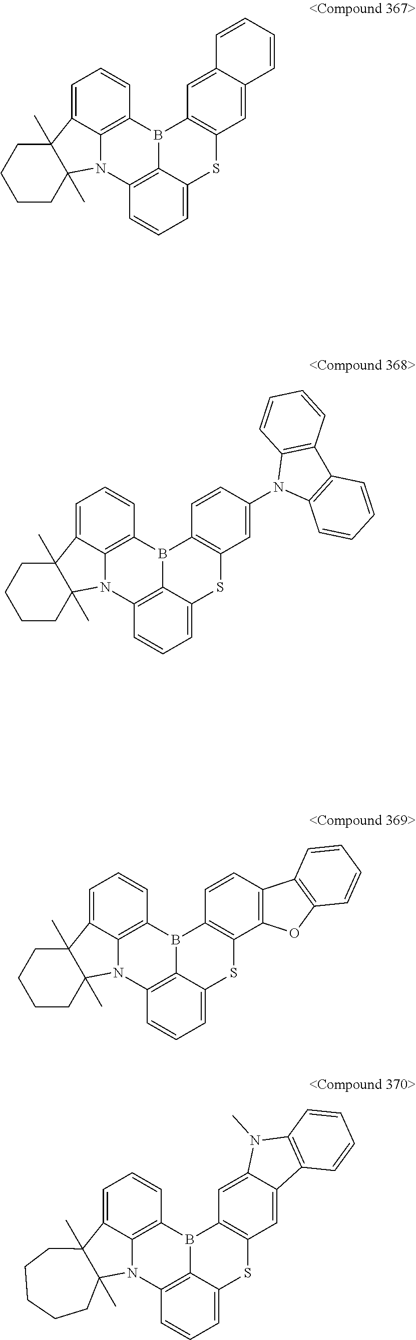 [SMILES: CC12CCCCC1(C)N1c3cccc4c3B(c3cc5ccccc5cc3S4)c3cccc2c31 ; CC12CCCCC1(C)N1c3cccc4c3B(c3ccc(-n5c6ccccc6c6ccccc65)cc3S4)c3cccc2c31 ; CC12CCCCC1(C)N1c3cccc4c3B(c3cccc2c31)c1ccc2c(oc3ccccc32)c1S4 ; Cn1c2ccccc2c2cc3c(cc21)B1c2cccc4c2N(c2cccc(c21)S3)C1(C)CCCCCC41C]

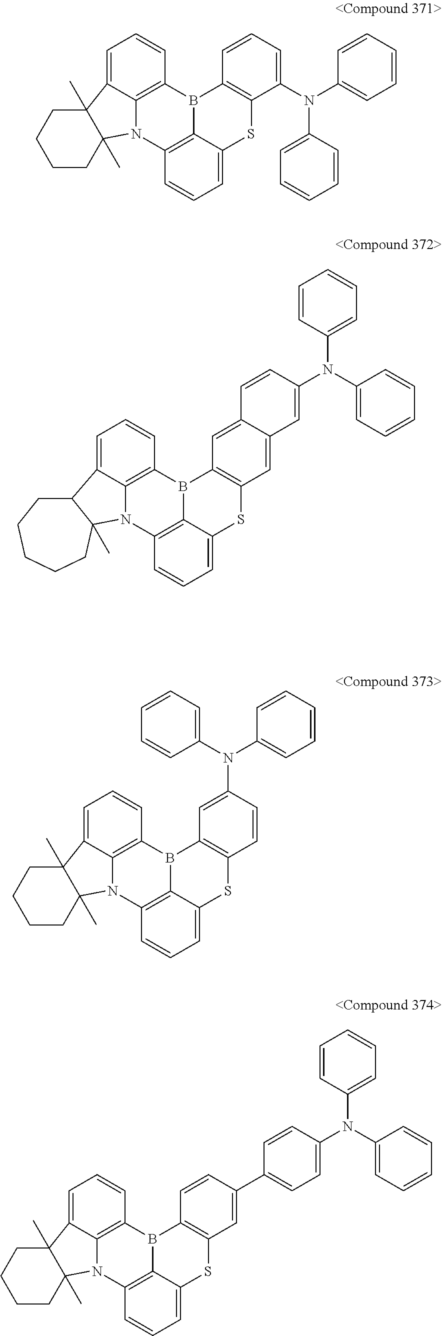 [SMILES: CC12CCCCC1(C)N1c3cccc4c3B(c3cccc(N(c5ccccc5)c5ccccc5)c3S4)c3cccc2c31 ; CC12CCCCCC1c1cccc3c1N2c1cccc2c1B3c1cc3ccc(N(c4ccccc4)c4ccccc4)cc3cc1S2 ; CC12CCCCC1(C)N1c3cccc4c3B(c3cc(N(c5ccccc5)c5ccccc5)ccc3S4)c3cccc2c31 ; CC12CCCCC1(C)N1c3cccc4c3B(c3ccc(-c5ccc(N(c6ccccc6)c6ccccc6)cc5)cc3S4)c3cccc2c31]

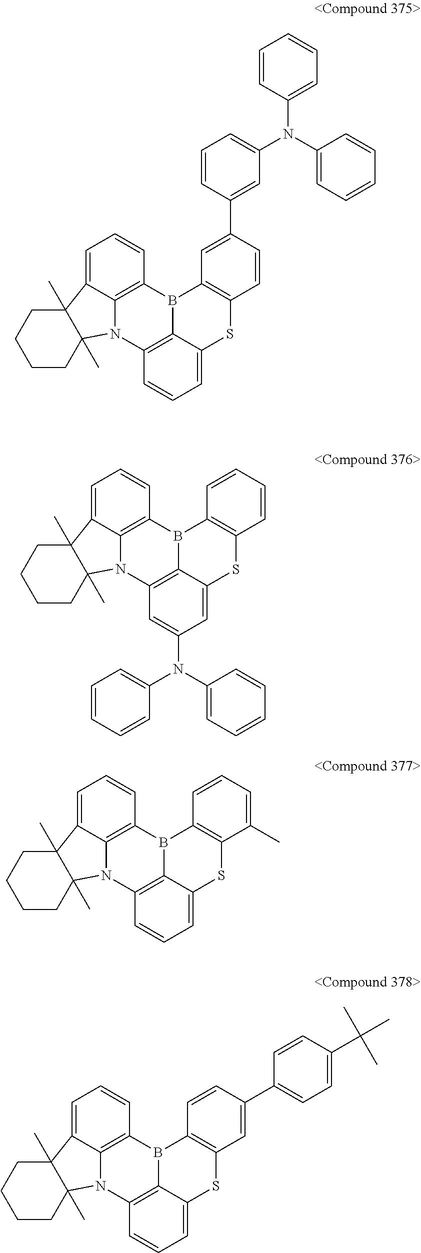

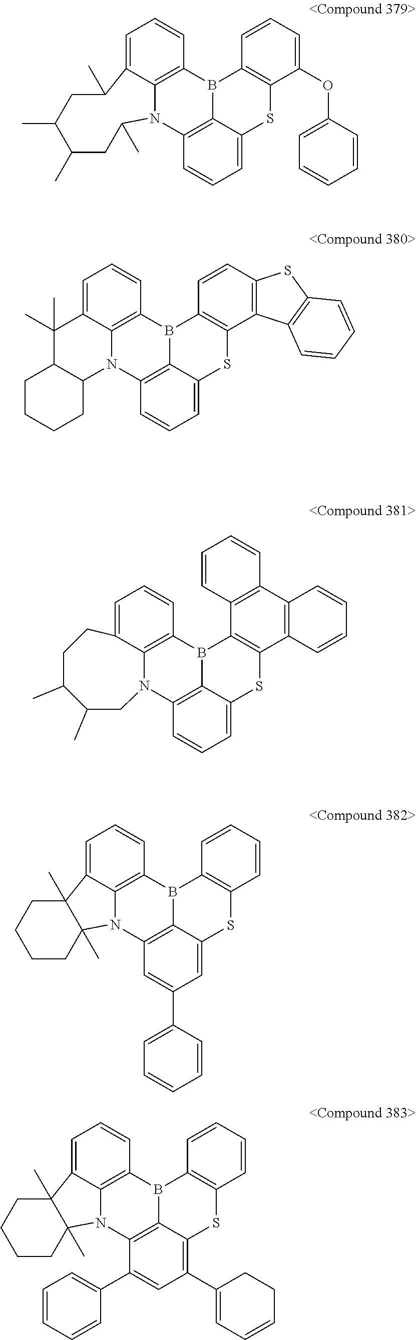 [SMILES: CC1CC(C)C(C)CC(C)N2c3cccc4c3B(c3cccc(Oc5ccccc5)c3S4)c3cccc1c32 ; CC1(C)c2cccc3c2N(c2cccc4c2B3c2ccc3sc5ccccc5c3c2S4)C2CCCCC21 ; CC1CCc2cccc3c2N(CC1C)c1cccc2c1B3c1c(c3ccccc3c3ccccc13)S2 ; CC12CCCCC1(C)N1c3cc(-c4ccccc4)cc4c3B(c3ccccc3S4)c3cccc2c31 ; CC12CCCCC1(C)N1c3c(cccc32)B2c3ccccc3Sc3c(C4=CC=CCC4)cc(-c4ccccc4)c1c32]

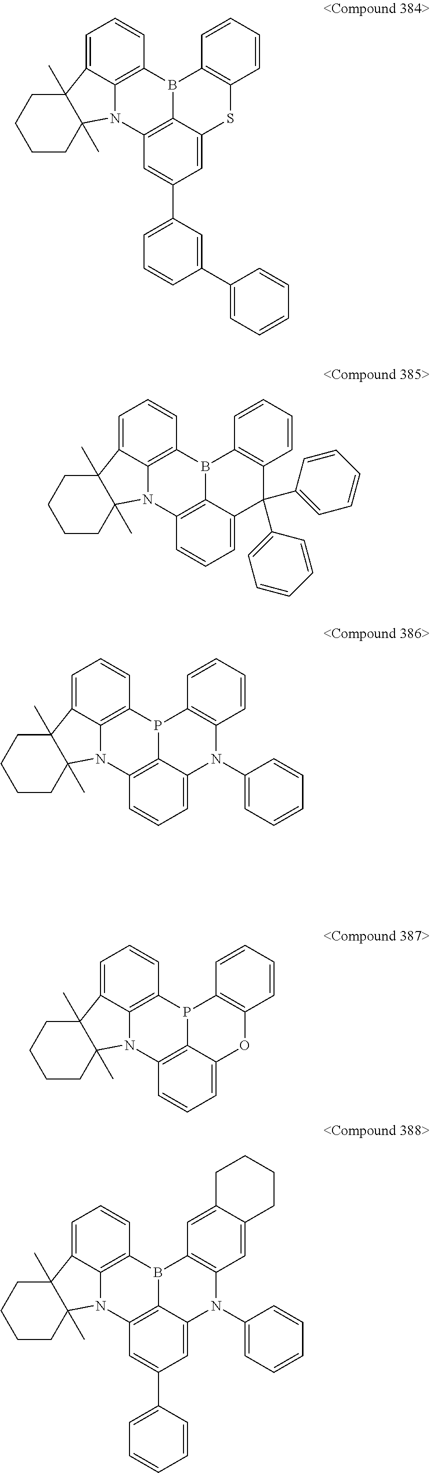 [SMILES: CC12CCCCC1(C)N1c3cc(-c4cccc(-c5ccccc5)c4)cc4c3B(c3ccccc3S4)c3cccc2c31 ; CC12CCCCC1(C)N1c3cccc4c3B(c3ccccc3C4(c3ccccc3)c3ccccc3)c3cccc2c31 ; CC12CCCCC1(C)N1c3cccc4c3P(c3ccccc3N4c3ccccc3)c3cccc2c31 ; CC12CCCCC1(C)N1c3cccc4c3P(c3ccccc3O4)c3cccc2c31 ; CC12CCCCC1(C)N1c3cc(-c4ccccc4)cc4c3B(c3cc5c(cc3N4c3ccccc3)CCCC5)c3cccc2c31]

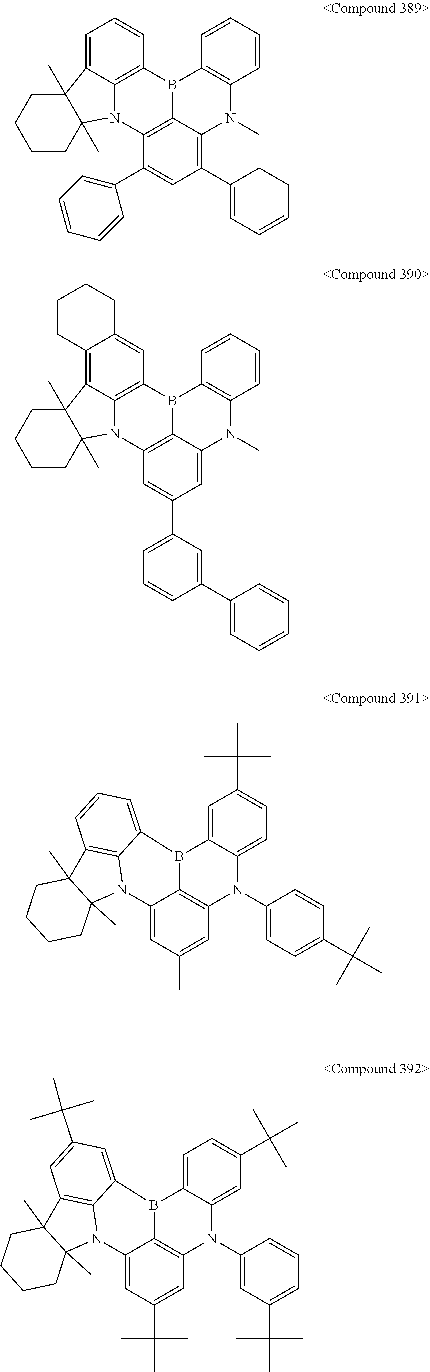

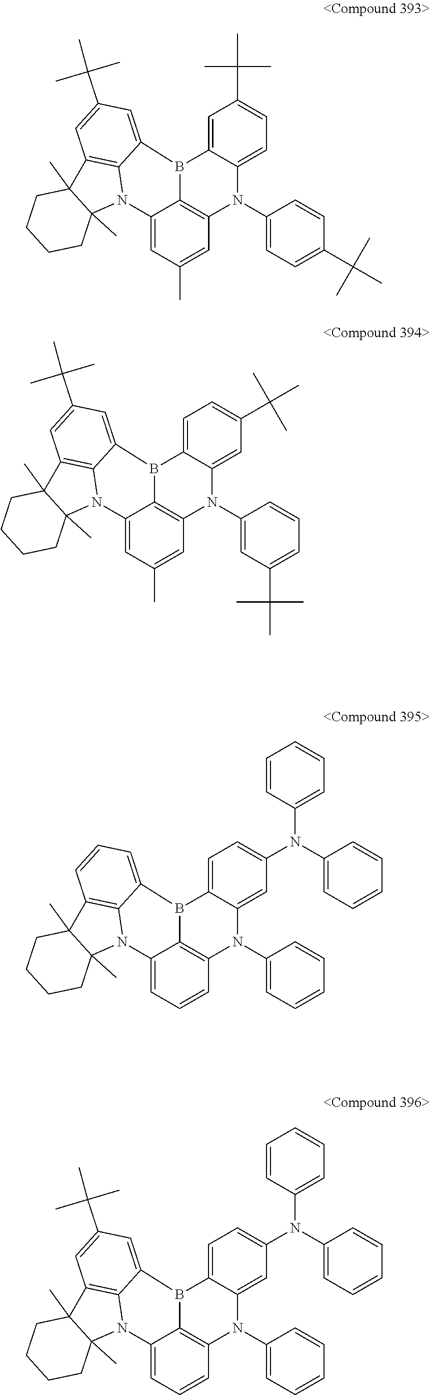 [SMILES: Cc1cc2c3c(c1)N1c4c(cc(C(C)(C)C)cc4C4(C)CCCCC14C)B3c1cc(C(C)(C)C)ccc1N2c1ccc(C(C)(C)C)cc1 ; Cc1cc2c3c(c1)N1c4c(cc(C(C)(C)C)cc4C4(C)CCCCC14C)B3c1ccc(C(C)(C)C)cc1N2c1cccc(C(C)(C)C)c1 ; CC12CCCCC1(C)N1c3cccc4c3B(c3ccc(N(c5ccccc5)c5ccccc5)cc3N4c3ccccc3)c3cccc2c31 ; CC(C)(C)c1cc2c3c(c1)C1(C)CCCCC1(C)N3c1cccc3c1B2c1ccc(N(c2ccccc2)c2ccccc2)cc1N3c1ccccc1]

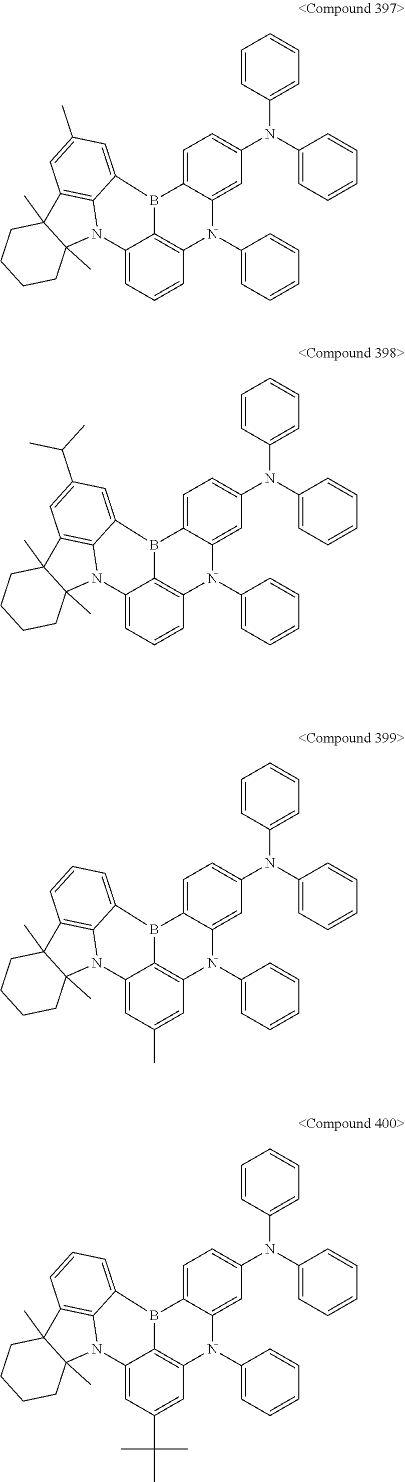 [SMILES: Cc1cc2c3c(c1)C1(C)CCCCC1(C)N3c1cccc3c1B2c1ccc(N(c2ccccc2)c2ccccc2)cc1N3c1ccccc1 ; CC(C)c1cc2c3c(c1)C1(C)CCCCC1(C)N3c1cccc3c1B2c1ccc(N(c2ccccc2)c2ccccc2)cc1N3c1ccccc1 ; Cc1cc2c3c(c1)N1c4c(cccc4C4(C)CCCCC14C)B3c1ccc(N(c3ccccc3)c3ccccc3)cc1N2c1ccccc1 ; CC(C)(C)c1cc2c3c(c1)N1c4c(cccc4C4(C)CCCCC14C)B3c1ccc(N(c3ccccc3)c3ccccc3)cc1N2c1ccccc1]

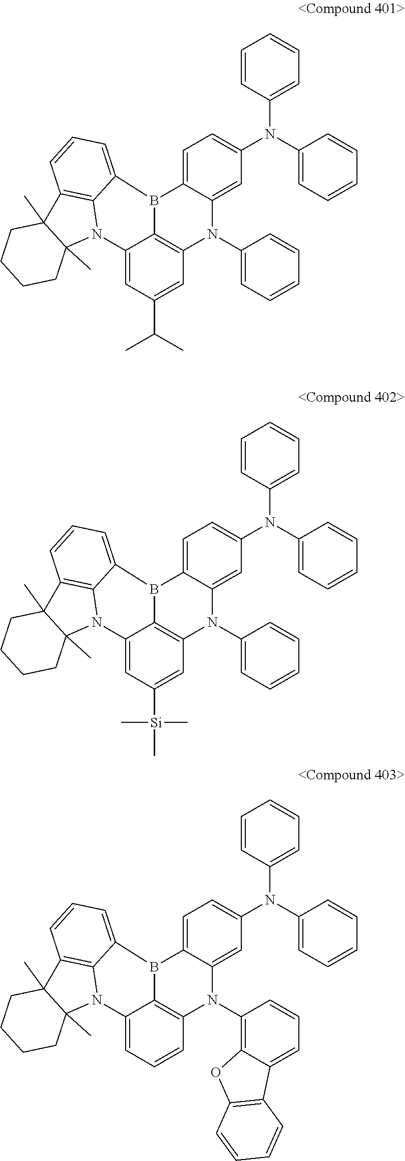 [SMILES: CC(C)c1cc2c3c(c1)N1c4c(cccc4C4(C)CCCCC14C)B3c1ccc(N(c3ccccc3)c3ccccc3)cc1N2c1ccccc1 ; CC12CCCCC1(C)N1c3cc([Si](C)(C)C)cc4c3B(c3ccc(N(c5ccccc5)c5ccccc5)cc3N4c3ccccc3)c3cccc2c31 ; CC12CCCCC1(C)N1c3cccc4c3B(c3ccc(N(c5ccccc5)c5ccccc5)cc3N4c3cccc4c3oc3ccccc34)c3cccc2c31]

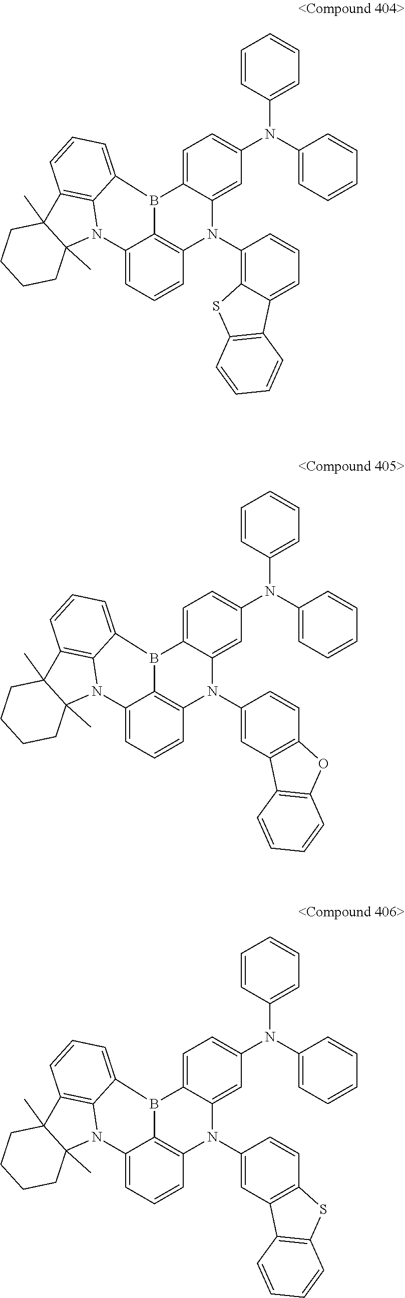 [SMILES: CC12CCCCC1(C)N1c3cccc4c3B(c3ccc(N(c5ccccc5)c5ccccc5)cc3N4c3cccc4c3sc3ccccc34)c3cccc2c31 ; CC12CCCCC1(C)N1c3cccc4c3B(c3ccc(N(c5ccccc5)c5ccccc5)cc3N4c3ccc4oc5ccccc5c4c3)c3cccc2c31 ; CC12CCCCC1(C)N1c3cccc4c3B(c3ccc(N(c5ccccc5)c5ccccc5)cc3N4c3ccc4sc5ccccc5c4c3)c3cccc2c31]

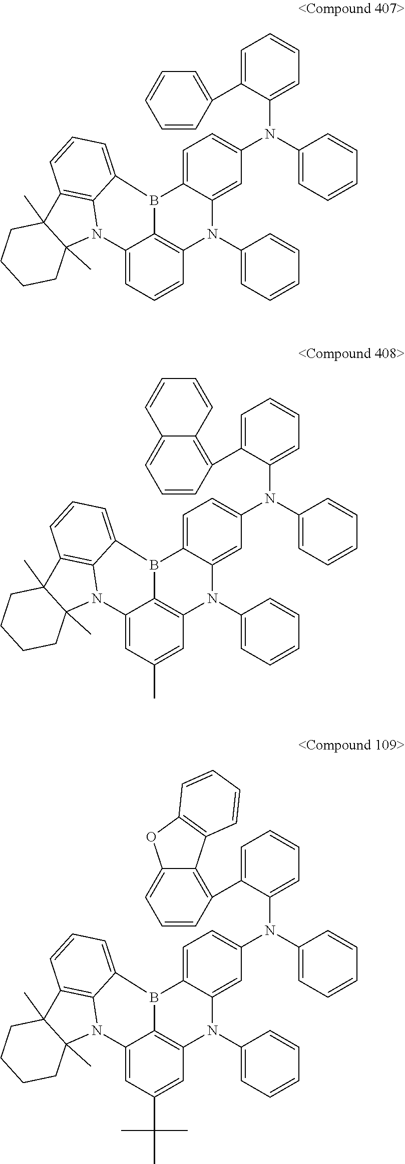 [SMILES: CC12CCCCC1(C)N1c3cccc4c3B(c3ccc(N(c5ccccc5)c5ccccc5-c5ccccc5)cc3N4c3ccccc3)c3cccc2c31 ; Cc1cc2c3c(c1)N1c4c(cccc4C4(C)CCCCC14C)B3c1ccc(N(c3ccccc3)c3ccccc3-c3cccc4ccccc34)cc1N2c1ccccc1 ; CC(C)(C)c1cc2c3c(c1)N1c4c(cccc4C4(C)CCCCC14C)B3c1ccc(N(c3ccccc3)c3ccccc3-c3cccc4oc5ccccc5c34)cc1N2c1ccccc1]

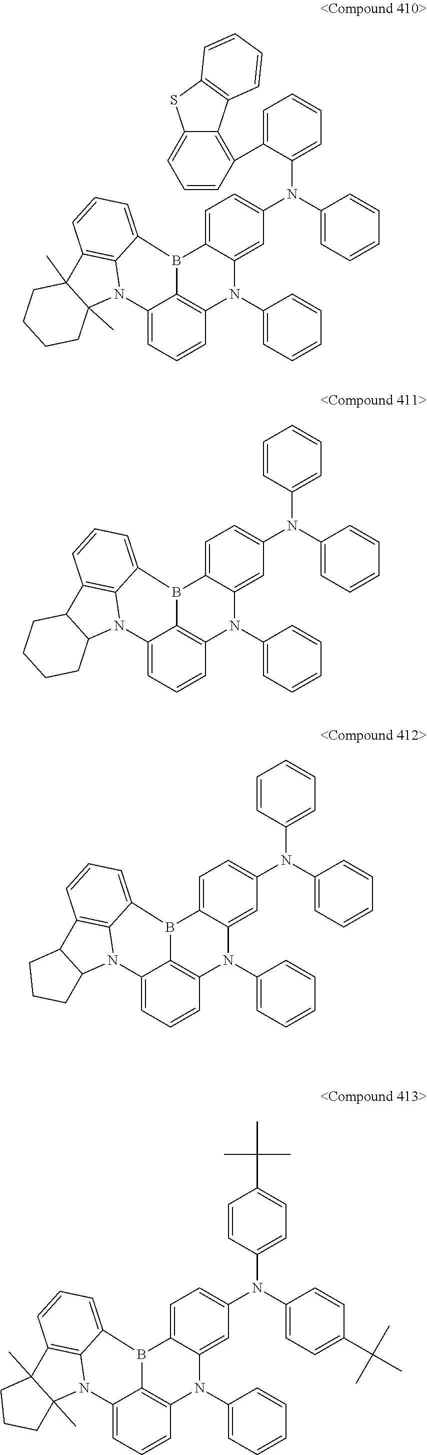 [SMILES: CC12CCCCC1(C)N1c3cccc4c3B(c3ccc(N(c5ccccc5)c5ccccc5-c5cccc6sc7ccccc7c56)cc3N4c3ccccc3)c3cccc2c31 ; c1ccc(N(c2ccccc2)c2ccc3c(c2)N(c2ccccc2)c2cccc4c2B3c2cccc3c2N4C2CCCCC32)cc1 ; c1ccc(N(c2ccccc2)c2ccc3c(c2)N(c2ccccc2)c2cccc4c2B3c2cccc3c2N4C2CCCC32)cc1 ; CC(C)(C)c1ccc(N(c2ccc(C(C)(C)C)cc2)c2ccc3c(c2)N(c2ccccc2)c2cccc4c2B3c2cccc3c2N4C2(C)CCCC32C)cc1]

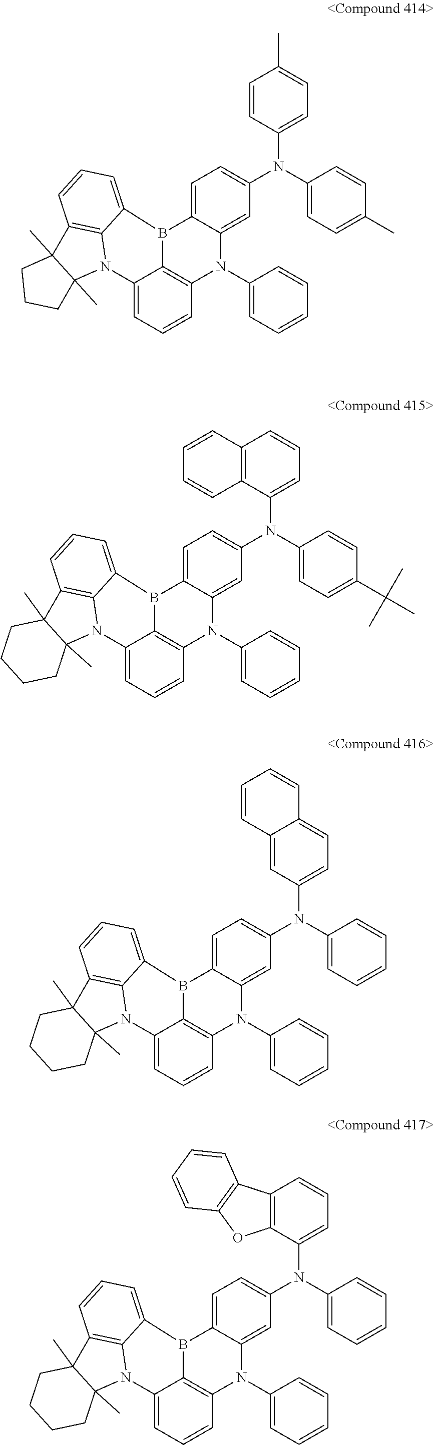 [SMILES: Cc1ccc(N(c2ccc(C)cc2)c2ccc3c(c2)N(c2ccccc2)c2cccc4c2B3c2cccc3c2N4C2(C)CCCC32C)cc1 ; CC(C)(C)c1ccc(N(c2ccc3c(c2)N(c2ccccc2)c2cccc4c2B3c2cccc3c2N4C2(C)CCCCC32C)c2cccc3ccccc23)cc1 ; CC12CCCCC1(C)N1c3cccc4c3B(c3ccc(N(c5ccccc5)c5ccc6ccccc6c5)cc3N4c3ccccc3)c3cccc2c31 ; CC12CCCCC1(C)N1c3cccc4c3B(c3ccc(N(c5ccccc5)c5cccc6c5oc5ccccc56)cc3N4c3ccccc3)c3cccc2c31]

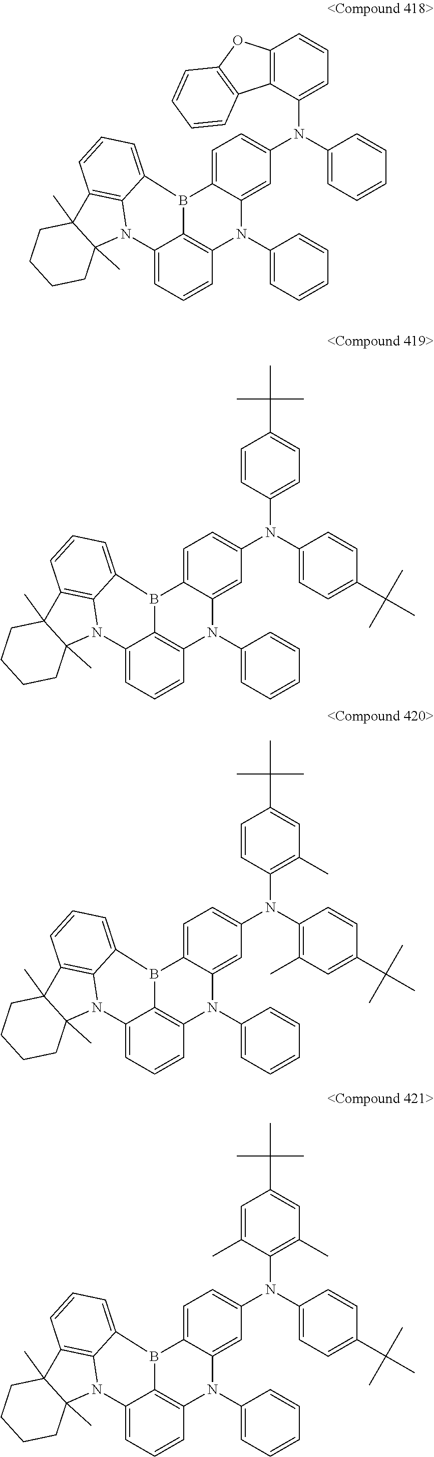

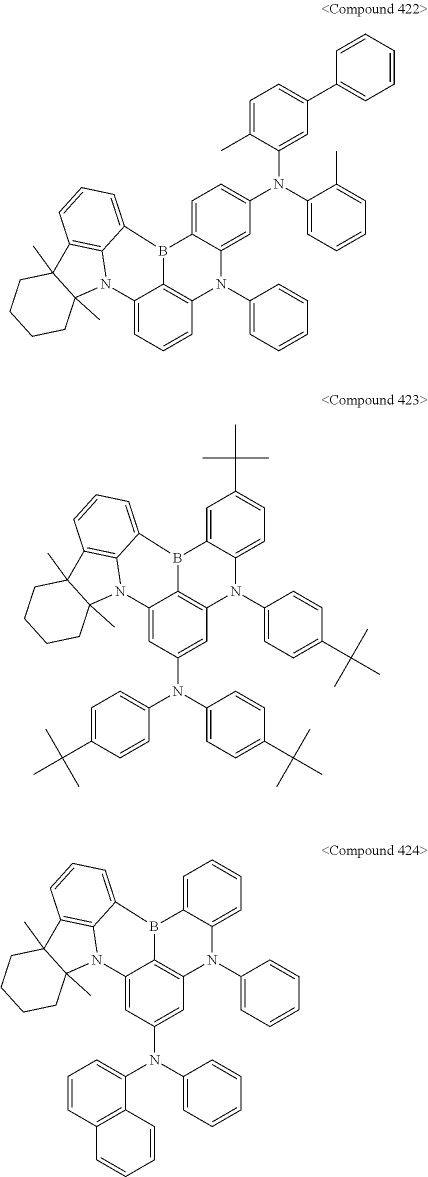 [SMILES: Cc1ccccc1N(c1ccc2c(c1)N(c1ccccc1)c1cccc3c1B2c1cccc2c1N3C1(C)CCCCC21C)c1cc(-c2ccccc2)ccc1C ; CC(C)(C)c1ccc(N(c2ccc(C(C)(C)C)cc2)c2cc3c4c(c2)N2c5c(cccc5C5(C)CCCCC25C)B4c2cc(C(C)(C)C)ccc2N3c2ccc(C(C)(C)C)cc2)cc1 ; CC12CCCCC1(C)N1c3cc(N(c4ccccc4)c4cccc5ccccc45)cc4c3B(c3ccccc3N4c3ccccc3)c3cccc2c31]

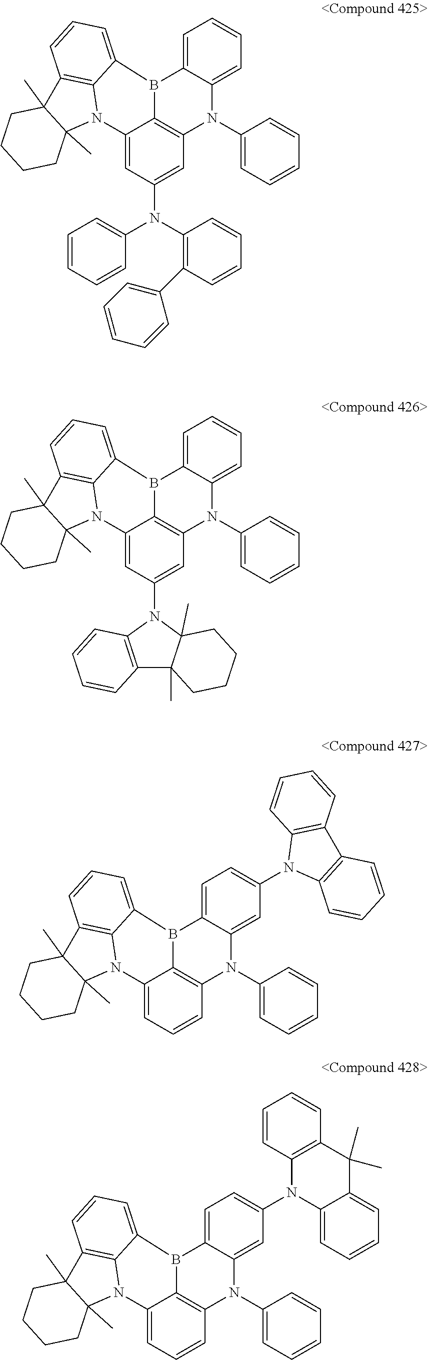 [SMILES: CC12CCCCC1(C)N1c3cc(N(c4ccccc4)c4ccccc4-c4ccccc4)cc4c3B(c3ccccc3N4c3ccccc3)c3cccc2c31 ; CC12CCCCC1(C)N(c1cc3c4c(c1)N1c5c(cccc5C5(C)CCCCC15C)B4c1ccccc1N3c1ccccc1)c1ccccc12 ; CC12CCCCC1(C)N1c3cccc4c3B(c3ccc(-n5c6ccccc6c6ccccc65)cc3N4c3ccccc3)c3cccc2c31 ; CC1(C)c2ccccc2N(c2ccc3c(c2)N(c2ccccc2)c2cccc4c2B3c2cccc3c2N4C2(C)CCCCC32C)c2ccccc21]

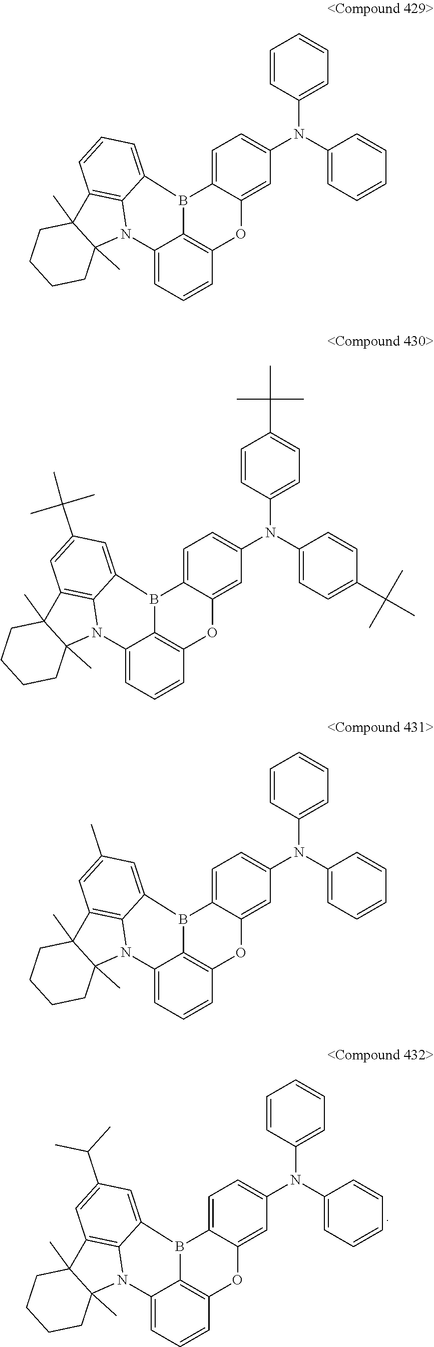 [SMILES: CC12CCCCC1(C)N1c3cccc4c3B(c3ccc(N(c5ccccc5)c5ccccc5)cc3O4)c3cccc2c31 ; CC(C)(C)c1ccc(N(c2ccc(C(C)(C)C)cc2)c2ccc3c(c2)Oc2cccc4c2B3c2cc(C(C)(C)C)cc3c2N4C2(C)CCCCC32C)cc1 ; Cc1cc2c3c(c1)C1(C)CCCCC1(C)N3c1cccc3c1B2c1ccc(N(c2ccccc2)c2ccccc2)cc1O3 ; CC(C)c1cc2c3c(c1)C1(C)CCCCC1(C)N3c1cccc3c1B2c1ccc(N(c2ccccc2)c2ccccc2)cc1O3]

The content of the dopant in the light-emitting layer may range from about 0.01 to 20 parts by weight, based on 100 parts by weight of the host, but is not limited thereto.

In addition to the above-mentioned dopants and hosts, the light-emitting layer may further include various hosts and dopant materials.

Below, the organic light-emitting diode of the present disclosure is explained with reference to the drawings.

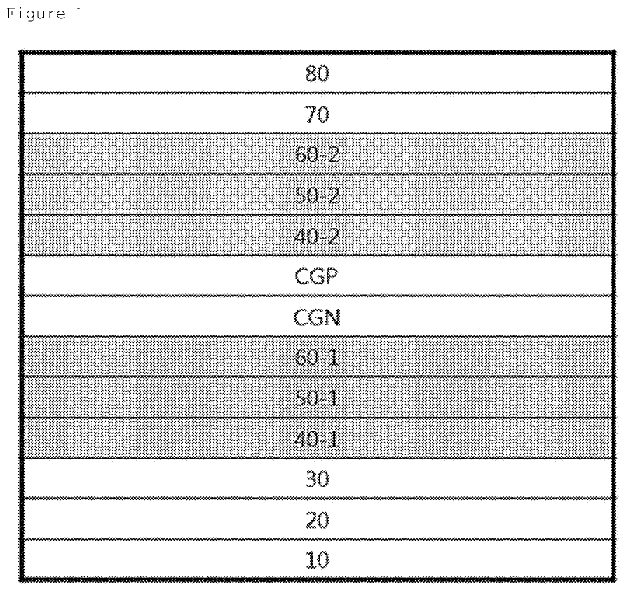

FIG. 1 is a schematic cross-sectional view of the structure of an organic light-emitting diode according to an embodiment of the present disclosure.

As shown in FIG. 1, the organic light-emitting diode according to an embodiment of the present disclosure comprises: an anode 20; a first emission part including a hole transport layer 40-1, a first organic light-emitting layer 50-1 containing a host and a dopant, and a first electron transport layer 60-1; a charge generation layer (CG) including a P-type charge generation layer (CGP) and an N-type charge generation layer (CGN); and a second emission part including a second hole transport layer 40-2, a second light-emitting layer 50-2 containing a host and a dopant, and a second electron transport layer 60-2; and a cathode 80,

In this structure, the charge generation layer (CG) including the P-type charge generation layer (CGP) and N-type charge generation layer (CGN) is located between the first emission part sequentially including a first hole transport layer, a first light-emitting layer, and a first electron transport layer and the second emission part sequentially including a second hole transport layer, a second light-emitting layer, and a second electron transport layer and forms a p-n junction structure through which the first emission part and the second emission part are joined to each other.

That is, the charge generation layer including a P-type charge generation layer (CGP) and an N-type charge generation layer (CGN) is provided as a p-n junction structure on the first electron transport layer wherein a material for the N-type charge generation layer may be the phenanthroline-based compound represented by Chemical Formula A according to the present disclosure.

Here, the N-type charge generation layer supplies electrons to the first electron transport layer of the first emission part and the first electron transport layer, in turn, supplies electrons the first light-emitting layer. Meanwhile, the P-type charge generation layer supplies holes to the second hole transport layer of the second emission part from which the electrons are, in turn, supplied to the second light-emitting layer.

In this regard, the first light-emitting layer of the first emission part; and the second light-emitting layer of the second emission part may employ the same or different hosts and the same or different dopants.

Furthermore, the organic light-emitting diode according to an embodiment of the present disclosure may include a hole injection layer 30 between the anode 20 and the first hole transport layer 40-1 and an electron injection layer 70 between the second electron transport layer 60-2 and the cathode 80.

Reference is made to FIG. 1 with regard to the organic light-emitting diode of the present disclosure and the fabrication method therefor.

First, a substrate 10 is coated with an anode electrode material to form an anode 20. So long as it is used in a typical organic electroluminescence device, any substrate may be used as the substrate 10. Preferable is an organic substrate or transparent plastic substrate that exhibits excellent transparency, surface smoothness, ease of handling, and waterproofness. As the anode electrode material, indium tin oxide (ITO), indium zinc oxide (IZO), tin oxide (SnO₂), or zinc oxide (ZnO), which are transparent and superior in terms of conductivity, may be used.

A hole injection layer material is applied on the anode 20 by thermal deposition in a vacuum or by spin coating to form a hole injection layer 30. Subsequently, thermal deposition in a vacuum or by spin coating may also be conducted to form a hole transport layer 40 with a hole transport layer material on the hole injection layer 30.

So long as it is typically used in the art, any material may be selected for the hole injection layer without particular limitations thereto. Examples include, but are not limited to, 2-TNATA [4,4′,4″-tris(2-naphthylphenyl-phenylamino)-triphenylamine], NPD [N,N′-di(1-naphthyl)-N,N′-diphenylbenzidine)], TPD [N,N′-diphenyl-N,N′-bis(3-methylphenyl)-1,1′-biphenyl-4,4′-diamine], and DNTPD [N,N′-diphenyl-N,N′-bis-[4-(phenyl-m-tolyl-amino)-phenyl]-biphenyl-4,4′-diamine].

Any material that is typically used in the art may be selected for the hole transport layers 40-1 and 40-2, without particular limitations thereto. Examples include, but are not limited to, N,N′-bis(3-methylphenyl)-N,N′-diphenyl-[1,1-biphenyl]-4,4′-diamine (TPD) and N,N′-di(naphthalen-1-yl)-N,N′-diphenylbenzidine (a-NPD).

Next, a first light-emitting layer 50-1 may be deposited on the first hole transport layer 40-1 by deposition in a vacuum or by spin coating.

Herein, the first light-emitting layer 50-1 may contain a host and a dopant and the materials are as described above.

In some embodiments of the present disclosure, the first light-emitting layer 50-1 particularly ranges in thickness from 50 to 2,000 Å.

Meanwhile, a first electron transport layer 60-1 is applied on the light-emitting layer by deposition in a vacuum and spin coating.

Materials for use in the electron transport layers 60-1 and 60-2 function to stably carry the electrons injected from the electron injection electrode (cathode), and may be electron transport materials known in the art. Examples of the electron transport materials known in the art include quinoline derivatives, particularly, tris(8-quinolinolate)aluminum (Alq₃), Liq, TAZ, BAlq, beryllium bis(benzoquinolin-10-olate) (Bebq₂), Compound 201, Compound 202, BCP, and oxadiazole derivatives such as PBD, BMD, and BND, but are not limited thereto:

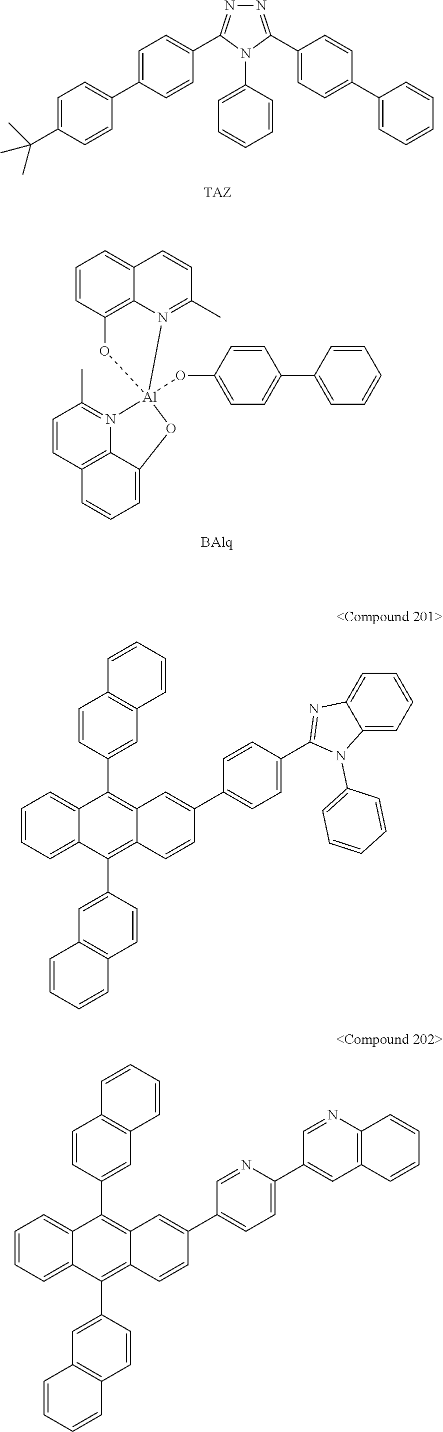

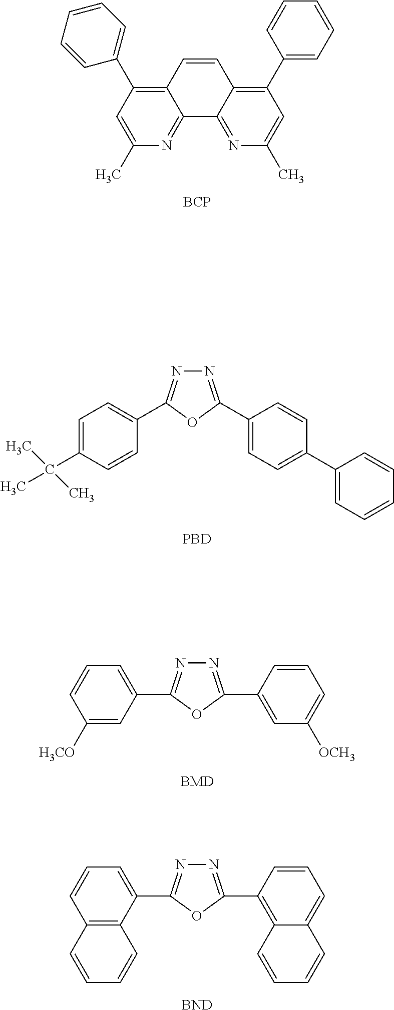

In addition, the charge generation layer (CG) sequentially includes a P-type charge generation layer (CGP) and an N-type charge generation layer (CGN) to form a p-n junction between the P-type charge generation layer and the N-type charge generation layer, so that holes and electrons are simultaneously generated between the P-type charge generation layer and the N-type charge generation layer. In this regard, the electrons are transported through the N-type charge generation layer in the direction toward the electron transport layer while the holes are transported in the direction toward the P-type charge generation layer, whereby holes or electrons can be easily generated to prevent the driving voltage for hole injection from being increased.

The charge generation layer (CG) may be deposited on the first electron transport layer (60-1) by deposition in a vacuum or by spin coating. The constituent material therefor includes the phenanthroline-based compound represented by Chemical Formula A according to the present disclosure as described in the foregoing.

In the organic light-emitting diode of the present disclosure, the P-type charge generation layer may be made of a metal or a P-type dopant-doped organic material. Herein, the metal may be at least one or an alloy of two or more metals selected from the group consisting of Al, Cu, Fe, Pb, Zn, Au, Pt, W, In, Mo, Ni, and Ti. In addition, the P-type dopant used for the P-type dopant-doped organic material and the host material for the P-type charge generation layer may employ any typically used materials. For instance, the P-type dopant may be one selected from F4-TCNQ, iodine, FeCl₃, FeF₃, and SbCl₅. In addition, the host for the P-type charge generation layer may be at least one selected from the group consisting of NPB, TPD, N,N,N′,N′-tetranaphthalenyl-benzidine (TNB), and HAT-CN.

In the organic light-emitting diode according to the present disclosure, the N-type charge generation layer comprising at least one of the phenanthroline-based compounds may contain an alkali metal- or alkaline earth metal-bearing metal compound as the dopant.

When the N-type charge generation layer is doped with an alkali metal or an alkaline earth metal, electron injection into the N-type charge generation layer can be improved. In detail, when an alkali metal or an alkaline earth metal is used as a dopant in the N-type charge generation layer, it is assumed that the energy level difference between the N-type charge generation layer and the P-type charge generation layer is reduced to improve the electron injection from the N-type charge generation layer into the first electron transport layer.

By way of example, the N-type charge generation layer may contain at least one selected from the group consisting of LiQ, LiF, NaF, KF, RbF, CsF, FrF, BeF₂, MgF₂, CaF₂, SrF₂, BaF₂, and RaF₂, but with no limitations thereto. The alkali metal or alkaline earth metal may be added in an amount of approximately 1 to 30% by weight, particularly in an amount of 0.5 to 15% by weight, and more particularly in an amount of 1 to 10% by weight, based on the phenanthroline compound according to the present disclosure, but with no limitations thereto.

Subsequently, a second emission part including a second hole transport layer 40-2, a second light-emitting layer 50-2 containing a host and a dopant, and a second electron transport layer 60-2 is positioned on the charge generation layer (CG). The second hole transport layer, the second light-emitting layer, and the second electron transport layer may employ the same materials as in the first hole transport layer (40-1), the first light-emitting layer 50-1, and the first electron transport layer 60-1 in the first emission part, as described above, and are deposited by deposition in a vacuum or by spin coating.

In the organic light-emitting diode of the present disclosure, an electron injection layer (EIL) that functions to facilitate electron injection from the cathode may be deposited on the second electron transport layer 60-2. The material for the EIL is not particularly limited.

Any material that is conventionally used in the art can be available for the electron injection layer 70 without particular limitations. Examples include CsF, NaF, LiF, Li₂O, and BaO. Deposition conditions for the electron injection layer may vary, depending on compounds used, but may be generally selected from condition scopes that are almost the same as for the formation of the hole injection layer.

The electron injection layer may range in thickness from about 1 Å to about 100 Å, and particularly from about 3 Å to about 90 Å. Given the thickness range for the electron injection layer, the diode can exhibit satisfactory electron injection properties without actually elevating a driving voltage.

In order to facilitate electron injection, the cathode 80 may be made of a material having a small work function, such as metal or metal alloy such as lithium (Li), magnesium (Mg), calcium (Ca), an alloy aluminum (Al) thereof, aluminum-lithium (Al—Li), magnesium-indium (Mg—In), and magnesium-silver (Mg—Ag). Alternatively, ITO or IZO may be employed to form a transparent cathode for an organic light-emitting diode.

In another embodiment of the present disclosure, the organic light-emitting diode may include two charge generation layers therein. In greater detail, the organic light-emitting diode according to the present disclosure may comprise an anode 20, a first hole transport layer 40-1, a first light-emitting layer 50-1 containing a host and a dopant, a first electron transport layer 60-1, a first charge generation layer (CG-1), a second hole transport layer 40-2, a second light-emitting layer 50-2 containing a host and a dopant, a second electron transport layer 60-2, a second charge generation layer (CG-2), a third hole transport layer 40-3, a third light-emitting layer 50-3 containing a host and a dopant, a third electron transport layer 60-3, and a cathode 80.

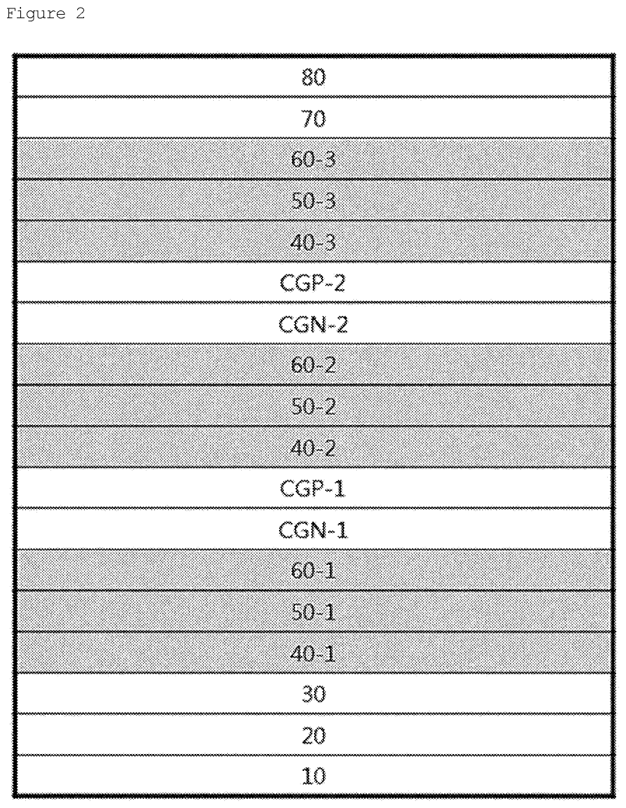

This structure is illustrated in FIG. 2.

FIG. 2 shows an illustrative structure of an organic light-emitting diode including two charge generation layers according to the present disclosure. As shown in FIG. 2, a first charge generation layer (CG-1) includes a P-type charge generation layer (CGP-1) and an N-type charge generation layer (CGN-1) and a second charge generation layer (CG-2) also includes a P-type charge generation layer (CGP-2) and an N-type charge generation layer (CGN-2).

In this structure, the first charge generation layer (CG-1) is located between the first emission part sequentially including a first hole transport layer 40-1, a first light-emitting layer 50-1, and a first electron transport layer 60-1 and the second emission part sequentially including a second hole transport layer 40-2, a second light-emitting layer 50-2, and a second electron transport layer 60-2 while the second charge generation layer (CG-2) is located between the second emission part sequentially including a second hole transport layer 40-2, a second light-emitting layer 50-2, and a second electron transport layer 60-2 and the third emission part sequentially including a third hole transport layer 40-3, a third light-emitting layer 50-3, and a third electron transport layer 60-3.

In the structure of the organic light-emitting diode, as shown in FIG. 2, the phenanthroline-based compound represented by Chemical Formula A according to the present disclosure is used as a material for the N-type charge generation layer (CGN-1) on the first electron transport layer 60-1 and the P-type charge generation layer (CGP-1) is interposed between the N-type charge generation layer and the second hole transport layer 40-2 to form a p-n junction. In addition, the phenanthroline-based compound represented by Chemical Formula A according to the present disclosure may be used as a material for the N-type charge generation layer (CGN-2) on the second electron transport layer 60-2 and the P-type charge generation layer (CGP-2) is interposed between the N-type charge generation layer and the third hole transport layer to form a p-n junction.

Subsequently, a third emission part including a third hole transport layer 40-3, a third light-emitting layer 50-3 containing a host and a dopant, and a third electron transport layer 60-3 is positioned on the second charge generation layer (CG-2).

The respective materials for the second hole transport layer, the second light-emitting layer, and the second electron transport layer in the second emission part and for the third hole transport layer, the third light-emitting layer, and the third electron transport layer in the third emission part may be the same as or different from the corresponding materials for the first hole transport layer 40-1, the first light-emitting layer 50-1, and the first electron transport layer 60-1 in the first emission part, and may be deposited by deposition in a vacuum or by spin coating.

Moreover, the organic light-emitting diode of the present disclosure may further comprise a light-emitting layer containing a blue, green, or red luminescent material that emits radiations in the wavelength range of 380 nm to 800 nm. That is, the light-emitting layer in the present disclosure has a multi-layer structure wherein the blue, green, or red luminescent material may be a fluorescent material or a phosphorescent material.

Furthermore, at least one selected from among the layers may be deposited using a single-molecule deposition process or a solution process.

Here, the deposition process is a process by which a material is vaporized in a vacuum or at a low pressure and deposited to form a layer, and the solution process is a method in which a material is dissolved in a solvent and applied for the formation of a thin film by means of inkjet printing, roll-to-roll coating, screen printing, spray coating, dip coating, spin coating, etc.

Also, the organic light-emitting diode of the present disclosure may be applied to a device selected from among flat display devices, flexible display devices, monochrome or grayscale flat illumination devices, and monochrome or grayscale flexible illumination devices.

A better understanding of the present disclosure may be obtained through the following examples which are set forth to illustrate, but are not to be construed as limiting the present disclosure.

### Synthesis Example 1: Synthesis of Compound 1

### Synthesis Example 1-1. Synthesis of <Intermediate 1-a>

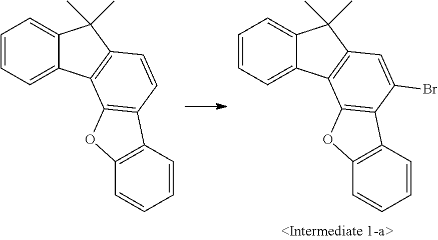 [SMILES: CC1(C)c2ccccc2-c2c1ccc1c2oc2ccccc21 ; CC1(C)c2ccccc2-c2c1cc(Br)c1c2oc2ccccc21]

In a 500-mL reactor, 7,7-dimethyl-7H-fluoreno[4,3-b]benzofuran (28.6 g, 97 mmol) and chloroform (300 mL) were stirred together. Then, N-bromosuccinimide (28.4 g, 97 mmol) was slowly added to the reactor. The mixture was mixed at room temperature for 4 hours. After completion of the reaction, distilled water was added dropwise to form brown crystals. Filtration and recrystallization of the crystals afforded <Intermediate 1-a> (27.8 g, yield 91%)

### Synthesis Example 1-2. Synthesis of <Intermediate 1-b>

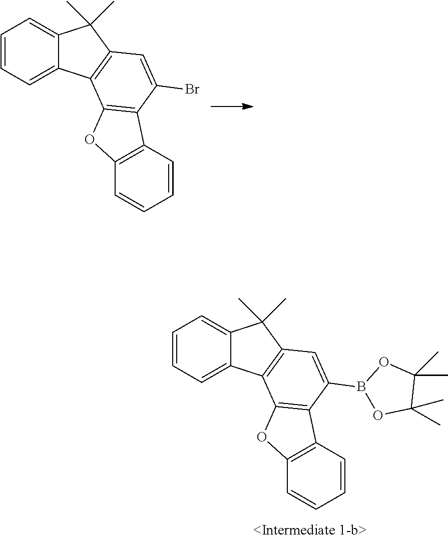 [SMILES: CC1(C)c2ccccc2-c2c1cc(Br)c1c2oc2ccccc21 ; CC1(C)c2ccccc2-c2c1cc(B1OC(C)(C)C(C)(C)O1)c1c2oc2ccccc21]

In a 500-mL reactor, <Intermediate 1-a> (14 g, 34 mmol), bis(pinacolato)diboron (10.4 g, 41 mmol), 1,1′-bis(diphenylphosphino)ferrocene palladium (II) dichloride (1.4 g, 1 mmol), potassium acetate (6.7 g, 68 mmol), and toluene (140 ml) were stirred together for 12 hours under reflux. After completion of the reaction, the solid matter was filtered off and the filtrate was concentrated in a vacuum. Recrystallization of the concentrate afforded <Intermediate 1-b> (12.5 g, yield 71%).

### Synthesis Example 1-3. Synthesis of <Compound 1>

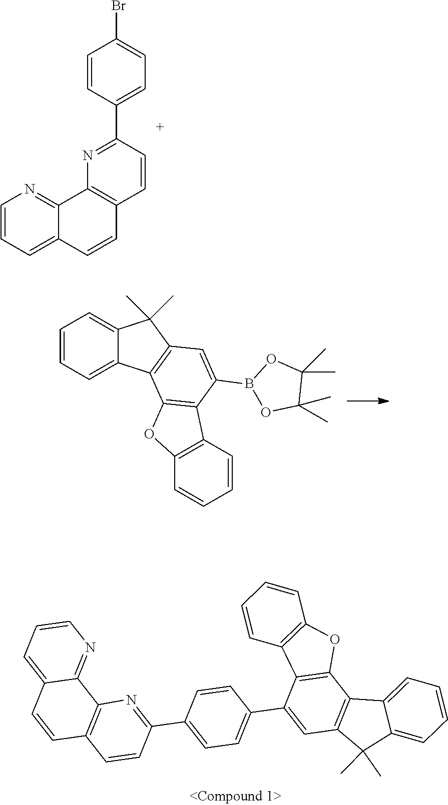 [SMILES: Brc1ccc(-c2ccc3ccc4cccnc4c3n2)cc1 ; CC1(C)c2ccccc2-c2c1cc(B1OC(C)(C)C(C)(C)O1)c1c2oc2ccccc21 ; CC1(C)c2ccccc2-c2c1cc(-c1ccc(-c3ccc4ccc5cccnc5c4n3)cc1)c1c2oc2ccccc21]

In a 2-L reactor, 2-(4-bromophenyl)-1,10-phenanthroline (10 g, 29.8 mmol), <Intermediate 1-b> (11 g, 48 mmol), potassium carbonate 6.2 g (92 mmol), tetrakis(triphenylphosphine) palladium (0.6 g, 1 mmol), H₂O (20 mL), ethanol (50 mL), and toluene (50 mL) were stirred together for 24 hours under reflux. After completion of the reaction, the reaction mixture was subjected to layer separation. The organic layer thus formed was concentrated in a vacuum. Purification by column chromatography afforded <Compound 1> (8.2 g, yield 51%).

MS (MALDI-TOF): m/z 538.20 [M⁺]

### Synthesis Example 2: Synthesis of Compound 10

### Synthesis Example 2-1. Synthesis of <Intermediate 2-a>

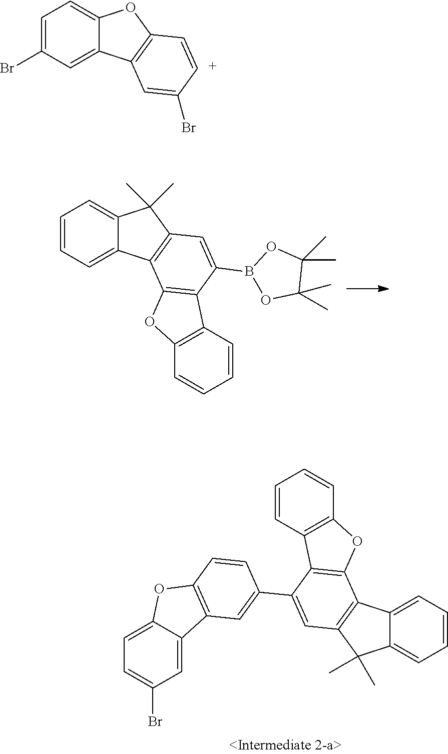 [SMILES: BrC1=Cc2c(oc3ccc(Br)cc23)C=[C+]1 ; CC1(C)c2ccccc2-c2c1cc(B1OC(C)(C)C(C)(C)O1)c1c2oc2ccccc21 ; CC1(C)c2ccccc2-c2c1cc(-c1ccc3oc4ccc(Br)cc4c3c1)c1c2oc2ccccc21]

The same procedure as in Synthesis Example 1-3 was carried out, with the exception of using 2,8-dibromodibenzofuran instead of 2-(4-bromophenyl)-1,10-phenanthroline, to afford <Intermediate 2-a>, yield 68%)

### Synthesis Example 2-2. Synthesis of <Compound 10>

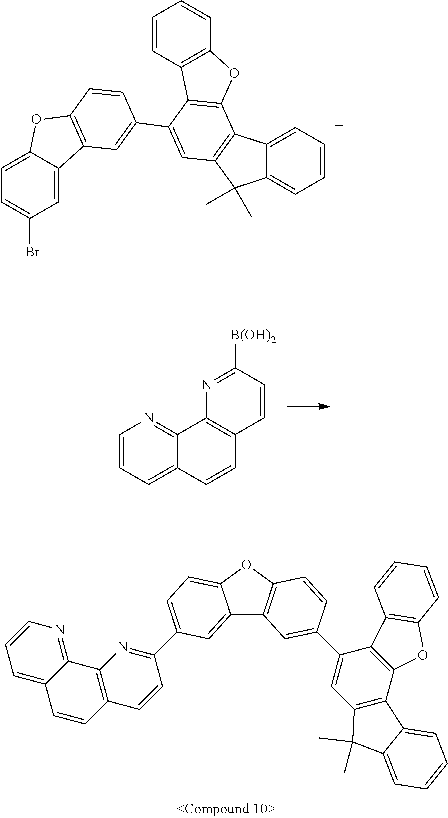 [SMILES: CC1(C)c2ccccc2-c2c1cc(-c1ccc3oc4ccc(Br)cc4c3c1)c1c2oc2ccccc21 ; OB(O)c1ccc2ccc3cccnc3c2n1 ; CC1(C)c2ccccc2-c2c1cc(-c1ccc3oc4ccc(-c5ccc6ccc7cccnc7c6n5)cc4c3c1)c1c2oc2ccccc21]

The same procedure as in Synthesis Example 1-3 was carried out, with the exception of using <Intermediate 2-a> and 1,10-phenanthrolin-2-yl boronic acid instead of 2-(4-bromophenyl)-1,10-phenanthroline and <Intermediate 1-b>, respectively, to afford <Compound 10>. (yield 58%)

MS (MALDI-TOF): m/z 628.22 [M⁺]

### Synthesis Example 3: Synthesis of Compound 22

### Synthesis Example 3-1. Synthesis of <Intermediate 3-a>

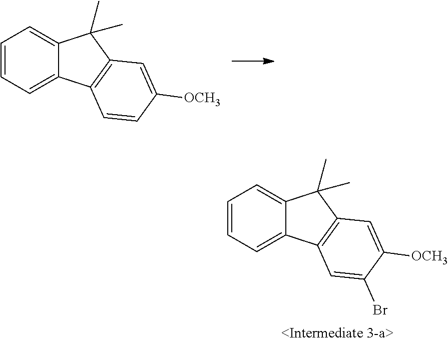 [SMILES: COc1ccc2c(c1)C(C)(C)c1ccccc1-2 ; COc1cc2c(cc1Br)-c1ccccc1C2(C)C]

In a round-bottom flask, 2-methoxy-9,9-dimethyl-9H-fluorene 30 g (133 mmol), N-bromosuccinimide 23.8 g (133 mmol), and dimethylformamide (600 ml) were stirred together at 50° C. for 12 hours under a nitrogen atmosphere. After completion of the reaction, the organic layer was concentrated in a vacuum. Purification by column chromatography afforded <Intermediate 3-a> (28 g, yield 70%).

### Synthesis Example 3-2. Synthesis of <Intermediate 3-b>

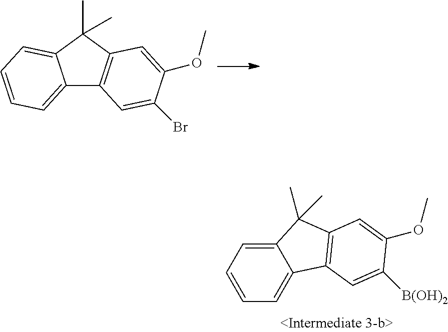 [SMILES: COc1cc2c(cc1Br)-c1ccccc1C2(C)C ; COc1cc2c(cc1B(O)O)-c1ccccc1C2(C)C]

Into a 300-mL flask was added <Intermediate 3-a> (17.6 g, 48.4 mmol), followed by tetrahydrofuran (200 ml) under a nitrogen atmosphere. After the temperature was lowered to −78° C., 1.6 M n-butyl lithium (36.3 ml, 58.1 mmol) was dropwise added slowly. One hour later, drops of trimethyl borate (7.0 ml, 62.9 mmol) were slowly added with the low temperature maintained, followed by stirring at room temperature. After completion of the reaction, the reaction mixture was subjected to layer separation. The organic layer thus formed was concentrated in a vacuum and recrystallized to afford <Intermediate 3-b> (18 g, yield 91%).

### Synthesis Example 3-3. Synthesis of <Intermediate 3-c>

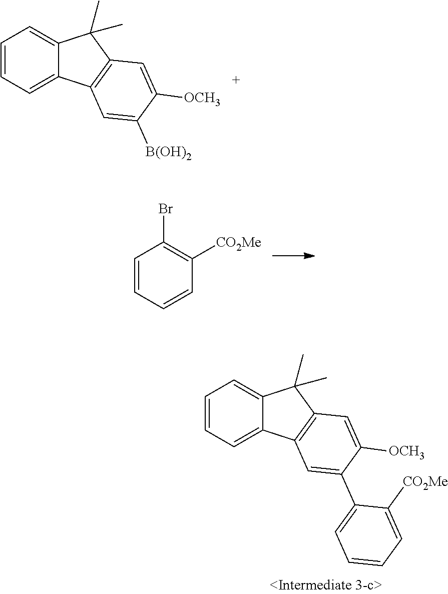 [SMILES: COc1cc2c(cc1B(O)O)-c1ccccc1C2(C)C ; COC(=O)c1ccccc1Br ; COC(=O)c1ccccc1-c1cc2c(cc1OC)C(C)(C)c1ccccc1-2]

The same procedure as in Synthesis Example 1-3 was carried out, with the exception of using 2-bromo-methylbenzoate and <Intermediate 3-b> instead of 2-(4-bromophenyl)-1,10-phenanthroline and <Intermediate 1-b>, to afford <Intermediate 3-c> (35 g, yield 67%).

### Synthesis Example 3-4. Synthesis of <Intermediate 3-d>

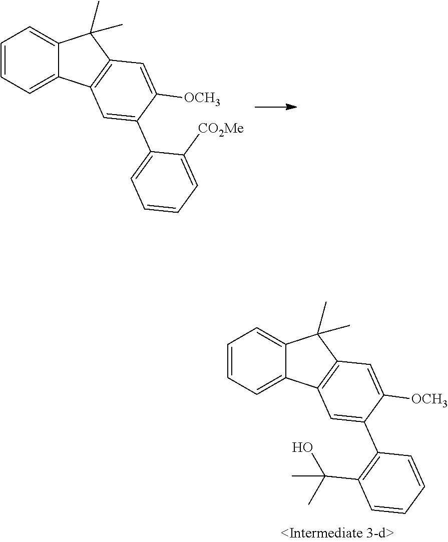 [SMILES: COC(=O)c1ccccc1-c1cc2c(cc1OC)C(C)(C)c1ccccc1-2 ; COc1cc2c(cc1-c1ccccc1C(C)(C)O)-c1ccccc1C2(C)C]

In a 1,000-mL flask, <Intermediate 3-c> (33.5 g, 110 mmol) was cooled to −10° C. and then drops of 3 M methyl magnesium bromide (85 mL, 254 mmol) were slowly added thereto. Subsequently, the mixture was heated to 40° C. and then stirred for 4 hours. Afterwards, the temperature was decreased to −10° C. and 2N HCl (70 mL) was dropwise added slowly. The addition of an aqueous ammonium chloride solution (70 mL) was followed by temperature elevation to room temperature. After completion of the reaction, the reaction mixture was washed with water. The organic layer formed by extraction with ethyl acetate was concentrated in a vacuum to afford <Intermediate 3-d> (31 g, yield 81%).

### Synthesis Example 3-5. Synthesis of <Intermediate 3-e>

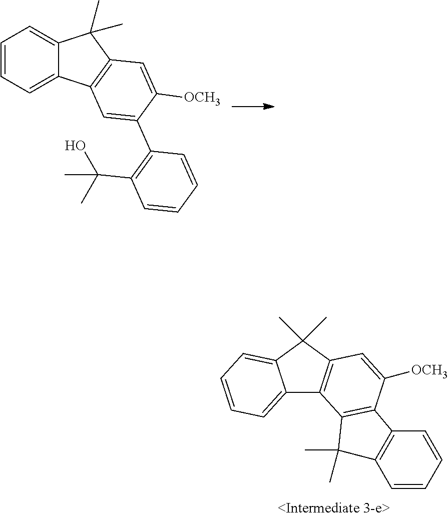 [SMILES: COc1cc2c(cc1-c1ccccc1C(C)(C)O)-c1ccccc1C2(C)C ; COc1cc2c(c3c1-c1ccccc1C3(C)C)-c1ccccc1C2(C)C]

In a 500-mL flask, <Intermediate 3-d> (27 g, 89.2 mmol) and phosphoric acid (70 mL) were stirred together at room temperature for 12 hour. After completion of the reaction, extraction with ethyl acetate and water was conducted. The organic layer thus formed was concentrated, followed by purification through column chromatography to afford <Intermediate 3-e> (20 g, yield 74%).

### Synthesis Example 3-6. Synthesis of <Intermediate 3-f>

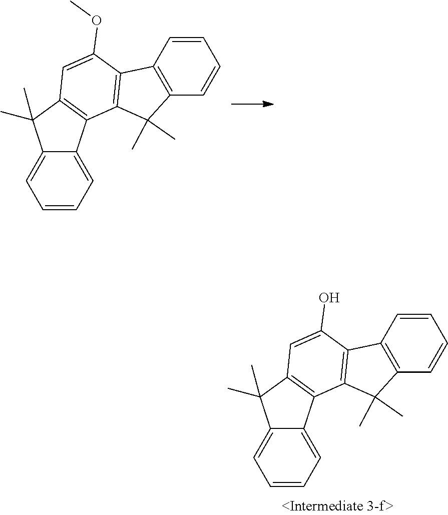 [SMILES: COc1cc2c(c3c1-c1ccccc1C3(C)C)-c1ccccc1C2(C)C ; CC1(C)c2ccccc2-c2c1cc(O)c1c2C(C)(C)c2ccccc2-1]

In a 500-mL reactor, <Intermediate 3-e> (20.4 g, 320 mmol), hydrogen bromide (20 g, 276 mmol), and acetic acid (200 ml) were stirred together for 15 hours under reflux. After completion of the reaction, extraction with ethyl acetate and water was conducted. The organic layer thus formed was concentrated. Purification by column chromatography using ethyl acetate and heptane afforded <Intermediate 3-f> (18 g, yield 91%).

### Synthesis Example 3-7. Synthesis of <Intermediate 3-g>

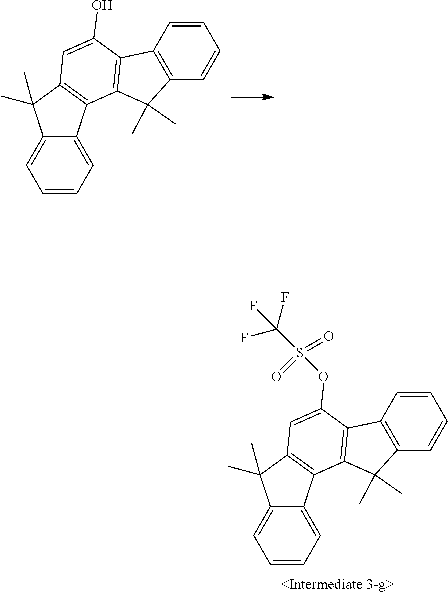 [SMILES: CC1(C)c2ccccc2-c2c1cc(O)c1c2C(C)(C)c2ccccc2-1 ; CC1(C)c2ccccc2-c2c1cc(OS(=O)(=O)C(F)(F)F)c1c2C(C)(C)c2ccccc2-1]

In a 500-mL reactor, <Intermediate 3-f> (18 g, 52 mmol), trifluoromethane sulfonic anhydride (17.5 g, 62 mmol), pyridine (4.9 g, 62 mmol), and methylene chloride (180 mL) were stirred together at room temperature for 5 hours. After completion of the reaction, extraction was conducted with ethyl acetate and water. The organic layer thus formed was concentrated. Purification by column chromatography afforded <Intermediate 3-g> (22.8 g, yield 94%).

### Synthesis Example 3-8. Synthesis of <Intermediate 3-h>

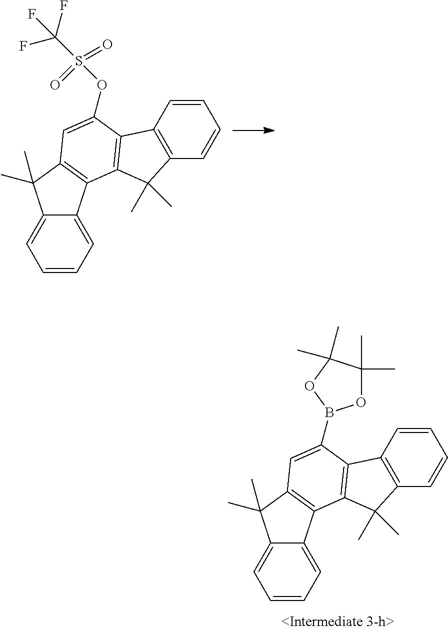 [SMILES: CC1(C)c2ccccc2-c2c1cc(OS(=O)(=O)C(F)(F)F)c1c2C(C)(C)c2ccccc2-1 ; CC1(C)c2ccccc2-c2c1cc(B1OC(C)(C)C(C)(C)O1)c1c2C(C)(C)c2ccccc2-1]

In a 250-mL flask, <Intermediate 3-g> (8.5 g, 26.7 mmol), bis(pinacolato)diboron (8.8 g, 34.7 mmol), (diphenylphosphinoferrocene) palladium dichloride (1 g, 1.3 mmol), potassium acetate (7.6 g, 80 mmol), and toluene (90 ml) were refluxed for 12 hours under a nitrogen atmosphere. After completion of the reaction, the reaction mixture was subjected to layer separation. The organic layer thus formed was concentrated in a vacuum, followed by purification through column chromatography to afford <Intermediate 3-h> (6.7 g, yield 75%)

### Synthesis Example 3-9. Synthesis of <Compound 22>

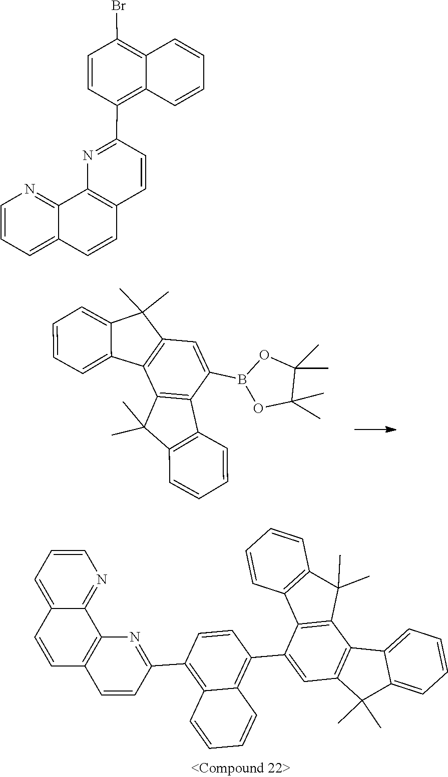

The same procedure as in Synthesis Example 1-3 was carried out, with the exception of using 2-(4-bromonaphthalene-1-yl)-1,10-phenanthroline and <Intermediate 3-h> instead of 2-(4-bromophenyl)-1,10-phenanthroline and <Intermediate 1-b>, respectively, to afford <Compound 22> (yield 57%).

MS (MALDI-TOF): m/z 614.27 [M⁺]

### Synthesis Example 4: Synthesis of Compound 64

### Synthesis Example 4-1. Synthesis of <Intermediate 4-a>

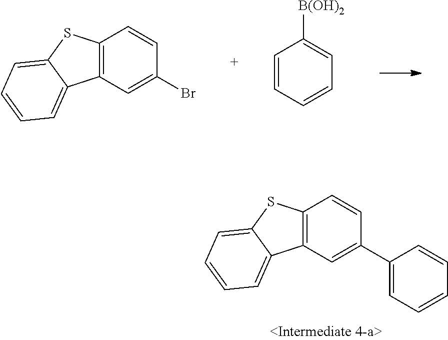 [SMILES: Brc1ccc2sc3ccccc3c2c1 ; OB(O)c1ccccc1 ; c1ccc(-c2ccc3sc4ccccc4c3c2)cc1]

The same procedure as in Synthesis Example 1-3 was carried out, with the exception of using 2-bromodibenzothiophene and phenyl boronic acid instead of 2-(4-bromophenyl)-1,10-phenanthroline and <Intermediate 1-b>, respectively, to afford <Intermediate 4-a>, yield 82%)

### Synthesis Example 4-2. Synthesis of <Intermediate 4-b>

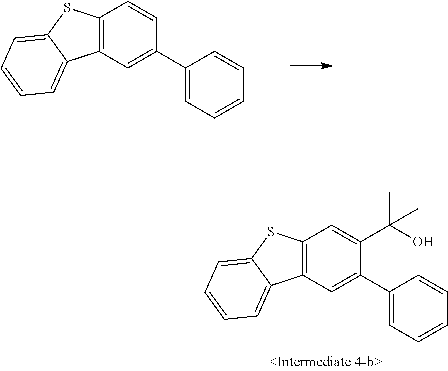 [SMILES: c1ccc(-c2ccc3sc4ccccc4c3c2)cc1 ; CC(C)(O)c1cc2sc3ccccc3c2cc1-c1ccccc1]

In a round-bottom flask, <Intermediate 4-a>50 g (192 mmol) and tetrahydrofuran (500 ml) were put under a nitrogen atmosphere and cooled to −78° C. before 1.6 M-butyl lithium (128 ml, 204 mmol) was dropwise added slowly. The mixture was stirred for 1 hour, slowly added with drops of acetone (14.3 g, 245 mmol), and then stirred again at room temperature for 6 hours. After completion of the reaction, an aqueous ammonium chloride solution (50 ml) was added to induce layer separation. The organic layer thus formed was concentrated in a vacuum, followed by purification through column chromatography to afford <Intermediate 4-b> (36.7 g, yield 60%).

### Synthesis Example 4-3. Synthesis of <Intermediate 4-c>

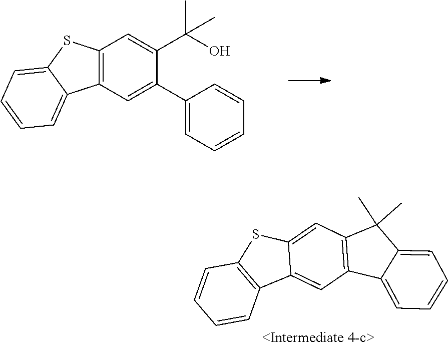 [SMILES: CC(C)(O)c1cc2sc3ccccc3c2cc1-c1ccccc1 ; CC1(C)c2ccccc2-c2cc3c(cc21)sc1ccccc13]

In a round-bottom flask, <Intermediate 4-b> (27 g, 84.8 mmol) and phosphoric acid (70 ml) were stirred together at room temperature for 12 hours under a nitrogen atmosphere. After completion of the reaction, extraction followed concentration. Purification by column chromatography afforded <Intermediate 4-c> (17.8 g, yield 70%).

### Synthesis Example 4-4. Synthesis of <Intermediate 4-d>

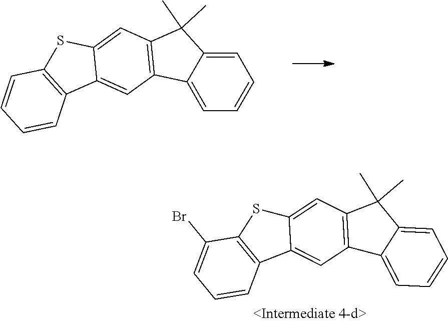 [SMILES: CC1(C)c2ccccc2-c2cc3c(cc21)sc1ccccc13 ; CC1(C)c2ccccc2-c2cc3c(cc21)sc1c(Br)cccc13]

In a 1-L reactor, <Intermediate 4-c> (15.3 g, 50.9 mmol) and dichloromethane (150 ml) were stirred together. Bromine (8.9 g, 56 mmol) was dropwise added slowly at room temperature, followed by stirring for 5 hours. After completion of the reaction, methanol was used for precipitation. Filtration of the precipitates afforded <Intermediate 4-d> (10.2 g, yield 53%).

### Synthesis Example 4-5. Synthesis of <Intermediate 4-e>

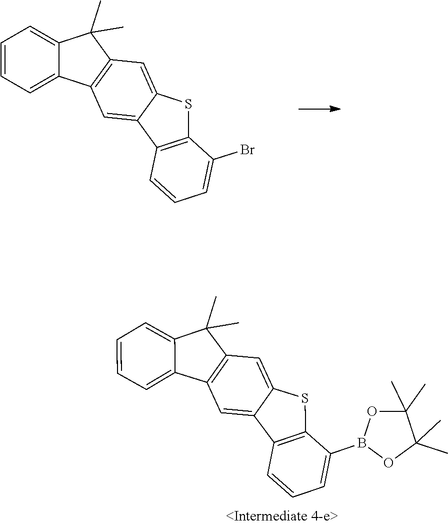 [SMILES: CC1(C)c2ccccc2-c2cc3c(cc21)sc1c(Br)cccc13 ; CC1(C)c2ccccc2-c2cc3c(cc21)sc1c(B2OC(C)(C)C(C)(C)O2)cccc13]

The same procedure as in Synthesis Example 1-2 was carried out, with the exception of using <Intermediate 4-d> instead of <Intermediate 1-a>, to afford <Intermediate 4-e> (yield 78%).

### Synthesis Example 4-6. Synthesis of <Compound 64>

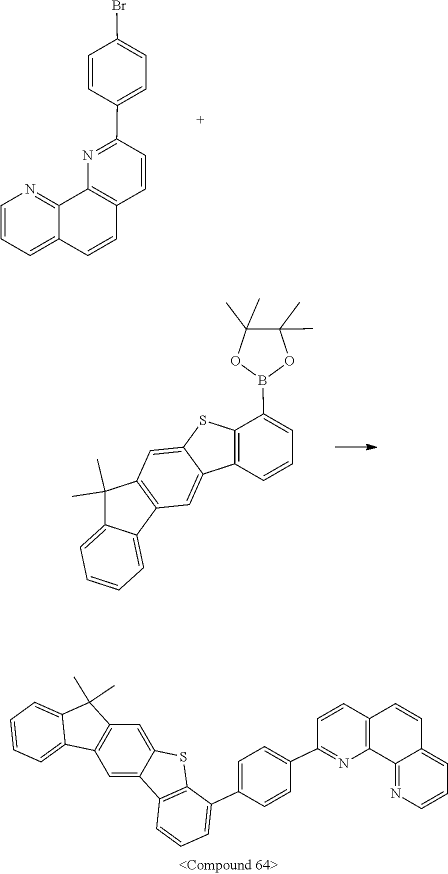

The same procedure as in Synthesis Example 1-3 was carried out, with the exception of using <Intermediate 1-b> instead of <Intermediate 4-e>, to afford <Compound 64> (yield 57%).

MS (MALDI-TOF): m/z 554.18 [M⁺]

### Synthesis Example 5: Synthesis of Compound 68

### Synthesis Example 5-1. Synthesis of <Intermediate 5-a>

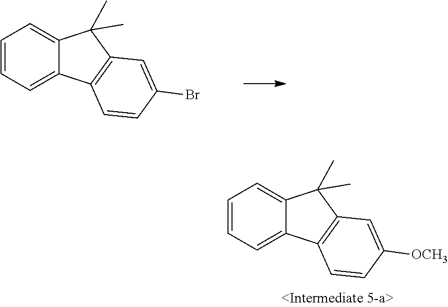 [SMILES: CC1(C)c2ccccc2-c2ccc(Br)cc21 ; COc1ccc2c(c1)C(C)(C)c1ccccc1-2]

In a round-bottom flask, 2-bromo-9,9-dimethylfluorene (50 g, 183 mmol), an aqueous sodium methoxide solution (59.3 g, 1098 mmol), copper iodine (10.4 g, 54.9 mmol), and methanol (200 ml) were together refluxed for 12 hours under a nitrogen atmosphere. After completion of the reaction, the organic layer was concentrated in a vacuum and purified through column chromatography to afford <Intermediate 5-a> (33.2 g yield 81%).

### Synthesis Example 5-2. Synthesis of <Intermediate 5-b>

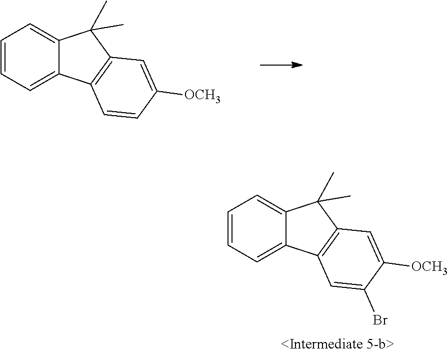 [SMILES: COc1ccc2c(c1)C(C)(C)c1ccccc1-2 ; COc1cc2c(cc1Br)-c1ccccc1C2(C)C]

In a round-bottom flask, <Intermediate 3-a> (30 g, 133 mmol), N-bromosuccinimide (23.8 g, 133 mmol), and dimethylformamide (600 ml) were stirred together at 50° C. for 12 hours under a nitrogen atmosphere. After completion of the reaction, the organic layer was concentrated in a vacuum and purified through column chromatography to afford <Intermediate 5-b> (28 g, yield 70%).

### Synthesis Example 5-3. Synthesis of <Intermediate 5-c>

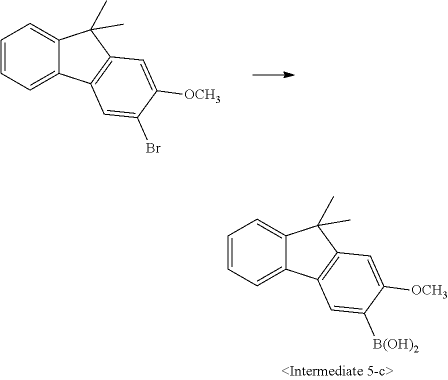 [SMILES: COc1cc2c(cc1Br)-c1ccccc1C2(C)C ; COc1cc2c(cc1B(O)O)-c1ccccc1C2(C)C]

In a round-bottom flask, <Intermediate 5-b> (49.7 g, 164 mmol) was added with tetrahydrofuran (500 ml) under a nitrogen atmosphere. After the temperature was lowered to −78° C., 1.6 M butyl lithium (123 ml, 197 mmol) was dropwise added slowly. One hour later, trimethyl borate (22 ml, 214 mmol) was slowly added with the low temperature maintained, followed by stirring at room temperature. After completion of the reaction, the reaction mixture was subjected to layer separation. The organic layer thus formed was concentrated in a vacuum and recrystallized to afford <Intermediate 5-c> (35.6 g, yield 81%).

### Synthesis Example 5-4. Synthesis of <Intermediate 5-d>

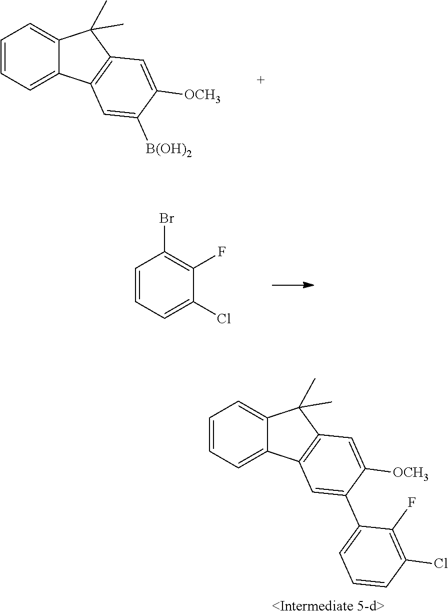 [SMILES: COc1cc2c(cc1B(O)O)-c1ccccc1C2(C)C ; Fc1c(Cl)cccc1Br ; COc1cc2c(cc1-c1cccc(Cl)c1F)-c1ccccc1C2(C)C]

The same procedure as in Synthesis Example 1-3 was carried out, with the exception of using 1-bromo-3-chloro-2-fluorobenzene and <Intermediate 5-c> instead of 2-(4-bromophenyl)-1,10-phenanthroline and <Intermediate 1-b>, respectively, to afford <Intermediate 5-d>, yield 52%)

### Synthesis Example 5-5. Synthesis of <Intermediate 5-e>

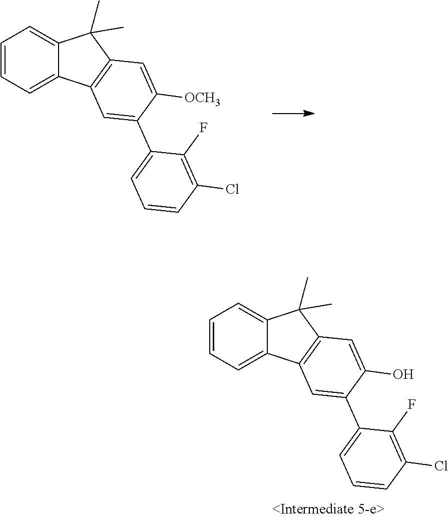 [SMILES: COc1cc2c(cc1-c1cccc(Cl)c1F)-c1ccccc1C2(C)C ; CC1(C)c2ccccc2-c2cc(-c3cccc(Cl)c3F)c(O)cc21]

In a round-bottom flask, a mixture of <Intermediate 5-d> (30 g, 85 mmol) and dichloromethane (300 ml) was cooled to 0° C. and slowly added with drops of a dilution of boron tribromide (63.9 g, 255 mmol) in dichloromethane (150 ml). The temperature was elevated to room temperature before the mixture was stirred for 6 hours. After completion of the reaction, the organic layer was concentrated in a vacuum and purified through column chromatography to afford <Intermediate 5-e> (21.3 g, yield 74%).

### Synthesis Example 5-6. Synthesis of <Intermediate 5-f>

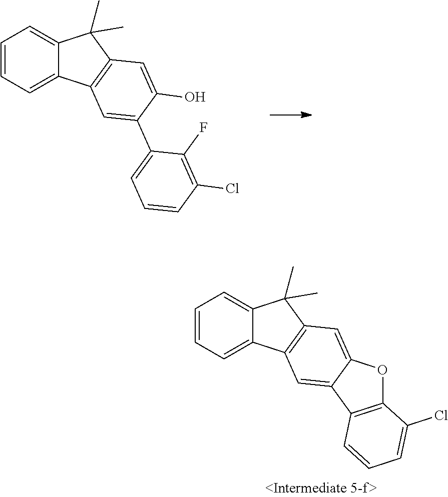 [SMILES: CC1(C)c2ccccc2-c2cc(-c3cccc(Cl)c3F)c(O)cc21 ; CC1(C)c2ccccc2-c2cc3c(cc21)oc1c(Cl)cccc13]

In a round-bottom flask, <Intermediate 5-e> (20 g, 59 mmol), potassium carbonate (13 g, 94.5 mmol), and 1-methyl-2-pyrrolidinone (200 ml) were stirred together at 150° C. for 12 hours. After completion of the reaction, the organic layer was concentrated in a vacuum and purified through column chromatography to afford <Intermediate 5-f> (13.5 g, yield 72%).

### Synthesis Example 5-7. Synthesis of <Intermediate 5-g>

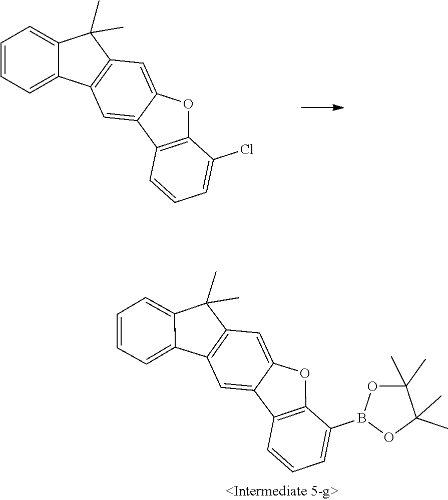 [SMILES: CC1(C)c2ccccc2-c2cc3c(cc21)oc1c(Cl)cccc13 ; CC1(C)c2ccccc2-c2cc3c(cc21)oc1c(B2OC(C)(C)C(C)(C)O2)cccc13]

The same procedure as in Synthesis Example 1-2 was carried out, with the exception of using <Intermediate 5-f> instead of <Intermediate 1-a>, to afford <Intermediate 5-g> (yield 77%).

### Synthesis Example 5-8. Synthesis of <Compound 68>

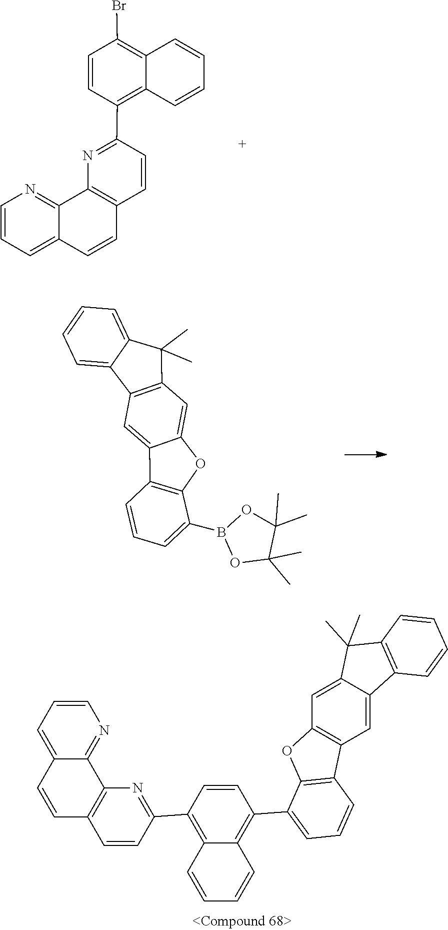 [SMILES: Brc1ccc(-c2ccc3ccc4cccnc4c3n2)c2ccccc12 ; CC1(C)c2ccccc2-c2cc3c(cc21)oc1c(B2OC(C)(C)C(C)(C)O2)cccc13 ; CC1(C)c2ccccc2-c2cc3c(cc21)oc1c(-c2ccc(-c4ccc5ccc6cccnc6c5n4)c4ccccc24)cccc13]

The same procedure as in Synthesis Example 1-3 was carried out, with the exception of using 2-(4-bromonaphthalene-1-yl)-1,10-phenanthroline and <Intermediate 5-g> instead of 2-(4-bromophenyl)-1,10-phenanthroline and <Intermediate 1-b>, respectively, to afford <Compound 68>. (yield 55%)

MS (MALDI-TOF): m/z 588.22 [M⁺]

### Synthesis Example 6: Synthesis of Compound 85

### Synthesis Example 6-1. Synthesis of <Intermediate 6-a>

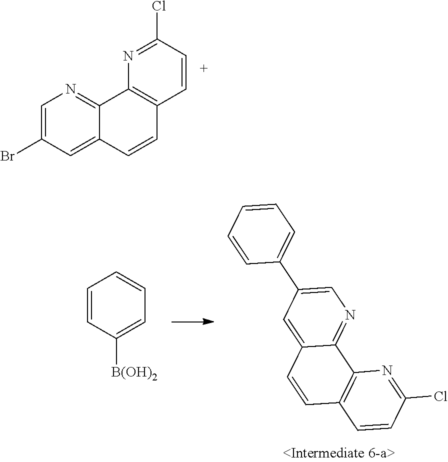 [SMILES: Clc1ccc2ccc3cc(Br)cnc3c2n1 ; OB(O)c1ccccc1 ; Clc1ccc2ccc3cc(-c4ccccc4)cnc3c2n1]

The same procedure as in Synthesis Example 1-3 was carried out, with the exception of using 8-bromo-2-chloro-1,10-phenanthroline and phenyl boronic acid instead of 2-(4-bromophenyl)-1,10-phenanthroline and <Intermediate 1-b>, respectively, to afford <Intermediate 6-a>, yield 73%)

### Synthesis Example 6-2. Synthesis of <Intermediate 6-b>

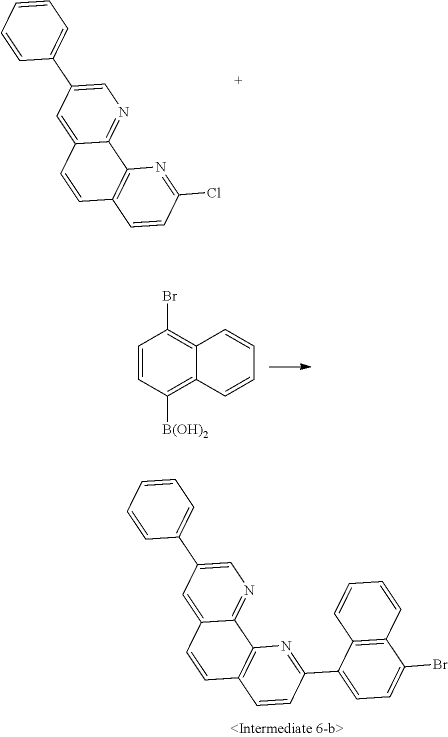 [SMILES: Clc1ccc2ccc3cc(-c4ccccc4)cnc3c2n1 ; OB(O)c1ccc(Br)c2ccccc12 ; Brc1ccc(-c2ccc3ccc4cc(-c5ccccc5)cnc4c3n2)c2ccccc12]

The same procedure as in Synthesis Example 1-3 was carried out, with the exception of using <Intermediate 6-a> and 4-bromo-1-naphthalene boronic acid instead of 2-(4-bromophenyl)-1,10-phenanthroline and <Intermediate 1-b>, respectively, to afford <Intermediate 6-b> (yield 78%).

### Synthesis Example 6-3. Synthesis of <Compound 85>

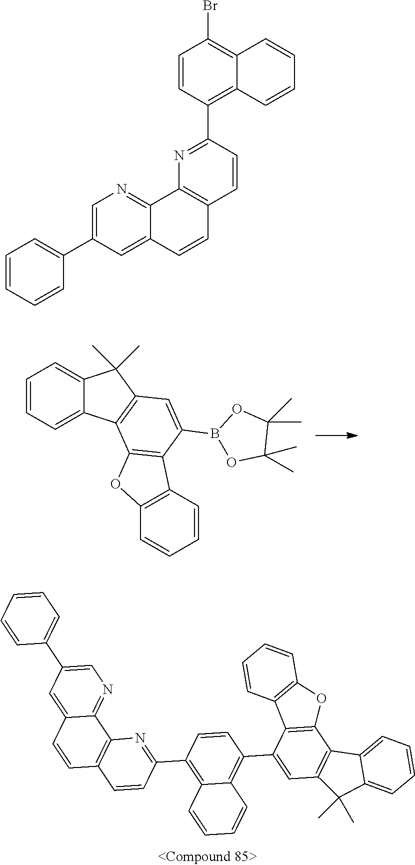 [SMILES: Brc1ccc(-c2ccc3ccc4cc(-c5ccccc5)cnc4c3n2)c2ccccc12 ; CC1(C)c2ccccc2-c2c1cc(B1OC(C)(C)C(C)(C)O1)c1c2oc2ccccc21 ; CC1(C)c2ccccc2-c2c1cc(-c1ccc(-c3ccc4ccc5cc(-c6ccccc6)cnc5c4n3)c3ccccc13)c1c2oc2ccccc21]

The same procedure as in Synthesis Example 1-3 was carried out, with the exception of using <Intermediate 6-b> instead of 2-(4-bromophenyl)-1,10-phenanthroline, to afford <Compound 85> (yield 49%).

MS (MALDI-TOF): m/z 651.24 [M⁺]

### Synthesis Example 7: Synthesis of Compound 23

### Synthesis Example 7-1. Synthesis of <Intermediate 7-a>

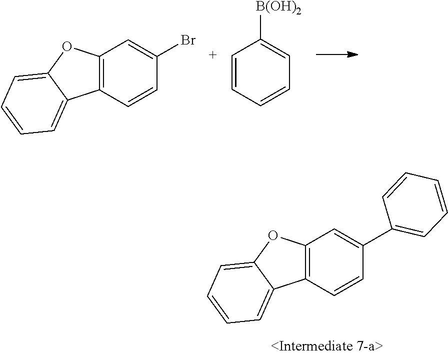 [SMILES: Brc1ccc2c(c1)oc1ccccc12 ; OB(O)c1ccccc1 ; c1ccc(-c2ccc3c(c2)oc2ccccc23)cc1]

The same procedure as in Synthesis Example 1-3 was carried out, with the exception of using 3-bromodibenzofuran and phenyl boronic acid instead of 2-(4-bromophenyl)-1,10-phenanthroline and <Intermediate 1-b>, respectively, to afford <Intermediate 7-a>, yield 76%)

### Synthesis Example 7-2. Synthesis of <Intermediate 7-b>

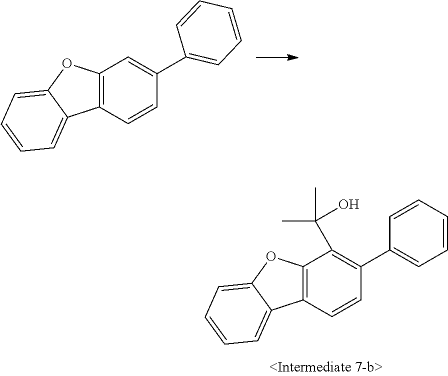 [SMILES: c1ccc(-c2ccc3c(c2)oc2ccccc23)cc1 ; CC(C)(O)c1c(-c2ccccc2)ccc2c1oc1ccccc12]

In a round-bottom flask, <Intermediate 7-a> (50 g, 204 mmol) and tetrahydrofuran (500 ml) were put under a nitrogen atmosphere and cooled to −78° C. before 1.6 M-butyl lithium (128 ml, 204 mmol) was dropwise added slowly. The mixture was stirred for 1 hour, slowly added with drops of acetone (14.3 g, 245 mmol), and then stirred again at room temperature for 6 hours. After completion of the reaction, an aqueous ammonium chloride solution (50 ml) was added to induce layer separation. The organic layer thus formed was concentrated in a vacuum, followed by purification through column chromatography to afford <Intermediate 7-b> (38.3 g, yield 62%).

### Synthesis Example 7-3. Synthesis of <Intermediate 7-c>

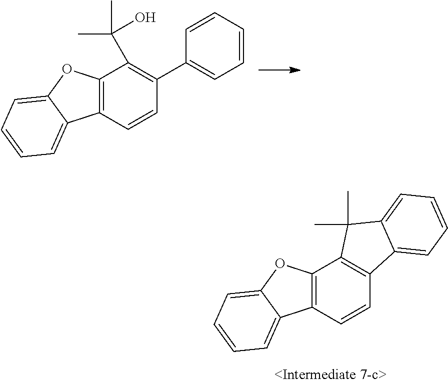 [SMILES: CC(C)(O)c1c(-c2ccccc2)ccc2c1oc1ccccc12 ; CC1(C)c2ccccc2-c2ccc3c(oc4ccccc43)c21]

The same procedure as in Synthesis Example 4-3 was carried out, with the exception of using <Intermediate 7-b> instead of <Intermediate 4-b>, to afford <Intermediate 7-c> (yield 69%).

### Synthesis Example 7-4. Synthesis of <Intermediate 7-d>

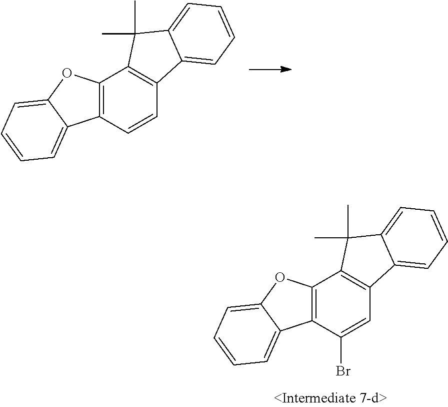 [SMILES: CC1(C)c2ccccc2-c2ccc3c(oc4ccccc43)c21 ; CC1(C)c2ccccc2-c2cc(Br)c3c(oc4ccccc43)c21]

In a 1-L reactor, <Intermediate 7-c> (14.5 g, 50.9 mmol) and dichloromethane (150 ml) were stirred together. At room temperature, bromine (8.9 g, 56 mmol) was dropwise added slowly, followed by stirring for 5 hours. After completion of the reaction, methanol was used for precipitation. The precipitates were filtered to afford <Intermediate 7-d> (9.4 g, yield 44%).

### Synthesis Example 7-5. Synthesis of <Compound 7-e>

The same procedure as in Synthesis Example 1-2 was carried out, with the exception of using <Intermediate 7-d> instead of <Intermediate 1-a>, to afford <Intermediate 7-e> (yield 77%).

### Synthesis Example 7-6. Synthesis of <Compound 23>

The same procedure as in Synthesis Example 1-3 was carried out, with the exception of using <Intermediate 7-e> instead of <Intermediate 1-b>, to afford <Compound 23>. (yield 57%)

MS (MALDI-TOF): m/z 538.20 [M⁺]

### Synthesis Example 8: Synthesis of Compound 70

### Synthesis Example 8-1. Synthesis of <Intermediate 8-a>

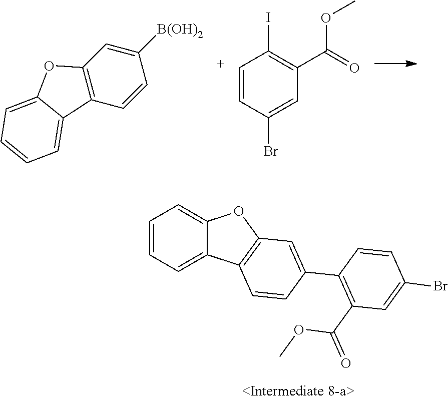 [SMILES: OB(O)c1ccc2c(c1)oc1ccccc12 ; COC(=O)c1cc(Br)ccc1I ; COC(=O)c1cc(Br)ccc1-c1ccc2c(c1)oc1ccccc12]

In a round-bottom flask, dibenzofuran-3-boronic acid (25 g, 118 mmol), methyl-5-bromo-2-iodobenzoate (40.5 g, 118 mmol), tetrakis(triphenylphosphine) palladium (2.7 g, 2.3 mmol), potassium carbonate (33 g, 237 mmol), toluene (200 ml), 1,4-dioxane (200 ml), and water (100 ml) were fluxed together for 12 hours under a nitrogen atmosphere. After completion of the reaction, the reaction mixture was subjected to layer separation. The organic layer thus formed was concentrated in a vacuum and purified through column chromatography to afford <Intermediate 8-a> (33.5 g, yield 74%).

### Synthesis Example 8-2. Synthesis of <Intermediate 8-b>

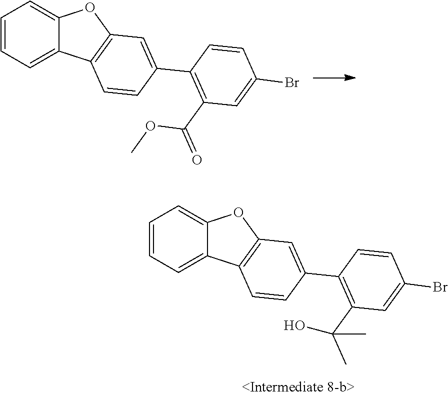 [SMILES: COC(=O)c1cc(Br)ccc1-c1ccc2c(c1)oc1ccccc12 ; CC(C)(O)c1cc(Br)ccc1-c1ccc2c(c1)oc1ccccc12]

In a round-bottom flask, tetrahydrofuran (150 ml) was added with <Intermediate 8-a> (33.5 g, 110 mmol). The temperature was lowered to −10° C. before 3 M methyl magnesium bromide (85 ml, 254 mmol) was dropwise added slowly. The mixture was heated to 40° C., stirred for 4 hours, and then cooled again to −10° C., followed by slowly adding drops of 2 N HCl (70 ml). An aqueous ammonium chloride solution (70 ml) was added before elevation to room temperature. After completion of the reaction, the reaction mixture was washed with water, concentrated in a vacuum, and purified through column chromatography to afford <Intermediate 8-b> (27 g, yield 80%).

### Synthesis Example 8-3. Synthesis of <Intermediate 8-c>

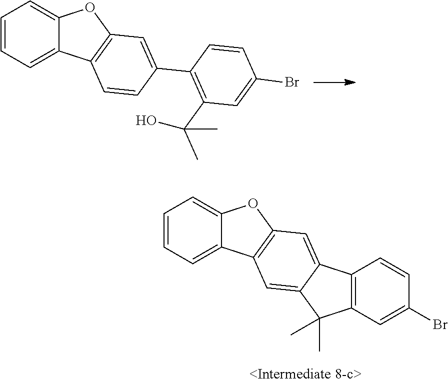

In a round-bottom flask, <Intermediate 8-b> (27 g, 89.2 mmol) and phosphoric acid (70 ml) were stirred together at room temperature for 12 hours under a nitrogen atmosphere. After completion of the reaction, extraction followed concentration. Purification by column chromatography afforded <Intermediate 8-c> (17.6 g, yield 70%).

### Synthesis Example 8-4. Synthesis of <Compound 8-d>

The same procedure as in Synthesis Example 1-2 was carried out, with the exception of using <Intermediate 8-c> instead of <Intermediate 1-a>, to afford <Compound 8-d> (yield 75%)

### Synthesis Example 8-5. Synthesis of <Compound 70>

The same procedure as in Synthesis Example 1-3 was carried out, with the exception of using 2-(4-bromonaphthalene-1-yl)-1,10-phenanthroline and <Intermediate 8-d> instead of 2-(4-bromophenyl)-1,10-phenanthroline and <Intermediate 1-b>, to afford <Compound 70>. (yield 55%)

MS (MALDI-TOF): m/z 588.22 [M⁺]

### Synthesis Example 9: Synthesis of Compound 100

### Synthesis Example 9-1. Synthesis of <Compound 100>

The same procedure as in Synthesis Example 3 was carried out, with the exception of using 3-methoxydibenzofuran instead of 2-methoxy-9,9-dimethyl-9H-fluorene used in Synthesis Example 3-1, to afford <Compound 100>. (yield 55%)

MS (MALDI-TOF): m/z 588.22 [M]⁺

### Synthesis Example 10: Synthesis of Compound 94

### Synthesis Example 10-1. Synthesis of <Intermediate 10-a>

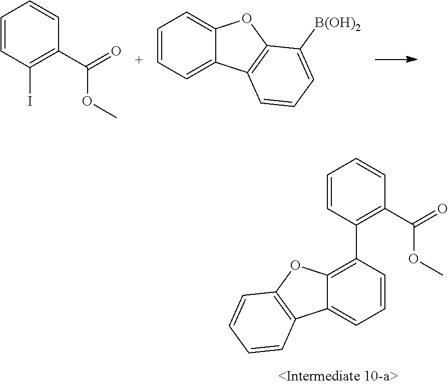

In a 500-mL round-bottom flask, a mixture of methyl 2-iodobenzoate (19.1 g, 73 mmol), 4-dibenzofuran boronic acid (18.7 g, 88 mmol), tetrakis(triphenylphosphine) palladium (1.7 g, 0.15 mmol), and potassium carbonate (20.2 g, 146.7 mmol) was added with toluene (125 mL), tetrahydrofuran (125 mL), and water (50 mL). The reactor was heated to 80° C. before stirring for 10 hours. After completion of the reaction, the reactor was cooled to room temperature. Extraction was conducted with ethyl acetate and the organic layer thus formed was isolated. The organic layer was concentrated in a vacuum and purified through column chromatography to afford <Intermediate 10-a>. (9.5 g, 43%)

### Synthesis Example 10-2. Synthesis of <Intermediate 10-b>

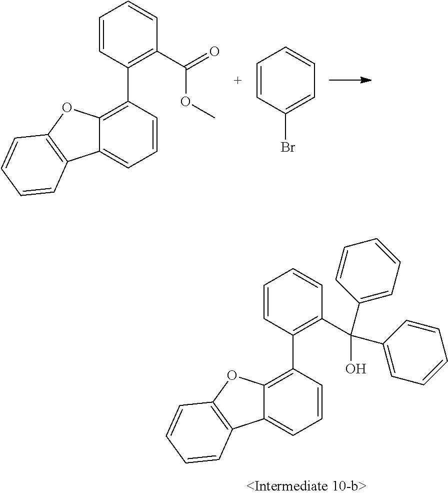

In a 2-L round-bottom flask, bromobenzene (13.2 g, 83.97 mmol) and tetrahydrofuran (250 ml) were stirred together at a low temperature under a nitrogen atmosphere. At −78° C., n-butyl lithium (ca. 58 ml) was dropwise added slowly over 2 hours, followed by <Intermediate 10-a> (9.4 g, 31.1 mmol). After completion of the reaction, water (100 ml) was added and stirred for 30 minutes. Extraction afforded <Intermediate 10-b> (3.2 g, 24%).

### Synthesis Example 10-3. Synthesis of <Intermediate 10-c>

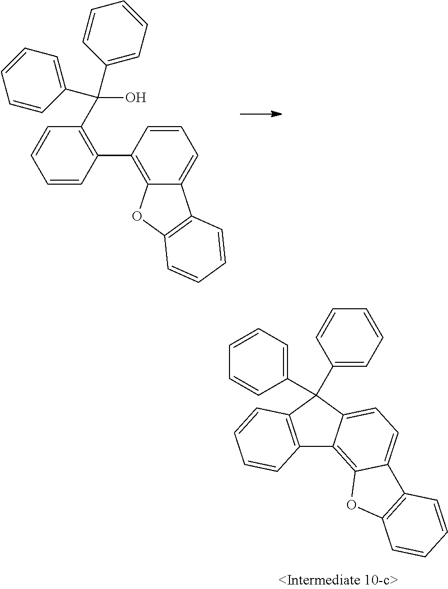 [SMILES: OC(c1ccccc1)(c1ccccc1)c1ccccc1-c1cccc2c1oc1ccccc12 ; c1ccc(C2(c3ccccc3)c3ccccc3-c3c2ccc2c3oc3ccccc32)cc1]

In a 2-L round-bottom flask, <Intermediate 10-b> (55.0 g, 129 mmol), acetic acid (500 ml), and sulfuric acid (10 ml) were stirred together for 5 hours under reflux. After completion of the reaction, the mixture was cooled to room temperature. The precipitates thus formed were filtered and washed with methanol to afford <Intermediate 10-c> (50 g, 95%).

### Synthesis Example 10-4. Synthesis of <Intermediate 10-d>

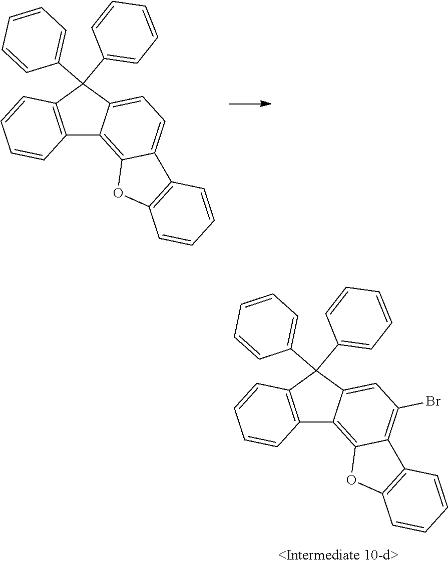 [SMILES: c1ccc(C2(c3ccccc3)c3ccccc3-c3c2ccc2c3oc3ccccc32)cc1 ; Brc1cc2c(c3oc4ccccc4c13)-c1ccccc1C2(c1ccccc1)c1ccccc1]

The same procedure as in Synthesis Example 1-1 was carried out, with the exception of using <Intermediate 10-c> instead of 7,7-dimethyl-7H-fluoreno[4,3-b]benzofuran, to afford <Intermediate 10-d> (yield 86%)

### Synthesis Example 10-5. Synthesis of <Intermediate 10-e>

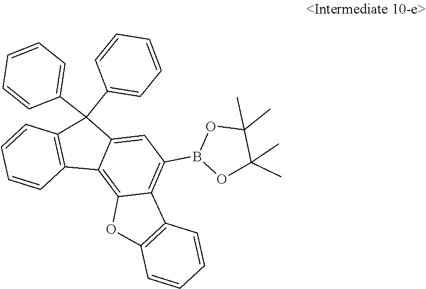 [SMILES: CC1(C)OB(c2cc3c(c4oc5ccccc5c24)-c2ccccc2C3(c2ccccc2)c2ccccc2)OC1(C)C]

The same procedure as in Synthesis Example 1-2 was carried out, with the exception of using <Intermediate 10-d> instead of <Intermediate 1-a>, to afford <Intermediate 10-e>. (yield 77%)

### Synthesis Example 10-6. Synthesis of <Compound 94>

The same procedure as in Synthesis Example 1-3 was carried out, with the exception of using 2-(3-bromophenyl)-1,10-phenanthroline and <Intermediate 10-e> instead of 2-(4-bromophenyl)-1,10-phenanthroline and <Intermediate 1-b>, respectively, to afford <Compound 94>. (yield 53%)

MS (MALDI-TOF): m/z 662.24 [M⁺]

### Examples 1 to 10: Fabrication of Organic Light-Emitting Diodes

Organic light-emitting diodes in a tandem structure with two blue layers stacked were fabricated in the following procedures. In this regard, each of the layers employed the same host and dopant, but the present disclosure is not limited thereby.

An ITO glass substrate was patterned to have a translucent area of 2 mm×2 mm and cleansed. The ITO glass was mounted in a vacuum chamber that was then set to have a base pressure of 1×10−7 torr. On the ITO glass substrate, the following organic layers were sequentially formed.

HATCN was formed as a hole injection layer at a thickness of 50 Å; NPD as a first hole transport layer at a thickness of 600 Å; BH+BD 3 wt % dopant as a first light-emitting layer at a thickness of 200 Å; [ET] as a first electron transport layer at a thickness of 250 Å; the compound, represented by Chemical Formula A according to the present disclosure, doped with 2% Li, as a N-type charge generation layer at a thickness of 100 A; and HATCN as a P-type charge generation layer at a thickness of 100 Å.

Subsequently, NPD was formed as a second hole transport layer at a thickness of 600 Å; BH+BD 3 wt % dopant as a second light-emitting layer at a thickness of 200 Å; [ET] as a second transport layer at a thickness of 250 Å; Liq as an electron injection layer at a thickness of 10 Å; and Al as a cathode at a thickness of 1000 Å.

The organic light-emitting diodes thus obtained were measured at 0.4 mA for luminescence properties:

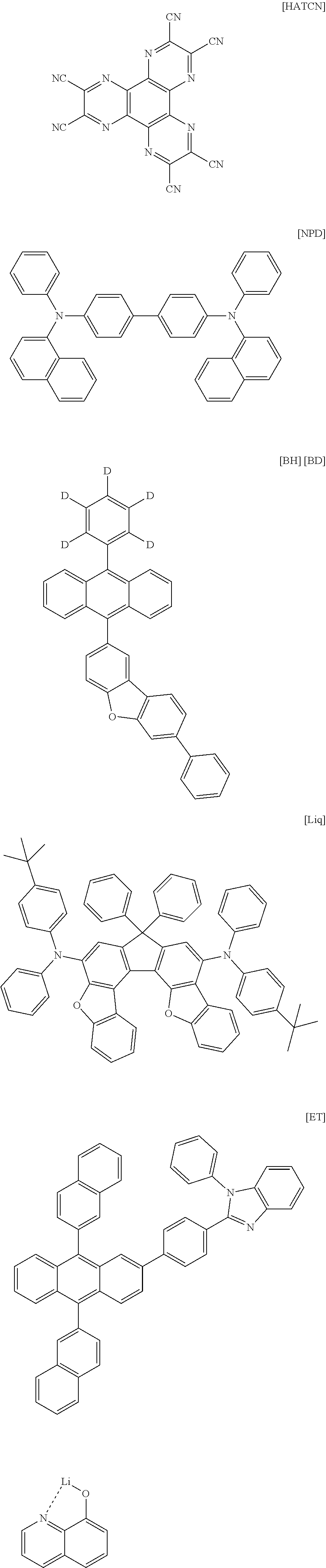

### Comparative Example 1

Organic light-emitting diodes were fabricated in the same manner as in Examples 1 to 10 with the exception of using [CGL1] to [CGL3] instead of the N-type charge generation layer compounds used. The organic light-emitting diodes were measured at 0.4 mA for luminescence properties. The structures of [CGL1] to [CGL3] are as follows:

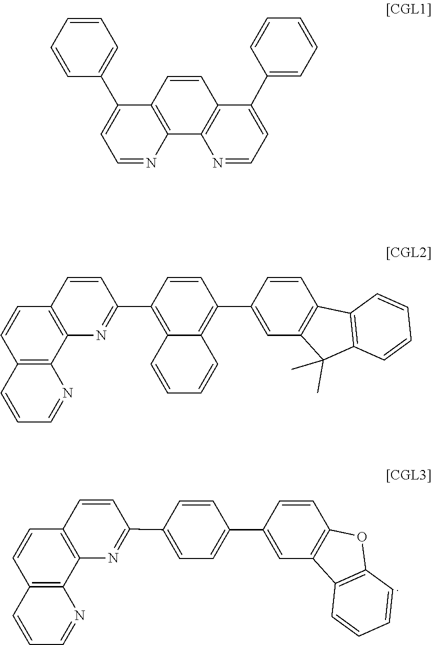 [SMILES: c1ccc(-c2ccnc3c2ccc2c(-c4ccccc4)ccnc23)cc1 ; CC1(C)c2ccccc2-c2ccc(-c3ccc(-c4ccc5ccc6cccnc6c5n4)c4ccccc34)cc21 ; c1cnc2c(c1)ccc1ccc(-c3ccc(-c4ccc5oc6ccccc6c5c4)cc3)nc12]

TABLE 1

| | | Driving | | |
|---|---|---|---|---|
| | N-type Charge | volt. | Lifespan | EQE |
| | Generation Layer | (V) | (T97) | (%) |
| Example 1 | Compound 1 | 9.4 | 195 | 25 |
| Example 2 | Compound 10 | 9.5 | 190 | 24 |
| Example 3 | Compound 22 | 9.5 | 190 | 24 |
| Example 4 | Compound 64 | 9.6 | 187 | 23 |
| Example 5 | Compound 68 | 9.6 | 180 | 23 |
| Example 6 | Compound 85 | 9.4 | 200 | 25 |
| Example 7 | Compound 23 | 9.6 | 183 | 23 |
| Example 8 | Compound 70 | 9.6 | 180 | 23 |
| Example 9 | Compound 100 | 9.5 | 185 | 24 |
| Example 10 | Compound 94 | 9.5 | 192 | 24 |
| C. Example 1 | CGL1 | 9.8 | 118 | 21 |
| C. Example 2 | CGL2 | 9.7 | 127 | 20 |
| C. Example 3 | CGL3 | 9.8 | 120 | 21 |

As is understood from the data of Table 1, the organic light-emitting diodes of the present disclosure exhibited excellent properties such as low driving voltages, longevity, and high emission efficiency, compared to conventional organic light-emitting diodes using the compounds of Comparative Examples 1 to 3.

### INDUSTRIAL APPLICABILITY

When used as a material for an N-type charge generation layer in an organic light-emitting diode with a tandem structure including a charge generation layer, the novel phenanthroline-based compound according to the present disclosure provides improved longevity and high emission efficiency for the organic light-emitting diode, compared to conventional materials. As such, the novel compounds find advantageous applications in the organic light-emitting diode field and related industrial fields.

1. An organic light-emitting diode, comprising:
a first electrode;
a second electrode facing the first electrode; and
an organic layer interposed between the first electrode and the second electrode,
wherein the organic layer includes a light-emitting layer and a charge generation layer,
the charge generation layer includes a P-type charge generation layer and an N-type charge generation layer, and
the N-type charge generation layer comprises at least one phenanthroline-based compound represented by Chemical Formula A: 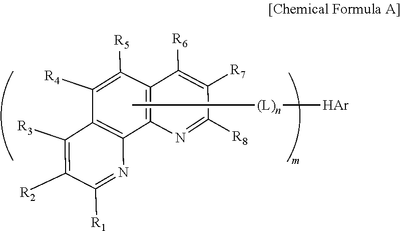
wherein,
L functions as a linker and is at least one selected from a single bond, a substituted or unsubstituted arylene of 6 to 18 carbon atoms, and a substituted or unsubstituted heteroarylene of 2 to 18 carbon atoms,
R₁ to R₈, which are same or different from each other, are each independently at least one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 6 to 30 carbon atoms, a cyano, and a halogen,
wherein any one of R₁ to R₈ is a single bond connected to the linker L,
n is an integer of 1 to 3 wherein when n is 2 or greater, the L's are same or different,
m is an integer of 1 or 2,
wherein when m is 2, the corresponding 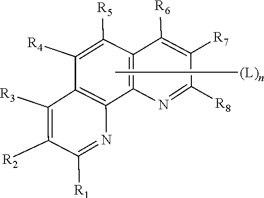
moieties are same or different,
HAr is a substituent represented by the following Structural Formula 1: 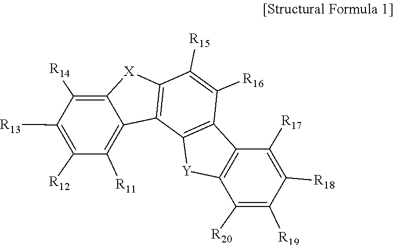
wherein,
R₁₁ to R₂₀, which are same or different, are each independently any one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 6 to 30 carbon atoms, a cyano, and a halogen,
wherein when m is 1, one of R₁ to R₂₀ is a single bond connected to the linker L,
wherein when m is 2, two of R₁ to R₂₀ are each a single bond connected to the linker L,
X and Y, which are same or different, are each independently at least one selected from CR₂₁R₂₂, NR₂₃, O and S,
wherein R₂₁ to R₂₃, which are same or different, are each independently at least one selected from a hydrogen atom, a deuterium atom, a substituted or unsubstituted alkyl of 1 to 30 carbon atoms, a substituted or unsubstituted cycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 5 to 30 carbon atoms, a cyano, and a halogen,
R₂₁ and R₂₂ may be linked to each other to form a mono- or polycyclic aliphatic or aromatic ring,
wherein the term “substituted” in the expression “substituted or unsubstituted” used for the compound of Chemical Formula A means having at least one substituent selected from the group consisting of a deuterium atom, a cyano, a halogen, an alkyl of 1 to 24 carbon atoms, a halogenated alkyl of 1 to 24 carbon atoms, cycloalkyl of 3 to 30 carbon atoms, an aryl of 6 to 24 carbon atoms, an arylalkyl of 7 to 24 carbon atoms, an alkylaryl of 7 to 24 carbon atoms, a heteroaryl of 2 to 50 carbon atoms, an alkylsilyl of 1 to 24 carbon atoms, and an arylsilyl of 6 to 24 carbon atoms.

2. The organic light-emitting diode of claim 1, wherein R₈ in Chemical Formula A is a single bond linked to the linker L.

3. The organic light-emitting diode of claim 1, wherein n and m are each 1.

4. The organic light-emitting diode of claim 1, wherein the linker L is a single bond, or any one selected from the following Structural Formulas 22 to 34: 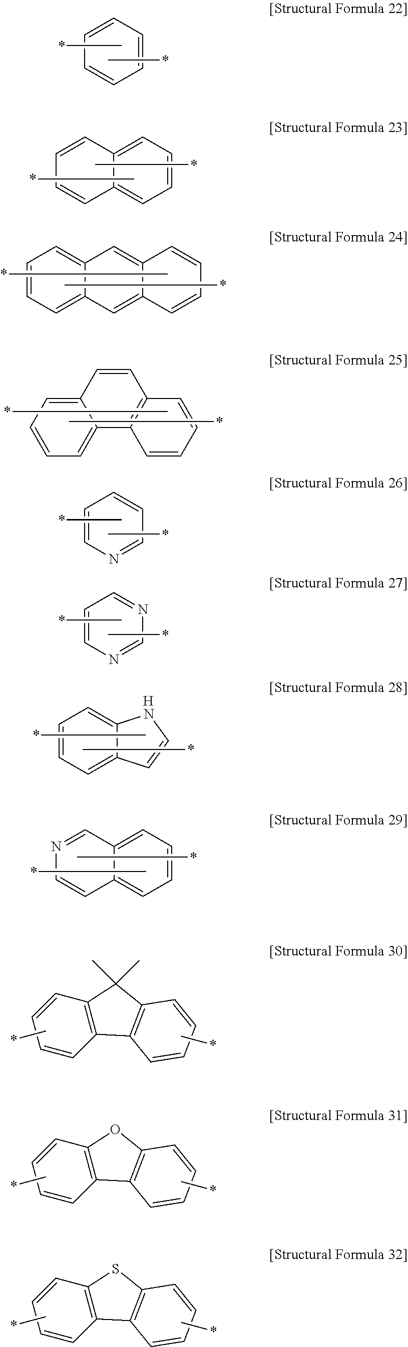 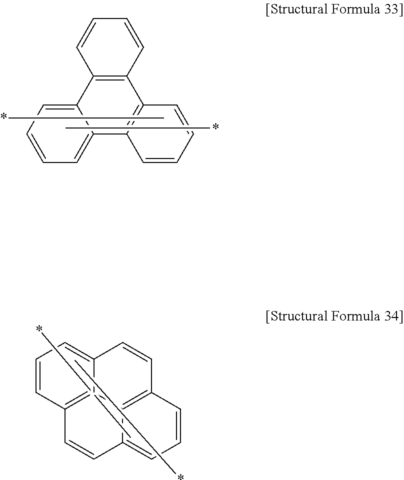
wherein hydrogen or deuterium may be positioned on each carbon atom of the aromatic rings.

5. The organic light-emitting diode of claim 1, wherein at least one of X and Y in [Structural Formula 1] in Chemical Formula A is CR₂₁R₂₂.

6. The organic light-emitting diode of claim 1, wherein R₂₁ to R₂₃ in [Structural Formula 1] are same or different and are each independently any one selected from a substituted or unsubstituted alkyl of 1 to 18 carbon atoms, a substituted or unsubstituted cycloalkyl of 3 to 18 carbon atoms, and a substituted or unsubstituted aryl of 6 to 18 carbon atoms.

7. The organic light-emitting diode of claim 1, wherein R16 in Structural Formula 1 is a single bond connected to the linker L.

8. The organic light-emitting diode of claim 1, wherein the phenanthroline-based compound is any one selected from the following <Compound 1>, <Compound 3>, <Compound 4> to <Compound 22>, <Compound 25>, <Compound 28> to <Compound 30>, <Compound 35> to <Compound 54>, <Compound 56> to <Compound 58>, <Compound 81> to <Compound 85>, <Compound 94> to <Compound 115>, <Compound 117>, <Compound 118>, <Compound 120>, <Compound 121>, <Compound 123>, <Compound 124> and <Compound 126>:

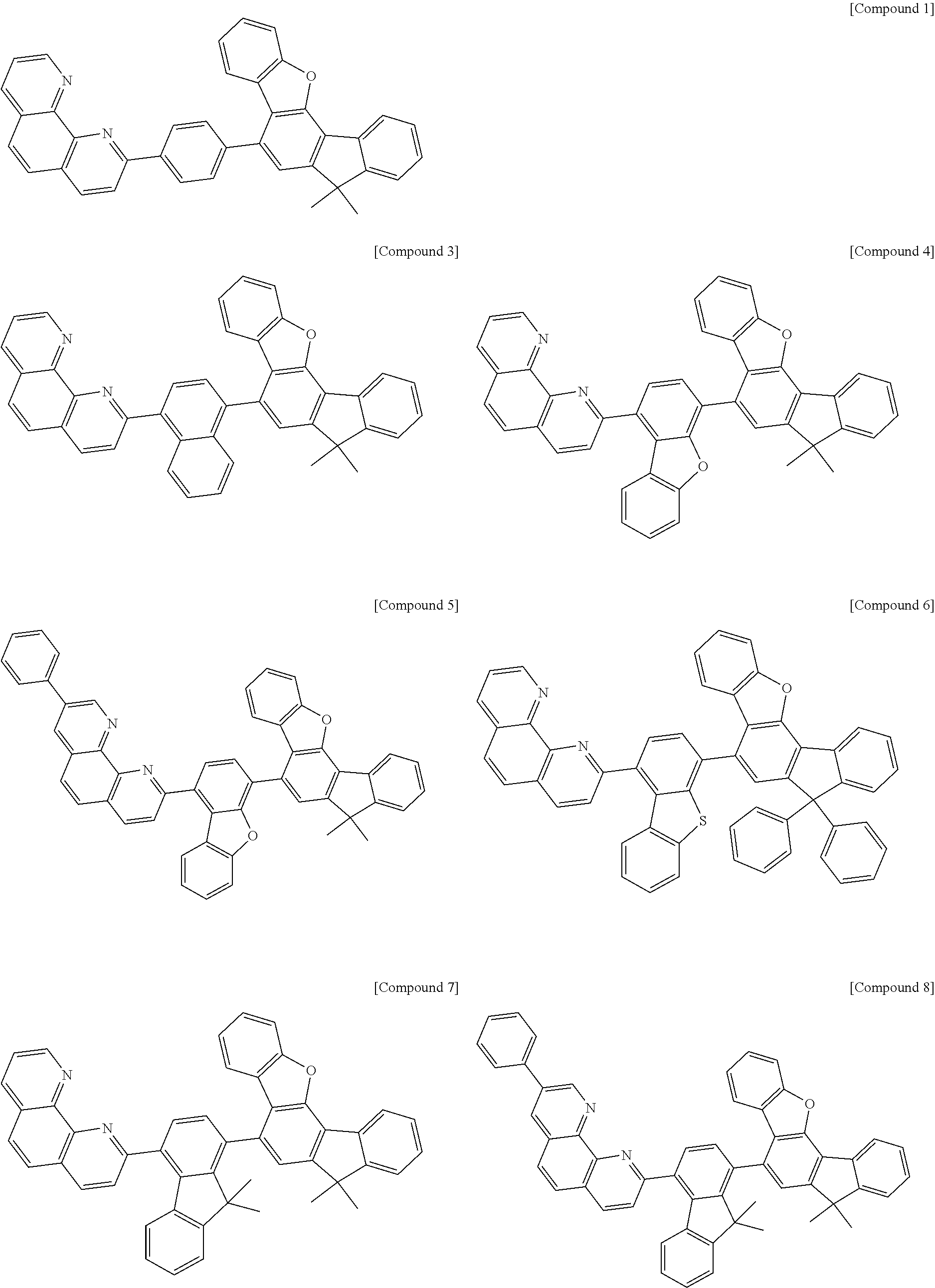

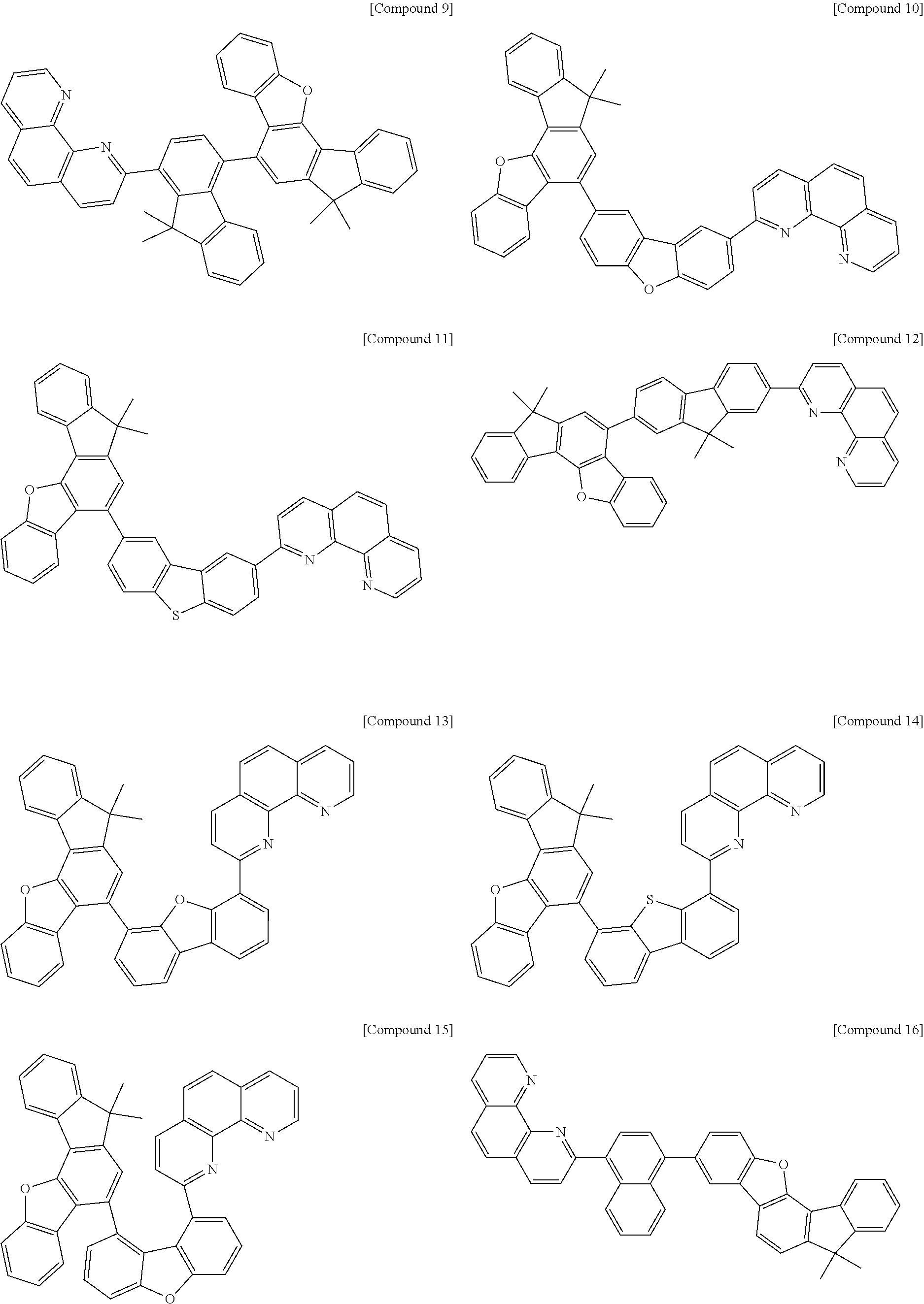

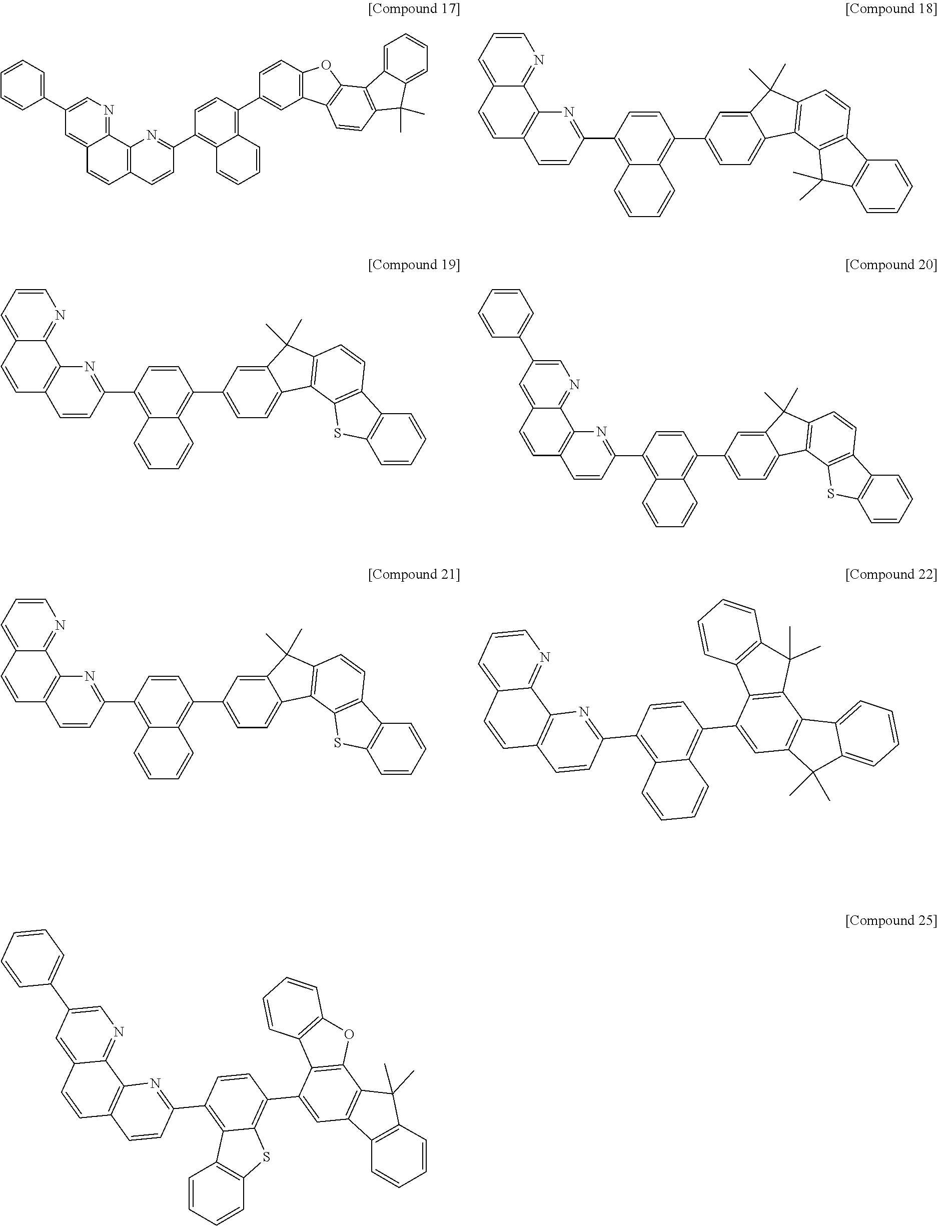

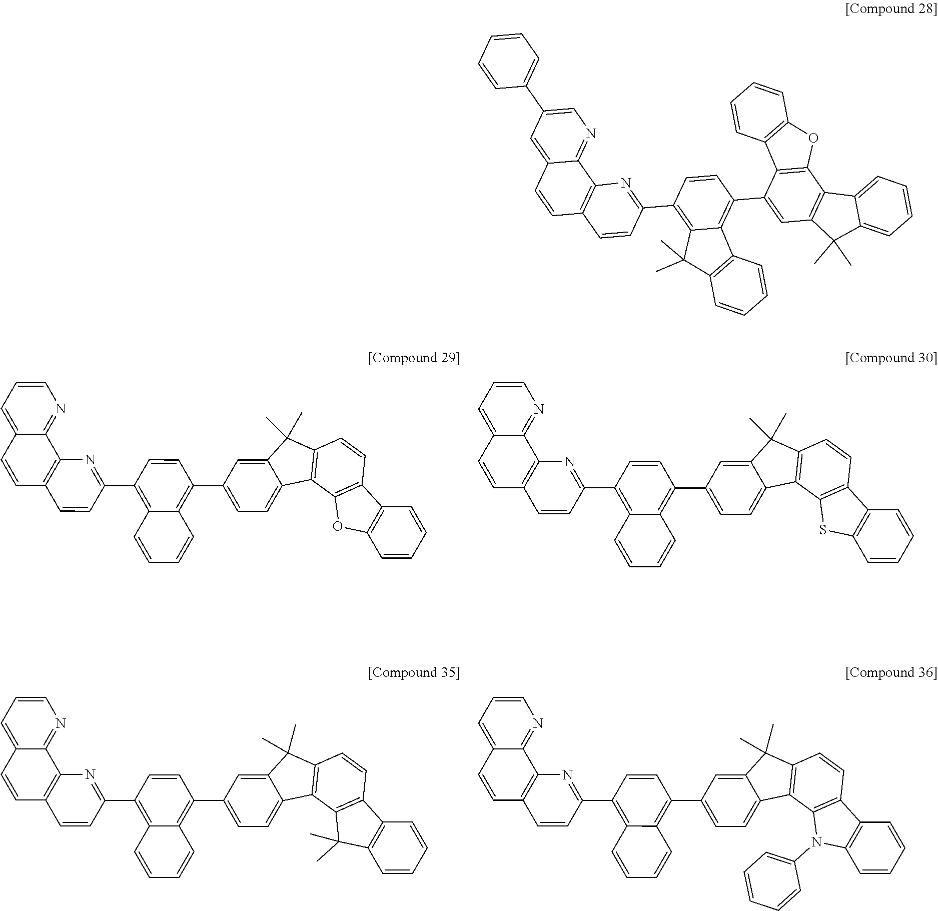 [SMILES: CC1(C)c2ccccc2-c2c1cc(-c1ccc(-c3ccc4ccc5cc(-c6ccccc6)cnc5c4n3)c3c1-c1ccccc1C3(C)C)c1c2oc2ccccc21 ; CC1(C)c2cc(-c3ccc(-c4ccc5ccc6cccnc6c5n4)c4ccccc34)ccc2-c2c1ccc1c2oc2ccccc21 ; CC1(C)c2cc(-c3ccc(-c4ccc5ccc6cccnc6c5n4)c4ccccc34)ccc2-c2c1ccc1c2sc2ccccc21 ; CC1(C)c2cc(-c3ccc(-c4ccc5ccc6cccnc6c5n4)c4ccccc34)ccc2-c2c1ccc1c2C(C)(C)c2ccccc2-1 ; CC1(C)c2cc(-c3ccc(-c4ccc5ccc6cccnc6c5n4)c4ccccc34)ccc2-c2c1ccc1c3ccccc3n(-c3ccccc3)c21]

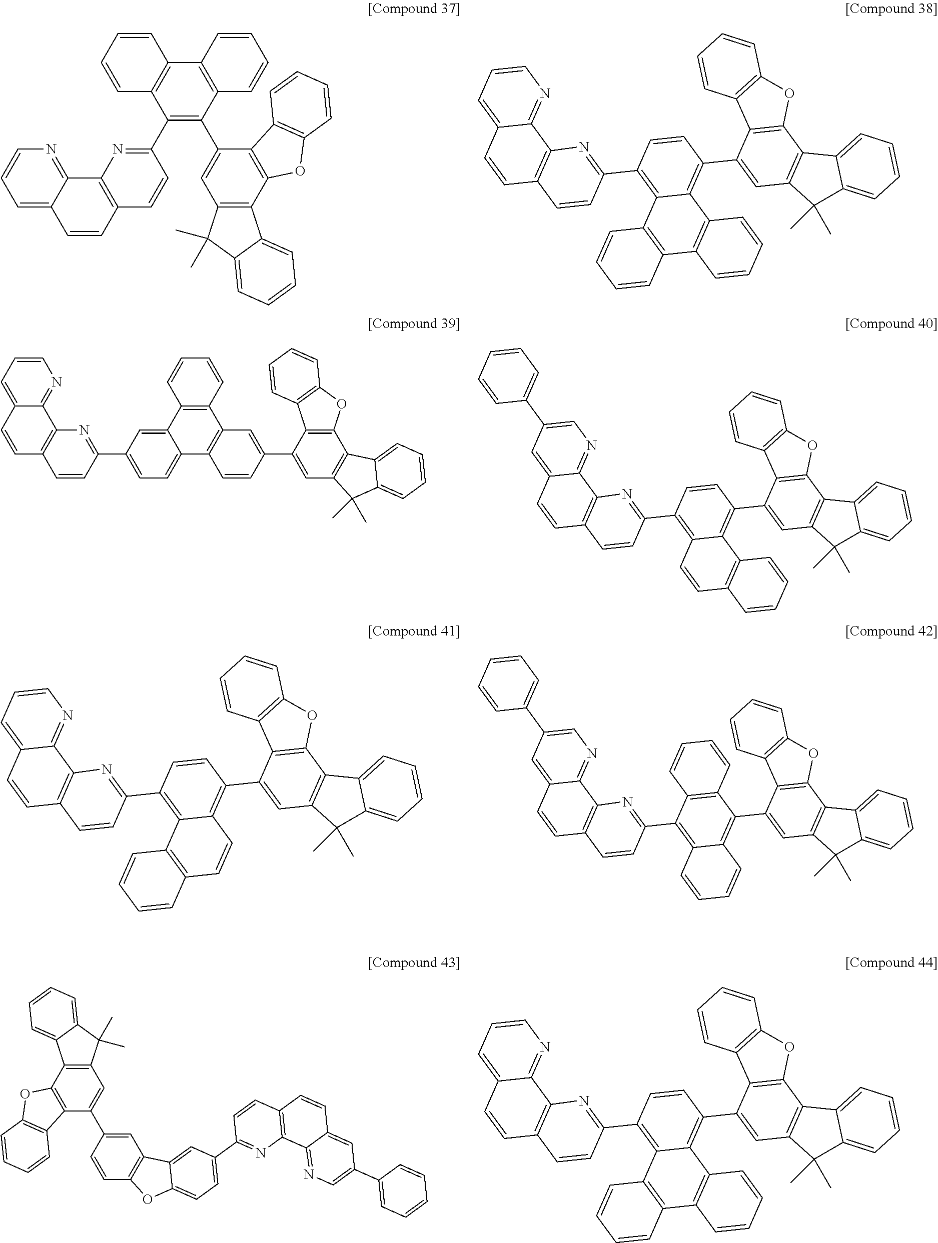

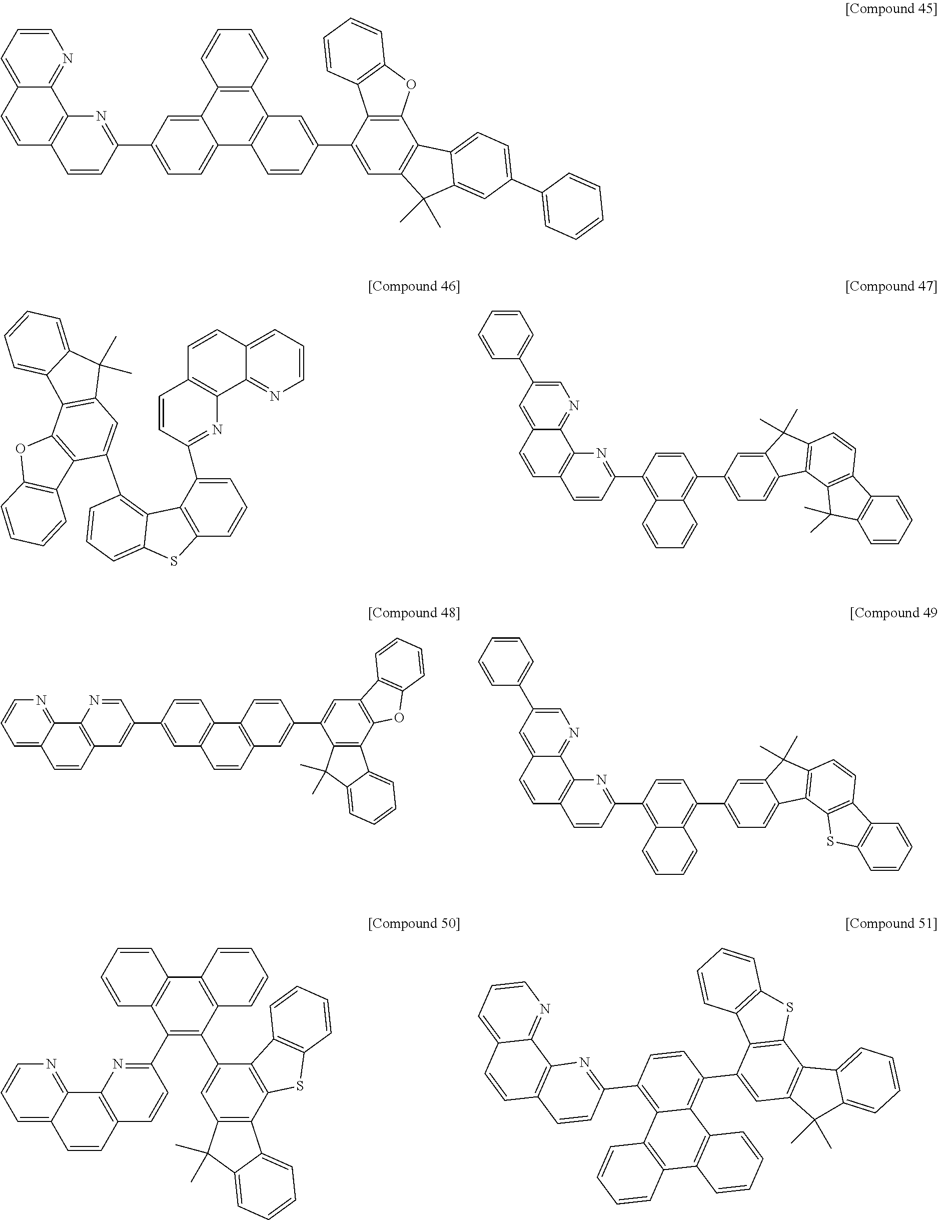

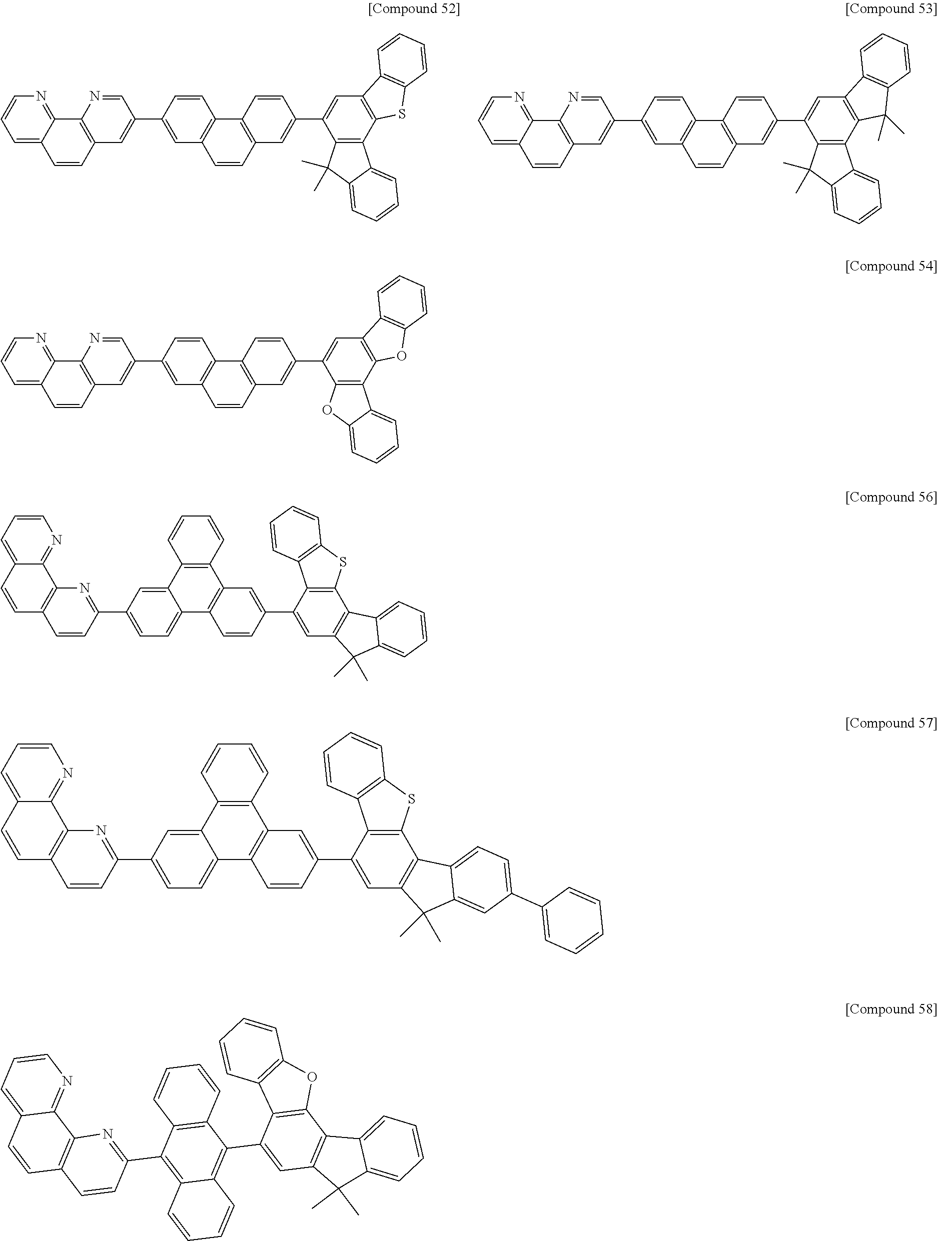

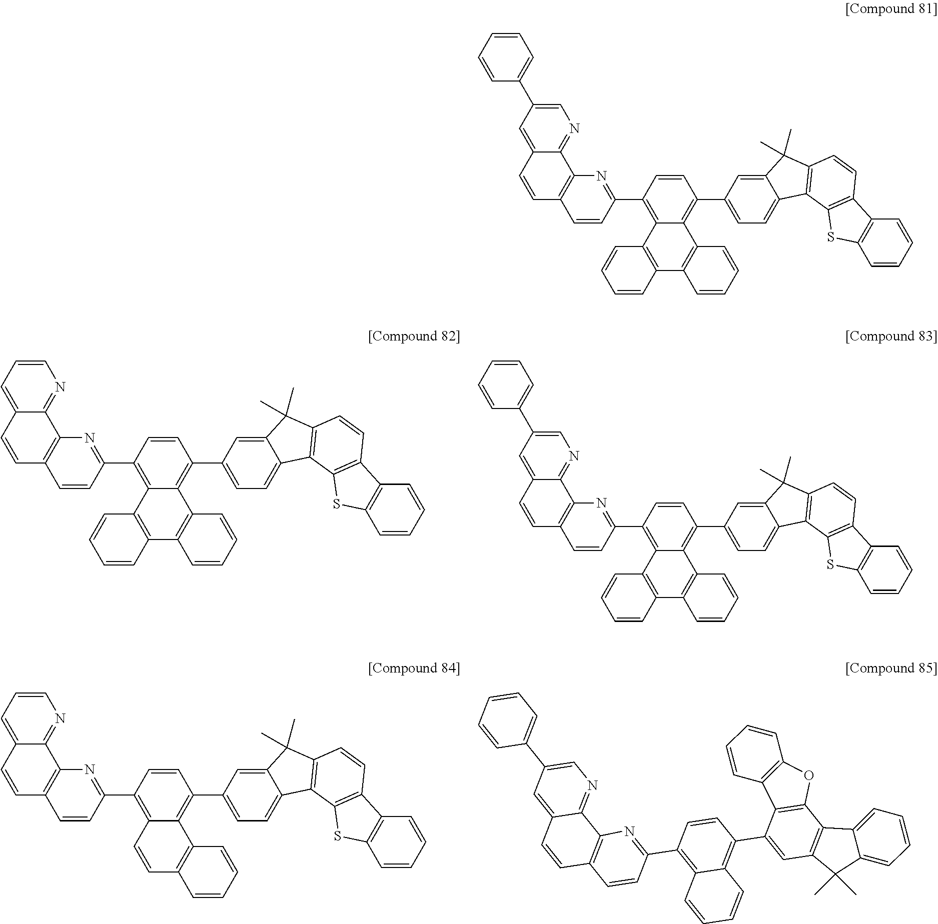 [SMILES: CC1(C)c2cc(-c3ccc(-c4ccc5ccc6cc(-c7ccccc7)cnc6c5n4)c4c5ccccc5c5ccccc5c34)ccc2-c2c1ccc1c2sc2ccccc21 ; CC1(C)c2cc(-c3ccc(-c4ccc5ccc6cccnc6c5n4)c4c5ccccc5c5ccccc5c34)ccc2-c2c1ccc1c2sc2ccccc21 ; CC1(C)c2cc(-c3ccc(-c4ccc5ccc6cc(-c7ccccc7)cnc6c5n4)c4c5ccccc5c5ccccc5c34)ccc2-c2c1ccc1c2sc2ccccc21 ; CC1(C)c2cc(-c3ccc(-c4ccc5ccc6cccnc6c5n4)c4ccc5ccccc5c34)ccc2-c2c1ccc1c2sc2ccccc21 ; CC1(C)c2ccccc2-c2c1cc(-c1ccc(-c3ccc4ccc5cc(-c6ccccc6)cnc5c4n3)c3ccccc13)c1c2oc2ccccc21]

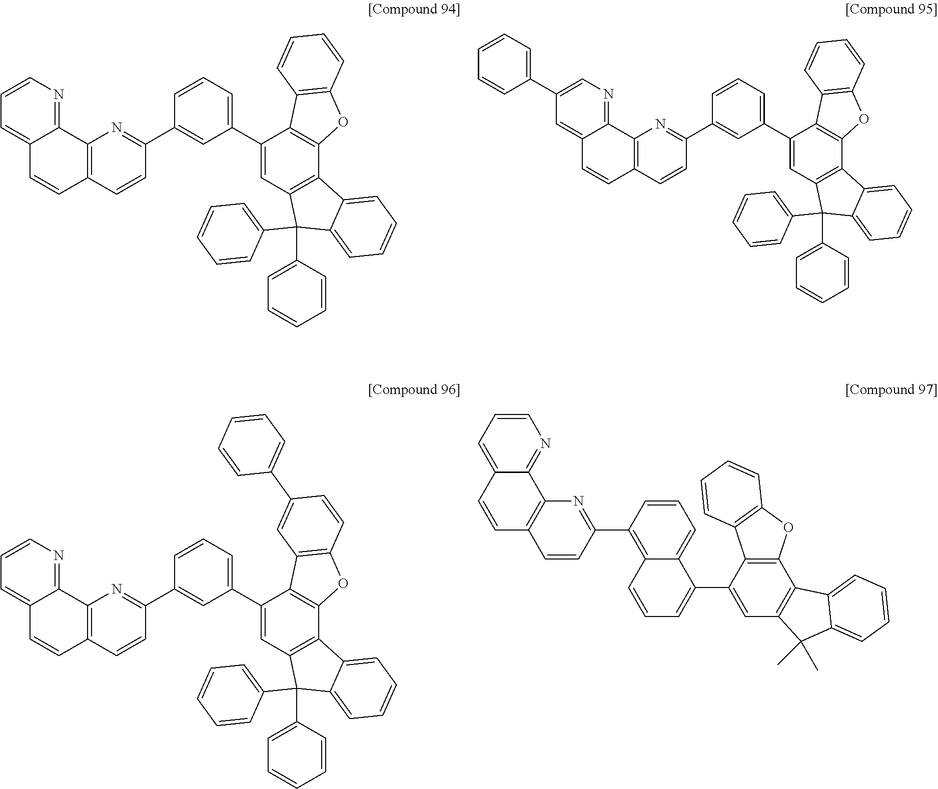 [SMILES: c1ccc(C2(c3ccccc3)c3ccccc3-c3c2cc(-c2cccc(-c4ccc5ccc6cccnc6c5n4)c2)c2c3oc3ccccc32)cc1 ; c1ccc(-c2cnc3c(ccc4ccc(-c5cccc(-c6cc7c(c8oc9ccccc9c68)-c6ccccc6C7(c6ccccc6)c6ccccc6)c5)nc43)c2)cc1 ; c1ccc(-c2ccc3oc4c5c(cc(-c6cccc(-c7ccc8ccc9cccnc9c8n7)c6)c4c3c2)C(c2ccccc2)(c2ccccc2)c2ccccc2-5)cc1 ; CC1(C)c2ccccc2-c2c1cc(-c1cccc3c(-c4ccc5ccc6cccnc6c5n4)cccc13)c1c2oc2ccccc21]

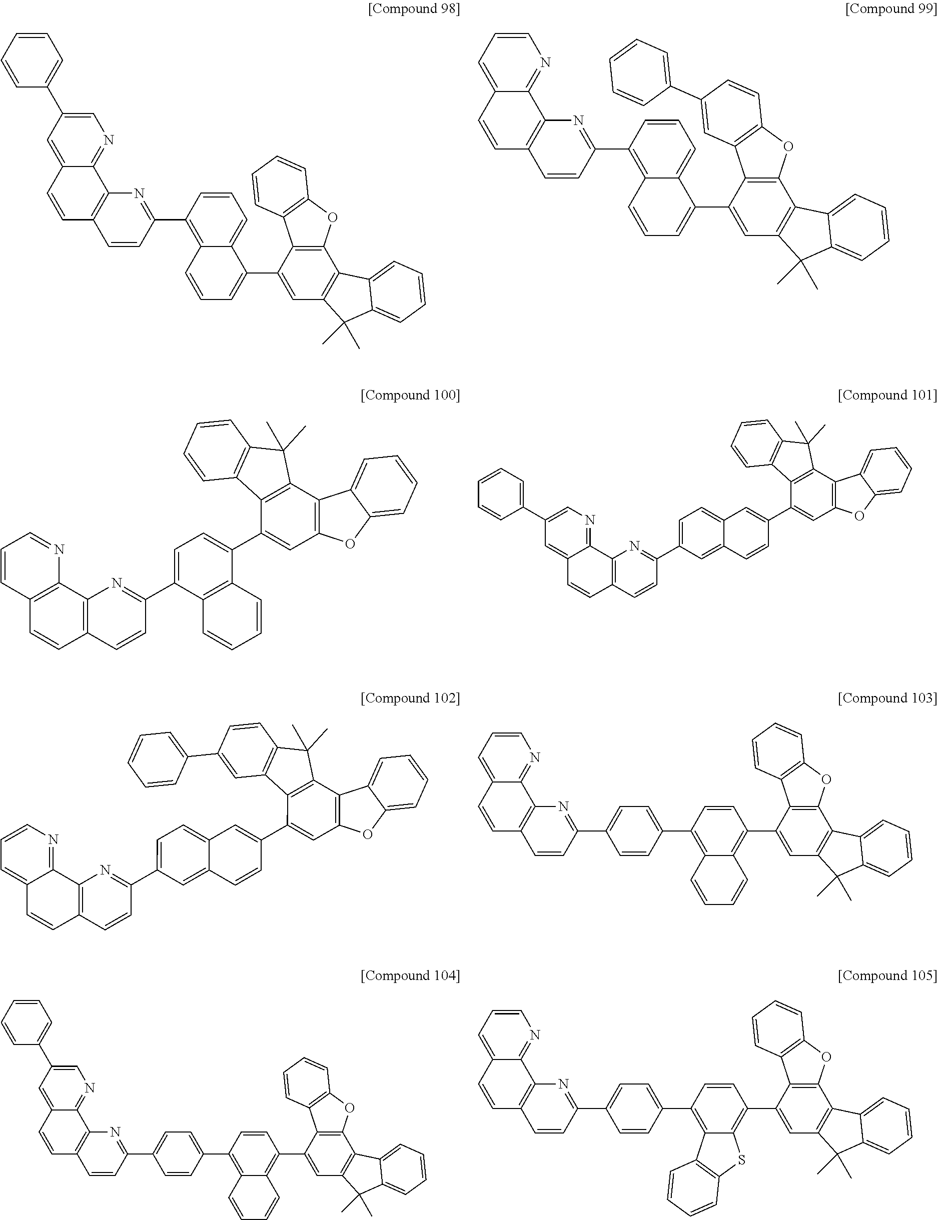

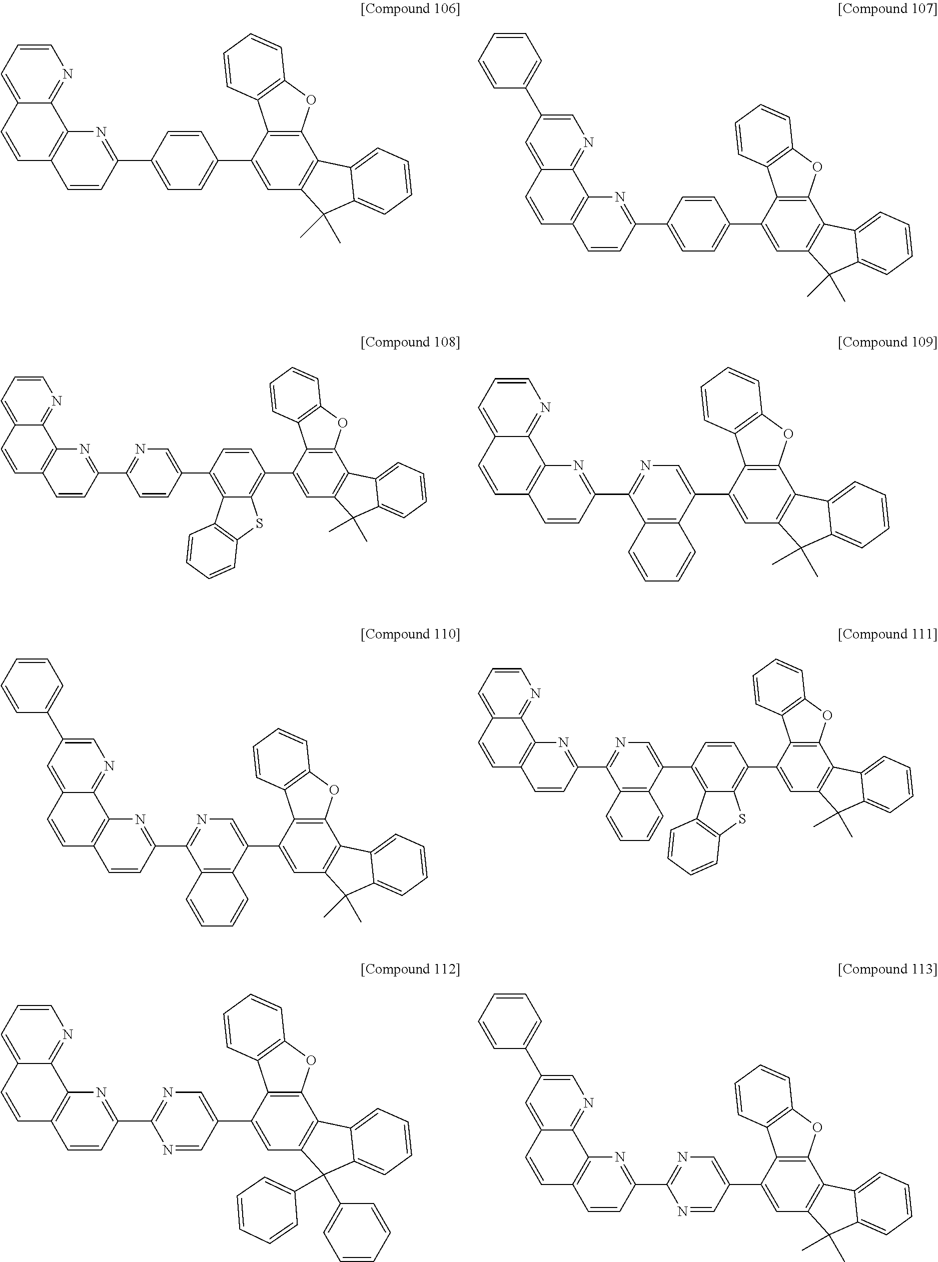

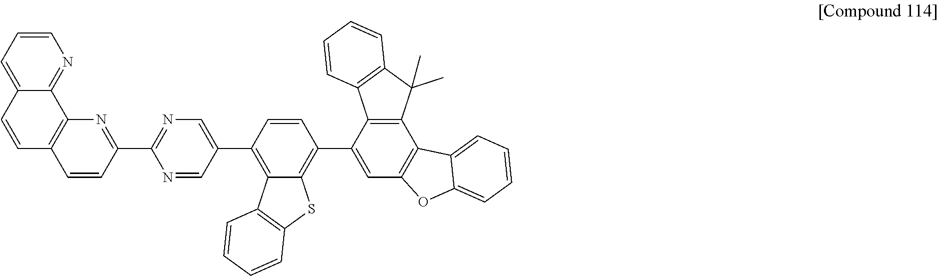 [SMILES: CC1(C)c2ccccc2-c2c(-c3ccc(-c4cnc(-c5ccc6ccc7cccnc7c6n5)nc4)c4c3sc3ccccc34)cc3oc4ccccc4c3c21]

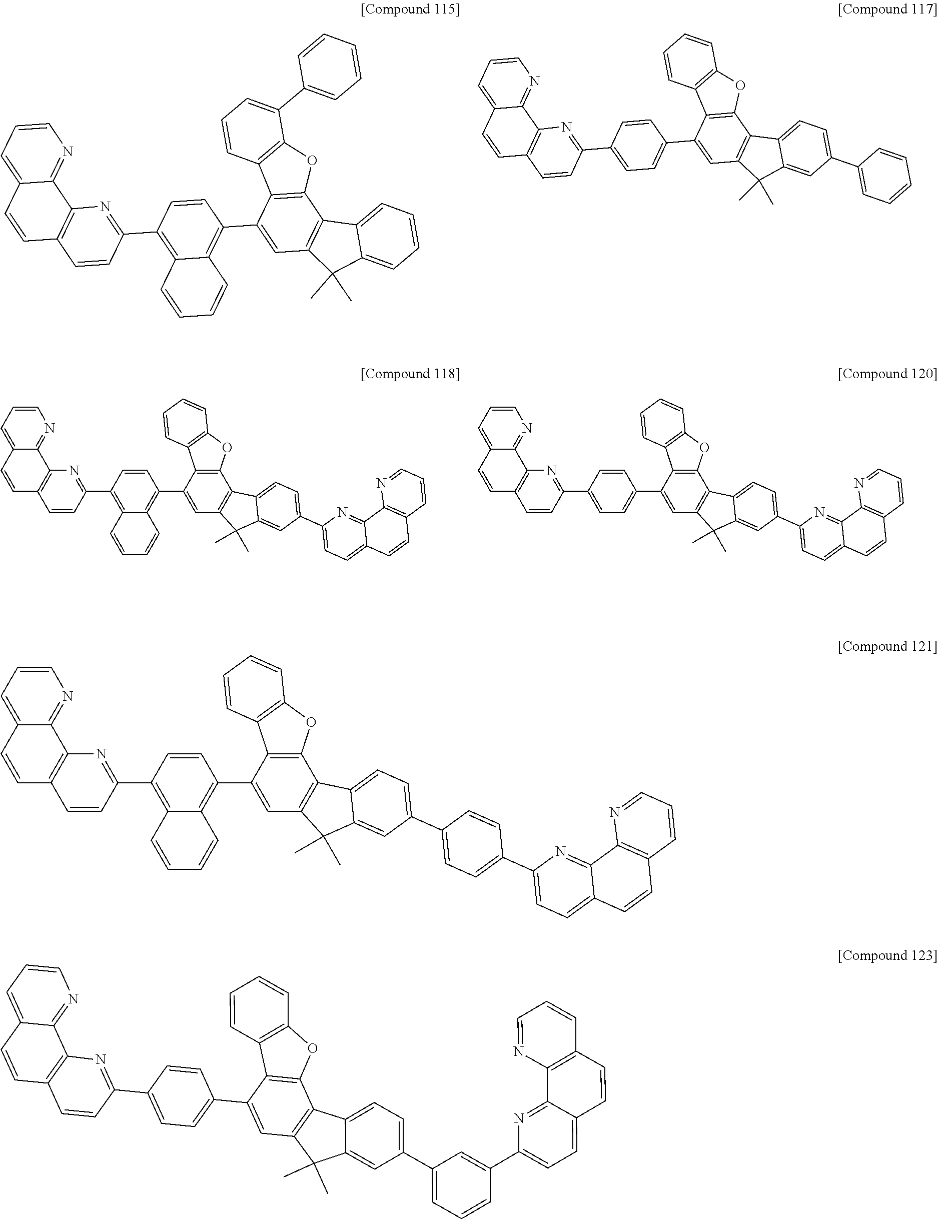

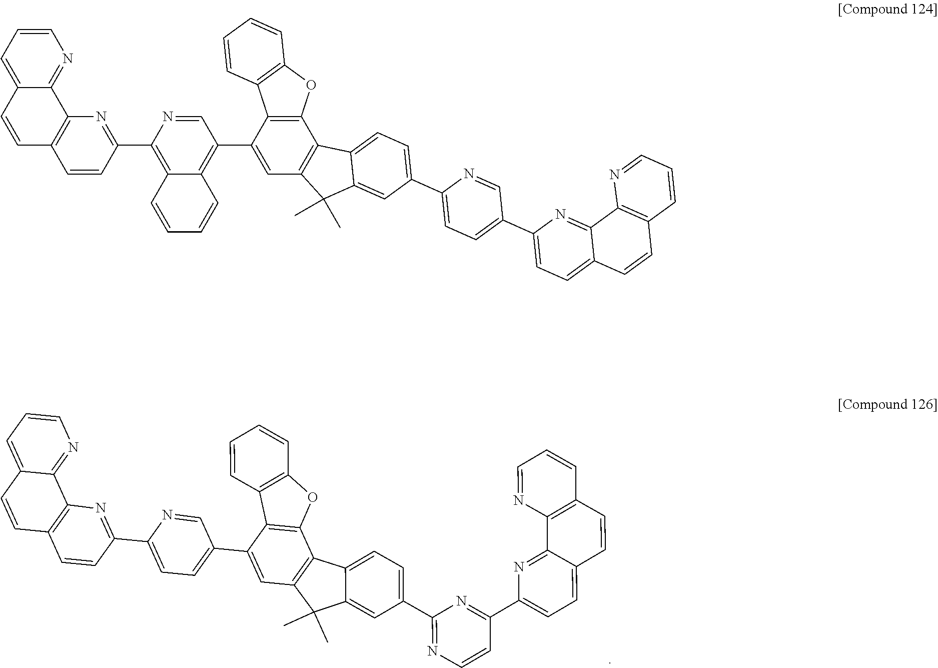 [SMILES: CC1(C)c2cc(-c3ccc(-c4ccc5ccc6cccnc6c5n4)cn3)ccc2-c2c1cc(-c1cnc(-c3ccc4ccc5cccnc5c4n3)c3ccccc13)c1c2oc2ccccc21 ; CC1(C)c2cc(-c3nccc(-c4ccc5ccc6cccnc6c5n4)n3)ccc2-c2c1cc(-c1ccc(-c3ccc4ccc5cccnc5c4n3)nc1)c1c2oc2ccccc21]

9. An organic light-emitting diode, comprising:
a first electrode;
a second electrode facing the first electrode; and
an organic layer interposed between the first electrode and the second electrode,
wherein the organic layer includes a first light-emitting layer, a charge generation layer, and a second light-emitting layer, and
the charge generation layer comprises at least one phenanthroline-based compound represented by Chemical Formula A: 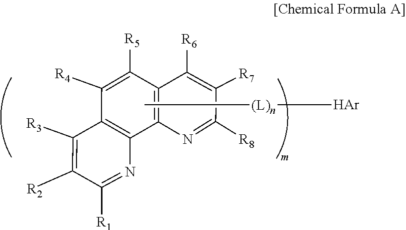
wherein,
L functions as a linker and is at least one selected from a single bond, a substituted or unsubstituted arylene of 6 to 18 carbon atoms, and a substituted or unsubstituted heteroarylene of 2 to 18 carbon atoms,
R₁ to R₈, which are same or different from each other, are each independently at least one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 6 to 30 carbon atoms, a cyano, and a halogen,
wherein any one of R₁ to R₈ is a single bond connected to the linker L,
n is an integer of 1 to 3 wherein when n is 2 or greater, the L's are same or different,
m is an integer of 1 or 2,
wherein when m is 2, the corresponding 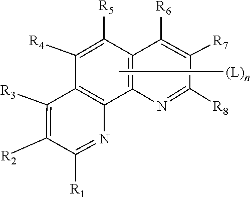
moieties are same or different,
HAr is substituent represented by the following Structural Formula 1: 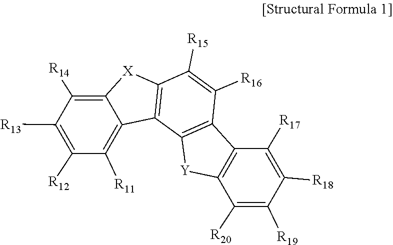
wherein,
R₁₁ to R₂₀, which are same or different, are each independently any one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 6 to 30 carbon atoms, a cyano, and a halogen,
wherein when m is 1, one of R₁₁ to R₂₀ is a single bond connected to the linker L,
wherein when m is 2, two of R₁₁ to R₂₀ are each a single bond connected to the linker L,
X and Y, which are same or different, are each independently at least one selected from CR₂₁R₂₂, NR₂₃, O and S,
wherein R₂₁ to R₂₃, which are same or different, are each independently at least one selected from a hydrogen atom, a deuterium atom, a substituted or unsubstituted alkyl of 1 to 30 carbon atoms, a substituted or unsubstituted cycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 5 to 30 carbon atoms, a cyano, and a halogen,
R₂₁ and R₂₂ may be linked to each other to form a mono- or polycyclic aliphatic or aromatic ring,
wherein the term “substituted” in the expression “substituted or unsubstituted” used for the compound of Chemical Formula A means having at least one substituent selected from the group consisting of a deuterium atom, a cyano, a halogen, an alkyl of 1 to 24 carbon atoms, a halogenated alkyl of 1 to 24 carbon atoms, cycloalkyl of 3 to 30 carbon atoms, an aryl of 6 to 24 carbon atoms, an arylalkyl of 7 to 24 carbon atoms, an alkylaryl of 7 to 24 carbon atoms, a heteroaryl of 2 to 50 carbon atoms, an alkylsilyl of 1 to 24 carbon atoms, and an arylsilyl of 6 to 24 carbon atoms.

10. The organic light-emitting diode of claim 9, wherein the organic layer further includes, between the second light-emitting layer and the second electrode, a second charge generation layer and a third light-emitting layer,
the second charge generation layer includes a P-type charge generation layer and an N-type charge generation layer, and
the phenanthroline-based compound represented by Chemical Formula A is used as a material for the N-type charge generation layer in the second charge generation layer.

11. An organic light-emitting diode, comprising:
a first electrode;
a second electrode facing the first electrode; and
an organic layer interposed between the first electrode and the second electrode,
wherein the organic layer includes a first light-emitting layer, a first charge generation layer, a second light-emitting layer, a second charge generation layer, and a third light-emitting layer, and
at least one of the first charge generation layer and the second charge generation layer comprises at least one phenanthroline-based compound represented by Chemical Formula A: 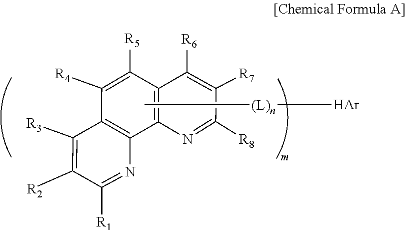
wherein,
L functions as a linker and is at least one selected from a single bond, a substituted or unsubstituted arylene of 6 to 18 carbon atoms, and a substituted or unsubstituted heteroarylene of 2 to 18 carbon atoms,
R₁ to R₈, which are same or different from each other, are each independently at least one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 6 to 30 carbon atoms, a cyano, and a halogen,
wherein any one of R₁ to R₈ is a single bond connected to the linker L,
n is an integer of 1 to 3 wherein when n is 2 or greater, the L's are same or different,
m is an integer of 1 or 2,
wherein when m is 2, the corresponding 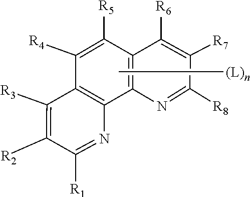
moieties are same or different,
HAr is substituent represented by the following Structural Formula 1: 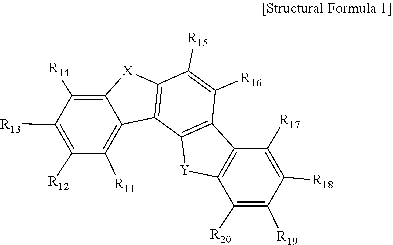
wherein,
R₁₁ to R₂₀, which are same or different, are each independently any one selected from a hydrogen atom, a deuterium atom, a substituted or unsubstituted alkyl of 1 to 30 carbon atoms, a substituted or unsubstituted aryl of 6 to 50 carbon atoms, a substituted or unsubstituted cycloalkyl of 3 to 30 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 6 to 30 carbon atoms, a cyano, and a halogen,
wherein when m is 1, one of R₁₁ to R₂₀ is a single bond connected to the linker L,
wherein when m is 2, two of R₁₁ to R₂₀ are each a single bond connected to the linker L,
X and Y, which are same or different, are each independently at least one selected from CR₂₁R₂₂, NR₂₃, O and S,
wherein R₂₁ to R₂₃, which are same or different, are each independently at least one selected from a hydrogen atom, a deuterium atom, a substituted or unsubstituted alkyl of 1 to 30 carbon atoms, a substituted or unsubstituted cycloalkyl of 3 to 30 carbon atoms, a substituted or unsubstituted aryl of 6 to 50 carbon atoms, a substituted or unsubstituted heteroaryl of 2 to 50 carbon atoms, a substituted or unsubstituted alkylsilyl of 1 to 30 carbon atoms, a substituted or unsubstituted arylsilyl of 5 to 30 carbon atoms, a cyano, and a halogen,
R₂₁ and R₂₂ may be linked to each other to form a mono- or polycyclic aliphatic or aromatic ring,
wherein the term “substituted” in the expression “substituted or unsubstituted” used for the compound of Chemical Formula A means having at least one substituent selected from the group consisting of a deuterium atom, a cyano, a halogen, an alkyl of 1 to 24 carbon atoms, a halogenated alkyl of 1 to 24 carbon atoms, cycloalkyl of 3 to 30 carbon atoms, an aryl of 6 to 24 carbon atoms, an arylalkyl of 7 to 24 carbon atoms, an alkylaryl of 7 to 24 carbon atoms, a heteroaryl of 2 to 50 carbon atoms, an alkylsilyl of 1 to 24 carbon atoms, and an arylsilyl of 6 to 24 carbon atoms.

12. The organic light-emitting diode of claim 1, wherein the light-emitting layer contains a host and a dopant.

13. The organic light-emitting diode of claim 9, wherein the first light-emitting layer and the second light-emitting layer contain same or different hosts and same or different dopants.

14. The organic light-emitting diode of claim 1, wherein the organic layer comprises at least one of a hole injection layer, a hole transport layer, a functional layer capable of both hole injection and hole transport, an electron blocking layer, a light-emitting layer, an electron transport layer, an electron injection layer, and a capping layer.

15. The organic light-emitting diode of claim 14, wherein at least one selected from among the layers is deposited using a single-molecule deposition process or a solution process.

16. The organic light-emitting diode of claim 1, wherein the organic light-emitting diode is used for a device selected from among a flat display device; a flexible display device; a monochrome or grayscale flat illumination; and a monochrome or grayscale flexible illumination device.
